(19) United States
(12) Patent Application Publication
(10) Pub. No.: US 2003/0211475 A1
(43) Pub. Date: Nov. 13, 2003

(54) Methods for identifying pathway-specific reporters and target genes, and uses thereof

(75) Inventor: Christopher J. Roberts, Seattle, WA (US)

Correspondence Address: PENNIE AND EDMONDS, 1155 AVENUE OF THE AMERICAS, NEW YORK, NY, 100362711

(73) Assignee: Rosetta Inpharmatics, Inc.

(21) Appl. No.: 09/946,290

(22) Filed: Sep. 5, 2001

Related U.S. Application Data
(63) Continuation of application No. 09/282,243, filed on Mar. 31, 1999, now abandoned.

(51) Int. Cl. C12Q 1/68; G06F 19/00; G01N 33/48; G01N 33/50

(52) U.S. Cl. 435/6; 702/20

(57) ABSTRACT

The present invention relates to methods for identifying one or more reporter genes for a particular biological pathway of interest. The reporter genes of this invention are particularly useful for analyzing the activity of particular biological pathways of interest, and may be further used in the design of drugs, drug therapies or other biological agents (e.g., insecticides, herbicides, fungicides, antibiotics, or antivirals) to target a particular biological pathway. The present invention also relates to methods for identifying one or more target genes for a particular biological pathway of interest. Target genes of the invention are useful as specific targets for drugs which may be designed to enhance, inhibit, or modulate a particular biological pathway. Methods to identify genes which modify the function or structure of a member (e.g., compound or gene product) of a particular biological pathway are provided.

### 1. INTRODUCTION

The present invention relates to methods for identifying one or more reporter genes for a particular biological pathway of interest. The reporter genes of this invention are particularly useful for analyzing the activity of particular biological pathways of interest, and may be further used in the design of drugs, drug therapies or other biological agents (e.g., insecticides, herbicides, fungicides, antibiotics, or antivirals) to target a particular biological pathway. The present invention also relates to methods for identifying one or more target genes for a particular biological pathway of interest. Target genes of the invention are useful as specific targets for drug which may be designed to enhance, inhibit, or modulate a particular biological pathway. Methods to identify gene which modifies the function or structure of a member (e.g., compound or gene product) of a particular biological pathway are provided.

The present invention provides examples of reporter genes and/or target genes which have been discovered by the methods of the invention. Specifically, the inventors have made the surprising discovery that five S. cerevisiae genes (previously of unknown function) form clustered co-regulated sets of genes and are reporters of the ergosterol-pathway. The methods of the invention are also exemplified in that the inventors have specifically discovered six S. cerevisiae reporter genes of the protein kinase C (PKC) pathway. Two of these genes are also novel target genes of the PKC pathway and provide targets for the development of PKC pathway-specific drugs, drug therapies, or other related biological or therapeutical agents. The methods of the invention are further exemplified by the discovery of four novel reporter genes of the S. cerevisiae Invasive Growth pathway. One of these genes also serves as a target gene in the Invasive Growth pathway, and may be used to develop Invasive Growth pathway-specific drugs, drug therapies, or other related biological or therapeutical agents.

### 2. BACKGROUND OF THE INVENTION

Citation of a reference herein shall not be construed as an admission that such reference is prior art to the present invention.

### 2.1. Microarray Technology

Within the past decade, several technologies have made it possible to monitor the expression level of a large number of transcripts at any one time (see, e.g., Schena et al., 1995, Quantitative monitoring of gene expression patterns with a complementary DNA micro-array, Science 270:467-470; Lockhart et al., 1996, Expression monitoring by hybridization to high-density oligonucleotide arrays, Nature Biotechnology 14:1675-1680; Blanchard et al., 1996, Sequence to array: Probing the genome's secrets, Nature Biotechnology 14, 1649; U.S. Pat. No. 5,569,588, issued Oct. 29, 1996 to Ashby et al. entitled “Methods for Drug Screening”). In organisms for which the complete genome is known, it is possible to analyze the transcripts of all genes within the cell. With other organisms, such as human, for which there is an increasing knowledge of the genome, it is possible to simultaneously monitor large numbers of the genes within the cell.

Such monitoring technologies have been applied to the identification of genes which are up regulated or down regulated in various diseased or physiological states, the analyses of members of signaling cellular states, and the identification of targets for various drugs. See, e.g., Friend and Hartwell, International Publication WO98/38329 dated Sep. 3, 1993; Stoughton and Friend, U.S. patent application Ser. No. 09/074,983, filed on filed on May 8, 1998; Friend and Hartwell, U.S. Provisional Application Serial No. 60/056,109, filed on Aug. 20, 1997; Friend and Stoughton, U.S. Provisional Application Serial Nos. 60/084,742 (filed on May 8, 1998), 60/090,004 (filed on Jun. 19, 1998) and 60/090,046 (filed on Jun. 19, 1998), all incorporated herein by reference for all purposes.

Levels of various constituents of a cell are known to change in response to drug treatments and other perturbations of the cell's biological state. Measurements of a plurality of such “cellular constituents” therefore contain a wealth of information about the effect of perturbations and their effect on the cell's biological state. Such measurements typically comprise measurements of gene expression levels of the type discussed above, but may also include levels of other cellular components such as, but by no means limited to, levels of protein abundances, or protein activity levels. The collection of such measurements is generally referred to as the “profile” of the cell's biological state.

The number of cellular constituents is typically on the order of a hundred thousand for mammalian cells. The profile of a particular cell is therefore typically of high complexity. Any one perturbing agent may cause a small or a large number of cellular constituents to change their abundances or activity levels. Thus, identifying the particular cellular constituents are associated with a particular biological pathway, provides a difficult and challenging task. Additionally, methods in the art do not provide a means by which all of the cellular constituents which are associated with a particular pathway of interest may be identified. Therefore, there is a need in the art for methods to identify groups of cellular constituents, which are associated with a particular biological pathway.

2.1.1. The Need for Reporter Genes

In order to monitor and study a particular biological pathway, it is necessary to have a “read-out” or reporter of the pathway that allows measurement of an alteration of the pathway. Many biological pathways, however, do not have reliable reporters associated with them. There is a need in the art for a method to identify reporters for a particular biological pathway of interest. Additionally, there is a need in the art for novel reporter genes which may be assigned to a particular biological pathway. The present invention provides such a reporters and methods of identifying such reporters.

2.1.2. Identification of Targets

Identification of targets for drug development is a laborious process that has had a low rate of success. Accordingly, there is a need in the art for novel targets for the development of novel drugs and therapies against biologic pathogens of interest. There is also a need in the art for novel targets for the development of novel drugs and therapies which can enhance, inhibit, or modulate a particular biological pathway of interest. Additionally, there is a need in the art for a method of screening potential drug targets that affords high throughput and the ability to assess multiple targets simultaneously. The present invention provides such a targets and methods to identify such targets.

### 2.2. Fungi and Disease

Fungi are eukaryotic microorganisms comprising a phylogenetic kingdom. The Kingdom Fungi is estimated to contain over 100,000 species and includes species of “yeast”, which is the common term for several families of unicellular fungi.

Although fungal infections were once unrecognized as a significant cause of disease, the extensive spread of fungal infections is a major concern in hospitals, health departments and research laboratories. According to a 1988 study nearly 40% of all deaths from hospital-acquired infections were caused by fungi, not bacteria or viruses (Sternberg, S., 1994, Science 266:1632-34).

Immunocompromised patients are particularly at risk of fungal infections. Patients with impaired immune systems due to AIDS, cancer chemotherapy, or those treated with immunosuppressive drugs used to prevent rejection in organ transplant are common hosts for fungal infections. Organisms including Cryptococcus, Candida, Histoplasma, Coccidioides, and many as 150 species of fungi have been linked to human or animal diseases (Sternberg, S., 1994, Science 266:1632-34). Under immunocompromised conditions, fungi that are normally harmless to the host when maintained in the gastrointestinal system, can be transferred to the bloodstream, eyes, brain, heart, kidneys, and other tissues leading to symptoms ranging in severity from white patches on the tongue, to fever, rupturing of the retina, blindness, pneumonia, heart failure, shock, or sudden catastrophic clotting of the blood (Sternberg, S., 1994, Science 266:1632-34). In susceptible burn victims, even baker's yeast, common in the human mouth and normally non-virulent, can lead to severe infection (Sternberg, S., 1994, Science 266:1632-34). Hospital transmission may also occur via catheters or other invasive equipment (Sternberg, S., 1994, Science 266:1632-34).

Fungal infections are not limited to individuals with compromised immune systems. Geological and meteorological events have been reported to trigger fungal outbreaks. Following a 1994 earthquake in California, tremors were estimated to have released infectious fungal spores from the soil triggering a 3-year statewide epidemic that lead to more than 4500 cases per year (Sternberg, S., 1994, Science 266:1632-34). Similarly, environmental cycles of droughts and heavy rains are believed to be associated with release infectious spores leading to epidemic infections (Sternberg, S., 1994, Science 266:1632-34).

The widespread dissipation of fungal infection coupled to the recognition of fungi as a significant disease factor creates an increasing need for antifungal agents. Existing antifungal therapies harbor many disadvantages as discussed in Section 2.1.2, and novel therapies and targets for therapy are needed.

2.2.1. Antifungal Agents and Need for Improvements

A useful antifungal agent must be toxic to the parasite, but not to the host. One way to achieve this goal is to target a structure or pathway that is unique to the pathogen. For example, successful antibacterial therapies often take advantage of the differences between the prokaryotic bacteria and the eukaryotic host. However, since fungal pathogens, like human cells, are eukaryotic, it has been more difficult to identify therapeutic agents that are unique to the pathogen. Among the targets exploited to date are the biochemical pathways for (1) membrane integrity; (2) ergosterol synthesis (reviewed in Handbook of Experimental Pharmacology, 1990, Springer-Verlag, Heidelberg, J F Ryley, eds.); (3) nucleic acid synthesis; and (4)cell wall synthesis.

However, antifungal agents and drugs currently used to treat fungal pathogens are lacking in both efficacy and safety. To date, only a limited number of therapeutic agents are available for the treatment of fungal infections. These drugs, however, often prove to be toxic to the host, or are accompanied by severe side effects. The commonly prescribed drug, Amphotericin B, a mainstay of antifungal therapy, includes such side effects as fever, chills, low blood pressure, headache, nausea, vomiting, inflammation of blood vessels and kidney damage (Sternberg, S., 1994, Science 266:1632-34). Further, many of the existing therapies act to inhibit or slow fungal growth, but do not kill the infecting fungal.

### 3. SUMMARY OF THE INVENTION

The present invention relates to methods for identifying one or more reporter genes for a particular biological pathway of interest. The reporter genes of this invention are particularly useful for analyzing the activity of particular biological pathways of interest, and may be further used in the design of drugs, drug therapies or other biological agents (e.g., insecticides, herbicides, fungicides, antibiotics, or antivirals) to target a particular biological pathway. The present invention also relates to methods for identifying one or more target genes for a particular biological pathway of interest. Target genes of the invention are useful as specific targets for drug which may be designed to enhance, inhibit, or modulate a particular biological pathway. Methods to identify gene which modifies the function or structure of a member (e.g., compound or gene product) of a particular biological pathway are provided.

The present invention provides examples of reporter genes and/or target genes which have been discovered by the methods of the invention. Specifically, the inventors have made the surprising discovery that five S. cerevisiae genes (previously of unknown function) form clustered co-regulated sets of genes and are reporters of the ergosterol-pathway. The methods of the invention are also exemplified in that the inventors have specifically discovered six S. cerevisiae reporter genes of the protein kinase C (PKC) pathway. Two of these genes are also novel target genes of the PKC pathway and provide targets for the development of PKC pathway-specific drugs, drug therapies, or other related biological or therapeutical agents. The methods of the invention are further exemplified by the discovery of four novel reporter genes of the S. cerevisiae Invasive Growth pathway. One of these genes also serves as a target gene in the Invasive Growth pathway, and may be used to develop Invasive Growth pathway-specific drugs, drug therapies, or other related biological or therapeutical agents.

The invention provides a method of identifying a reporter gene for a particular biological pathway in a cell comprising identifying a gene which clusters to a geneset associated with the biological pathway, wherein said gene which clusters to the geneset associated with the particular biological pathway is a reporter gene.

In one embodiment the geneset associated with the particular biological pathway is identified by a method comprising identifying one or more genes in a geneset which are associated with the particular biological pathway, wherein said geneset having one or more genes associated with the particular biological pathway is a geneset associated with the particular biological pathway.

In another embodiment the geneset associated with the particular biological pathway is identified by identifying a geneset which is activated or inhibited by perturbations which target the biological pathway, wherein a geneset which is activated or inhibited by perturbations which target the biological pathway is a geneset associated with the particular biological pathway.

In one embodiment the method further comprises identifying a gene which clusters specifically to a geneset associated with the particular biological pathway, wherein said gene which clusters specifically to the geneset associated with the particular biological pathway is a reporter gene.

In one embodiment the reporter gene is further identified as a gene whose expression is not altered by perturbations which effect other biological pathways, said other biological pathways being different from said particular biological pathway.

In another embodiment the geneset is provided by a method comprising: (a) measuring changes in expression of a plurality of genes in the cell in response to a plurality of perturbations to the cell; and (b) grouping or re-ordering said plurality of genes into one or more co-varying sets, wherein said one or more co-varying sets comprise said geneset. In a further embodiment said plurality of genes are grouped or re-ordered into one or more co-varying sets by means of a pattern recognition algorithm. In another embodiment the pattern recognition algorithm is a clustering algorithm. In a further embodiment the clustering algorithm analyzes arrays or matrices, said arrays or matrices representing said measured changes in expression of the plurality of genes in the cell in response to the plurality of perturbations to the cell, wherein said analysis determines dissimilarities between individual genes.

In one embodiment the plurality of perturbations to the cell are also grouped or re-ordered according to their similarity. In another embodiment said plurality of perturbations to the cell are grouped or re-oredered by means of a pattern recognition algorithm. In a further embodiment the pattern recognition algorithm is a clustering algorithm.

In one embodiment of the invention, the clustering algorithm analyzes arrays or matrices, said arrays or matrices representing said measured changes in expression of the plurality of genes in the cell in response to the plurality of perturbations to the cell. In another embodiment the reporter gene is further identified as has a high level of induction. In another embodiment the expression of the reporter gene is further identified to change by at least a factor of two in response to perturbations of the particular biological pathway.

In a further embodiment expression of the reporter gene is further identified to change by at least a factor of 10 in response to perturbations to the particular biological pathway. In another embodiment the expression of the reporter gene is further identified to change by at least a factor of 100 in response to perturbations to the particular biological pathway.

In one embodiment the expression of the reporter gene is further identified to change in response to slight perturbations to the particular biological pathway.

In another embodiment the perturbation to the particular biological pathway comprises exposure to a drug, and said reporter gene is further identified to change in response to low levels of exposure to the drug.

In one embodiment the reporter gene is further identified to respond to perturbations targeted to the entire particular biological pathway. In one embodiment the reporter gene is further identified to respond to perturbations directed to one or more portions of the particular biological pathway. In another embodiment the reporter gene is further identified to respond to perturbations targeted to early steps of the particular biological pathway. In another embodiment the reporter gene is further identified to respond to perturbations targeted to late steps of the particular biological pathway. In yet another embodiment the reporter gene is further identified by identifying a gene which kinetically induces quickly in response to perturbations to the particular biological pathway.

In another embodiment the reporter gene is further identified by identifying a gene which reaches steady state within about eight hours after a perturbation to the particular biological pathway. In a further embodiment the reporter gene is further identified by identifying a gene which reaches steady state within about six hours after a perturbation to the particular biological pathway. In another embodiment the reporter gene is further identified by identifying a gene which is induced within about two hours after a perturbation to the particular biological pathway.

In still another embodiment the reporter gene is further identified by identifying a gene which is induced within about 90 minutes after a perturbation to the particular biological pathway. In another embodiment the reporter gene is further identified by identifying a gene which is induced within about 60 minutes after a perturbation to the particular biological pathway. In a further embodiment the reporter gene is further identified by identifying a gene which is induced within about 30 minutes after a perturbation to the particular biological pathway. In one embodiment the reporter gene is further identified by identifying a gene which is induced within about 10 minutes after a perturbation to the particular biological pathway. In another embodiment the reporter gene is further identified by identifying a gene which is induced within about 7 minutes after a perturbation to the particular biological pathway.

The invention provides a method of identifying a target gene for a particular biological pathway in a cell comprising identifying a gene which clusters to a geneset associated with the particular biological pathway, wherein said gene which clusters to a geneset associated with the particular biological pathway and is identified as a gene which is necessary for normal function of said particular biological pathway.

In one embodiment the geneset associated with the particular biological pathway is identified by a method comprising identifying one or more genes in a geneset which are associated with the particular biological pathway, wherein said geneset having one or more genes associated with the particular biological pathway is a geneset associated with the particular biological pathway. In another embodiment the geneset associated with the particular biological pathway is identified by identifying a geneset which is activated or inhibited by perturbations which target the biological pathway, wherein a geneset which is activated or inhibited by perturbations which target the biological pathway is a geneset associated with the particular biological pathway.

In one embodiment the genesets are provided by a method comprising: (a) measuring changes in expression of a plurality of genes in the cell in response to a plurality of perturbations to the cell; and (b) grouping or re-ordering said plurality of genes into one or more co-varying sets, wherein said one or more co-varying sets comprise said genesets.

In one embodiment said plurality of genes are grouped or re-ordered into one or more co-varying sets by means of a pattern recognition algorithm. In another embodiment the pattern recognition algorithm is a clustering algorithm.

In one embodiment the clustering algorithm analyzes arrays of matrices, said arrays or matrices representing said measured changes in expression of the plurality of genes in the cell in response to the plurality of perturbations to the cell, wherein said analysis determines dissimilarities between individual genes.

In one embodiment the plurality of perturbations to the cell are also grouped or re-ordered according to their similarity. In another embodiment the plurality of perturbations to the cell are grouped or re-ordered by means of a pattern recognition algorithm.

In one embodiment the pattern recognition algorithm is a clustering algorithm. In another embodiment the clustering algorithm analyzes arrays of matrices, said arrays or matrices representing said measured changes in expression of the plurality of genes in the cell in response to the plurality of perturbations to the cell.

In one embodiment the reporter gene is a reporter for the ergosterol-pathway, and the reporter gene is selected from the group consisting of: YHR039C (as depicted in FIG. 2, as set forth in SEQ ID NO:1), YLW100W (as depicted in FIG. 4, as set forth in SEQ ID NO:3), YPL272C (as depicted in FIG. 6, as set forth in SEQ ID NO:5), YGR131W (as depicted in FIG. 8, as set forth in SEQ ID NO:7), and YDR453C (as depicted in FIG. 10, as set forth in SEQ ID NO:9).

In another embodiment the reporter gene is a reporter for the PKC-pathway, and the reporter gene is selected from the group consisting of: SLT2(YHR030C) (as depicted in FIGS. 17A-B, as set forth in SEQ ID NO:11), YKR161C (as depicted in FIGS. 19A-B, as set forth in SEQ ID NO:13), PIR3(YKL163W) (as depicted in FIGS. 21A-B, as set forth in SEQ ID NO:15), YPK2(YMR104C) (as depicted in FIGS. 23A-B, as set forth in SEQ ID NO:17), YLR194C (as depicted in FIGS. 25A-B, as set forth in SEQ ID NO:19), and ST1(YDR055W) (as depicted in FIGS. 27A-B, as set forth in SEQ ID NO:21).

In another embodiment the reporter gene is a reporter for the Invasive Growth pathway, and the reporter gene selected from the group consisting of KSS1(YGR040W) (as depicted in FIG. 29, as set forth in SEQ ID NO:23), PGU1(YJR153W) (as depicted in FIG. 31, as set forth in SEQ ID NO:25), YRL042C (as depicted in FIG. 33, as set forth in SEQ ID NO:27), and SVS1(YPL163C) (as depicted in FIG. 35, as set forth in SEQ ID NO:29).

In another embodiment the biological pathway is selected from the group consisting of: a signaling pathway, a control pathway, a mating pathway, a cell cycle pathway, a cell division pathway, a cell repair pathway, a small molecule synthesis pathway, a protein synthesis pathway, a DNA synthesis pathway, a RNA synthesis pathway, a DNA repair pathway, a stress-response pathway, a cytoskeletal pathway, a steroid pathway, a receptor-mediated signal transduction pathway, a transcriptional pathway, a translational pathway, an immune response pathway, a heat-shock pathway, a motility pathway, a secretion pathway, an endocytotic pathway, a protein sorting pathway, a phagocytic pathway, a photosynthetic pathway, an excretion pathway, an electrical response pathway, a pressure-response pathway, a protein modification pathway, a small-molecule response pathway, a toxic-molecule response pathway, and a transformation pathway.

In one embodiment the target gene of the PKC-pathway is selected from the group consisting of: SLT2(YHR030C) (as depicted in FIGS. 17A-B, as set forth in SEQ ID NO:11), and YKR161C (as depicted in FIGS. 19A-B, as set forth in SEQ ID NO:13).

The invention provides a method for determining whether a molecule affects the function or activity of an ergosterol pathway in a cell comprising: (a) contacting the cell with, or recombinantly expressing within a cell the molecule; and (b) determining whether the expression of one or more of the genes selected from the group consisting of: YHR039C (as depicted in FIG. 2, as set forth in SEQ ID NO:1), YLW100W (as depicted in FIG. 4, as set forth in SEQ ID NO:3), YPL272C (as depicted in FIG. 6, as set forth in SEQ ID NO:5), YGR131W (as depicted in FIG. 8, as set forth in SEQ ID NO:7), and YDR453C (as depicted in FIG. 10, as set forth in SEQ ID NO:9) is changed relative to said expression in the absence of the molecule. In a further embodiment the method is a method for determining whether the molecule inhibits ergosterol synthesis such that a cell contacted with the molecule exhibits a lower level of ergosterol than a cell which is not contacted with said molecule. In another embodiment step (b) comprises determining whether YPL272c expression increases.

The invention provides a kit comprising in one or more containers a) a substance selected from the group consisting of an antibody against an ergosterol-pathway protein, a gene probe capable of hybridizing to RNA of an ergosterol-pathway gene, and pairs of gene primers capable of priming amplification of at least a portion of an ergosterol-pathway gene, and b) a molecule known to be capable of perturbing the ergosterol pathway.

The invention provides a method for identifying a molecule that activates the ergosterol pathway in yeast comprising contacting a yeast cell with one or more candidate molecules, and detecting a change in the RNA expression of a reporter gene for the ergosterol-pathway relative to the expression of the reporter gene in a yeast cell not contacted by the one or more candidate molecules, wherein the reporter gene is selected from the group consisting of: YHR039C (as depicted in FIG. 2, as set forth in SEQ ID NO:1), YLW100W (as depicted in FIG. 4, as set forth in SEQ ID NO:3), YPL272C (as depicted in FIG. 6, as set forth in SEQ ID NO:5), YGR131W (as depicted in FIG. 8, as set forth in SEQ ID NO:7), and YDR453C (as depicted in FIG. 10, as set forth in SEQ ID NO:9).

The invention provides a method for identifying a molecule that activates the ergosterol pathway in yeast comprising contacting a yeast cell with one or more candidate molecules, and detecting a change in the protein expression of a reporter gene for the ergosterol-pathway relative to the expression of the reporter gene in a yeast cell not contacted by the one or more candidate molecules, wherein the reporter gene is selected from the group consisting of: YHR039C (as depicted in FIG. 2, as set forth in SEQ ID NO:1), YLW100W (as depicted in FIG. 4, as set forth in SEQ ID NO:3), YPL272C (as depicted in FIG. 6, as set forth in SEQ ID NO:5), YGR131W (as depicted in FIG. 8, as set forth in SEQ ID NO:7), and YDR453C (as depicted in FIG. 10, as set forth in SEQ ID NO:9). In one embodiment the fungal cell is a transgenic cell.

The invention provides a method for identifying a molecule that modulates the expression of an ergosterol-pathway gene selected from the group consisting of YHR039C (as depicted in FIG. 2, as set forth in SEQ ID NO:1), YLW100W (as depicted in FIG. 4, as set forth in SEQ ID NO:3), YPL272C (as depicted in FIG. 6, as set forth in SEQ ID NO:5), YGR131W (as depicted in FIG. 8, as set forth in SEQ ID NO:7), and YDR453C (as depicted in FIG. 10, as set forth in SEQ ID NO:9), comprising recombinantly expressing in a fungal cell one or more candidate molecules, and detecting the expression of said ergosterol-pathway gene; wherein an increase or decrease in the gene expression relative to the expression in the absence of candidate molecules indicates that the molecules modulates ergosterol-pathway gene expression. In one embodiment the fungal cell is a transgenic cell.

The invention provides a method for identifying a molecule that modulates the activity of an ergosterol-pathway protein selected from the group consisting of YHR039C (as depicted in FIG. 3, as set forth in SEQ ID NO:2), YLW100W (as depicted in FIG. 5, as set forth in SEQ ID NO:4), YPL272C (as depicted in FIG. 7, as set forth in SEQ ID NO:6), YGR131W (as depicted in FIG. 9, as set forth in SEQ ID NO:8), and YDR453C (as depicted in FIG. 1, as set forth in SEQ ID NO:10), comprising contacting a fungal cell with one or more candidate molecules, detecting said protein; wherein an increase or decrease in the protein level relative to the level in the absence of candidate molecules indicates that the molecule modulates ergosterol-pathway gene expression.

The invention provides a method of identifying a molecule that binds to a ligand selected from the group consisting of (i) an S. cerevisiae ergosterol-pathway protein selected from the group consisting of YHR039C (as depicted in FIG. 3, as set forth in SEQ ID NO:2), YLW100W (as depicted in FIG. 5, as set forth in SEQ ID NO:4), YPL272C (as depicted in FIG. 7, as set forth in SEQ ID NO:6), YGR131W (as depicted in FIG. 9, as set forth in SEQ ID NO:8), and YDR453C (as depicted in FIG. 11, as set forth in SEQ ID NO:10), (ii) a fragment of the S. cerevisiae ergosterol-pathway protein, and (iii) a nucleic acid encoding the S. cerevisiae ergosterol-pathway protein or fragment, the method comprising: (a) contacting the ligand with a plurality of molecules under conditions conducive to binding between the ligand and the molecules; and (b) identifying a molecule within the plurality that binds to the ligand.

The invention provides a method for determining whether a molecule affects the function or activity of an PKC pathway in a cell comprising: (a) contacting the cell with, or recombinantly expressing within a cell the molecule; and (b) determining whether the expression of one or more of the genes selected from the group consisting of: SLT2(YHR030C) (as depicted in FIGS. 17A-B, as set forth in SEQ ID NO:11), YKR161C (as depicted in FIGS. 19A-B, as set forth in SEQ ID NO:13), PIR3(YKL163W) (as depicted in FIGS. 21A-B, as set forth in SEQ ID NO:15), YPK2(YMR104C) (as depicted in FIGS. 23A-B, as set forth in SEQ ID NO:17), YLR194C (as depicted in FIGS. 25A-B, as set forth in SEQ ID NO:19), and ST1(YDR055W) (as depicted in FIGS. 27A-B, as set forth in SEQ ID NO:21) is changed relative to said expression in the absence of the molecule. In one embodiment step (b) comprises determining whether SLT2 expression increases.

The invention provides a kit comprising in one or more containers a) a substance selected from the group consisting of an antibody against a PKC-pathway protein, a gene probe capable of hybridizing to RNA of a PKC-pathway gene, and pairs of gene primers capable of priming amplification of at least a portion of a PKC-pathway gene, and b)a molecule known to be capable of perturbing the PKC pathway.

The invention provides a method for identifying a molecule that activates the PKC pathway in yeast comprising contacting a yeast cell with one or more candidate molecules, and detecting a change in the RNA expression of a reporter gene for the PKC-pathway relative to the expression of the reporter gene in a yeast cell not contacted by the one or more candidate molecules, wherein the reporter gene is selected from the group consisting of: SLT2(YHR030C) (as depicted in FIGS. 17A-B, as set forth in SEQ ID NO:11), YKR161C (as depicted in FIGS. 19A-B, as set forth in SEQ ID NO:13), PIR3(YKL163W) (as depicted in FIGS. 21A-B, as set forth in SEQ ID NO:15), YPK2(YMR104C) (as depicted in FIGS. 23A-B, as set forth in SEQ ID NO:17), YLR194C (as depicted in FIGS. 25A-B, as set forth in SEQ ID NO:19), and ST1(YDR055W) (as depicted in FIGS. 27A-B, as set forth in SEQ ID NO:21).

The invention provides a method for identifying a molecule that activates the PKC pathway in yeast comprising contacting a yeast cell with one or more candidate molecules, and detecting a change in the protein expression of a reporter gene for the PKC-pathway relative to the expression of the reporter gene in a yeast cell not contacted by the one or more candidate molecules, wherein the reporter gene is selected from the group consisting of: SLT2(YHR030C) (as depicted in FIGS. 17A-B, as set forth in SEQ ID NO:11), YKR161C (as depicted in FIGS. 19A-B, as set forth in SEQ ID NO:13), PIR3(YKL163W) (as depicted in FIGS. 21A-B, as set forth in SEQ ID NO:15), YPK2(YMR104C) (as depicted in FIGS. 23A-B, as set forth in SEQ ID NO:17), YLR194C (as depicted in FIGS. 25A-B, as set forth in SEQ ID NO:19), and ST1(YDR055W) (as depicted in FIGS. 27A-B, as set forth in SEQ ID NO:21). In one embodiment the fungal cell is a transgenic cell.

The invention provides a method for identifying a molecule that modulates the expression of a PKC-pathway gene selected from the group consisting of SLT2(YHR030C) (as depicted in FIGS. 17A-B, as set forth in SEQ ID NO:11), YKR161C (as depicted in FIGS. 19A-B, as set forth in SEQ ID NO:13), PIR3(YKL163W) (as depicted in FIGS. 21A-B, as set forth in SEQ ID NO:15), YPK2(YMR104C) (as depicted in FIGS. 23A-B, as set forth in SEQ ID NO:17), YLR194C (as depicted in FIGS. 25A-B, as set forth in SEQ ID NO:19), and ST1(YDR055W) (as depicted in FIGS. 27A-B, as set forth in SEQ ID NO:21), comprising recombinantly expressing in a fungal cell one or more candidate molecules, and detecting the expression of said PKC-pathway gene; wherein an increase or decrease in the gene expression relative to the expression in the absence of candidate molecules indicates that the molecules modulates PKC-pathway gene expression. In one embodiment the fungal cell is a transgenic cell.

The invention provides a method for identifying a molecule that modulates the activity of a PKC-pathway protein selected from the group consisting of SLT2(YHR030C) (as depicted in FIG. 18, as set forth in SEQ ID NO:12), YKR161C (as depicted in FIG. 20, as set forth in SEQ ID NO:14), PIR3(YKL163W) (as depicted in FIG. 22, as set forth in SEQ ID NO:16), YPK2(YMR104C) (as depicted in FIG. 24, as set forth in SEQ ID NO:18), YLR194C (as depicted in FIG. 26, as set forth in SEQ ID NO:20), and ST1(YDR055W) (as depicted in FIG. 28, as set forth in SEQ ID NO:22), comprising contacting a fungal cell with one or more candidate molecules, detecting said protein; wherein an increase or decrease in the protein level relative to the level in the absence of candidate molecules indicates that the molecule modulates PKC-pathway gene expression.

The invention provides a method of identifying a molecule that binds to a ligand selected from the group consisting of (i) an S. cerevisiae PKC-pathway protein selected from the group consisting of SLT2(YHR030C) (as depicted in FIG. 18, as set forth in SEQ ID NO:12), YKR161C (as depicted in FIG. 20, as set forth in SEQ ID NO:14), PIR3(YKL163W) (as depicted in FIG. 22, as set forth in SEQ ID NO:16), YPK2(YMR104C) (as depicted in FIG. 24, as set forth in SEQ ID NO:18), YLR194C (as depicted in FIG. 26, as set forth in SEQ ID NO:20), and ST1(YDR055W) (as depicted in FIG. 28, as set forth in SEQ ID NO:22), (ii) a fragment of the S. cerevisiae PKC-pathway protein, and (iii) a nucleic acid encoding the S. cerevisiae PKC-pathway protein or fragment, the method comprising: (a) contacting the ligand with a plurality of molecules under conditions conducive to binding between the ligand and the molecules; and (b) identifying a molecule within the plurality that binds to the ligand.

The invention provides a method for determining whether a molecule affects the function or activity of an S. cerevisiae Invasive Growth pathway in a cell comprising: (a) contacting the cell with, or recombinantly expressing within a cell the molecule; and (b) determining whether the expression of one or more of the genes selected from the group consisting of: KSS1(YGR040W) (as depicted in FIG. 29, as set forth in SEQ ID NO:23), PGU1(YJR153W) (as depicted in FIG. 31, as set forth in SEQ ID NO:25), YRL042C (as depicted in FIG. 33, as set forth in SEQ ID NO:27), and SVS1(YPL163C) (as depicted in FIG. 35, as set forth in SEQ ID NO:29), is changed relative to said expression in the absence of the molecule. In one embodiment, step (b) comprises determining whether KSS1(YGR040W) (as depicted in FIG. 29, as set forth in SEQ ID NO:23), expression increases.

The invention provides a kit comprising in one or more containers a) a substance selected from the group consisting of an antibody against an S. cerevisiae Invasive Growth pathway protein, a gene probe capable of hybridizing to RNA of an Invasive Growth pathway gene, and pairs of gene primers capable of priming amplification of at least a portion of an Invasive Growth pathway gene, and b)a molecule known to be capable of perturbing the Invasive Growth pathway.

The invention provides a method for identifying a molecule that activates the Invasive Growth pathway in yeast comprising contacting a yeast cell with one or more candidate molecules, and detecting a change in the RNA expression of a reporter gene for the Invasive Growth pathway relative to the expression of the reporter gene in a yeast cell not contacted by the one or more candidate molecules, wherein the reporter gene is selected from the group consisting of KSS1(YGR040W) (as depicted in FIG. 29, as set forth in SEQ ID NO:23), PGU1(YJR153W) (as depicted in FIG. 31, as set forth in SEQ ID NO:25), YRL042C (as depicted in FIG. 33, as set forth in SEQ ID NO:27), and SVS1(YPL163C) (as depicted in FIG. 35, as set forth in SEQ ID NO:29).

The invention provides a method for identifying a molecule that activates the Invasive Growth pathway in yeast comprising contacting a yeast cell with one or more candidate molecules, and detecting a change in the protein expression of a reporter gene for the Invasive Growth pathway relative to the expression of the reporter gene in a yeast cell not contacted by the one or more candidate molecules, wherein the reporter gene is selected from the group consisting of: KSS1(YGR040W) (as depicted in FIG. 29, as set forth in SEQ ID NO:23), PGU1(YJR153W) (as depicted in FIG. 31, as set forth in SEQ ID NO:25), YRL042C (as depicted in FIG. 33, as set forth in SEQ ID NO:27), and SVS1(YPL163C) (as depicted in FIG. 35, as set forth in SEQ ID NO:29). In one embodiment the fungal cell is a transgenic cell.

The invention provides a method for identifying a molecule that modulates the expression of an Invasive Growth pathway gene selected from the group consisting of KSS1(YGR040W) (as depicted in FIG. 29, as set forth in SEQ ID NO:23), PGU1(YJR153W) (as depicted in FIG. 31, as set forth in SEQ ID NO:25), YRL042C (as depicted in FIG. 33, as set forth in SEQ ID NO:27), and SVS1(YPL163C) (as depicted in FIG. 35, as set forth in SEQ ID NO:29), comprising recombinantly expressing in a fungal cell one or more candidate molecules, and detecting the expression of said Invasive Growth pathway gene; wherein an increase or decrease in the gene expression relative to the expression in the absence of candidate molecules indicates that the molecules modulates Invasive Growth pathway gene expression. In one embodiment the fungal cell is a transgenic cell.

The invention provides a method for identifying a molecule that modulates the activity of an Invasive Growth pathway protein selected from the group consisting of KSS1(YGR040W) (as depicted in FIG. 30, as set forth in SEQ ID NO:24), PGU1(YJR153W) (as depicted in FIG. 32, as set forth in SEQ ID NO:26), YRL042C (as depicted in FIG. 34, as set forth in SEQ ID NO:28), and SVS1(YPL163C) (as depicted in FIG. 36, as set forth in SEQ ID NO:30), comprising contacting a fungal cell with one or more candidate molecules, detecting said protein; wherein an increase or decrease in the protein level relative to the level in the absence of candidate molecules indicates that the molecule modulates Invasive Growth pathway gene expression.

The invention provides a method of identifying a molecule that binds to a ligand selected from the group consisting of (i) an S. cerevisiae Invasive Growth pathway protein selected from the group consisting of KSS1(YGR040W) (as depicted in FIG. 30, as set forth in SEQ ID NO:24), PGU1(YJR153W) (as depicted in FIG. 32, as set forth in SEQ ID NO:26), YRL042C (as depicted in FIG. 34, as set forth in SEQ ID NO:28), and SVS1(YPL163C) (as depicted in FIG. 36, as set forth in SEQ ID NO:30), (ii) a fragment of the S. cerevisiae Invasive Growth pathway protein, and (iii) a nucleic acid encoding the S. cerevisiae Invasive Growth pathway protein or fragment, the method comprising (a) contacting the ligand with a plurality of molecules under conditions conducive to binding between the ligand and the molecules; and (b) identifying a molecule within the plurality that binds to the ligand.

### 4. BRIEF DESCRIPTION OF THE DRAWINGS

FIG. 1 Schematic diagram of the method by which reporter genes and/or target genes are identified

FIG. 2 DNA sequence of S. cerevisiae YHR039C ergosterol-pathway gene. The nucleic acid sequence of YHR039C is set forth in SEQ ID NO:1.

FIG. 3 The amino acid sequence of the protein encoded by S. cerevisiae YHR039C ergosterol-pathway gene. The amino acid sequence of YHR039C is set forth in SEQ ID NO:2.

FIG. 4 DNA sequence of S. cerevisiae YLR100W ergosterol-pathway gene. The nucleic acid sequence of YLR100W is set forth in SEQ ID NO:3.

FIG. 5 The amino acid sequence of the protein encoded by S. cerevisiae YLR100W ergosterol-pathway gene. The amino acid sequence of YLR100W is set forth in SEQ ID NO:4.

FIG. 6 DNA sequence of S. cerevisiae YPL272C ergosterol-pathway gene. The nucleic acid sequence of YPL272C is set forth in SEQ ID NO:5.

FIG. 7 The amino acid sequence of the protein encoded by S. cerevisiae YPL272C ergosterol-pathway gene. The amino acid sequence of YPL272C is set forth in SEQ ID NO:6.

FIG. 8 DNA sequence of S. cerevisiae YGR131W ergosterol-pathway gene. The nucleic acid sequence of YGR131W is set forth in SEQ ID NO:7.

FIG. 9 The amino acid sequence of the protein encoded by S. cerevisiae YGR131W ergosterol-pathway gene. The amino acid sequence of YGR131W is set forth in SEQ ID NO: 8.

FIG. 10 DNA sequence of S. cerevisiae YDR453C ergosterol-pathway gene. The nucleic acid sequence of YDR453C is set forth in SEQ ID NO:9.

FIG. 11 The amino acid sequence of the protein encoded by S. cerevisiae YDR453C ergosterol-pathway gene. The amino acid sequence of YDR453C is set forth in SEQ ID NO:10.

FIG. 12 Ergosterol Biosynthetic Pathway. The various steps in the synthesis of ergosterol in S. cerevisiae are shown, beginning with 2 acetyl-CoA. The genes encoding enzymes in the pathway are shown in green. Antifungal agents that inhibit specific steps in the pathway are shown in bold.

FIG. 13 Clotrimazole Titration Plot. This plot shows the complexity of the drug signature and demonstrates genes which are induced or repressed in response to drug treatment. An example of a gene which is induced to a high level is labeled YPL272C.

FIG. 14 Cluster analysis of ergosterol-pathway genes. When the signature of yeast mutant strains deleted in a number of ergosterol-pathway genes are compared certain the genes cluster on the same branch. The genes Y4R039C, YLR100W, and YGL001C co-clustered and are reporters of the ergosterol-pathway. The genes YPL272C, YGR131W, and YDR453C co-clustered and are also reporters of the ergosterol-pathway. Clustering analysis of yeast genes reveals relationships between different genes, and demonstrates that several genes behave similarly to several known ERG genes.

FIG. 15 PKC pathway of yeast as induced by pheromone or cell wall integrety stimulus.

FIG. 16 Results of two-dimensional cluster analysis which was used in to identify the reporter genes and target genes of the PKC pathway.

FIGS. 17A-B DNA sequence of S. cerevisiae SL2(YHR030C) PKC-pathway gene. The nucleic acid sequence of SL2(YHR030C) is set forth in SEQ ID NO:11.

FIG. 18 The amino acid sequence of the protein encoded by S. cerevisiae SL2(YHR030C) PKC-pathway gene. The amino acid sequence of SL2(YHR030C) is set forth in SEQ ID NO:12.

FIGS. 19A-B DNA sequence of S. cerevisiae YKL161C PKC-pathway gene. The nucleic acid sequence of YKL161C is set forth in SEQ ID NO:13.

FIG. 20 The amino acid sequence of the protein encoded by S. cerevisiae YKL161C PKC-pathway gene. The amino acid sequence of YKL161C is set forth in SEQ ID NO:14.

FIGS. 21A-B DNA sequence of S. cerevisiae PIR3(YKL163W) PKC-pathway gene. The nucleic acid sequence of PIR3(YKL163W) is set forth in SEQ ID NO:15.

FIG. 22 The amino acid sequence of the protein encoded by S. cerevisiae PIR3(YKL163W) PKC-pathway gene. The amino acid sequence of PIR3(YKL163W) is set forth in SEQ ID NO:16.

FIGS. 23A-B DNA sequence of S. cerevisiae YPK2(YMR104C) PKC-pathway gene. The nucleic acid sequence of YPK2(YMR104C) is set forth in SEQ ID NO:17.

FIG. 24 The amino acid sequence of the protein encoded by S. cerevisiae YPK2(YMR104C) PKC-pathway gene. The amino acid sequence of YPK2(YMR104C) is set forth in SEQ ID NO:18.

FIGS. 25A-B DNA sequence of S. cerevisiae YLR194C PKC-pathway gene. The nucleic acid sequence of YLR194C is set forth in SEQ ID NO:19.

FIG. 26 The amino acid sequence of the protein encoded by S. cerevisiae YLR194C PKC-pathway gene. The amino acid sequence of YLR194C is set forth in SEQ ID NO:20.

FIGS. 27A-B DNA sequence of S. cerevisiae PST1(YDR055C) PKC-pathway gene. The nucleic acid sequence of PST1(YDR055C) is set forth in SEQ ID NO:21.

FIG. 28 The amino acid sequence of the protein encoded by S. cerevisiae PST1(YDR055C) PKC-pathway gene. The amino acid sequence of PST1(YDR055C) is set forth in SEQ ID NO:22.

FIG. 29 DNA sequence of S. cerevisiae KSS1(YGR040W) Invasive Growth pathway gene. The nucleic acid sequence of KSS1(YGR040W) is set forth in SEQ ID NO:23.

FIG. 30 The amino acid sequence of the protein encoded by S. cerevisiae KSS1(YGR040W) Invasive Growth pathway gene. The amino acid sequence of KSS1(YGR040W) is set forth in SEQ ID NO:24.

FIG. 31 DNA sequence of S. cerevisiae PGU1(YJR153W) Invasive Growth pathway gene. The nucleic acid sequence of PGU1(YJR153W) is set forth in SEQ ID NO:25.

FIG. 32 The amino acid sequence of the protein encoded by S. cerevisiae PGU1(YJR153W) Invasive Growth pathway gene. The amino acid sequence of PGU1(YJR153W) is set forth in SEQ ID NO:26.

FIG. 33 DNA sequence of S. cerevisiae YHR042C Invasive Growth pathway gene. The nucleic acid sequence of YHR042C is set forth in SEQ ID NO:27.

FIG. 34 The amino acid sequence of the protein encoded by S. cerevisiae YHR042C Invasive Growth pathway gene. The amino acid sequence of YHR042C is set forth in SEQ ID NO:28.

FIG. 35 DNA sequence of S. cerevisiae SVS1(YPL163C) Invasive Growth pathway gene. The nucleic acid sequence of SVS1(YPL163C) is set forth in SEQ ID NO:29.

FIG. 36 The amino acid sequence of the protein encoded by S. cerevisiae SVS1(YPL163C) Invasive Growth pathway gene. The amino acid sequence of SVS1(YPL163C) is set forth in SEQ ID NO:30.

### 5. DETAILED DESCRIPTION OF THE INVENTION

The present invention relates, in part, to methods for identifying one or more reporter genes and/or target genes for a particular biological pathway of interest. The reporter genes of this invention are particularly useful for analyzing the activity of particular biological pathways of interest, and may be further used in the design of drugs, drug therapies or other biological agents (e.g., insecticides, herbicides, fungicides, antibiotics or antivirals) to target a particular biological pathway. The present invention also relates to methods for identifying one or more target genes for a particular biological pathway of interest. Target genes of the invention are useful as specific targets for drug which may be designed to enhance, inhibit, or modulate a particular biological pathway. Methods to identify gene which modifies the function or structure of a member (e.g., compound or gene product) of a particular biological pathway are provided.

The present invention provides examples of reporter genes and/or target genes which have been discovered by the methods of the invention. Specifically, the inventors have made the surprising discovery that five S. cerevisiae genes (previously of unknown function) form clustered co-regulated sets of genes and are reporters of the ergosterol-pathway. The methods of the invention are also exemplified in that the inventors have specifically discovered six S. cerevisiae reporter genes of the protein kinase C (PKC) pathway. Two of these genes are also novel target genes of the PKC pathway and provide targets for the development of PKC pathway-specific drugs, drug therapies, or other related biological or therapeutical agents. The methods of the invention are further exemplified by the discovery of four novel reporter genes of the S. cerevisiae Invasive growth pathway. One of these genes also serves as a target gene for the Invasive Growth pathway, and may be used to develop Invasive Growth pathway-specific drugs, drug therapies, or other related biological or therapeutical agents.

As described herein, the inventors developed a strategy to search the genome of an organism for cellular constituents which function in a biological pathway of interest. Specifically, the inventors have developed a strategy to search the genome of an organism for reporter genes and/or target genes of a biological pathway of interest. In one embodiment, as described herein, the inventors developed a strategy to search the genome of S. cerevisiae for genes which function in a biological pathway of interest. Any pathway of interest may be examined by the methods of the invention. In specific embodiments, the methods of the invention are illustrated by way of the ergosterol-pathway, the PKC pathway, and the Invasive-Growth pathway. Additionally, the genome of any species may be used in the methods of the invention, so long as the genome of the species is at least partially sequenced. In several embodiments of the invention, 20-30%, 30-40%, or 40-60%, of the sequence of the genome of the species examined by the methods of the invention is known. In preferred embodiments of the invention, 60-75%, 75-85%, or 85-90%, of the sequence of the genome of the species examined by the methods of the invention is known. In highly preferred embodiments of the invention, 90-95%, 95-98%, or 98% or more of the sequence of the genome of the species examined by the methods of the invention is known. In a most preferred embodiment of the invention, the entire sequence of the genome of the species examined by the methods of the invention is known.

The methods described herein relate to DNA microarray technology as described in Section 5.1 et seq., and in U.S. patent Ser. No. 09/179,569, filed Oct. 27, 1998 now pending, and U.S. patent Ser. No. 09/220,275 filed Dec. 23, 1998, now pending, and U.S. patent Ser. No.09/220,142, filed Dec. 23, 1998 now pending, which are incorporated herein by reference in their entirety. The reporter genes and target genes of the invention constitute very useful tools for probing the function, regulation, activation, and inhibition of their corresponding pathways. Biochemical and genetic analysis of pathways involving the reporters and particularly the targets of the invention can be expected to lead to the discovery of new drug targets, therapeutic proteins, diagnostics, and prognostics useful in the treatment of diseases and clinical problems, for example, those associated with the activation or inactivation of a particular pathway.

Methods for biochemical analysis of pathways of the invention are provided. Such methods may yield results of importance to human disease. For example, systematic identification of participants in the ergosterol-pathway, or components regulating synthesis of ergosterol provide leads to the identification of drug targets, therapeutic proteins, diagnostics, or prognostics useful for treatment or management of fungal infections.

The invention is illustrated by way of examples set forth in Section 6 below which disclose, inter alia, the characterization of reporters and targets of the invention including reporter genes of the S. cerevisiae ergosterol-pathway, PKC-pathway, and Invasive Growth pathway using DNA microarray technology.

For clarity of disclosure, and not by way of limitation, the detailed description of the invention is divided into the subsections which follow.

### 5.1. Characterization Procedures

The present invention relates, in part, to methods for identifying one or more reporter genes for a particular biological pathway of interest. As used herein, a reporter gene refers to any gene for which a change in it expression and/or activity of its encoded RNA or protein is indicative of a changes in the activity of a particular biological pathway of pathway of interest. Thus, the reporter genes of this invention are useful for analyzing the activity of particular biological pathways of interest, e.g., in the design of drugs, drug therapies or other biological agents (e.g., insecticides, herbicides, fungicides, antibiotics or antivirals) to target particular biological pathways.

The present invention also relates, in part, to methods for identifying one or more target genes for a particular biological pathway of interest. As used herein, a target gene refers to any gene whose expression and/or activity is necessary for normal activity or function of the pathway. Thus, the target genes of this invention are useful as targets for drugs designed to enhance, inhibit, or modulate a particular biological pathway. Thus, the target genes of this invention are useful targets for design of drugs, drug therapies or other biological agents (e.g., insecticides, herbicides, fungicides, antibiotics or antivirals) directed to a particular biological pathway.

Biological pathways, as used herein, refer to collections of cellular constituents (e.g., protein abundances or activities, protein phosphorylation, RNA species abundances such as mRNA species abundances, or DNA species abundances such as abundances of cDNA species derived from mRNA—as used herein the term “cellular constituent” is not intended to refer to known subcellular organelles such as mitochondria, lysozomes, etc.) which are related in that each cellular constituent in the collection is influenced according to some biological mechanism by one or more other cellular constituents in the collection. Biological pathways of the present invention therefore include well-known biochemical synthetic pathways including, for example, the yeast ergosterol pathway, in which, e.g., molecules are broken down to provide cellular energy stores or in which protein or nucleic acid precursors or other cellular compounds are synthesized. Signaling and control pathways typically include primary or intermediate signaling molecules, as well as proteins participating in the signal or control cascades usually characterizing these pathways. In signaling pathways, binding of a signal molecule to a receptor usually directly influences the abundances of intermediate signaling molecules and indirectly influences, e.g., the degree of phosphorylation (or other modification) of pathway proteins. Both of these effects in turn influence activities of cellular proteins that are key effectors of the cellular processes initiated by the signal, for example, by affecting the transcriptional state of the cell. Control pathways, such as those controlling the timing and occurrence of the cell cycle, are similar. Here, multiple, often ongoing, cellular events are temporally coordinated, often with feedback control, to achieve a consistent outcome, such as cell division with chromosome segregation. This coordination is a consequence of functioning of the pathway, often mediated by mutual influences of proteins on each other's degree of phosphorylation or other modification. Biological pathways of the invention also include, but are not limited to: signaling pathways, control pathways, mating pathways, cell cycle pathways, cell division pathways, cell repair pathways, small molecule synthesis pathways, protein synthesis pathways, DNA synthesis pathways, RNA synthesis pathways, DNA repair pathways, stress-response pathways, cytoskeletal pathways, steroid pathways, receptor-mediated signal transduction pathways, transcriptional pathways, translational pathways, immune response pathways, heat-shock pathways, motility pathways, secretion pathways, endocytotic pathways, protein sorting pathways, phagocytic pathways, photosynthetic pathways, excretion pathways, electrical response pathways, pressure-response pathways, protein modification pathways, small-molecule response pathways, toxic-molecule response pathway transformation pathways, etc. Specifically, the invention herein is illustrated in subsection 6, by way of reporter genes which have been discovered for the ergosterol-pathway and the protein kinase C pathway. Other, well known control pathways seek to maintain optimal levels of cellular metabolites in the face of a fluctuating environment. Further examples of cellular pathways operating according to understood mechanisms are well known and will therefore be readily apparent to those of skill in the art.

The methods of the invention may be used to identify reporter genes or target genes in any cell type from any species of organism. In one preferred embodiment, the methods of the invention are used to identify reporter genes and target genes in S. cerevisiae. However, in other preferred embodiments the methods of the invention are used to identify reporter genes and/or target genes in other cell types including prokaryotic and eukaryotic, vertebrate and invertebrate, and in other species, including plant, animal, insect, worm, funus, yeast, fish, and bird species. In one preferred embodiment the methods of the invention identify one or more reporter genes and or-target genes in a mammalian species of interest (e.g. mouse, rat, rabbit, dog, cat, horse, sheep, pig, cattle, etc.). In one particularly preferred embodiment, the methods of the invention identify one or more reporter genes and/or target genes in a human. In another preferred embodiment the methods of the invention identify one or more reporter genes and/or target genes in a species which is amenable to genetic manipulation of the entire organism (e.g., fly or worm). In other embodiments, the methods of the invention identify one or more reporter genes and/or target genes in other species described herein.

The reporter genes of the present invention comprise genes whose genetic transcripts (i.e., mRNA transcripts or cDNA molecules produced from mRNA transcripts) “co-vary” and/or are “co-regulated.” Specifically, the reporter genes of the invention increase or decrease the abundance of their transcripts under some set of conditions which is associated with a particular biological pathway of interest and/or with other genes which are associated with the particular biological pathway of interest.

The target genes of the present invention comprise genes whose genetic transcripts (i.e., mRNA transcripts or cDNA molecules produced from mRNA transcripts) “co-vary” and/or are “co-regulated.” Specifically, the target genes of the invention increase or decrease the abundance of their transcripts under some set of conditions which is associated with a particular biological pathway of interest and/or with other genes which are associated with the particular biological pathway of interest. Further, target genes of the invention are those genes of a geneset who expression and/or activity are necessary for the activity or function of the pathway. Methods for identifying such co-varying genes are described generally and in detail in U.S. patent application Ser. No. 09/179,569, filed Oct. 27, 1998, now pending, in U.S. patent application Ser. No.09/220,275, filed Dec. 23, 1998, now pending, and in U.S. patent application Ser. No. 09/220,142 filed Dec. 23, 1998, now pending each of which are incorporated herein by reference in their entirety. These methods are described below as they particularly pertain to identifying reporter genes. Specifically, subsection 5.1.1 describes methods such as cluster analysis which may be used to identify covarying genesets. Such cluster analysis methods are preferably applied to measurements of the “transcriptional state” of a cell; i.e., to measurements of abundances of genetic transcripts (mRNA or cDNA) of a cell. Most preferably, the transcriptional state of a cell is measured using polynucleotide microarrays. Accordingly, subsection 5.1.2-5.1.5 describe methods of measuring the transcriptional state using microarrays, including methods of construction microarrays, methods of hybridizing polynucleotide samples (e.g., from cells) to microarrays, and signal detection on microarrays. Subsection 5.1.6 describes other, less preferred methods by which the transcriptional state of a cell may be measured.

Although for simplicity the disclosure often makes reference to single cells (e.g., “RNA is isolated from a cell exposed to a particular drug”), it will be understood by those of skill in the art that more often any particular step of the invention will be carried out using a plurality of genetically similar cells, e.g., from a cultured cell line. Such similar cells are referred to herein as a “cell type.” Such cells may be either from naturally single celled organisms (e.g., E. coli or S. cerevisiae) or derived from multi-cellular higher organisms (e.g. from plant or animal organisms, including mammalian organisms such as a human cell line).

5.1.1. Cluster Analysis

In a preferred aspect of the invention, the reporter genes and/or target genes may be identified by methods using cluster analysis. The cluster analysis technique is based in the principal that in general, cellular constituents (e.g., gene transcripts) will respond in a coordinated fashion in response to a particular stimulus, treatment, or biological state. Therefore, subsets of cellular constituents will typically change together, e.g., by increasing or decreasing their abundances and/or activities, under some set of conditions which preferably include the conditions or perturbations of interest to a user of the present invention (e.g., treatment with antifungal compounds).

Further, the abundances and/or activities of individual cellular constituents are not all regulated independently. Rather, individual cellular constituents from a cell will typically share one or more regulatory elements with other cellular constituents from the same cell. For example, and not by way of limitation, in embodiments where the cellular constituents comprise genetic transcripts, the rates of transcription are generally regulated by regulator sequence patterns, i.e., transcription factor binding sites. Typically, several genes within a cell may share one or more transcription factor binding sites. Such cellular constituents are therefore said to be “co-regulated,” and comprise co-regulated cellular constituent sets or “co-regulated sets.” For example, and not by way of limitation, genes tend to increase or decrease their rates of transcription together when they possess similar transcription factor binding sites. Such a mechanism accounts for the coordinated responses of genes to particular signaling inputs. For example, see Madhani and Fink, 1998, Transactions in Genetics 14:151-155; and Arnone and Davidson, 1997, Development 124:1851-1864. For instance, individual genes which synthesize different components of a necessary protein or cellular structure are generally co-regulated. Also, duplicated genes (see, e.g., Wagner, 1996, Biol. Cybern. 74:557-567) are co-regulated to the extent that genetic mutations have not led to functional divergence in their regulatory regions. Further, because genetic regulatory sequences are modular (see, e.g., Yuh et al., 1998, Science 279:1896-1902), the more regulatory “modules” two genes have in common, the greater the variety of conditions under which they will be co-regulated in their transcription rates. Physical separation between modules along the chromosome is also an important determinant since co-activators are often involved.

In particularly preferred embodiments of the present invention, the cellular constituents in a biological profile comprise genetic transcripts such as mRNA abundances, or abundances of cDNA molecules produced from mRNA transcripts. In such embodiments, the co-regulated sets comprise genes which are generally co-regulated to some extent. Such co-regulated sets are referred to herein as “genesets.” Thus, in particularly preferred embodiments of the present invention, the co-regulated cellular constituent sets are genesets. In one specific embodiment of the present invention, the geneset comprises genes of the ergosterol-pathway. In another specific embodiment of the present invention, the geneset comprises genes of the PKC-pathway. In another specific embodiment of the present invention, the geneset comprises genes of the Invasive Growth pathway.

In a specific embodiment of the invention, when the genome of the organism of interest has been sequenced, the number of ORF's can be determined and mRNA coding regions identified by analysis of the DNA sequence. For example, the genome of Saccharomyces cerevisiae has been completely sequenced, and is reported to have approximately 6275 ORFs longer than 99 amino acids. Analysis of the ORFs indicates that there are 5885 ORFs that are likely to encode protein products (Goffeau et al., 1996, Science 274:546-567). However, many of these genes do not have a known function, nor are they associated with a known function. The invention herein provides methods for assigning function to such ORFs, by the methods of the invention including cluster analysis.

### 5.2. Pathway Response Profiles & Perturbations

In one aspect of the invention, gene expression change in response to a large number of perturbations is used to construct a clustering tree for the purpose of defining genesets. Preferably, the perturbations should target different pathways. In order to measure expression responses to the pathway perturbation, biological samples are subjected to perturbations to pathways of interest. The samples exposed to the perturbation and samples not exposed to the perturbation are used to construct transcript arrays, which are measured to find the mRNAs with modified expression and the degree of modification due to exposure to the perturbation. Thereby, the perturbation-response profile is obtained.

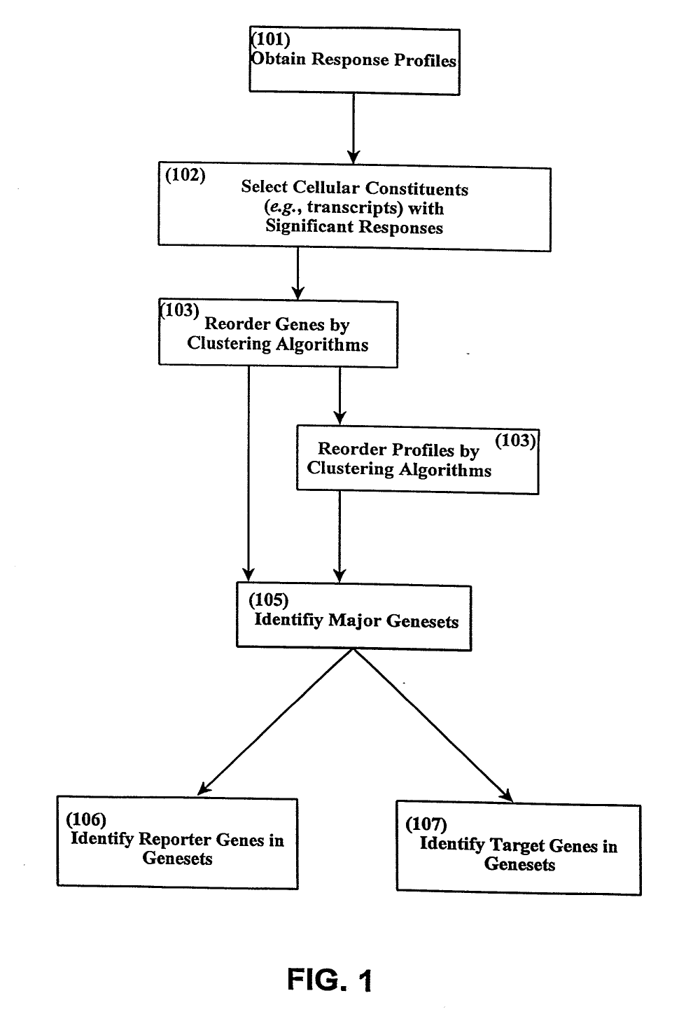

FIG. 1 illustrates an overview of the method by which reporter genes and/or target genes are identified. The methods analyze a plurality of “response profiles” which are preferably obtained or provided (FIG. 1, 101) from measurements of the transcriptional or translational state of a cell (e.g., measurements of mRNA abundances or of abundances of cDNA derived from mRNA) under a variety of different experimental conditions. More precisely, the transcriptional or translational state of the cell in response to a plurality of different perturbations to the cell is measured. In preferred embodiments, the transcriptional or translational state of the cell is measured in response to at least ten different perturbations to the cell, more preferably in response to at least 100 perturbations, still more preferably in response to at least 400 perturbations, and yet more preferably in response to over 1,000 different perturbations.

Perturbations to the cell may comprise, for example, exposure to one or more drugs at one or more levels (i.e., at one or more concentrations of the drug). Perturbations may also comprise genetic alterations to the cell such as genetic “knockouts” wherein one or more genes are deleted and/or no longer expressed in the cell. Other possible genetic alterations include regulated expression of one or more genes in the cell, wherein the level of expression of the one or more genes is altered (e.g., increased or decreased) in a controlled manner, e.g., by means of a titratable promoter system. Such perturbations, as well as others which may be used to identify reporter genes and/or target genes, are described, in detail in subsection 5.3 below.

Perturbations to the cell may further comprise changes in one or more aspects of the physical environment of the cell. Such environmental changes can include, for example, changes in the temperature (e.g., a temperature elevation of 10° C.) or exposure to moderate doses of radiation. Other exemplary environmental changes include changes in the nutritional environment, such as the presence or absence of particular sugars, amino acids, and so forth.

In preferred embodiments, some of the perturbations are perturbations which are known to affect a particular biological pathway of interest; i.e., the biological pathway for which one or more reporter genes and/or target genes are to be identified. In some preferred embodiments, about 5-50%, preferably about 10-30%, more preferably about 10-25%, still more preferably about 10-20%, and most preferably about 10-15% of the perturbations are perturbations which are known to affect a particular biological pathway of interest.

At least two genes (i.e. at least two mRNA or cDNA species) are measured in response to each perturbation. Preferably, at least 10 genes are measured in response to each perturbation, more preferably more than 100 genes, still more preferably more than 1,000 genes, and most preferably more than 10,000 genes. Preferably mRNA or cDNA abundances are measured for more that 10% of the genes of the cell being analyzed. More preferably, mRNA or cDNA abundances are measured for more than 25%, more than 50%, more than 75%, more than 80%, more than 90%, more than 95%, or more than 99% of the genes of the cell being analyzed. Most preferably, mRNA or cDNA abundances are measured for all of the genes of the cell being analyzed. In preferred embodiment, some of the genes measured in response to each perturbation are genes which are known to be involved in a particular biological pathway of interest, i.e., the biological pathway for which one or more reporeter genes are to be identified. In some preferred embodiments, about 5-50%, preferably about 10-30%, more preferably about 10-25%, still more preferably about 10-20%, and most preferably about 10-15% of the genes measured in response to each perturbation are genes which are known to be involved in a particular biological pathway of interest.

In preferred embodiments, the response profiles analyzed by the methods of the invention are optionally screened, before the analysis, to select only those cellular constituents that have a significant response in some fraction of the profiles (FIG. 1, 102). In particular, although the profiles may cover up to ˜10⁵ genes, in most perturbations a large part or evan a majority of these genes will not change significantly, or the changes may be small and dominated by experimental error. Accordingly, in most embodiments, it will be unhelpful and cumbersome to use these genes in to identify reporter genes according to the methods of this invention. Thus, they are preferably deleted from all profiles.

In certain embodiment, only genes that have a response greater than or equal to two standard errors in more than N profiles are selected for subsequent analysis, where N may be one or more and is preferably selected by the user. Preferably, N will tend to be larger for larger sets of response profiles. For example, in one preferred embodiment N may be approximately equal to the square root of the number of response profiles analyzed.

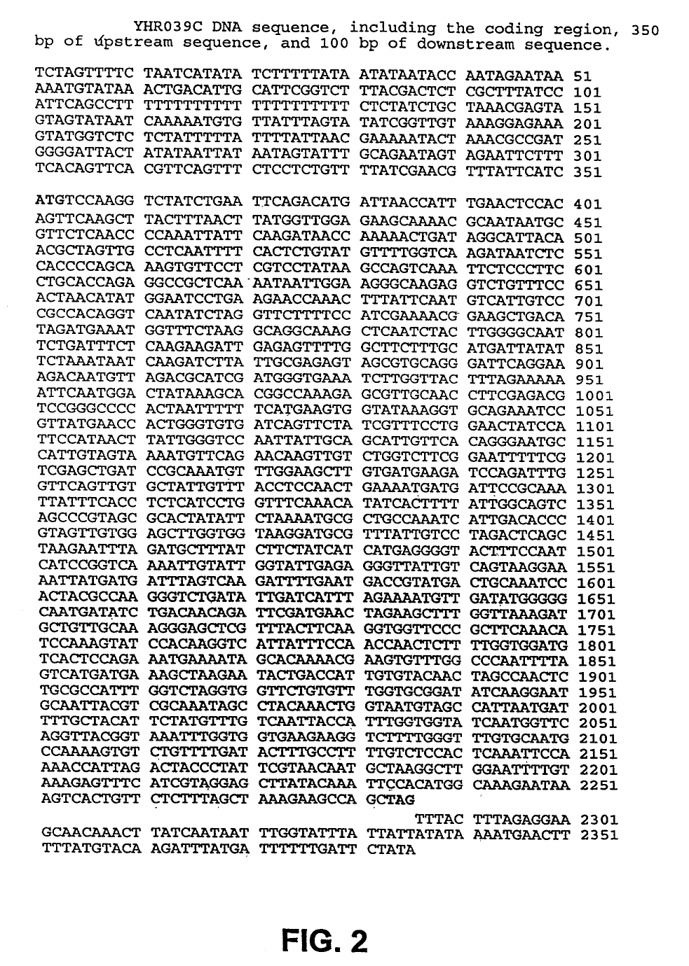

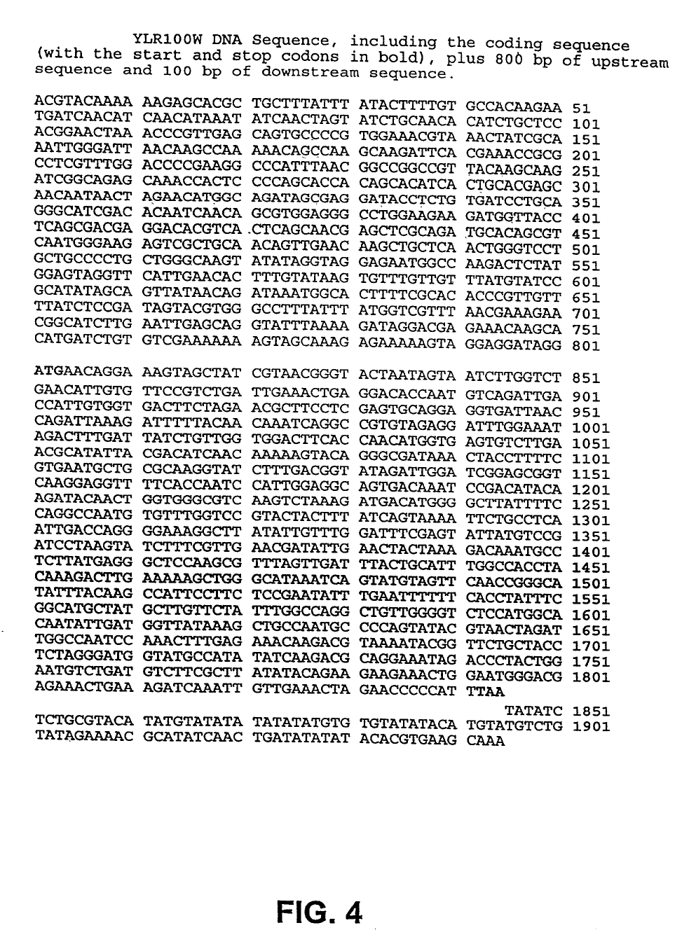

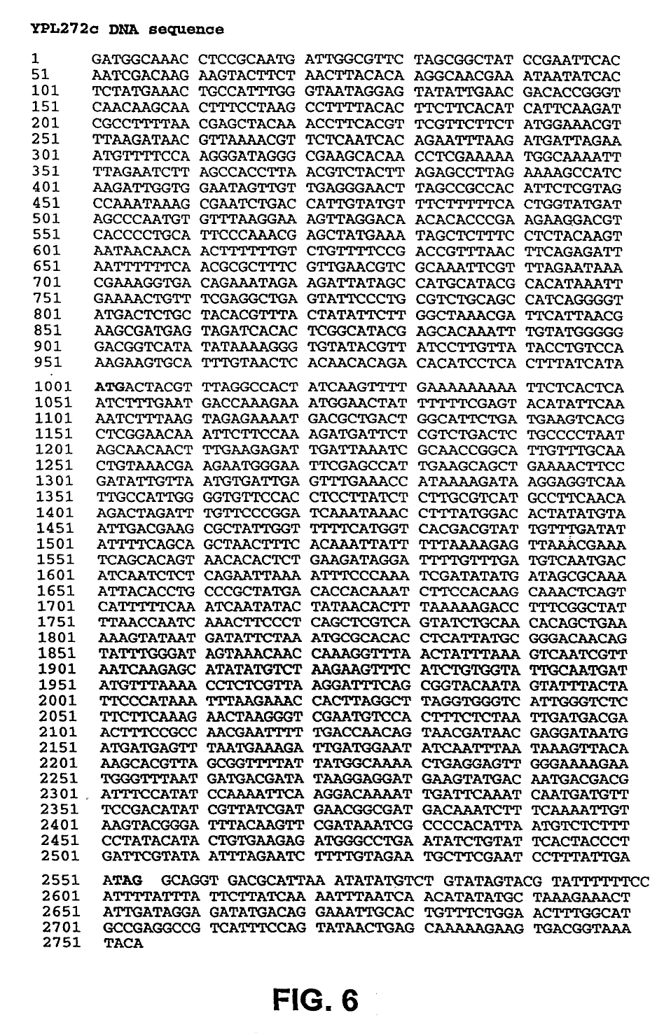

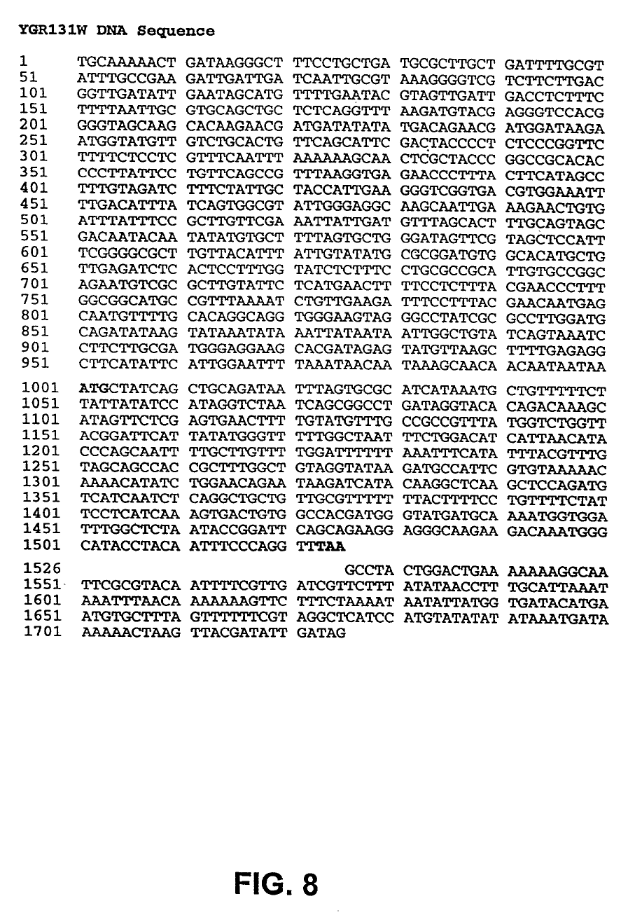

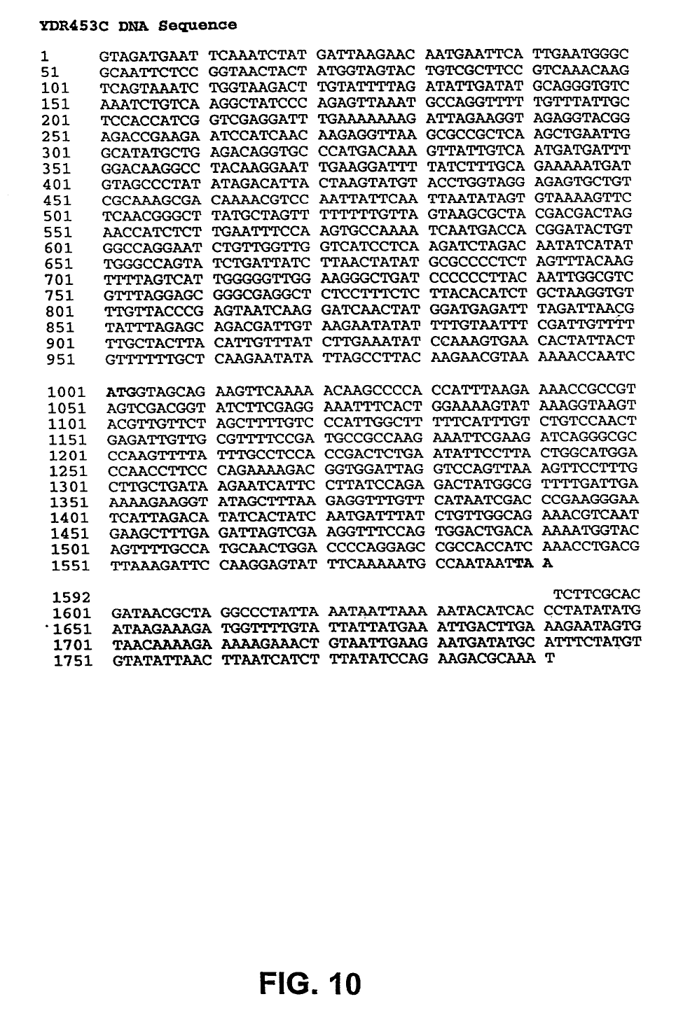

The invention provides a method for determining whether a molecule affects the function or activity of an ergosterol pathway in a cell comprising: (a) contacting the cell with, or recombinantly expressing within a cell the molecule; and (b) determining whether the expression of one or more of the genes selected from the group consisting of: YHR039C (as depicted in FIG. 2, as set forth in SEQ ID NO:1), YLW100W (as depicted in FIG. 4, as set forth in SEQ ID NO:3), YPL272C (as depicted in FIG. 6, as set forth in SEQ ID NO:5), YGR131W (as depicted in FIG. 8, as set forth in SEQ ID NO:7), and YDR453C (as depicted in FIG. 10, as set forth in SEQ ID NO:9) is changed relative to said expression in the absence of the molecule.

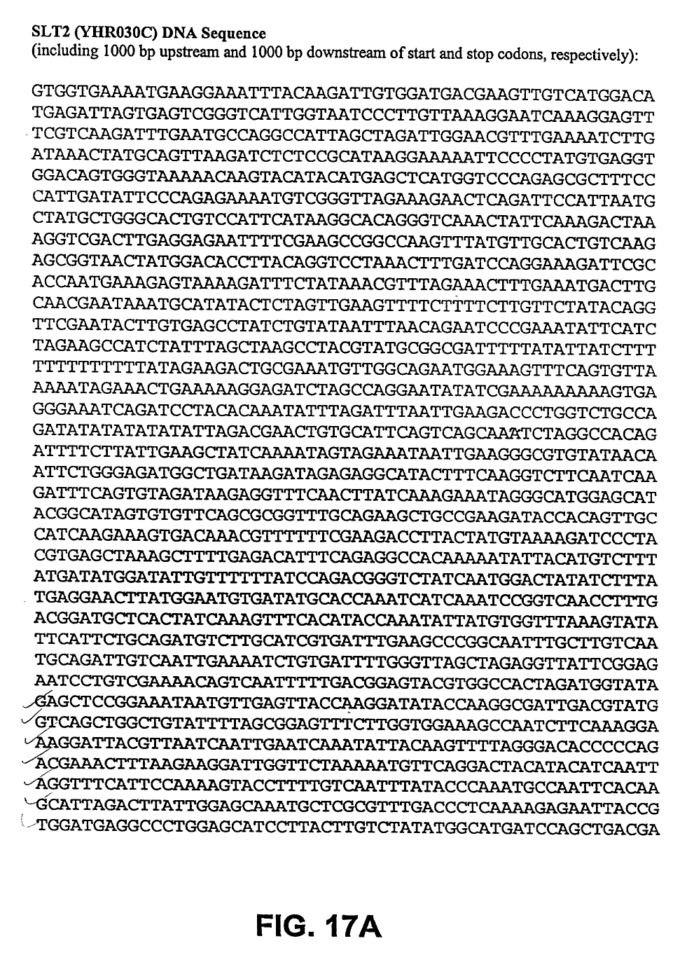

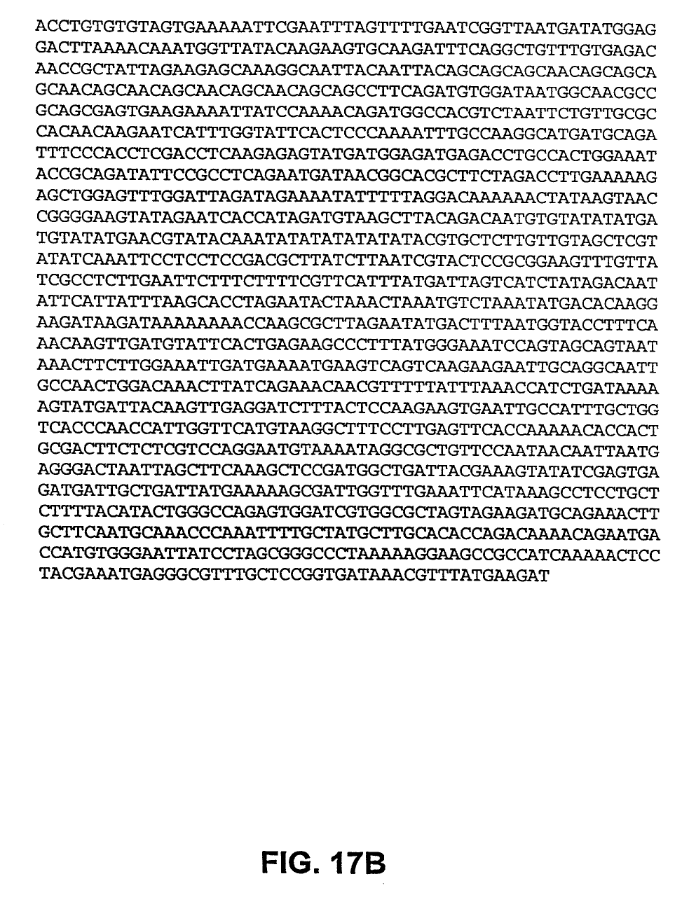

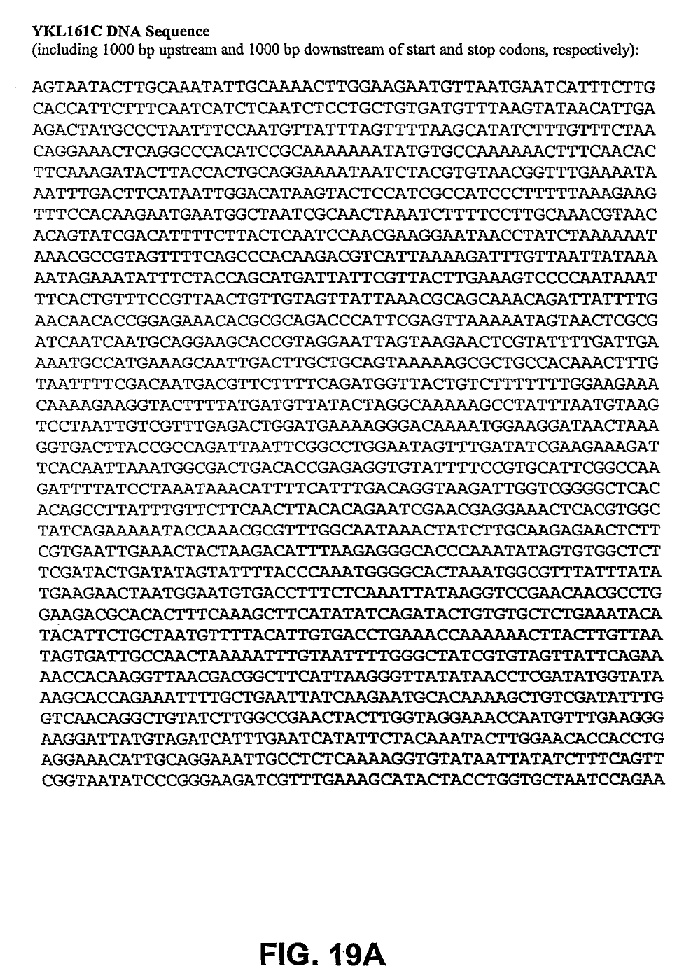

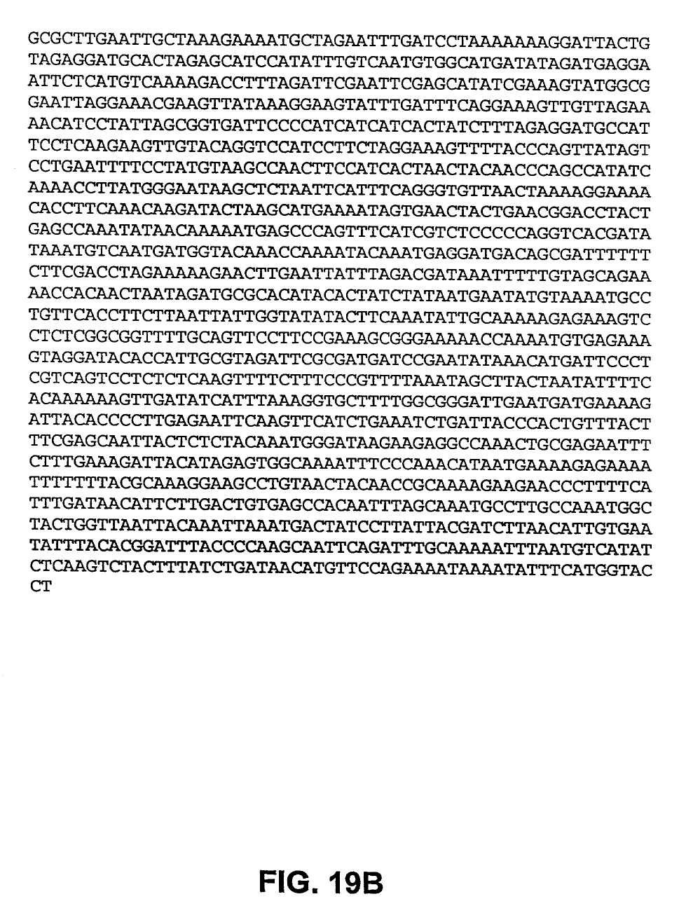

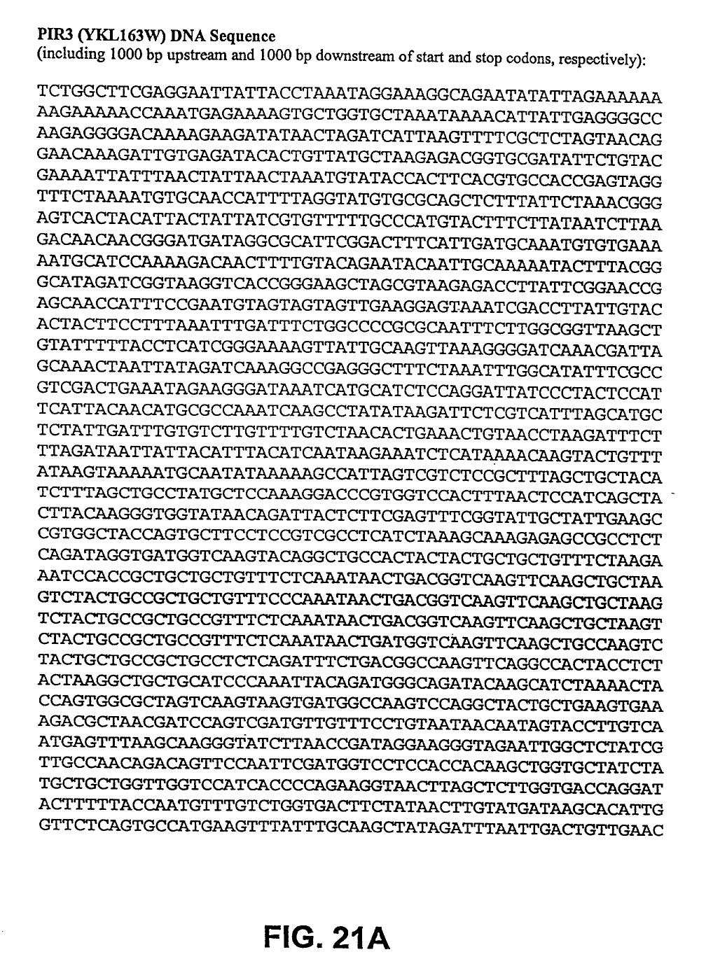

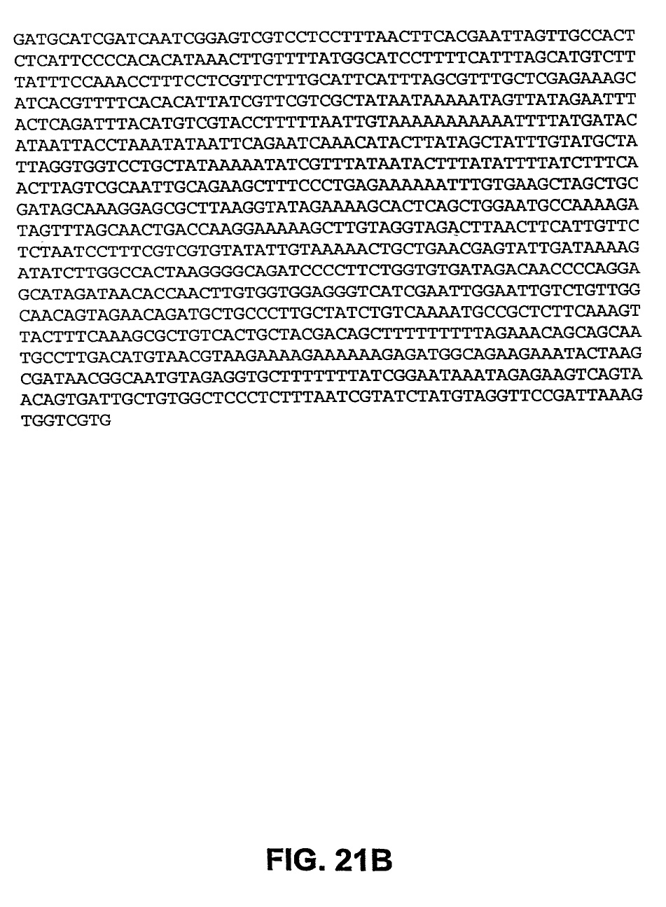

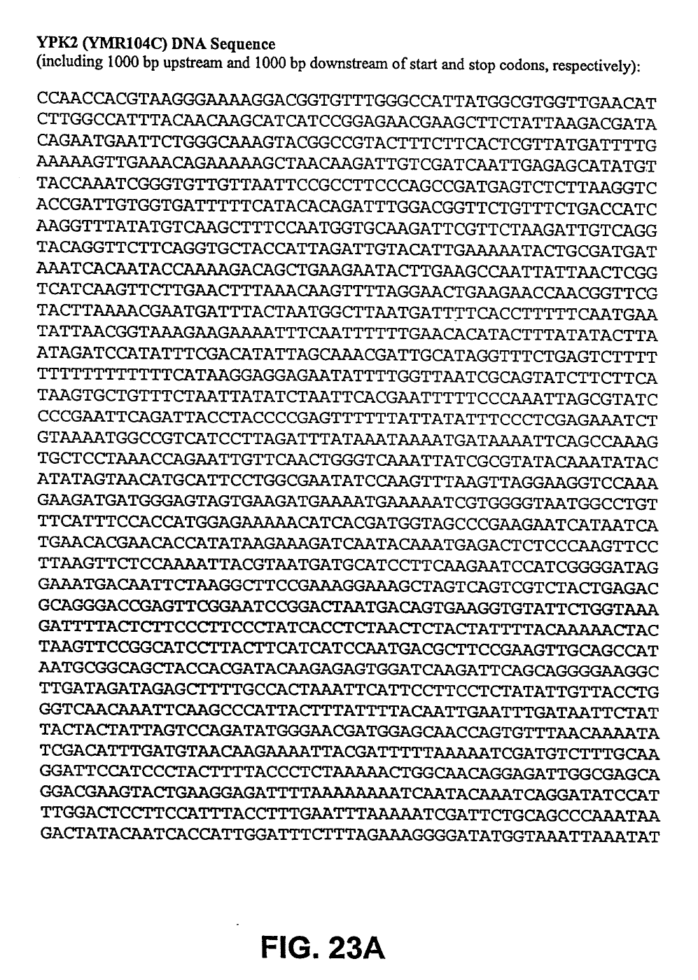

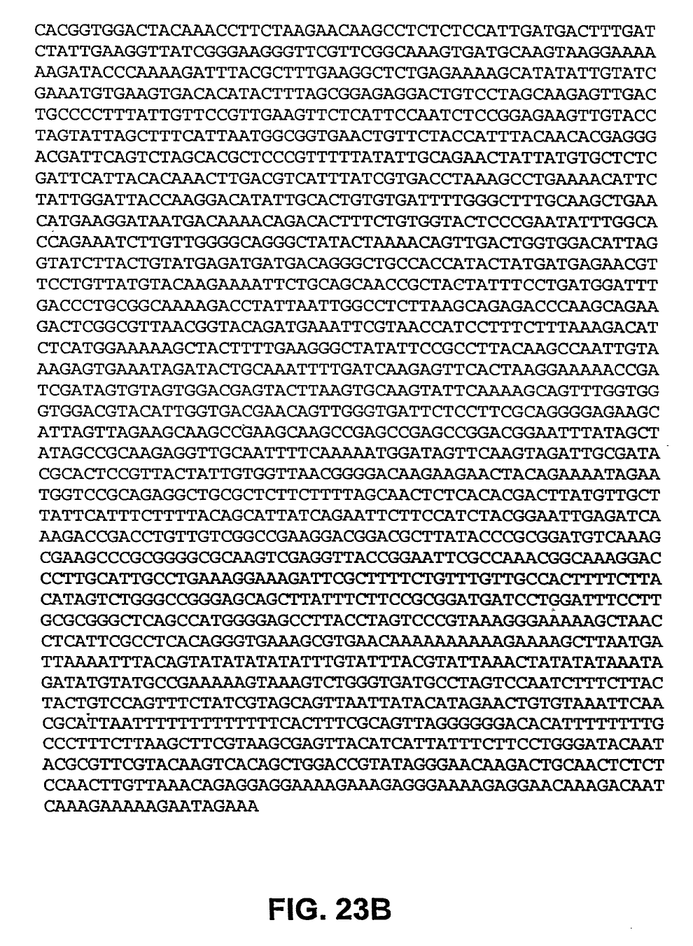

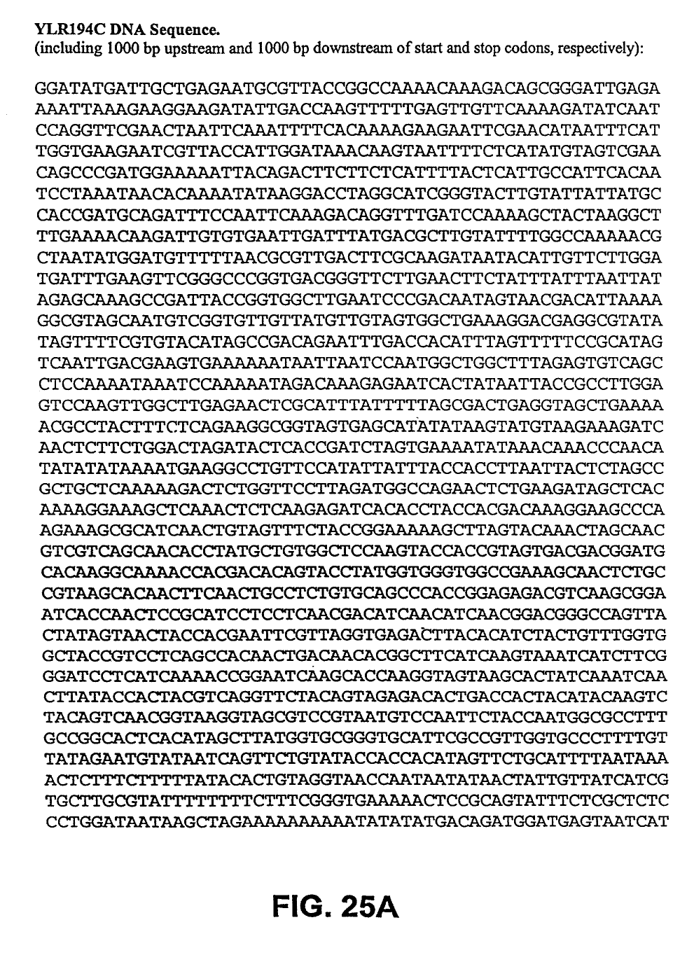

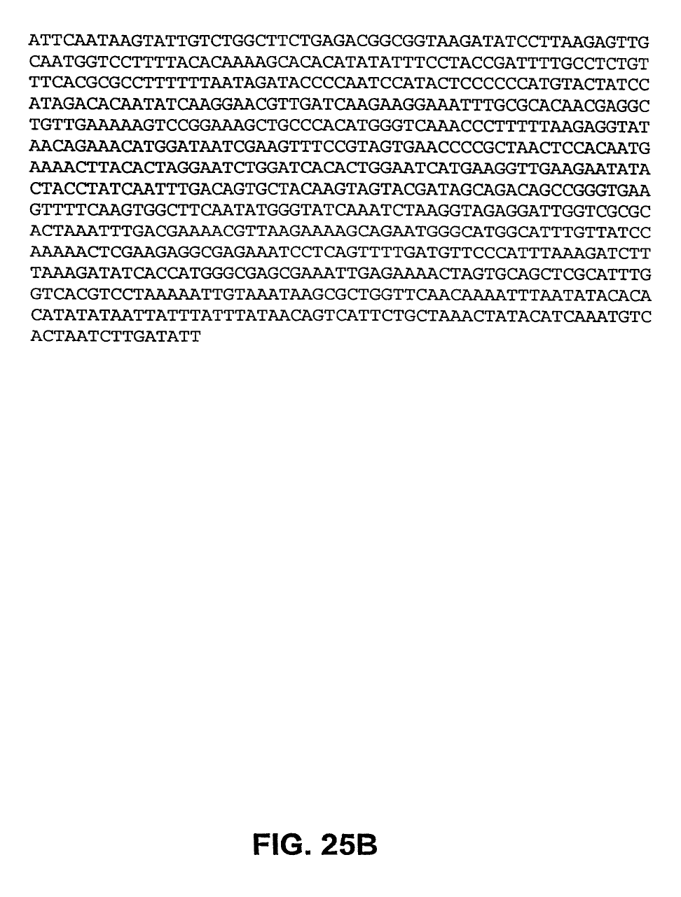

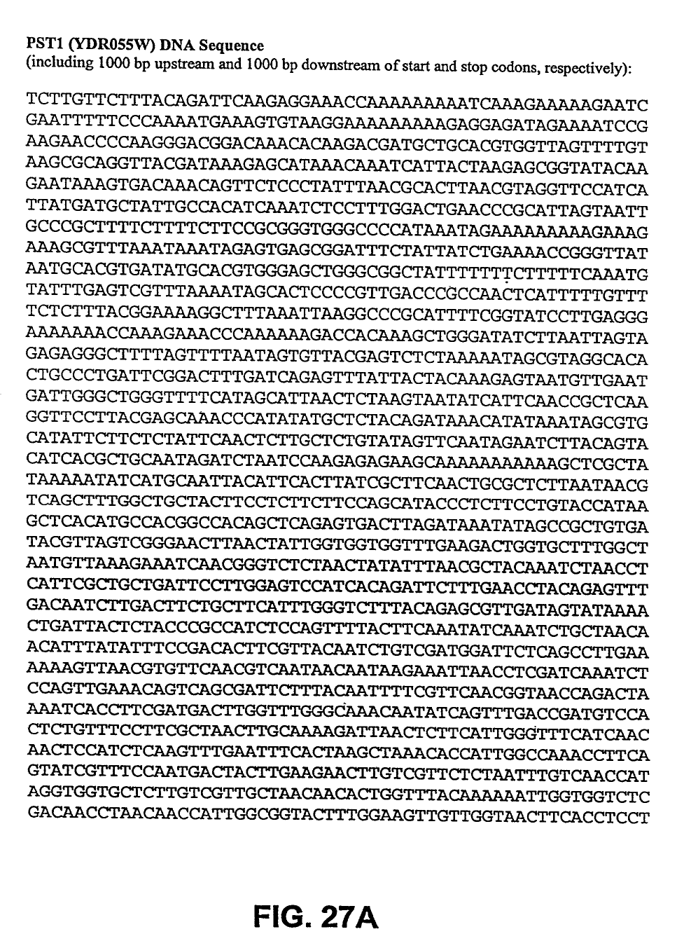

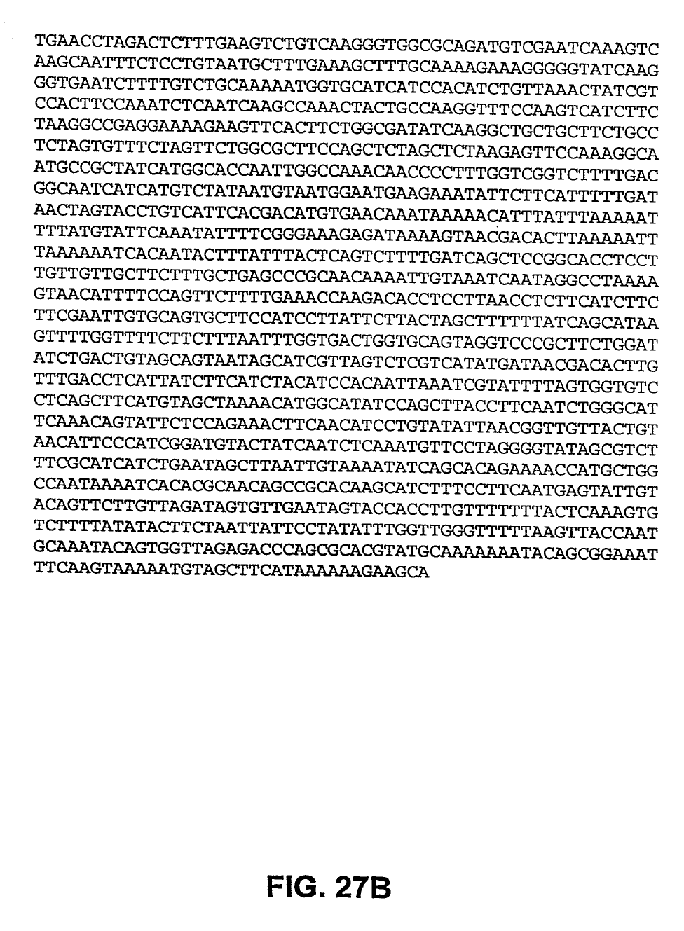

The invention provides a method for determining whether a molecule affects the function or activity of an PKC pathway in a cell comprising: (a) contacting the cell with, or recombinantly expressing within a cell the molecule; and (b) determining whether the expression of one or more of the genes selected from the group consisting of: SLT2(YHR030C) (as depicted in FIGS. 17A-B, as set forth in SEQ ID NO:11), YKR161C (as depicted in FIGS. 19A-B, as set forth in SEQ ID NO:13), PIR3(YKL163W) (as depicted in FIGS. 21A-B, as set forth in SEQ ID NO:15), YPK2(YMR104C) (as depicted in FIGS. 23A-B, as set forth in SEQ ID NO:17), YLR194C (as depicted in FIGS. 25A-B, as set forth in SEQ ID NO:19), and ST1(YDR055W) (as depicted in FIGS. 27A-B, as set forth in SEQ ID NO:21) is changed relative to said expression in the absence of the molecule.

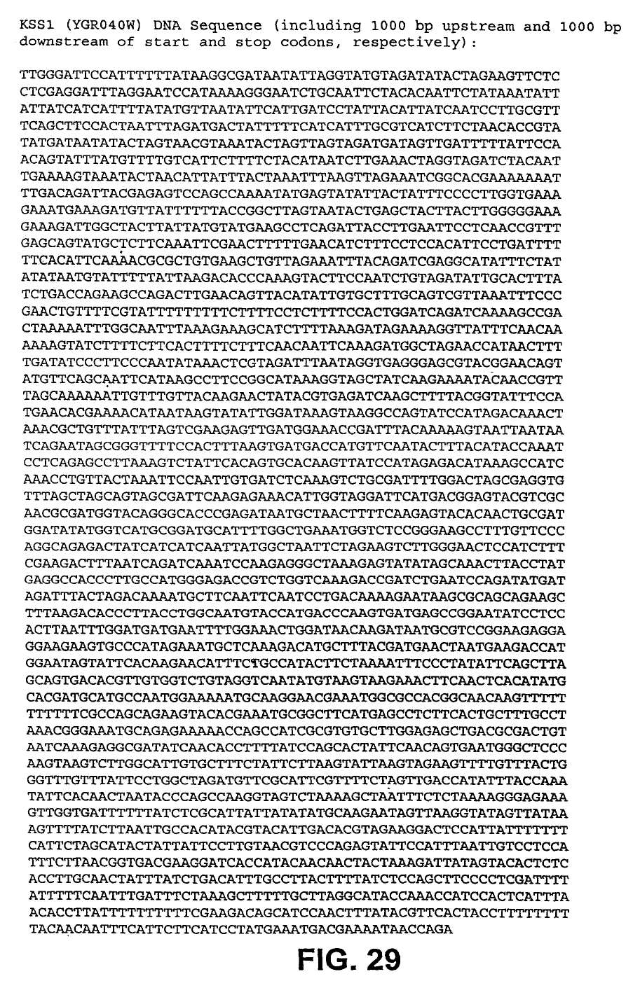

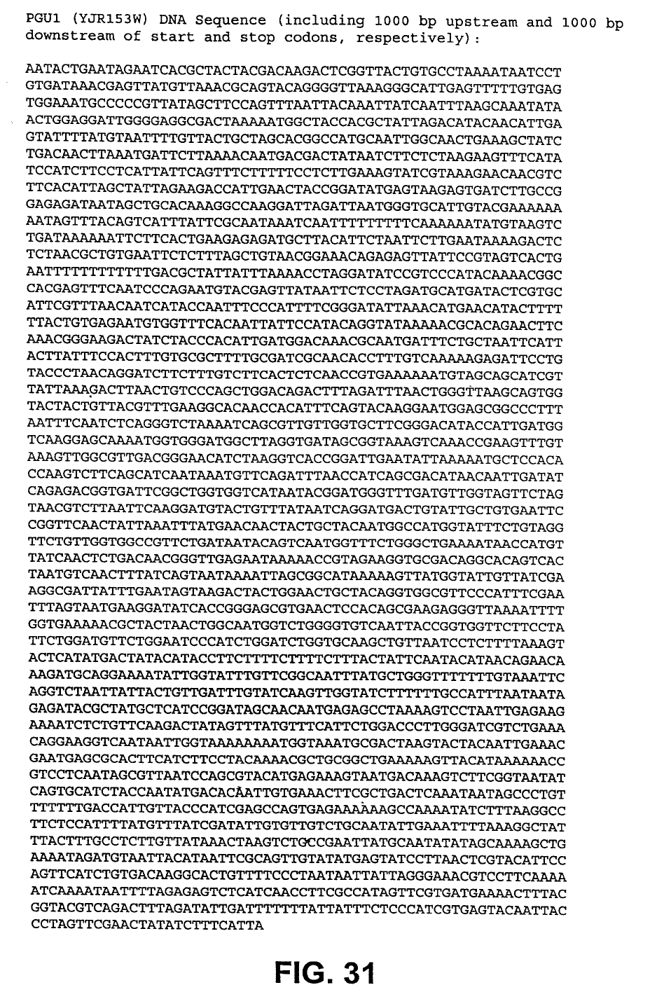

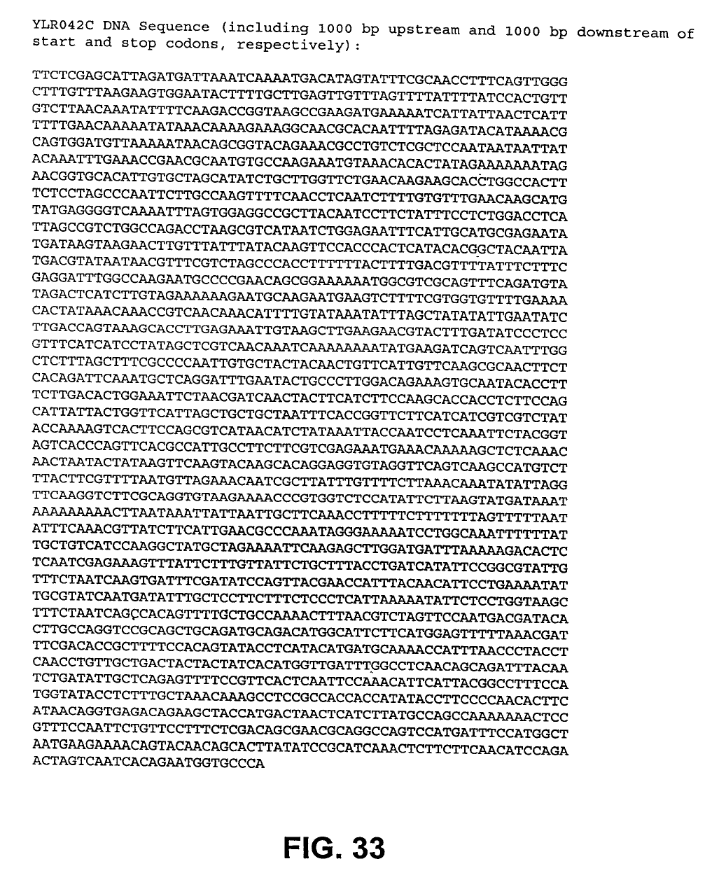

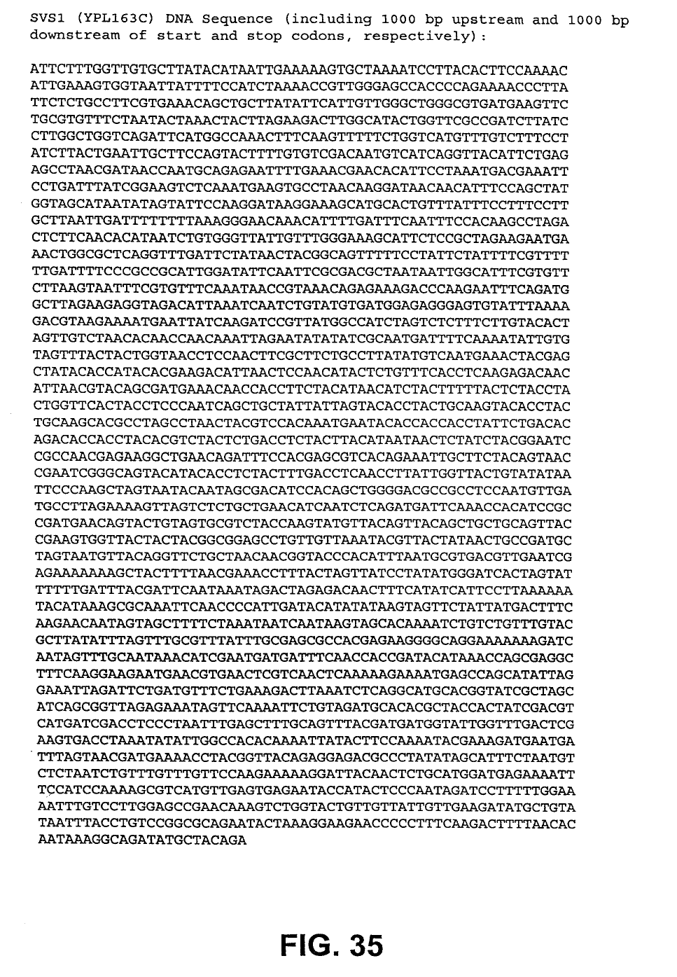

The invention provides a method for determining whether a molecule affects the function or activity of an S. cerevisiae Invasive Growth pathway in a cell comprising: (a) contacting the cell with, or recombinantly expressing within a cell the molecule; and (b) determining whether the expression of one or more of the genes selected from the group consisting of: KSS1(YGR040W) (as depicted in FIG. 29, as set forth in SEQ ID NO:23), PGU1(YJR153W) (as depicted in FIG. 31, as set forth in SEQ ID NO:25), YRL042C (as depicted in FIG. 33, as set forth in SEQ ID NO:27), and SVS1(YPL163C) (as depicted in FIG. 35, as set forth in SEQ ID NO:29), is changed relative to said expression in the absence of the molecule.

5.2.1. Cluster Analysis Algorithms

Response profiles having been thus obtained and, optionally, screened to selected genes with significant responses, the genes and/or the individual response profiles are each grouped according to their similarities (FIG. 1, 103 and 104). In particular, the genes being analyzed according to the methods of the present invention are grouped or re-ordered into co-varying sets (FIG. 1, 103). Likewise, a similar grouping may be optionally performed to group the response profiles according to their similarity (FIG. 1, 104). The steps of grouping the genes and grouping the response profiles may be performed in any order; i.e., the genes may be grouped first Preferably the genes and/or response profiles are each grouped by means of a pattern recognition procedure or algorithm, most preferably by means of a clustering procedure or algorithm. Such algorithms are well known to those of skill in the art, and are reviewed, e.g., by Fukunaga, 1990, Statistical Pattern Recognition, 2nd Ed., London: Academic Press; Everitt, 1974, Cluster Analysis, London: Heinemann Educ. Books; Hartigan, 1975, Clusterin g Algorithms, New York: Wiley; Sneath & Sokal, 1973, Numerical Taxonomy, Freeman; and Anderberg, 1973, Cluster Analysis for Applications, New York: Academic Press, each of which is incorporated herein, by reference, in its entirety. Such algorithms include, for example, hierarchical agglomerative clustering algorithms, the “k-means” algorithm of Hartigan (supra), and model-based clustering algorithms such as hclust by MathSoft, Inc. In one preferred embodiment, the clustering analysis of the present invention is done using a hierarchical clustering algorithm, most preferably the hclust algorithm (see, e.g., ‘hclust’ routine from the software package S-Plus, MathSoft, Inc., Cambridge Mass.).

The clustering algorithms used in the present invention operate on tables of data containing gene expression measurements such as those described above. Specifically, the data tables analyzed by the clustering methods of the present invention comprise an m×k array or matrix wherein m is the total number of experimental conditions or perturbations and k is the number of genes measured and/or analyzed.

The clustering algorithms of the invention analyze such arrays or matrices to determine dissimilarities between the individual genes or between individual response profiles. For example, the dissimilarity between two genes i and j may be expressed mathematically as the “distance” Iᵢⱼ. A variety of distance metrics which are known to those skilled in the art which may be used in the clustering algorithms of the invention. For example, in one embodiment, the euclidian distance is determined according to the formula

$${I}_{i , j} = {[ {\sum }_{n} {( {v}_{i}^{( n )} - {v}_{j}^{( n )} )}^{2} ]}^{1 / 2} \qquad ( 1 )$$

wherein vᵢ⁽ⁿ⁾ and vⱼ⁽ⁿ⁾ are the response of genes i and j respectively to the perturbation n. In other embodiment, the Euclidian distance in Equation 1 above is squared to place progressively greater weight on cellular constituents that are further apart. In alternative embodiments, the distance measure Iᵢⱼ is the Manhattan distance provided by

$${I}_{i , j} = {\sum }_{n} \vert {v}_{i}^{( n )} - {v}_{j}^{( n )} \vert \qquad ( 2 )$$

In certain other embodiments the response profile data is categorical (i.e., each the measured changes in gene expression is represented as either 1 or 0 in each profile), and the distance measure is preferably a percent disagreement defined by:

$${I}_{i , j} = \frac{( No . of {v}_{i}^{( n )} \neq {v}_{j}^{( n )} )}{N} \qquad ( 3 )$$

wherein N is the total number of response profiles.

In particularly preferred embodiments, the distance is defined as Iᵢⱼ=1−rᵢⱼ, wherein rᵢⱼ is the “correlation coefficient” or normalized “dot product” between the genes i and j. In particular, rᵢⱼ is preferably defined by

$${r}_{i , j} = \frac{{v}_{i} \cdot {v}_{j}}{\vert {v}_{i} ‖ {v}_{j} \vert} \qquad ( 4 )$$

wherein the dot product vᵢ·vⱼ is provided by the expression

$${v}_{i} \cdot {v}_{j} = {\sum }_{n} ( {v}_{i}^{( n )} \times {v}_{j}^{( n )} ) \qquad \text{(5)}$$

and |vᵢ|=(vᵢ·vᵢ)^{1/2}; |vᵢ|=(vᵢ·vᵢ)^{1/2}.

In still other embodiments, the distance measure may be the Chebychev distance, the power distance, or the percent disagreement; all of which are well known in the art. Most preferably the distance measure is appropriate to the biological questions being asked, i.e., for identifying co-regulated and/or co-varying genesets and, in particular, for identifying reporter genes and/or target genes within such genesets. Thus, in another particularly preferred embodiment, the correlation coefficient comprises a weighted dot product between genes i and j defined by the equation

$${r}_{i , j} = \frac{{\sum }_{n} \frac{{v}_{i}^{( n )} {v}_{j}^{( n )}}{{σ}_{i}^{( n )} {σ}_{i}^{( n )}}}{{[ {\sum }_{n} {( \frac{{v}_{i}^{( n )}}{{σ}_{i}^{( n )}} )}^{2} {\sum }_{n} {( \frac{{v}_{j}^{( n )}}{{σ}_{j}^{( n )}} )}^{2} ]}^{1 / 2}} \qquad ( 6 )$$

wherein σᵢ⁽ⁿ⁾ and σⱼ⁽ⁿ⁾ are the standard errors associated with the measurement of genes i and j respectively in experiment n.

The correlation coefficients of Equations 4 and 6 are bonded between values of +1, which indicates that the two genes are perfectly correlated and essentially identical in their response to perturbations, and −1, which indicates that the two genes are “anti-correlated” or “anti-sense” (i.e., opposites). Thus, these correlation coefficients are particularly preferable in embodiments of the invention where the responses all have the same sign. However, in other embodiments it is preferable to identify genesets which are co-regulated or involved in the same biological response or pathways but which comprise similar and anti-correlated responses. In such embodiments, it is preferable to use the absolute value of Equation 4 or 6, i.e., |rᵢⱼ|, as the correlation coefficient.

In still other embodiments, the relationships between co-regulated and/or co-varying genesets may be even more complex, such as in instances wherein multiple biological pathways (e.g., signaling pathways) converge on the same cellular constituent to produce different outcomes. In such embodiments, it is preferable to use a correlation coefficient rᵢⱼ=rᵢⱼ^{(change)} which is capable of identifying co-varying and/or co-regulated genes irrespective of the sign. The correlation specified by Equation 7 below is particularly useful in such embodiments.

$${R}_{i , j}^{( charge )} = \frac{{\sum }_{n} \vert \frac{{v}_{i}^{( n )}}{{σ}_{i}^{( n )}} \vert \vert \frac{{v}_{j}^{( n )}}{{σ}_{j}^{( n )}} \vert}{{[ {\sum }_{n} {( \frac{{v}_{i}^{( n )}}{{σ}_{i}^{( n )}} )}^{2} {\sum }_{n} {( \frac{{v}_{j}^{( n )}}{{σ}_{j}^{( n )}} )}^{2} ]}^{1 / 2}} \qquad ( 7 )$$

The cluster analysis methods may also be applied “two-dimensionally” in order to perform two-dimensional (2D) clustering analysis on the response profiles. Specifically, the clustering methods of the invention may be used both to cluster genes in co-varying genesets, and cluster response profiles into sets of similar response profiles, i.e., perturbations that produce similar transcriptional responses. Such dual clustering is referred to herein as “two-dimensional clustering” or “two-dimensional cluster analysis”. Distance metrics will be apparent to those skilled in the art for clustering the response profiles which are similar to those described above for clustering of genes. For example, one skilled in the art will readily appreciate that a suitable correlation coefficient r^{(m,n)} for evaluating two response profiles m and n may be provided by a formula analogous to Equation 4 above:

$${r}^{( m , n )} = \frac{{v}^{( n )} \cdot {v}^{( m )}}{\vert {v}^{( n )} ‖ {v}^{( m )} \vert} \qquad ( 8 )$$

wherein the dot product v⁽ⁿ⁾·v^{(m)} is defined in a manner analogous to Equation 5 above, by the formula

$${v}^{( n )} \cdot {v}^{( m )} = {\sum }_{i} ( {v}_{i}^{( n )} \times {v}_{j}^{( n )} ) \qquad ( 9 )$$

where vᵢ⁽ⁿ⁾ and vᵢ^{(m)} are the response of gene i to the perturbations n and m, respectively.

Generally, the clustering algorithms used in the methods of the invention also use one or more linkage rules to group cellular constituents into one or more sets or “clusters.” For example, single linkage or the nearest neighbor method determines the distance between the two closest objects (i.e., between the two closest genes) in a data table. By contrast, complete linkage methods determine the greatest distance between any two objects (i.e., cellular constituents) in different clusters or sets. The unweighted pair-group average evaluates the “distance” between two clusters or sets by determining the average distance between all pairs of objects (i.e., genes) in the two clusters. Alternatively, the weighted pair-group average evaluates the distance between two clusters or sets by determining the weighted average distance between all pairs of objects in the two clusters, wherein the weighing factor is proportional to the size of the respective clusters. Other linkage rules, such as the unweighted and weighted pair-group centroid and Ward's method, are also useful for certain embodiments of the present invention (see, e.g., Ward, 1963, J. Am. Stat. Assn. 58:236; Hartigan, 1975, Clustering Algorithms, New York: Wiley; each of which is incorporated herein by reference in its entirety).

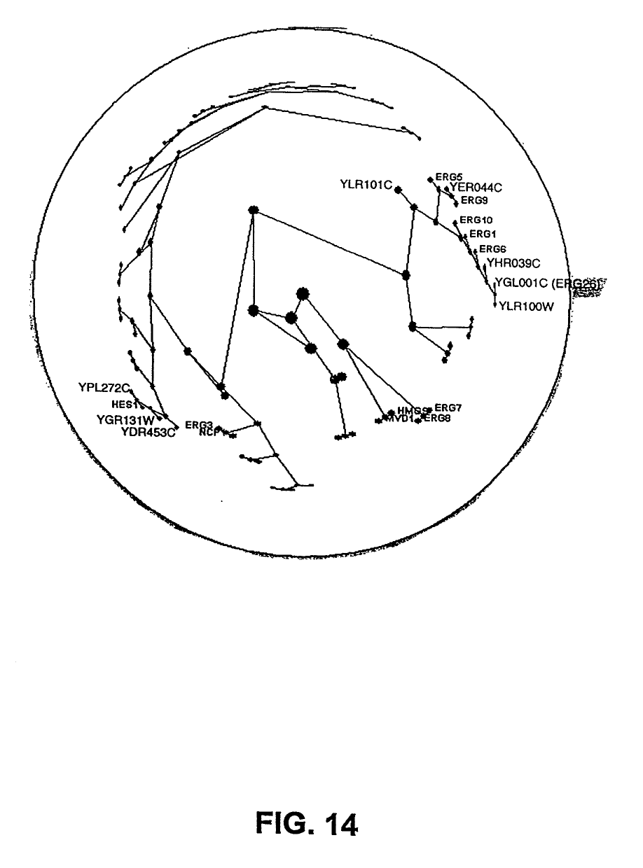

Once a clustering algorithm has grouped the genes from the data table into sets or cluster (i.e., into genesets) by application of linkage rules such as those described supra, a clustering “tree” may be generated to illustrate the genesets so determined. FIG. 14 illustrates an exemplary clustering tree generated by the hclust clustering algorithm upon analysis of a 34×185 table of response profile data using the distance metric Iᵢⱼ=1−rᵢⱼ. The measured response data comprise the logarithm to the base 10 of the ratio between abundances of each transcript in the pair conditions (i.e., perturbation and no perturbation) comprising each experiment n.

Genesets may be readily defined based on the branchings of a clustering tree or diagram such as the one illustrated in FIG. 14. In particular, genesets may be defined based on the many smaller branchings of a clustering tree, or, optionally, larger genesets may be defined corresponding to the larger branches of a clustering tree. Preferably, the choice of branching level at which genesets are defined matches the number of distinct response pathways expected. In embodiments wherein little or no information is available to indicate the number of pathways, the genesets should be defined according to the branching level wherein the branches of the clustering tree are “truly distinct.”

“Truly distinct,” as used herein, is defined, e.g., by a minimum distance value between the individual branches. Typically, the distance values between truly distinct genesets are in the range of 0.2 to 0.4, where a distance of zero corresponds to perfect correlation and a distance of unity corresponds to no correlation. However, distances between truly distinct genesets may be larger in certain embodiments, e.g., wherein there is poorer quality data or fewer experiments in the response profile data. Alternatively, in other embodiments, e.g., having better quality data or more experiments in the profile dataset, the distance between truly distinct genesets may be less than 0.2.

5.2.2. Reporter Genes

Once genesets have been identified, e.g., by means of the above-described cluster analysis methods, reporter genes may be readily identified by anyone who is reasonably skilled in the art. In particular, any gene which clusters to a geneset associated with a particular biological effect or biological pathway is potentially useful as a reporter gene for that biological effect or biological pathway. Genesets associated with a particular biological effect or pathway can be readily identified, e.g., by identifying other genes in the geneset which are associated with the particular biological effect or pathway. Further, the members of a geneset associated with a particular biological effect or pathway will tend to be activated (or inhibited) by perturbations (i.e., in response profiles) which target a particular biological effect or pathway. Thus, geneset associated with a particular biological effect or pathway can also be identified by identifying genesets that respond (i.e., whose members are activated or inhibited) to perturbations that target the particular biological effect or pathway.

Preferably, the reporter genes of the invention also have one or more of the following characteristics. First, the reporter genes of the invention should be highly specific for the biological effect or pathway of interest. In particular, the reporter genes of the present invention should cluster specifically to genesets associated with the biological effect or pathway of interest, and their expression should not be altered, or, less preferably, should only be slightly altered, by perturbations which target other biological effects or pathways.

Second, the reporter genes of the invention preferably have a high level of induction. In particular, the reporter genes of the invention are preferably expressed at high levels, and their level of expression changes significantly in response to perturbations of the biological effect or pathway of interest. For example, in one embodiment, expression of a reporter genes of the invention changes at least two fold in response to a perturbation to the biological effect or pathway of interest. In a more preferred embodiment, expression of a reporter gene of the invention changes by at least ten fold in response to a perturbation to the biological effect or pathway of interest. Most preferably, a reporter gene of the invention will change by a factor of one hundred or more in response to a perturbation to the biological effect or pathway of interest.

The reporter genes of the invention are also preferably sensitive to perturbations to the biological effect or pathway of interest. In particular, preferably the reporter genes of the invention are perturbed (i.e., their expression is up-regulated or down-regulated) at measurable levels in response to only slight perturbations to the biological effect or pathway of interest, such as in response to low doses of a drug which targets the biological effect or pathway of interest. More preferably, the reporter genes of the invention are more sensitive to perturbations to the biological effect or pathway of interest than are other genes in the geneset for that biological effect or pathway.

In most embodiments, the reporter genes of the invention are preferably general reporters for the entire biological effect or pathway of interest. More specifically, the reporter genes preferably cluster, and therefore respond, to perturbations targeted to the entire biological effect or pathway of interest and not just to particular portions thereof (e.g., to early or late steps of a particular biological pathway). However, one skill of the art can readily appreciate that in certain embodiments it will be useful to identify reporter genes for a particular part of a biological effect or pathway of interest. Accordingly, in such embodiments, the reporter genes identified are preferably specific for those particular portions of the biological effect or pathway that are of interest.

Finally, in certain embodiments, the reporter genes of the invention are genes which kinetically induce quickly, and therefore respond quickly to perturbations of the biological effect or pathway of interest. For example, in most embodiments, changes in the reporter genes of the invention will preferably reach steady state within about eight hours after a perturbation (e.g., after exposure to a drug which targets a biological effect or pathway of interest). More preferably, a reporter gene of the invention induces within about six hours after a perturbation. In other preferred embodiments, a reporter gene of the invention induces within about 2 hours, within about ninety minutes, within about sixty minutes, within about thirty minutes, within about ten minutes, or within about seven minutes after a perturbation.

Other embodiments of the invention provides methods for using combinations of genes to construct a more specific reporter for a particular biological pathway in which it is desired to increase the specificity of a particular pathway reporter system. In this embodiment, more than one gene, or cellular constituent in the same biological pathway is used as a reporter for that pathway. By way of example, a reporter gene of the Invasive Growth pathway such as PGU1, and a second gene in the same pathway such as SVS1, may be detected simultaneously as a reporter for the Invasive Growth pathway. Such co-detection can serve to increase the sensitivity of a reporter of a particular biological pathway. Alternatively, for example, the promoter from a first gene of the Invasive Growth pathway, such as PGU1 may be fused to a marker such as GFP (green fluorescent protein), and a the promoter from a second gene in the same pathway such as SVS1, could be fused to BFP (blue fluorescent protein). Detection of the both proteins makers simultaneuosly can thus provides a higher sensitivity. Thus in this embodiment, the reporter of the pathway is a combination of two or more genes. In other embodiment of the invention, a 2-3, 3-5, 5-10 genes are detected simultaneously as a reporter system for a particular biological pathway.

The invention provides a method of identifying a reporter gene for a particular biological pathway in a cell comprising identifying a gene which clusters to a geneset associated with the biological pathway, wherein said gene which clusters to the geneset associated with the particular biological pathway is a reporter gene.

In one embodiment the reporter gene is a reporter for the ergosterol-pathway, and the reporter gene is selected from the group consisting of: YHR039C (as depicted in FIG. 2, as set forth in SEQ ID NO:1), YLW100W (as depicted in FIG. 4, as set forth in SEQ ID NO:3), YPL272C (as depicted in FIG. 6, as set forth in SEQ ID NO:5), YGR131W (as depicted in FIG. 8, as set forth in SEQ ID NO:7), and YDR453C (as depicted in FIG. 10, as set forth in SEQ ID NO:9).

In another embodiment the reporter gene is a reporter for the PKC-pathway, and the reporter gene is selected from the group consisting of: SLT2(YHR030C) (as depicted in FIGS. 17A-B, as set forth in SEQ ID NO:11), YKR161C (as depicted in FIGS. 19A-B, as set forth in SEQ ID NO:13), PIR3(YKL163W) (as depicted in FIGS. 21A-B, as set forth in SEQ ID NO:15), YPK2(YMR104C) (as depicted in FIGS. 23A-B, as set forth in SEQ ID NO:17), YLR194C (as depicted in FIGS. 25A-B, as set forth in SEQ ID NO:19), and ST1(YDR055W) (as depicted in FIGS. 27A-B, as set forth in SEQ ID NO:21).

In another embodiment the reporter gene is a reporter for the Invasive Growth pathway, and the reporter gene selected from the group consisting of KSS1(YGR040W) (as depicted in FIG. 29, as set forth in SEQ ID NO:23), PGU1(YJR153W) (as depicted in FIG. 31, as set forth in SEQ ID NO:25), YRL042C (as depicted in FIG. 33, as set forth in SEQ ID NO:27), and SVS1(YPL163C) (as depicted in FIG. 35, as set forth in SEQ ID NO:29).

5.2.3. Target Genes

Once genesets have been identified, e.g., by means of the above-described cluster analysis methods, target genes may be readily identified in the following manner. Any gene which clusters to a geneset associated with a particular biological effect or biological pathway may be considered a potential target gene and may further be tested to examine whether the expression and/or activity of the gene is necessary for normal activity or function of the pathway. A gene whose expression and/or activity is necessary for normal activity or function of the pathway is therefore useful as a target for drugs designed to enhance, inhibit, or modulate the particular biological pathway. Any method known in the art may be used to examine the necessity of a particular gene to the activity or function of an associated biological pathway. For example, by way of illustration, potential target gene, such as a potential ergosterol-pathway target gene may be validated as a target gene in the following manner.

Once a potential target gene has been identified (e.g., by clustering analysis as described herein), the gene may be examined by mutational analysis to determine whether the gene is essential. Methods for mutational analysis are commonly known in the art. If the potential ergosterol-pathway target gene is essential for normal growth of the yeast, such a gene is a target gene. Such a gene would constitute a preferred target for antifungal or fungicidal drug development. Further, additional genetic analysis may be performed in order to construct and characterize a conditional allele of the gene in order to determine the effects of gene product inhibition, particularly whether the cell dies upon shifting to the restrictive condition, or whether the cell can recover upon shifting back to the permissive condition. Any method known in the art may be used to construct a conditional allele, for example, a temperature sensitive allele, or promoter replacement may be performed so that expression may be regulated. The construction of a conditional allele also allows for the determination of the terminal phenotype, contributing to an understanding of the function of the gene. If, for example, the potential ergosterol-pathway gene is determined not to be essential in S. cerevisiae, or if a severe growth defect does not result from deletion of the gene, the gene is not a preferred target gene for the development of a pathway-specific drug such as an antifungal agent.

Another way in which a potential target gene may be validated is by searching the sequence database for a homolog genes. For example, in the case of an S. cerevisiae target gene, a database from the yeast Candida may serve as a database for which to compare sequence. Alternatively, a search of all sequence databases may be performed to uncover sequence motifs that will reveal potential activities of the gene. Specifically, by way of example computer programs for determining homology include but are not limited to TBLASTN, BLASTP, FASTA, TFASTA, and CLUSTALW (Pearson and Lipman, 1988, Proc. Natl. Acad. Sci. USA 85(8):2444-8; Altschul et al., 1990, J. Mol. Biol. 215(3):403-10; Thompson, et al., 1994, Nucleic Acids Res. 22(22):4673-80; Higgins, et al., 1996, Methods Enzymol 266:383-402; Altschul, et al., 1990, J. Mol. Biol. 215(3):403-10). If, for example, a homolog of the S. cerevisiae target gene is found in Candida, the Candida gene may be analyzed as above to determine whether the homolog is essential in Candida, and would constitute a validated target.

The invention provides a method of identifying a target gene for a particular biological pathway in a cell comprising identifying a gene which clusters to a geneset associated with the particular biological pathway, wherein said gene which clusters to a geneset associated with the particular biological pathway and is identified as a gene which is necessary for normal function of said particular biological pathway.

### 5.3. Perturbation Methods

Methods for perturbation of biological pathways at various levels of a cell are increasingly widely known and applied in the art. Any such methods that are capable of specifically targeting and controllably modifying (e.g., either by a graded increase or activation or by a graded decrease or inhibition) specific cellular constituents (e.g., gene expression, RNA concentrations, protein abundances, protein activities, or so forth) can be employed in performing pathway perturbations. Controllable modifications of cellular constituents consequentially controllably perturb pathways originating at the modified cellular constituents. Such pathways originating at specific cellular constituents are preferably employed to represent drug action in this invention. Preferable modification methods are capable of individually targeting each of a plurality of cellular constituents and most preferably a substantial fraction of such cellular constituents.

The following methods are exemplary of those that can be used to modify cellular constituents and thereby to produce pathway perturbations which generate the pathway responses used in the steps of the methods of this invention as previously described. This invention is adaptable to other methods for making controllable perturbations to pathways, and especially to cellular constituents from which pathways originate.

Pathway perturbations are preferably made in cells of cell types derived from any organism for which genomic or expressed sequence information is available and for which methods are available that permit controllably modification of the expression of specific genes. Genome sequencing is currently underway for several eukaryotic organisms, including humans, nematodes, Arabidopsis, and flies. In a preferred embodiment, the invention is carried out using a yeast, with Saccharomyces cerevisiae most preferred because the sequence of the entire genome of a S. cerevisiae strain has been determined. In addition, well-established methods are available for controllably modifying expression of year genes. A preferred strain of yeast is a S. cerevisiae strain for which yeast genomic sequence is known, such as strain S288C or substantially isogeneic derivatives of it (see, e.g., Dujon et al., 1994, Nature 369:371-378; Bussey et al., 1995, Proc. Natl. Acad. Sci. U.S.A. 92:3809-3813; Feldmann et al., 1994, E.M.B.O. J. 13:5795-5809; Johnston et al., 1994, Science 265:2077-2082; Galibert et al., 1996, E.M.B.O. J. 15:2031-2049). However, other strains may be used as well. Yeast strains are available, e.g., from American Type Culture Collection, 10801 University Boulevard, Manassas, Va. 20110-2209. Standard techniques for manipulating yeast are described in C. Kaiser, S. Michaelis, & A. Mitchell, 1994, Methods in Yeast Genetics: A Cold Spring Harbor Laboratory Course Manual, Cold Spring Harbor Laboratory Press, New York; and Sherman et al., 1986, Methods in Yeast Genetics: A Laboratory Manual, Cold Spring Harbor Laboratory, Cold Spring Harbor. N.Y.

The exemplary methods described in the following include use of titratable expression systems, use of transfection or viral transduction systems, direct modifications to RNA abundances or activities, direct modifications of protein abundances, and direct modification of protein activities including use of drugs (or chemical moieties in general) with specific known action.

5.3.1. Titratable Expression Systems

Any of the several known titratable, or equivalently controllable, expression systems available for use in the budding yeast Saccharomyces cerevisiae are adaptable to this invention (Mumberg et al., 1994, Nucl. Acids Res. 22:5767-5768). Usually, gene expression is controlled by transcriptional controls, with the promoter of the gene to be controlled replaced on its chromosome by a controllable, exogenous promoter. The most commonly used controllable promoter in yeast is the GAL1 promoter (Johnston et al., 1984, Mol Cell. Biol. 8:1440-1448). The GAL1 promoter is strongly repressed by the presence of glucose in the growth medium, and is gradually switched on in a graded manner to high levels of expression by the decreasing abundance of glucose and the presence of galactose. The GAL1 promoter usually allows a 5-100 fold range of expression control on a gene of interest.

Other frequently used promoter systems include the MET25 promoter (Kerjan et al., 1986, Nuc. Acids. Res. 14:7861-7871), which is induced by the absence of methionine in the growth medium, and the CUP1 promoter, which is induced by copper (Mascorro-Gallardo et al., 1996, Gene 172:169-170). All of these promoter systems are controllable in that gene expression can be incrementally controlled by incremental changes in the abundances of a controlling moiety in the growth medium.

One disadvantage of the above listed expression systems is that control of promoter activity (effected by, e.g., changes in carbon source, removal of certain amino acids), often causes other changes in cellular physiology which independently alter the expression levels of other genes. A recently developed system for yeast, the Tet system, alleviates this problem to a large extent (Gari et al., 1997, Yeast 13:837-848). The Tet promoter, adopted from mammalian expression systems (Gossen et al., 1995, Proc. Nat. Acad. Sci. USA 89:5547-5551) is modulated by the concentration of the antibiotic tetracycline or the structurally related compound doxycycline. Thus, in the absence of doxycycline, the promoter induces a high level of expression, and the addition of increasing levels of doxycycline causes increased repression of promoter activity. Intermediate levels gene expression can be achieved in the steady state by addition of intermediate levels of drug. Furthermore, levels of doxycycline that give maximal repression of promoter activity (10 micrograms/ml) have no significant effect on the growth rate on wild type yeast cells (Gari et al., 1997, Yeast 13:837-848).

In mammalian cells, several means of titrating expression of genes are available (Spencer, 1996, Trends Genet. 12:181-187). As mentioned above, the Tet system is widely used, both in its original form, the “forward” system, in which addition of doxycycline represses transcription, and in the newer “reverse” system, in which doxycycline addition stimulates transcription (Gossen et al., 1995, Proc. Natl. Acad. Sci. USA 89:5547-5551; Hoffmann et al., 1997, Nucl. Acids. Res. 25:1078-1079; Hofmann et al., 1996, Proc. Natl. Acad. Sci. USA 83:5185-5190; Paulus et al., 1996, Journal of Virology 70:62-67). Another commonly used controllable promoter system in mammalian cells is the ecdysone-inducible system developed by Evans and colleagues (No et al., 1996, Proc. Nat. Acad. Sci. USA 93:3346-3351), where expression is controlled by the level of muristerone added to the cultured cells. Finally, expression can be modulated using the “chemical-induced dimerization” (CID) system developed by Schreiber, Crabtree, and colleagues (Belshaw et al., 1996, Proc. Nat. Acad. Sci. USA 93:4604-4607; Spencer, 1996, Trends Genet. 12:181-187) and similar systems in yeast. In this system, the gene of interest is put under the control of the CID-responsive promoter, and transfected into cells expressing two different hybrid proteins, one comprised of a DNA-binding domain fused to FKBP12, which binds FK506. The other hybrid protein contains a transcriptional activation domain also fused to FKBP12. The CID inducing molecule is FK1012, a homodimeric version of FK506 that is able to bind simultaneously both the DNA binding and transcriptional activating hybrid proteins. In the graded presence of FK1012, graded transcription of the controlled gene is activated.

For each of the mammalian expression systems described above, as is widely known to those of skill in the art, the gene of interest is put under the control of the controllable promoter, and a plasmid harboring this construct along with an antibiotic resistance gene is transfected into cultured mammalian cells. In general, the plasmid DNA integrates into the genome, and drug resistant colonies are selected and screened for appropriate expression of the regulated gene. Alternatively, the regulated gene can be inserted into an episomal plasmid such as pCEP4 (Invitrogen, Inc.), which contains components of the Epstein-Barr virus necessary for plasmid replication.

In a preferred embodiment, titratable expression systems, such as the ones described above, are introduced for use into cells or organisms lacking the corresponding endogenous gene and/or gene activity, e.g., organisms in which the endogenous gene has been disrupted or deleted. Methods for producing such “knock outs” are well known to those of skill in the art, see e.g., Pettitt et al., 1996, Development 122:4149-4157; Spradling et al., 1995, Proc. Natl. Acad. Sci. USA, 92:10824-10830; Ramirez-Solis et al., 1993, Methods Enzymol. 225:855-878; and Thomas et al., 1987, Cell 51:503-512.

5.3.2. Transfection Systems for Mammalian Cells

Transfection or viral transduction of target genes can introduce controllable perturbations in biological pathways in mammalian cells. Preferably, transfection or transduction of a target gene can be used with cells that do not naturally express the target gene of interest. Such non-expressing cells can be derived from a tissue not normally expressing the target gene or the target gene can be specifically mutated in the cell. The target gene of interest can be cloned into one of many mammalian expression plasmids, for example, the pcDNA3.1 +/− system (Invitrogen, Inc.) or retroviral vectors, and introduced into the non-expressing host cells. Transfected or transduced cells expressing the target gene may be isolated by selection for a drug resistance marker encoded by the expression vector. The level of gene transcription is monotonically related to the transfection dosage. In this way, the effects of varying levels of the target gene may be investigated.

A particular example of the use of this method is the search for drugs that target the src-family protein tyrosine kinase, lck, a key component of the T cell receptor activation pathway (Anderson et al., 1994, Adv. Immunol. 56:171-178). Inhibitors of this enzyme are of interest as potential immunosuppressive drugs (Hanke J H, 1996, J. Biol Chem 271(2):695-701). A specific mutant of the Jurkat T cell line (JcaM1) is available that does not express lck kinase (Straus et al., 1992, Cell 70:585-593). Therefore, introduction of the lck gene into JCaM1 by transfection or transduction permits specific perturbation of pathways of T cell activation regulated by the lck kinase. The efficiency of transfection or transduction, and thus the level of perturbation, is dose related. The method is generally useful for providing perturbations of gene expression or protein abundances in cells not normally expressing the genes to be perturbed.

5.3.3. Methods of Modifying RNA Abundances or Activities

Methods of modifying RNA abundances and activities currently fall within three classes, ribozymes, antisense species, and RNA aptamers (Good et al., 1997, Gene Therapy 4: 45-54). Controllable application or exposure of a cell to these entities permits controllable perturbation of RNA abundances.

Ribozymes are RNAs which are capable of catalyzing RNA cleavage reactions. (Cech, 1987, Science 236:1532-1539; PCT International Publication WO 90/11364, published Oct. 4, 1990; Sarver et al., 1990, Science 247: 1222-1225). “Hairpin” and “hammerhead” RNA ribozymes can be designed to specifically cleave a particular target mRNA. Rules have been established for the design of short RNA molecules with ribozyme activity, which are capable of cleaving other RNA molecules in a highly sequence specific way and can be targeted to virtually all kinds of RNA. (Haseloff et al., 1988, Nature 334:585-591; Koizumi et al., 1988, FEBS Lett. 228:228-230; Koizumi et al., 1988, FEBS Lett. 239:285-288). Ribozyme methods involve exposing a cell to, inducing expression in a cell, etc. of such small RNA ribozyme molecules. (Grassi and Marini, 1996, Annals of Medicine 28: 499-510; Gibson, 1996, Cancer and Metastasis Reviews 15: 287-299).

Ribozymes can be routinely expressed in vivo in sufficient number to be catalytically effective in cleaving mRNA, and thereby modifying mRNA abundances in a cell. (Cotten et al., 1989, EMBO J. 8:3861-3866). In particular, a ribozyme coding DNA sequence, designed according to the previous rules and synthesized, for example, by standard phosphoramidite chemistry, can be ligated into a restriction enzyme site in the anticodon stem and loop of a gene encoding a tRNA, which can then be transformed into and expressed in a cell of interest by methods routine in the art. Preferably, an inducible promoter (e.g., a glucocorticoid or a tetracycline response element) is also introduced into this construct so that ribozyme expression can be selectively controlled. tDNA genes (i.e., genes encoding tRNAs) are useful in this application because of their small size, high rate of transcription, and ubiquitous expression in different kinds of tissues. Therefore, ribozymes can be routinely designed to cleave virtually any mRNA sequence, and a cell can be routinely transformed with DNA coding for such ribozyme sequences such that a controllable and catalytically effective amount of the ribozyme is expressed. Accordingly the abundance of virtually any RNA species in a cell can be perturbed.

In another embodiment, activity of a target RNA (preferable mRNA) species, specifically its rate of translation, can be controllably inhibited by the controllable application of antisense nucleic acids. An “antisense” nucleic acid as used herein refers to a nucleic acid capable of hybridizing to a sequence-specific (e.g., non-poly A) portion of the target RNA, for example its translation initiation region, by virtue of some sequence complementarity to a coding and/or non-coding region. The antisense nucleic acids of the invention can be oligonucleotides that are double-stranded or single-stranded, RNA or DNA or a modification or derivative thereof, which can be directly administered in a controllable manner to a cell or which can be produced intracellularly by transcription of exogenous, introduced sequences in controllable quantities sufficient to perturb translation of the target RNA.

Preferably, antisense nucleic acids are of at least six nucleotides and are preferably oligonucleotides (ranging from 6 to about 200 oligonucleotides). In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof, single-stranded or double-stranded. The oligonucleotide can be modified at the base moiety, sugar moiety, or phosphate backbone. The oligonucleotide may include other appending groups such as peptides, or agents facilitating transport across the cell membrane (see, e.g., Letsinger et al., 1989, Proc. Natl. Acad. Sci. U.S.A. 86: 6553-6556; Lemaitre et al., 1987, Proc. Natl. Acad. Sci. U.S.A. 84: 648-652; PCT Publication No. WO 88/09810, published Dec. 15, 1988), hybridization-triggered cleavage agents (see, e.g., Krol et al., 1988, BioTechniques 6: 958-976) or intercalating agents (see, e.g., Zon, 1988, Pharm. Res. 5: 539-549).

In a preferred aspect of the invention, an antisense oligonucleotide is provided, preferably as single-stranded DNA. The oligonucleotide may be modified at any position on its structure with constituents generally known in the art.

The antisense oligonucleotides may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

In another embodiment, the oligonucleotide comprises at least one modified sugar moiety selected from the group including, but not limited to, arabinose, 2-fluoroarabinose, xylulose, and hexose.

In yet another embodiment, the oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the oligonucleotide is a 2-α-anomeric oligonucleotide. An α-anomeric oligonucleotide forms specific double-stranded hybrids with complementary RNA in which, contrary to the usual β-units, the strands run parallel to each other (Gautier et al., 1987, Nucl Acids Res. 15: 6625-6641).

The oligonucleotide may be conjugated to another molecule, e.g., a peptide, hybridization triggered cross-linking agent, transport agent, hybridization-triggered cleavage agent, etc.

The antisense nucleic acids of the invention comprise a sequence complementary to at least a portion of a target RNA species. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a target RNA it may contain and still form a stable duplex (or triplex, as the case may be). One skilled in the art can ascertain a tolerable degree of mismatch by use of standard procedures to determine the melting point of the hybridized complex. The amount of antisense nucleic acid that will be effective in the inhibiting translation of the target RNA can be determined by standard assay techniques.

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16: 3209), methylphosphonate oligonucleotides can be prepared by use of controlled pore glass polymer supports (Sarin et al., 1988, Proc. Natl. Acad. Sci. U.S.A. 85: 7448-7451), etc. In another embodiment, the oligonucleotide is a 2′-0-methylribonucleotide (Inoue et al., 1987, Nucl. Acids Res. 15: 6131-6148), or a chimeric RNA-DNA analog (Inoue et al., 1987, FEBS Lett. 215: 327-330).

The synthesized antisense oligonucleotides can then be administered to a cell in a controlled manner. For example, the antisense oligonucleotides can be placed in the growth environment of the cell at controlled levels where they may be taken up by the cell. The uptake of the antisense oligonucleotides can be assisted by use of methods well known in the art.

In an alternative embodiment, the antisense nucleic acids of the invention are controllably expressed intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequences encoding the antisense RNAs can be by any promoter known in the art to act in a cell of interest. Such promoters can be inducible or constitutive. Most preferably, promoters are controllable or inducible by the administration of an exogenous moiety in order to achieve controlled expression of the antisense oligonucleotide. Such controllable promoters include the Tet promoter. Less preferably usable promoters for mammalian cells include, but are not limited to: the SV40 early promoter region (Bernoist and Chambon, 1981, Nature 290: 304-310), the promoter contained in the 3′ long terminal repeat of Rous sarcoma virus (Yamamoto et al., 1980, Cell 22: 787-797), the herpes thymidine kinase promoter (Wagner et al., 1981, Proc. Natl. Acad. Sci. U.S.A. 78: 1441-1445), the regulatory sequences of the metallothionein gene (Brinster et al., 1982, Nature 296: 39-42), etc.

Therefore, antisense nucleic acids can be routinely designed to target virtually any mRNA sequence, and a cell can be routinely transformed with or exposed to nucleic acids coding for such antisense sequences such that an effective and controllable amount of the antisense nucleic acid is expressed. Accordingly the translation of virtually any RNA species in a cell can be controllably perturbed.

Finally, in a further embodiment, RNA aptamers can be introduced into or expressed in a cell. RNA aptamers are specific RNA ligands for proteins, such as for Tat and Rev RNA (Good et al., 1997, Gene Therapy 4: 45-54) that can specifically inhibit their translation.

In specific embodiments of the invention methods of modifying RNA abundances and activities are used to modify an RNA corresponding to a target gene or reporter gene of the invention. In other specific embodiments of the invention, a ribozymes, antisense species, and RNA aptamers directed to a target gene of the invention is used as a drug or therapeutic agent.

5.3.4. Methods of Modifying Protein Abundances

Methods of modifying protein abundances include, inter alia, those altering protein degradation rates and those using antibodies (which bind to proteins affecting abundances of activities of native target protein species). Increasing (or decreasing) the degradation rates of a protein species decreases (or increases) the abundance of that species. Methods for controllably increasing the degradation rate of a target protein in response to elevated temperature and/or exposure to a particular drug, which are known in the art, can be employed in this invention. For example, one such method employs a heat-inducible or drug-inducible N-terminal degron, which is an N-terminal protein fragment that exposes a degradation signal promoting rapid protein degradation at a higher temperature (e.g., 37° C.) and which is hidden to prevent rapid degradation at a lower temperature (e.g., 23° C.) (Dohmen et al., 1994, Science 263:1273-1276). Such an exemplary degron is Arg-DHFR^{ts}, a variant of murine dihydrofolate reductase in which the N-terminal Val is replaced by Arg and the Pro at position 66 is replaced with Leu. According to this method, for example, a gene for a target protein, P, is replaced by standard gene targeting methods known in the art (Lodish et al., 1995, Molecular Biology of the Cell, Chpt. 8, New York: W. H. Freeman and Co.) with a gene coding for the fusion protein Ub-Arg-DHFR^{ts}-P (“Ub” stands for ubiquitin). The N-terminal ubiquitin is rapidly cleaved after translation exposing the N-terminal degron. At lower temperatures, lysines internal to Arg-DHFR^{ts} are not exposed, ubiquitination of the fusion protein does not occur, degradation is slow, and active target protein levels are high. At higher temperatures (in the absence of methotrexate), lysines internal to Arg-DHFR^{ts} are exposed, ubiquitination of the fusion protein occurs, degradation is rapid, and active target protein levels are low. Heat activation of degradation is controllably blocked by exposure methotrexate. This method is adaptable to other N-terminal degrons which are responsive to other inducing factors, such as drugs and temperature changes.

Target protein abundances and also, directly or indirectly, their activities can also be decreased by (neutralizing) antibodies. By providing for controlled exposure to such antibodies, protein abundances/activities can be controllably modified. For example, antibodies to suitable epitopes on protein surfaces may decrease the abundance, and thereby indirectly decrease the activity, of the wild-type active form of a target protein by aggregating active forms into complexes with less or minimal activity as compared to the wild-type unaggregated wild-type form. Alternately, antibodies may directly decrease protein activity by, e.g., interacting directly with active sites or by blocking access of substrates to active sites. Conversely, in certain cases, (activating) antibodies may also interact with proteins and their active sites to increase resulting activity. In either case, antibodies (of the various types to be described) can be raised against specific protein species (by the methods to be described) and their effects screened. The effects of the antibodies can be assayed and suitable antibodies selected that raise or lower the target protein species concentration and/or activity. Such assays involve introducing antibodies into a cell (see below), and assaying the concentration of the wild-type amount or activities of the target protein by standard means (such as immunoassays) known in the art. The net activity of the wild-type form can be assayed by assay means appropriate to the known activity of the target protein.

Antibodies can be introduced into cells in numerous fashions, including, for example, microinjection of antibodies into a cell (Morgan et al., 1988, Immunology Today 9:84-86) or transforming hybridoma mRNA encoding a desired antibody into a cell (Burke et al., 1984, Cell 36:847-858). In a further technique, recombinant antibodies can be engineering and ectopically expressed in a wide variety of non-lymphoid cell types to bind to target proteins as well as to block target protein activities (Biocca et al., 1995, Trends in Cell Biology 5:248-252). Preferably, expression of the antibody is under control of a controllable promoter, such as the Tet promoter. A first step is the selection of a particular monoclonal antibody with appropriate specificity to the target protein (see below). Then sequences encoding the variable regions of the selected antibody can be cloned into various engineered antibody formats, including, for example, whole antibody, Fab fragments, Fv fragments, single chain Fv fragments (V_{H} and V_{L} regions united by a peptide linker) (“ScFv” fragments), diabodies (two associated ScFv fragments with different specificities), and so forth (Hayden et al., 1997, Current Opinion in Immunology 9:210-212). Intracellularly expressed antibodies of the various formats can be targeted into cellular compartments (e.g., the cytoplasm, the nucleus, the mitochondria, etc.) by expressing them as fusions with the various known intracellular leader sequences (Bradbury et al., 1995, Antibody Engineering, vol. 2, Borrebaeck ed., IRL Press, pp 295-361). In particular, the ScFv format appears to be particularly suitable for cytoplasmic targeting.

Antibody types include, but are not limited to, polyclonal, monoclonal, chimeric, single chain, Fab fragments, and an Fab expression library. Various procedures known in the art may be used for the production of polyclonal antibodies to a target protein. For production of the antibody, various host animals can be immunized by injection with the target protein, such host animals include, but are not limited to, rabbits, mice, rats, etc. Various adjuvants can be used to increase the immunological response, depending on the host species, and include, but are not limited to, Freund's (complete and incomplete), mineral gels such as aluminum hydroxide, surface active substances such as lysolecithin, pluronic polyols, polyanions, peptides, oil emulsions, dinitrophenol, and potentially useful human adjuvants such as bacillus Cahnette-Guerin (BCG) and corynebacterium parvum.

For preparation of monoclonal antibodies directed towards a target protein, any technique that provides for the production of antibody molecules by continuous cell lines in culture may be used. Such techniques include, but are not restricted to, the hybridoma technique originally developed by Kohler and Milstein (1975, Nature 256: 495-497), the trioma technique, the human B-cell hybridoma technique (Kozbor et al., 1983, Immunology Today 4: 72), and the EBV hybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. U.S.A. 80: 2026-2030), or by transforming human B cells with EBV virus in vitro (Cole et al., 1985, in Monoclonal Antibodies and Cancer Therapy, Alan R. Liss, Inc., pp. 77-96). In fact, according to the invention, techniques developed for the production of “chimeric antibodies” (Morrison et al., 1984, Proc. Natl. Acad. Sci. U.S.A. 81: 6851-6855; Neuberger et al., 1984, Nature 312:604-608; Takeda et al., 1985, Nature 314: 452-454) by splicing the genes from a mouse antibody molecule specific for the target protein together with genes from a human antibody molecule of appropriate biological activity can be used; such antibodies are within the scope of this invention.

Additionally, where monoclonal antibodies are advantageous, they can be alternatively selected from large antibody libraries using the techniques of phage display (Marks et al., 1992, J. Biol. Chem. 267:16007-16010). Using this technique, libraries of up to 10¹² different antibodies have been expressed on the surface of fd filamentous phage, creating a “single pot” in vitro immune system of antibodies available for the selection of monoclonal antibodies (Griffiths et al., 1994, EMBO J. 13:3245-3260). Selection of antibodies from such libraries can be done by techniques known in the art, including contacting the phage to immobilized target protein, selecting and cloning phage bound to the target, and subcloning the sequences encoding the antibody variable regions into an appropriate vector expressing a desired antibody format.

According to the invention, techniques described for the production of single chain antibodies (U.S. Pat. No. 4,946,778) can be adapted to produce single chain antibodies specific to the target protein. An additional embodiment of the invention utilizes the techniques described for the construction of Fab expression libraries (Huse et al., 1989, Science 246: 1275-1281) to allow rapid and easy identification of monoclonal Fab fragments with the desired specificity for the target protein.

Antibody fragments that contain the idiotypes of the target protein can be generated by techniques known in the art. For example, such fragments include, but are not limited to: the F(ab′)₂ fragment which can be produced by pepsin digestion of the antibody molecule; the Fab′ fragments that can be generated by reducing the disulfide bridges of the F(ab′)₂ fragment, the Fab fragments that can be generated by treating the antibody molecule with papain and a reducing agent, and Fv fragments.

In the production of antibodies, screening for the desired antibody can be accomplished by techniques known in the art, e.g., ELISA (enzyme-linked immunosorbent assay). To select antibodies specific to a target protein, one may assay generated hybridomas or a phage display antibody library for an antibody that binds to the target protein.

5.3.5. Methods of Modifying Protein Activities

Methods of directly modifying protein activities include, inter alia, dominant negative mutations, specific drugs (used in the sense of this application) or chemical moieties generally, and also the use of antibodies, as previously discussed.

Dominant negative mutations are mutations to endogenous genes or mutant exogenous genes that when expressed in a cell disrupt the activity of a targeted protein species. Depending on the structure and activity of the targeted protein, general rules exist that guide the selection of an appropriate strategy for constructing dominant negative mutations that disrupt activity of that target (Hershkowitz, 1987, Nature 329:219-222). In the case of active monomeric forms, over expression of an inactive form can cause competition for natural substrates or ligands sufficient to significantly reduce net activity of the target protein. Such over expression can be achieved by, for example, associating a promoter, preferably a controllable or inducible promoter, of increased activity with the mutant gene. Alternatively, changes to active site residues can be made so that a virtually irreversible association occurs with the target ligand. Such can be achieved with certain tyrosine kinases by careful replacement of active site serine residues (Perlmutter et al., 1996, Current Opinion in Immunology 8:285-290).

In the case of active multimeric forms, several strategies can guide selection of a dominant negative mutant. Multimeric activity can be controllably decreased by expression of genes coding exogenous protein fragments that bind to multimeric association domains and prevent multimer formation. Alternatively, controllable over expression of an inactive protein unit of a particular type can tie up wild-type active units in inactive multimers, and thereby decrease multimeric activity (Nocka et al., 1990, EMBO J. 9:1805-1813). For example, in the case of dimeric DNA binding proteins, the DNA binding domain can be deleted from the DNA binding unit, or the activation domain deleted from the activation unit. Also, in this case, the DNA binding domain unit can be expressed without the domain causing association with the activation unit. Thereby, DNA binding sites are tied up without any possible activation of expression. In the case where a particular type of unit normally undergoes a conformational change during activity, expression of a rigid unit can inactivate resultant complexes. For a further example, proteins involved in cellular mechanisms, such as cellular motility, the mitotic process, cellular architecture, and so forth, are typically composed of associations of many subunits of a few types. These structures are often highly sensitive to disruption by inclusion of a few monomeric units with structural defects. Such mutant monomers disrupt the relevant protein activities and can be controllably expressed in a cell.

In addition to dominant negative mutations, mutant target proteins that are sensitive to temperature (or other exogenous factors) can be found by mutagenesis and screening procedures that are well-known in the art.

Also, one of skill in the art will appreciate that expression of antibodies binding and inhibiting a target protein can be employed as another dominant negative strategy.

5.3.6. Drugs of Specific Known Action

Additionally, activities of certain proteins can be controllably altered by exposure to exogenous drugs or ligands. In a preferable case, a drug is known that interacts with only one target protein in the cell and alters the activity of only that one target protein. Graded exposure of a cell to varying amounts of that drug thereby causes graded perturbations of pathways originating at that protein. The alteration can be either a decrease or an increase of activity. Less preferably, a drug is known and used that alters the activity of only a few (e.g., 2-5) target proteins with separate, distinguishable, and non-overlapping effects. Graded exposure to such a drug causes graded perturbations to the several pathways originating at the target proteins.

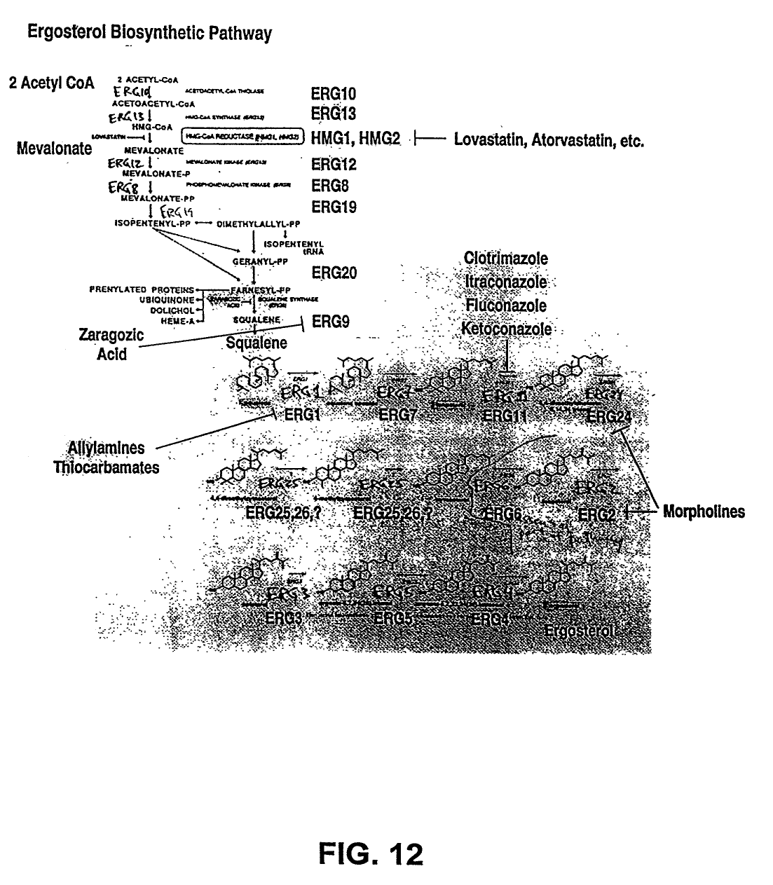

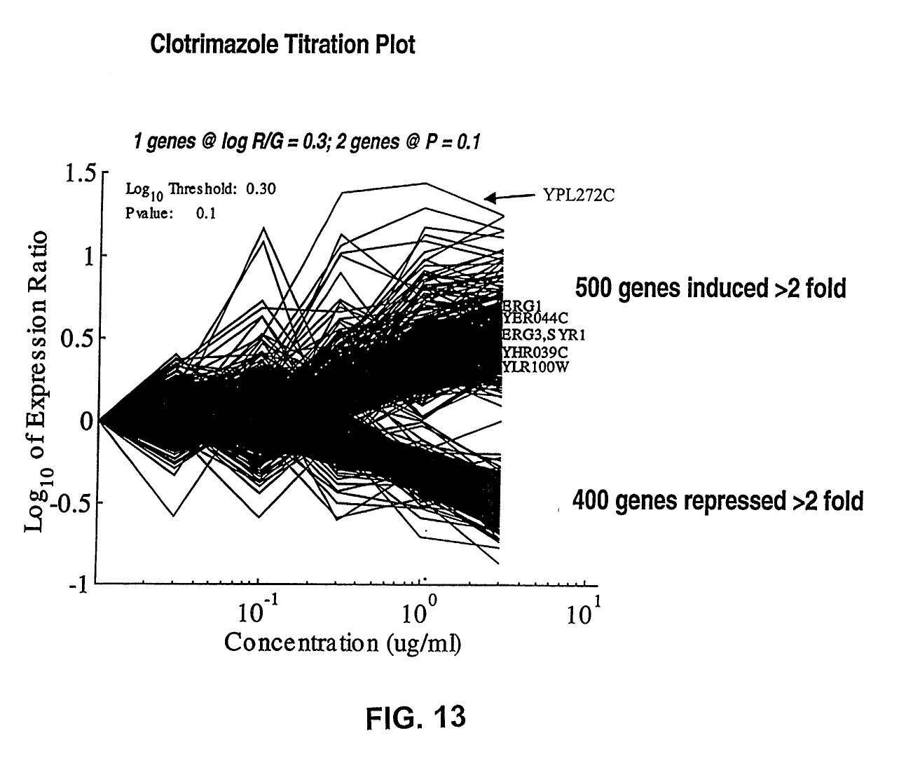

In a specific embodiment of the invention, when the pathway of interest is the yeast ergosterol-pathway, a known drug which acts as an inhibitor of ergosterol-biosynthesis may be used to perturb the pathway. Ergosterol is the primary membrane sterol in fungi and in some trypanosomes. Ergosterol serves a structural role comparable to that of cholesterol in mammalian cells, and is essential for the integrity and structure of the fungal cell membrane. As depicted in FIG. 12, the ergosterol synthesis pathway contains at least 18 genes designated ERG1 though EGR26. Several different classes of antifungal agents exist which target the ergosterol-pathway. Such drugs or agents may be used in connection with the methods of the invention. In one embodiment, the a known antifungal drug is used to perturb the ergosterol-pathway. Such drugs include but are not limited to the following.

The polyenes are a class of drugs that bind to ergosterol in the fungal membrane, causing the cells to become leaky and die (Hamilton-Miller, J., 1973, Bacteriol. Rev. 37:166). Polyenes and derivatives, include drugs such as amphotericin B, nystatin, and pimaricin.

Azoles are a second class of drug which target the ergosterol-pathway. Azoles act to inhibit C-14 demethylation of an ergosterol precursor called lanosterol. Normally in the synthesis of the ergosterol, the EGR11 gene product acts to demethylate C-14 of lanosterol. Azoles inhibit this process leading to a C-14 methylsterol product. Consequently, incorporation of these altered products into the fungal membrane in place of ergosterol, leads to reduced membrane fluidity, reduced fungal growth, and reduced invasiveness. Azoles, include drugs such as clotrimazole, intraconazole, fluconazole, miconazole, econazole, sulconazole, and ketoconazole.

A third class of ergosterol-pathway drug are the allylamines-thiocarbamates which act to inhibit squalene epoxidase, the ERG1 gene product. Allylamines-thiocarbamates derivatives include naftifine, tolnaftate, and terbinafine.

The morpholines are a forth class of drug that affect ergosterol synthesis. Morpholines, such as amorolfine, act to block two separate steps of the ergosterol synthesis pathway. Morpholines inhibit C-14 sterol reduction by the ERG24 gene product. Morpholines also inhibit isomerization of sterol Δ8→7 by the ERG2 gene product.

As will be appreciated by one skilled in the art, any known drug associated with a particular biological pathway of interest may be used in connection with the methods of the invention, for example, as an agent to perturb the particular biological pathway.

### 5.4. Preparing the Microarray

The invention herein provides methods of using microarray technology to identify reporter genes and target genes of a particular biological pathway. Microarray may be prepared by any method known in the art, including but not limited to the preparation methods described herein below.

5.4.1. Binding Sites on the Microarrays

As noted above, the “binding site” to which a particular polynucleotide molecule specifically hybridizes according to the invention is usually a complementary polynucleotide sequence. In one embodiment, the binding sites of the microarray are DNA or DNA “mimics” (e.g., derivatives and analogues) corresponding to at least a portion of each gene in an organism's genome. In another embodiment, the binding sites of the microarray are complementary RNA or RNA mimics.

DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates.

DNA can be obtain, e.g., by polymerase chain reaction (“PCR”) amplification of gene segments from genomic DNA, cDNA (e.g., by RT-PCR), or clones sequences. PCR primers are preferably chosen based on known sequences of the genes or cDNA that result in amplification of unique fragments (e.g, fragments that do not share more than 10 bases of contiguous identical sequence with any other fragment on the microarray). Computer programs that are well known in the art are useful in the design of primer with the required specificity and optimal amplification properties, such as Oligo version 5.0 (National Biosciences). Typically, each binding site of the microarray will be between about 20 bases and about 12,000 bases, and usually between about 300 bases and about 2,000 bases in length, and still more usually between about 300 bases and about 800 bases in length. PCR methods are well known in the art, and are described, for example, in Innis et al., eds., 1990, PCR Protocols: A Guide to Methods and Applications, Academic Press Inc., San Diego, Calif. It will be apparent to one skilled in the art that controlled robotic systems are useful for isolating and amplifying nucleic acids. In a specific embodiment of the invention, PCR methods are used to amplify ORFs of S. cerevisiae yeast genome. In a further preferred specific embodiment, amplification of yeast genome is performed such that each of the known or predicted ORFs in the yeast genome is prepared.

An alternative means for generating the polynucleotide binding sites of the microarray is by synthesis of synthetic polynucleotides or oligonucleotides, e.g., using N-phosphonate or phosphoramidite chemistries (Froehler et al., 1986, Nucleic Acid Res. 14:5399-5407; McBrid et al., 1983, Tetrahedron Lett. 24:246-248). Synthetic sequences are typically between about 15 and about 500 bases in length, more typically between about 20 and about 50 bases. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholn et al., 1993, Nature 363:566-568; U.S. Pat. No. 5,539,083).

In alternative embodiments, the hybridization sites (i.e., the binding sites) are made from plasmid or phage clones of genes, cDNAs (e.g., expressed sequence tags), or inserts therefrom (Nguyen et al., 1995, Genomics 29:207-209).

5.4.2. Attaching Binding Sites to the Solid Surface

Solid supports on which binding sites of microarrays may be immobilized are well-known in the art and include filter materials, such as nitrocellulose, cellulose acetate, nylon, and polyester, among others, as well as non-porous materials, such as glass, plastic (e.g., polypropylene),polyacrylamide, and silicon. In general, non-porous supports, and glass in particular, are preferred. The solid support may also be treated in such a way as to enhance binding of oligonucleotides thereto, or to reduce non-specific binding of unwanted substances thereto. For example, it is often desirable to treat a glass support with polylysine or silane to facilitate attachment of binding sites such as oligonucleotides to the glass. A preferred method for attaching binding sites such as nucleic acids to a surface is by printing on glass plates, as is described generally by Schena et al., 1995, Science 270:467-470. This method is especially useful for preparing microarrays of cDNA (See also, DeRisi et al., 1996, Nature Genetics 14:457-460; Shalon et al., 1996, Genome Res. 6:689-645; and Schena et al., 1995, Proc. Natl. Acad. Sci. U.S.A. 93:10539-11286). Blanchard discloses the use of an ink jet printer for oligonucleotide synthesis (U.S. application Ser. No. 09/008,120, filed Jan. 16, 1998).

Methods of immobilizing binding sites on the solid support may include direct touch, micropipetting (Yershov, K et al., Genetics 93: 4913, 1996), or the use of controlled electric fields to direct a given oligonucleotide to a specific spot in the array (U.S. Pat. No. 5,605,662 issued to Heller et al.). In a specific embodiment, DNA is typically immobilized at a density of 100 to 10,000 oligonucleotides per cm² and preferably at a density of about 1000 oligonucleotides per cm²

In a preferred embodiment, binding sites (e.g., oligonucleotides) are synthesized directly on said support (Maskos, U et al., 1993, Nucl. Acids Res. 21: 2267; Fodor, S. P et al., 1991, Science 281:767; Blanchard et al., 1996, Biosens. Bioelectron. 11: 687). Among methods of synthesizing oligonucleotides directly on a solid support, particularly preferred method are photolithography (see e.g., Fodor, supra., and McGall et al.,1996, Proc. Natl. Acad. Sci. (USA) 93: 13555, 1996) and most preferred, piezoelectric printing (see e.g., Blanchard, supra).

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). When these methods are used, oligonucleotides (e.g., 20-mers) of known sequence are synthesized directly on a surface such as a derivatized glass slides. Usually, the array produced is redundant, with several oligonucleotide molecules per RNA. Oligonucleotide binding sites can be chosen to detect alternatively spliced mRNAs.

Other methods for making microarrays, e.g., by masking (Maskos and Southern, 1992, Nuc. Acids. Res. 20:1679-1684), may also be used. In principle, any type of array, for example, dot blots on a nylon hybridization membrane (see Sambrook et al., supra) could be used. However, as will be recognized by those skilled in the art, very small arrays will frequently be preferred because hybridization volumes will be smaller.

5.4.3. Target Polynucleotides Molecules

As described, supra, the polynucleotide molecules which may be analyzed by the present invention may be from any source, including naturally occurring nucleic acid molecules, as well as synthetic nucleic acid molecules. In a preferred embodiment, the polynucleotide molecules analyzed by the invention comprise RNA, including, but by no means limited to, total cellular RNA, poly(A)⁺ messenger RNA (mRNA), fractions thereof, or RNA transcribed from cDNA. In a specific embodiment, Cellular RNA or DNAs from two cell populations (e.g., RNA of S. cerevisiae untreated or treated with a specific drug) are analyzed by incubating both populations of RNAs with the microarray. In a specific embodiment of the invention, S. cerevisiae concentrated or treated with a drug or agent known to alter the ergosterol pathway (e.g., clotrimazole). In yet another specific embodiment, S. cerevisiae containing a deletion mutation is used to identify gene function. Methods for preparing total and poly(A)⁺ RNA are well known in the art, and are described generally, e.g., in Sambrook et al., supra. In one embodiment, RNA is extracted from cells of the various types of interest in this invention using guanidinium thiocyanate lysis followed by CsCl centrifugation (Chirgwin et al., 1979, Biochemistry 18:5294-5299). Poly (A)⁺ RNA is selected by selection with oligo-dT cellulose. Cells of interest include, but are by no means limited to, wild-type cells, drug-exposed wild-type cells, modified cells, diseased cells, and, in particular, cancer cells.

In one embodiment, RNA can be fragmented by methods known in the art, e.g., by incubation with ZnCl₂, to generate fragments of RNA. In one embodiment, isolated mRNA can be converted to antisense RNA synthesized by in vitro transcription of double-stranded cDNA in the presence of labeled dNTPs (Lockhart et al., 1996, Nature Biotechnology 14:1675).

In other embodiments, the polynucleotide molecules to be analyzed may be DNA molecules such as fragmented genomic DNA, or PCR products of amplified mRNA or cDNA. In a preferred embodiment of the invention the polynucleotide molecules to be analyzed are cDNAs which are reverse transcribed from mRNAs. In a specific embodiment of the invention the polynucleotide molecules analyzed are cDNAs reverse transcribed from cDNAs of fungal cell treated with antifungal drugs.

5.4.4. Hybridization Polynucleotides to Microarrays

Nucleic acid hybridization and wash conditions are chosen so that the polynucleotide molecules to be analyzed by the invention “specifically bind” or “specifically hybridize” to the complementary polynucleotide sequences of the array, preferably to a specific array site, wherein its complementary DNA is located.

Arrays containing double-stranded binding site DNA situated thereon are preferably subjected to denaturing conditions to render the DNA single-stranded prior to contacting with the target polynucleotide molecules. Arrays containing single-stranded binding site DNA (e.g., synthetic oligodeoxyribonucleic acids) may need to be denatured prior to contacting with the target polynucleotide molecules, e.g., to remove hairpins or dimers which form due to self complementary sequences.

Optimal hybridization conditions will depend on the length (e.g., oligomer versus polynucleotide greater than 200 bases) and type (e.g.; RNA or DNA) of binding site and target nucleic acids. General parameters for specific (i.e., stringent) hybridization conditions are described in Sambrook et al. (supra), and in Ausubel et al., 1987, Current Protocols in Molecular Biology, Greene Publishing and Wiley-Interscience, New York. When the cDNA microarrays of Schena et al. (Shena et al., 1996, Proc. Natl. Acad. Sci. U.S.A. 93:10614) are used, typical hybridization conditions are hybridization in 5×SSC plus 0.2% SDS at 65° C. for four hours, followed by washes at 25° C. in high stringency wash buffer (0.1×SSC plus 0.2% SDS) (Shena et al., 1996, Proc. Natl. Acad. Sci. U.S.A. 93:10614). Useful hybridization conditions are also provided, e.g., Tijessen, 1993, Hybridization With Nucleic Acid Probes, Elsevier Science Publishers B.V.; and Kricka, 1992, Nonisotopic DNA Probe Techniques, Academic Press, San Diego, Calif.

In a another specific embodiment, use of a nucleic acid which is hybridizable to an S. cerevisiae nucleic acid or to its reverse complement, or to a nucleic acid encoding an ergosterol derivative, or to its reverse complement, under conditions of low stringency is provided. By way of example and not limitation, procedures using such conditions of low stringency are as follows (see also Shilo and Weinberg, 1981, Proc. Natl. Acad. Sci. U.S.A. 78, 6789-6792). Arrays containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μ/ml denatured salmon sperm DNA. Hybridizations are carried out in the same solution with the following modifications: 0.02% PVP, 0.02% Ficoll, 0.2% BSA, 100 μ/ml salmon sperm DNA, 10% (wt/vol) dextran sulfate, and 5-20×10⁶ cpm ³²P-labeled probe is used. Arrays are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Arrays are blotted dry and visualized. If necessary, arrays are washed for a third time at 65-68° C. and re-visualized. Other conditions of low stringency which may be used are well known in the art (e.g., as employed for cross-species hybridizations).

In another specific embodiment, use of a nucleic acid which is hybridizable to an ergosterol nucleic acid, or its reverse complement, under conditions of high stringency is provided. By way of example and not limitation, procedures using such conditions of high stringency are as follows. Prehybridization of arrays containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Arrays are hybridized for 48 h at 65° C. in prehybridization mixture containing 100 μg/ml denatured salmon sperm DNA and 5-20×10⁶ cpm of ³²P-labeled probe. Washing of arrays is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency which may be used are well known in the art.

In another specific embodiment, use of a nucleic acid which is hybridizable to an ergosterol nucleic acid, or its reverse complement, under conditions of moderate stringency is provided. Selection of appropriate conditions for such stringencies is well known in the art (see e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, © 1987-1997, Current Protocols, © 1994-1997 John Wiley and Sons, Inc.).

In another embodiment, after hybridization, stringency conditions are as follows. Each array is washed two times each for 30 minutes each at 45° C. in 40 mM sodium phosphate, pH 7,2, 5% SDS, 1 mM EDTA, 0.5% bovine serum albumin, followed by four washes each for 30 minutes in sodium phosphate, pH 7.2, 1% SDS, 1 mM EDTA, and subsequently each array is treated differently as described below for low, medium, or high stringency hybridization conditions. For low stringency hybridization, arrays are not washed further. For medium stringency hybridization, membranes are additionally subjected to four washes each for 30 minutes in 40 mM sodium phosphate, pH 7.2, 1% SDS, 1 mM EDTA at 55° C. For high stringency hybridization, following the washes for low stringency, membranes are additionally subjected to four washes each for 30 minutes in 40 mM sodium phosphate, pH 7.2, 1% SDS, 1 mM EDTA at 55° C., followed by four washes each for 30 minutes in sodium phosphate, pH 7.2, 1% SDS, 1 mM EDTA at 65° C.

Use of nucleic acids encoding derivatives and analogs of ergosterol-pathway proteins, and ergosterol antisense nucleic acids for antifungal therapies or drug targets are additionally provided.

Use of fragments of ergosterol nucleic acids comprising regions conserved between (i.e., with homology to) other ergosterol nucleic acids, of the same or different species, are also provided.

5.4.5. Signal Detection on Hybridized Microarrays and Data Analysis

It will be appreciated that when cDNA complementary to the mRNA of a cell is made and hybridized to a microarray under suitable hybridization conditions, the level of hybridization to the site in the array corresponding to any particular gene will reflect the prevalence in the cell of mRNA transcribed from that gene. For example, when detectably labeled (e.g., with a fluorophore) cDNA complementary to the total cellular mRNA is hybridized to a microarray, the site on the array corresponding to a gene (i.e., capable of specifically binding the product of the gene) that is not transcribed in the cell will have little or no signal (e.g., fluorescent signal), and a gene for which the encoded mRNA is prevalent will have a relatively strong signal.

In preferred embodiments, cDNAs from two different cells (e.g. untreated and drug treated) are hybridized to the binding sites of the microarray. In the case of drug responses, one cell is exposed to a drug and another cell of the same type is not exposed to the drug. The cDNA derived from each of the two cell types are differently labeled so that they can be distinguished. In one embodiment, for example, cDNA from a cell treated with a drug is synthesized using a fluorescein-labeled dNTP, and cDNA from a second cell, not drug-exposed, is synthesized using a rhodamine-labeled dNTP. When the two cDNAs are mixed and hybridized to the microarray, the relative intensity of signal from each cDNA set is determined for each site on the array, and any relative difference in abundance of a particular mRNA is thereby detected.

In the example described above, the cDNA from the drug-treated cell will fluoresce green when the fluorophore is stimulated, and the cDNA from the untreated cell will fluoresce red. As a result, when the drug treatment has no effect, either directly or indirectly, on the relative abundance of a particular mRNA in a cell, the mRNA will be equally prevalent in both cells, and, upon reverse transcription, red-labeled and green-labeled cDNA will be equally prevalent. When hybridized to the microarray, the binding site(s) for that species of RNA will emit wavelength characteristic of both fluorophores. In contrast, when the drug-exposed cell is treated with a drug that, directly or indirectly, increases the prevalence of the mRNA in the cell, the ratio of green to red fluorescence will increase. When the drug decreases the mRNA prevalence, the ratio will decrease.

The use of a two-color fluorescence labeling and detection scheme to define alterations in gene expression has been described, (See, e.g., Shena et al., 1995, Science 270:467-470). An advantage of using cDNA labeled with two different fluorophores is that a direct and internally controlled comparison of the mRNA levels corresponding to each arrayed gene in two cell states can be made, and variations due to minor differences in experimental conditions (e.g., hybridization conditions) will not affect subsequent analyses. However, it will be recognized that it is also possible to use cDNA from a single cell, and compare, for example, the absolute amount of a particular mRNA in, e.g., a drug-treated or pathway-perturbed cell and an untreated cell.

When fluorescently labeled probes are used, the fluorescence emissions at each site of a transcript array can be, preferably, detected by scanning confocal laser microscopy (see e.g., Fodor, S., et al., 1993, Nature 364:555). In one embodiment, a separate scan, using the appropriate excitation line, is carried out for each of the two fluorophores used. Among fluorescent dyes that may be used to label DNA and RNA are fluorescein, lissamine, Cy3, Cy5, phycoerythrin, and rhodamine 110. Cy3 and Cy5 are particularly preferred. In a specific embodiment, where the sample to be hybridized is a cDNA, labeling is accomplished by incorporating fluoresecently-labeled deoxynucleotide triphosphates (dNTPs), such as Cy3 or Cy5-dUTP, during in vitro reverse transcription. Fluorescently-labeled dNTPs are commercially available from sources such as Amersham Pharmacia Biotech, Piscataway, N.J. Alternatively, cDNAs are labeled indirectly by incorporating biotinylated nucleotides during cDNA synthesis, followed by the addition of fluorescently-labeled avidin or streptavidin. Biotinylated dNTPS are available from Enzo (Farmingdale, N.Y.) and Boehringer Mannheim (Indianapolis, Ind.), while fluorescently-labeled avidin and streptavidin are available from Becton Dickinson (Mountain View, Calif.) and Molecular Probes (Eugene, Oreg.). Methods of reverse transcription and labeling are well-known in the art and are described for example, in Ausbel, F. et al., eds., 1994, Current Protocols in Molecular Biology, New York; DeRisi, J., 1997, Science 278:680-86; and Schena, M, et al., 1996, Proc. Natl. Acad Sci.,USA, 93:10614-19.

Alternatively, a laser can be used that allows simultaneous specimen illumination at wavelengths specific to the two fluorophores and emissions from the two fluorophores can be analyzed simultaneously (see Shalon et al., 1996, Genome Res. 6:639-645). In a preferred embodiment, the arrays are scanned with a laser fluorescent scanner with a computer controlled X-Y stage and a microscope objective. Although simultaneous hybridization of differentially labeled cDNA samples is preferred, use of a single label to perform hybridizations sequentially rather than simultaneously, may also be performed. Sequential excitation of the two fluorophores is achieved with a multi-line, mixed gas laser, and the emitted light is split by wavelength and detected with two photomultiplier tubes. Such fluorescence laser scanning devices are described, e.g., in Schena et al., 1996, Genome Res. 6:639-645. Alternatively, the fiber-optic bundle described by Ferguson et al., 1996, Nature Biotech. 14:1681-1684, may be used to monitor mRNA abundance levels at a large number of sites simultaneously.

In one embodiment, where the sample to be hybridized is mRNA, labeling is accomplished by incorporating fluoresecently-labeled ribonucleotides or biotinylated ribonucleotides during in vitro transcription, as described in Lockhart, D. J. et al., 1996, Nature Biotech. 14:1675-80.

Although it is preferred to use fluorescent labels, other labels may also be employed, such as radioisotopes, enzymes, and luminescers. Such methods are well-known to those of skill in the art.

To probe a DNA microarray, the labeled samples are hybridized to the microarray under a fixed set of conditions, such as sample concentration, temperature, buffer and salt concentration, incubation time, etc (see e.g. Section 5.4.4, herein). After washing to remove unbound sample, the microarray is excited with specific wavelengths of light and scanned to detect fluorescence. Typically, two samples, each labeled with a different fluor, are hybridized simultaneously to permit differential expression measurements. When neither sample hybridizes to a given spot in the array, no fluorescence is detected. When only one sample hybridizes to a given spot, the color of the resulting fluorescence will correspond to that of the fluor used to label the hybridizing sample (e.g., green when the sample was labeled with fluorescein, or red, if the sample was labeled with rhodamine). When both samples hybridize to the same spot, an combinatorial color is produced (e.g., yellow if the samples were labeled with fluorescein and rhodamine). Then, applying methods of pattern recognition and data analysis as described herein and in U.S. patent application Ser. No. 09/179,569, filed Oct. 27, 1998, now pending, in U.S. patent application Ser. No. 09/220,275, filed Dec. 23, 1998, now pending, and in U.S. patent application Ser. No. 09/220,142 filed Dec. 23, 1998, now pending each of which are incorporated herein by reference in their entirety, it is possible to quantify differences in gene expression between the samples.

Signals are recorded and, in a preferred embodiment, analyzed by computer, e.g., using a 12 bit analog to digital board. In one embodiment, the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluorophores may be made. For any particular hybridization site on the transcript array, a ratio of the emission of the two fluorophores can be calculated. The ratio is independent of the absolute expression level of the cognate gene, but is useful for genes whose expression is significantly modulated by drug administration, gene deletion, or any other tested event.

According to the method of the invention, the relative abundance of an mRNA in two cells or cell lines is scored as a perturbation and its magnitude determined (i.e., the abundance is different in the two sources of mRNA tested) or as not perturbed (i.e., the relative abundance is the same, see U.S. patent Ser. No. 09/179,569, filed Oct. 27, 1998, U.S. patent Ser. No. 09/220,142, filed Dec. 23, 1998 now pending, U.S. patent Ser. No. 09/220,275 filed Dec. 23, 1998, which are incorporated herein by reference in their entirety). As used herein, a difference between the two sources of RNA of at least a factor of about 25% (i.e., RNA is 25% more abundant in one source than in the other source), more usually about 50%, even more often by a factor of about 2 (i.e., twice as abundant), 3 (three times as abundant), or 5 (five times as abundant) is scored as a perturbation. Present detection methods allow reliable detection of difference of an order of about 3-fold to about 5-fold, but more sensitive methods are expected to be developed.

Preferably, in addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

5.4.6. Other Methods of Transcriptional State Measurement

The transcriptional state of a cell may be measured by other gene expression technologies known in the art. Several such technologies produce pools of restriction fragments of limited complexity for electrophoretic analysis, such as methods combining double restriction enzyme digestion with phasing primers (see, e.g., European Patent O 534858 A1, filed Sep. 24, 1992, by Zabeau et al.), or methods selecting restriction fragments with sites closest to a defined mRNA end (see e.g., Prashar et al., 1996, Proc. Natl. Acad. Sci. U.S.A. 93:659-663). Other methods statistically sample cDNA pools, such as by sequencing sufficient bases (e.g., 20-50 bases) in each of multiple cDNAs to identify each cDNA, or by sequencing short tags (e.g., 9-10 bases) which are generated at known positions relative to a defined mRNA end (see e.g., Velculescu, 1995, Science 270:484-487).

Such methods and systems of measuring transcriptional state, although less preferable than microarrays, may, nevertheless, be used in the present invention.

5.4.7. Measurement of Other Aspects of Biological State

In various embodiments of the present invention, aspects of the biological state other than the transcriptional state, such as the translational state, the activity state, or mixed aspects can be measured in order to obtain drug and pathway responses. Details of these embodiments are described in this section.

5.4.7.1. Embodiments Based on Translational State Measurements

Measurement of the translational state may be performed according to several methods. For example, whole genome monitoring of protein (i.e., the “proteome,” Goffeau et al., supra) can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to the action of a drug of interest. Methods for making monoclonal antibodies are well known (see, e.g., Harlow and Lane, 1988, Antibodies: A Laboratory Manual, Cold Spring Harbor, N.Y., which is incorporated in its entirety for all purposes). In a preferred embodiment, monoclonal antibodies are raised against synthetic peptide fragments designed based on genomic sequence of the cell. With such an antibody array, proteins from the cell are contacted to the array and their binding is assayed with assays known in the art.

Alternatively, proteins can be separated by two-dimensional gel electrophoresis systems. Two-dimensional gel electrophoresis is well-known in the art and typically involves iso-electric focusing along a first dimension followed by SDS-PAGE electrophoresis along a second dimension. See, e.g., Hames et al., 1990, Gel Electrophoresis of Proteins: A Practical Approach, IRL Press, New York; Shevchenko et al., 1996, Proc. Nat'l Acad. Sci. USA 93:1440-1445; Sagliocco et al., 1996, Yeast 12:1519-1533; Lander, 1996, Science 274:536-539. The resulting electropherograms can be analyzed by numerous techniques, including mass spectrometric techniques, western blotting and immunoblot analysis using polyclonal and monoclonal antibodies, and internal and N-terminal micro-sequencing. Using these techniques, it is possible to identify a substantial fraction of all the proteins produced under given physiological conditions, including in cells (e.g., in yeast) exposed to a drug, or in cells modified by, e.g., deletion or over-expression of a specific gene.

5.4.7.2. Embodiments Based on Other Aspects of the Biological State

Even though methods of this invention are illustrated by embodiments involving gene expression profiles, the methods of the invention are applicable to any cellular constituent that can be monitored.

In particular, where activities of proteins relevant to the characterization of a perturbation, such as drug action, can be measured, embodiments of this invention can be based on such measurements. Activity measurements can be performed by any functional, biochemical, or physical means appropriate to the particular activity being characterized. Where the activity involves a chemical transformation, the cellular protein can be contacted with the natural substrate(s), and the rate of transformation measured. Where the activity involves association in multimeric units, for example association of an activated DNA binding complex with DNA, the amount of associated protein or secondary consequences of the association, such as amounts of mRNA transcribed, can be measured. Also, where only a functional activity is known, for example, as in cell cycle control, performance of the function can be observed. However known and measured, the changes in protein activities form the response data analyzed by the foregoing methods of this invention.

In alternative and non-limiting embodiments, response data may be formed of mixed aspects of the biological state of a cell. Response data can be constructed from, e.g., changes in certain mRNA abundances, changes in certain protein abundances, and changes in certain protein activities.

### 5.5. Drug Development with Target Genes

The invention provides methods for the identification of target genes which may be used for the development of drugs and therapeutic agents that target a pathway of interest. By way of example, the invention is illustrated in terms of an ergosterol-pathway target gene; however, one skilled in the art will appreciate that the methods described herein may be applied to any pathway of interest and used for the development of drugs and/or therapeutic agents which target the pathway of interest. For example, one pathway of interest is the ergosterol-pathway of yeast. As described above, a target gene, for a pathway such as the ergosterol-pathway may be identified by the methods of the invention, (e.g., by using cluster analysis followed by validation of the gene as a target). Target genes of the ergosterol-pathway, may be used in controlling fungal infection of human, animal, or plant species. For example, the proteins encoded by a novel target gene of the ergosterol-pathway provide targets for antifungal and fungicidal agents. For example, a drug may be developed to inhibit an essential ergosterol-pathway target gene or the protein encoded by such a gene. Inhibition of an essential target gene or protein thus modifies the growth, reproduction, and/or survival of a fungus containing the essential target gene, and thus is used as antifungal or fungicidal agent. In yet another embodiment, the drug of therapeutic agent is a dominant negative form of an ergosterol-pathway protein, which inactivates the protein encoded by the target gene of the ergosterol-pathway and may be used as an antifungal or fungicidal agent. In yet another embodiment, antisense ergosterol-pathway nucleic acids may be used to inactivate an essential target gene, and therefore provide an antifungal or fungicidal agent. Further, as will be appreciated by one skilled in the art, when a target gene is discovered by the methods of the invention, such a target may be found in species other than that which the target gene was first discovered, and may provide useful drug targets in such species. For example, if a target gene of the ergosterol-pathway is discovered in S. cerevisiae this gene is not only a target for antifungal or fungicidal drug development against the S. cerevisiae, but may lead to the development of antifungal or fungicidal agents for other fungal species as well.

Fungi which may used or tested in connection with the methods of the invention include but are not limited to: Cryptococcus species, including Cryptococcus neoformans; Blastomyces species, including Blastomyces dermatitidis; Aiellomyces species, including Aiellomyces dermatitidis; Histoplasfria species, including Histoplasfria capsulatum; Coccidioldes species, including Coccidioides immitis; Candids species, including C. albicans, C. tropicalis, C. parapsilosis, C. guilliermondii, and C. krusei, Aspergillus species, including A. fumigatus, A. flavus, and A. niger, Rhizopus species; Rhizomucor species; Cunninghammella species; Apophysomyces species, including A. saksenaea, A. mucor, A. absidia; Sporothrix species, including Sporothrix schenckii; Paracoccidloides species, including Paracoccidioides brasiliensis; Pseudallescheria species, including Pseudallescheria boydii; Torulopsis species, including Torulopsis glabrata; Dermatophyres species; Histoplasma species; Pneumocystis species; Blastomyces species; Peniciilium species; Microsporum species; Epidermophyton species; Trichophytom species; Saccharomyces species, including S. cerevisiae; Schizomyces species, including S. pombe; Trichosporon species; Rhodotorula species; and Malassezia species.

Tests for antifungal activities can be any method known in the art. Such methods may include contacting one or more test fungal cells with the potential antifungal drug and measuring the growth inhibition or death of the fungal cells. A drug which exhibits a high rate of killing of the test fungus at low dose is a preferred antifungal drug. In one embodiment, the antifungal drug kills 50-75% of the test fungal cells. In another embodiment, the antifungal drug kills 75-85% of the test fungal cells. In a preferred embodiment, the antifungal drug kills 85-95% of the test fungal cells. In a more preferred embodiment, the antifungal drug kills 95-99% of the test fungal cells. In a most preferred embodiment, the antifungal drug kills 100% of the test fungal cells. In other embodiments of the invention, the dose of the drug is in the range of 1-10 nM, 10-100 nM, 100-1000 nM, 1-10 μM, 10-100 μM, or 10-100 μM.

As will be appreciated by one skilled in the art, any target gene may be tested for its requirement for normal activity of a pathway in order to develop a drug or therapeutic directed to the pathway in which that target gene is involved. Further, it will be appreciated that targets which are found in one species may also be a target in other species, and may be validated by the methods of the invention.

### 5.6. Expression of Reporter Genes and/or Target Genes

The nucleotide sequence coding for reporter gene or target gene of the invention or a functionally active analog or fragment or other derivative thereof may be used for example for the preparation of an assay in which to screen potential drugs which bind to, or enhance, inhibit, or modulate the activity of such a protein, and are described herein below. In one embodiment, the sequence can be inserted into an appropriate expression vector, i.e., a vector which contains the necessary elements for the transcription and translation of the inserted protein-coding sequence. The necessary transcriptional and translational signals can also be supplied by the native ergosterol-pathway gene and/or its flanking regions. A variety of host-vector systems may be utilized to express the protein-coding sequence. These include but are not limited to mammalian cell systems infected with virus (e.g., vaccinia virus, adenovirus, etc.); insect cell systems infected with virus (e.g., aculovirus); microorganisms such as yeast containing yeast vectors, or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used. In yet another embodiment, a fragment of an reporter or target protein comprising one or more domains of the reporter or target protein is expressed.

In a specific embodiment, a vector is used that comprises a promoter operably linked to a nucleic acid of a reporter gene or target gene, one or more origins of replication, and, optionally, one or more selectable markers (e.g., an antibiotic resistance gene).

In other specific embodiments, the reporter or target protein, fragment, analog, or derivative may be expressed as a fusion, or chimeric protein product (comprising the protein, fragment, analog, or derivative joined via a peptide bond to a heterologous protein sequence (of a different protein)). A chimeric protein may include fusion of the reporter or target protein, fragment, analog, or derivative to a second protein or at least a portion thereof, wherein a portion is one (preferably 10, 15, or 20) or more amino acids of said second protein. Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer.

The invention provides a method for identifying a molecule that modulates the expression of an ergosterol-pathway gene selected from the group consisting of YHR039C (as depicted in FIG. 2, as set forth in SEQ ID NO:1), YLW100W (as depicted in FIG. 4, as set forth in SEQ ID NO:3), YPL272C (as depicted in FIG. 6, as set forth in SEQ ID NO:5), YGR131W (as depicted in FIG. 8, as set forth in SEQ ID NO:7), and YDR453C (as depicted in FIG. 10, as set forth in SEQ ID NO:9), comprising recombinantly expressing in a fungal cell one or more candidate molecules, and detecting the expression of said ergosterol-pathway gene; wherein an increase or decrease in the gene expression relative to the expression in the absence of candidate molecules indicates that the molecules modulates ergosterol-pathway gene expression.

The invention provides a method for identifying a molecule that modulates the expression of a PKC-pathway gene selected from the group consisting of SLT2(YHR030C) (as depicted in FIGS. 17A-B, as set forth in SEQ ID NO:11), YKR161C (as depicted in FIGS. 19A-B, as set forth in SEQ ID NO:13), PIR3(YKL163W) (as depicted in FIGS. 21A-B, as set forth in SEQ ID NO:15), YPK2(YMR104C) (as depicted in FIGS. 23A-B, as set forth in SEQ ID NO:17), YLR194C (as depicted in FIGS. 25A-B, as set forth in SEQ ID NO:19), and ST1(YDR055W) (as depicted in FIGS. 27A-B, as set forth in SEQ ID NO:21), comprising recombinantly expressing in a fungal cell one or more candidate molecules, and detecting the expression of said PKC-pathway gene; wherein an increase or decrease in the gene expression relative to the expression in the absence of candidate molecules indicates that the molecules modulates PKC-pathway gene expression.

The invention provides a method for identifying a molecule that modulates the expression of an Invasive Growth pathway gene selected from the group consisting of KSS1(YGR040W) (as depicted in FIG. 29, as set forth in SEQ ID NO:23), PGU1(YJR153W) (as depicted in FIG. 31, as set forth in SEQ ID NO:25), YRL042C (as depicted in FIG. 33, as set forth in SEQ ID NO:27), and SVS1(YPL163C) (as depicted in FIG. 35, as set forth in SEQ ID NO:29), comprising recombinantly expressing in a fungal cell one or more candidate molecules, and detecting the expression of said Invasive Growth pathway gene; wherein an increase or decrease in the gene expression relative to the expression in the absence of candidate molecules indicates that the molecules modulates Invasive Growth pathway gene expression.

### 5.7. Structure of Reporter and/or Target Genes and Proteins

The structure of reporter or target genes and proteins of the invention can be analyzed by various methods known in the art. Such analysis may be useful, for example, in the design of antifungal or fungicidal agents of the invention. Some examples of such methods are described below.

5.7.1. Genetic Analysis

The cloned DNA or cDNA corresponding to a reporter or target gene can be analyzed by methods including but not limited to Southern hybridization (Southern, 1975, J. Mol. Biol. 98:503-517), Northern hybridization (see e.g., Freeman et al., 1983, Proc. Natl. Acad. Sci. U.S.A. 80:4094-4098), restriction endonuclease mapping (Maniatis, 1982, Molecular Cloning, A Laboratory Manual, Cold Spring Harbor Laboratory Press, Cold Spring Harbor, N.Y.), and DNA sequence analysis. Accordingly, this invention provides for the use of nucleic acid probes recognizing a reporter or target gene. For example, polymerase chain reaction (PCR; U.S. Pat. Nos. 4,683,202, 4,683,195 and 4,889,818; Gyllenstein et al., 1988, Proc. Natl. Acad. Sci. U.S.A. 85:7652-7656; Ochman et al., 1988, Genetics 120:621-623; Loh et al., 1989, Science 243:217-220) followed by Southern hybridization with an a reporter or target gene-specific probe can allow the detection of a reporter or target gene in DNA from various cell types. In one specific embodiment, the cell types are from different species within the same phylogenetic kingdom. Methods of amplification other than PCR are commonly known and can also be employed. In one embodiment, Southern hybridization can be used to determine the genetic linkage of a reporter or target gene. Northern hybridization analysis can be used to determine the expression of a gene assigned to the a particular biological pathway by the methods disclosed herein. Various cell types, at various states of development or activity can be tested for gene expression. The stringency of the hybridization conditions for both Southern and Northern hybridization can be manipulated to ensure detection of nucleic acids with the desired degree of relatedness to the specific a reporter or target gene probe used. Modifications of these methods and other methods commonly known in the art can be used.

Restriction endonuclease mapping can be used to roughly determine the genetic structure of a reporter or target gene. Restriction maps derived by restriction endonuclease cleavage can be confirmed by DNA sequence analysis. Restriction endonucleases may also be used to digest DNA sequences which are attached to microarrays.

DNA sequence analysis can be performed by any techniques known in the art, including but not limited to the method of Maxam and Gilbert (1980, Meth. Enzymol. 65:499-560), the Sanger dideoxy method (Sanger et al., 1977, Proc. Natl. Acad. Sci. U.S.A. 74:5463), the use of T7 DNA polymerase (Tabor and Richardson, U.S. Pat. No. 4,795,699), or use of an automated DNA sequencer (e.g., Applied Biosystems, Foster City, Calif.). In a specific embodiment, DNA sequencing is used to confirm the sequence of a microarray binding partner or probe.

5.7.2. Protein Analysis

The amino acid sequence of an ergosterol-pathway protein can be derived by deduction from the DNA sequence, or alternatively, by direct sequencing of the protein, e.g., with an automated amino acid sequencer. In a preferred embodiment, S. cerevisiae protein sequences are obtained thru the Saccharomyces Genome Database (www.Stratford.edu/Saccharomyces).

A reporter-gene or target-gene protein sequence can be further characterized by a hydrophilicity analysis (Hopp and Woods, 1981, Proc. Natl. Acad. Sci. U.S.A. 78:3824). A hydrophilicity profile can be used to identify the hydrophobic and hydrophilic regions of the protein encoded by a reporter gene or target gene and the corresponding regions of the gene sequence which encode such regions.

Structural prediction analysis (Chou and Fasman, 1974, Biochemistry 13:222) can also be done, to identify regions of a protein encoded by a reporter gene or target gene, that assume specific secondary structures, which may be useful in the design of therapeutics which target specific biological-pathway proteins.

Manipulation, translation, and secondary structure prediction, open reading frame prediction and plotting, as well as determination of sequence homologies, can also be accomplished using computer software programs available in the art.

Other methods of structural analysis can also be employed. These include but are not limited to X-ray crystallography (Engstom, 1974, Biochem. Exp. Biol. 11:7-13), nuclear magnetic resonance spectroscopy (Clore and Gonenborn, 1989, CRC Crit. Rev. Biochem. 24:479-564) and computer modeling (Fletterick and Zoller, 1986, Computer Graphics and Molecular Modeling, in Current Communications in Molecular Biology, Cold Spring Harbor Laboratory Press, Cold Spring Harbor, N.Y.).

The invention further relates to the use of proteins encoded by reporter genes or target genes, derivatives (including but not limited to fragments), analogs, and molecules of reporter or target proteins.

The production and use of fragments, derivatives, and analogs related to an reporter or target protein are within the scope of the present invention. In a specific embodiment, the derivative or analog is functionally active, i.e., capable of exhibiting one or more functional activities associated with a full-length, wild-type reporter or target protein. As one example, such derivatives or analogs which have the desired re-clustering activity can be assigned to a biological-pathway. As yet another example, such derivatives or analogs which have the desired co-clustering activity can be used for targets for the development of drugs directed to such a target, such as an antifungal or fungicidal agent directed to a target gene in the ergosterol-pathway. Derivatives or analogs that retain, or alternatively lack or inhibit, a desired biological-pathway protein property-of-interest (e.g., binding to a specific biological pathway protein binding partner), can be used as inducers, or inhibitors, respectively, of such property and its physiological correlates. A specific embodiment relates to a dominant negative form of an ergosterol-pathway protein fragment that can bind and inhibit ergosterol-pathway protein. Derivatives or analogs of an ergosterol-pathway protein can be tested for the desired activity by procedures known in the art, including but not limited to the assays described below.

In particular, reporter or target protein derivatives can be made by altering the sequences by substitutions, additions (e.g., insertions) or deletions. Due to the degeneracy of nucleotide coding sequences, other DNA sequences which encode substantially the same amino acid sequence as the reporter or target gene may be used in the practice of the present invention. These include but are not limited to nucleotide sequences comprising all or portions of a reporter or target gene which is altered by the substitution of different codons that encode a functionally equivalent amino acid residue within the sequence, thus producing a silent change.

In a specific embodiment of the invention, use of proteins consisting of or comprising a fragment of reporter or target protein consisting of at least 10 (continuous) ammo acids of the reporter or target protein is provided. In other embodiments, the fragment consists of at least 20 or at least 50 amino acids of the reporter or target protein. In specific embodiments, such fragments are not larger than 35, 100 or 200 amino acids. Use of derivatives or analogs of reporter or target proteins include but are not limited to those molecules comprising regions that are substantially homologous to the reporter or target protein or fragment thereof (e.g., in various embodiments, at least 60% or 70% or 80% or 90% or 95% identity over an amino acid sequence of identical size or when compared to an aligned sequence in which the alignment is done by a computer homology program known in the art) or whose encoding nucleic acid is capable of hybridizing to a coding reporter or target gene sequence, under high stringency, moderate stringency, or low stringency conditions.

Specifically, by way of example computer programs for determining homology may include but are not limited to TBLASTN, BLASTP, FASTA, TFASTA, and CLUSTALW (Pearson and Lipman, 1988, Proc. Natl. Acad. Sci. USA 85(8):2444-8; Altschul et al., 1990, J. Mol. Biol. 215(3):403-10; Thompson, et al., 1994, Nucleic Acids Res. 22(22):4673-80; Higgins, et al., 1996, Methods Enzymol 266:383-402; Altschul, et al., 1990, J. Mol. Biol. 215(3):403-10).

Specifically, Basic Local Alignment Search Tool (BLAST) (www.ncbi.nlm.nih.gov) (Altschul et al., 1990, J. of Molec. Biol., 215:403-410, “The BLAST Algorithm; Altschul et al., 1997, Nuc. Acids Res. 25:3389-3402) is a heuristic search algorithm tailored to searching for sequence similarity which ascribes significance using the statistical methods of Karlin and Altschul 1990, Proc. Nat'l Acad. Sci. USA, 87:2264-68; 1993, Proc. Nat'l Acad. Sci. USA 90:5873-77. Five specific BLAST programs perform the following tasks: 1) The BLASTP program compares an amino acid query sequence against a protein sequence database; 2) The BLASTN program compares a nucleotide query sequence against a nucleotide sequence database; 3) The BLASTX program compares the six-frame conceptual translation products of a nucleotide query sequence (both strands) against a protein sequence database; 4) The TBLASTN program compares a protein query sequence against a nucleotide sequence database translated in all six reading frames (both strands); 5) The TBLASTX program compares the six-frame translations of a nucleotide query sequence against the six-frame translations of a nucleotide sequence database.

Smith-Waterman (database: European Bioinformatics Institute wwwz.ebi.ac.uk/bic_sw/) (Smith-Waterman, 1981, J. of Molec. Biol., 147:195-197) is a mathematically rigorous algorithm for sequence alignments.

FASTA (see Pearson et al., 1988, Proc. Nat'l Acad. Sci. USA, 85:2444-2448) is a heuristic approximation to the Smith-Waterman algorithm. For a general discussion of the procedure and benefits of the BLAST, Smith-Waterman and FASTA algorithms see Nicholas et al., 1998, “A Tutorial on Searching Sequence Databases and Sequence Scoring Methods” (www.psc.edu) and references cited therein.

The reporter or target derivatives and analogs of the invention can be produced by various methods known in the art. The manipulations which result in their production can occur at the gene or protein level. For example, a cloned reporter or target gene sequence can be modified by any of numerous strategies known in the art (Sambrook et al., 1989, Molecular Cloning, A Laboratory Manual, 2d ed., Cold Spring Harbor Laboratory Press, Cold Spring Harbor, N.Y.). The sequence can be cleaved at appropriate sites with restriction endonuclease(s), followed by further enzymatic modification if desired, isolated, and ligated in vitro.

Additionally, an reporter or target gene nucleic acid sequence can be mutated in vitro or in vivo, to create and/or destroy translation, initiation, and/or termination sequences, or to create variations in coding regions and/or to form new restriction endonuclease sites or destroy preexisting ones, to facilitate further in vitro modification. Any technique for mutagenesis known in the art can be used, including but not limited to, chemical mutagenesis, in vitro site-directed mutagenesis (Hutchinson et al., 1978, J. Biol. Chem. 253:6551), use of TAB® linkers (Pharmacia), PCR with primers containing a mutation, etc.

Manipulations of an reporter or target protein sequence may also be made at the protein level. Included within the scope of the invention are reporter or target protein fragments or other derivatives or analogs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

In addition, analogs and derivatives of a reporter or target protein can be chemically synthesized. For example, a peptide corresponding to a portion of a reporter or target protein which comprises the desired domain, or which mediates the desired activity in vitro, can be synthesized by use of a peptide synthesizer. Furthermore, if desired, nonclassical amino acids or chemical amino acid analogs can be introduced as a substitution or addition into the reporter or target sequence. Non-classical amino acids include but are not limited to the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general. Furthermore, the amino acid can be D (dextrorotary) or L (levorotary).

In a specific embodiment, an reporter or target protein derivative is a chimeric or fusion protein comprising a reporter or target protein or fragment thereof (preferably consisting of at least a domain or motif of the reporter or target protein, or at least 10 amino acids of the reporter or target protein) joined at its amino- or carboxy-terminus via a peptide bond to an amino acid sequence of a different protein. In specific embodiments, the amino acid sequence of the different protein is at least 6, 10, 20 or 30 continuous amino acids of the different proteins or a portion of the different protein that is functionally active. In one embodiment, such a chimeric protein is produced by recombinant expression of a nucleic acid encoding the protein (comprising an reporter or target-coding sequence joined in-frame to a coding sequence for a different protein). Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. Chimeric genes comprising portions of a reporter or target gene fused to any heterologous protein-encoding sequences may be constructed. A specific embodiment relates to a chimeric protein comprising a fragment of reporter or target protein of at least six amino acids, or a fragment that displays one or more functional activities of the reporter or target protein.

### 5.8. Identification of Compounds with Binding Capacity

This invention provides screening methodologies useful in the identification of proteins and other compounds which bind to, or otherwise directly interact with, the reporter or target genes and proteins. Screening methodologies are well known in the art The proteins and compounds include endogenous cellular components which interact with the identified genes and proteins in vivo and which, therefore, may provide new targets for pharmaceutical and therapeutic interventions, as well as recombinant, synthetic, and otherwise exogenous compounds which may have binding capacity and, therefore, may be candidates for pharmaceutical agents. Thus, in one series of embodiments, cell lysates may be screened for proteins or other compounds which bind to one of the normal or mutant reporter or target genes and proteins.

Alternatively, any of a variety of exogenous compounds, both naturally occurring and/or synthetic (e.g., libraries of small molecules or peptides), may be screened for binding capacity.

As will be apparent to one of ordinary skill in the art, there are numerous other methods of screening individual proteins or other compounds, as well as large libraries of proteins or other compounds (e.g., phage display libraries) to identify molecules which bind to reporter or target proteins of the invention. All of these methods comprise the step of mixing a reporter or target protein or fragment with test compounds, allowing time for any binding to occur, and assaying for any bound complexes. All such methods are enabled by the present disclosure of substantially pure reporter or target proteins, substantially pure functional domain fragments, fusion proteins, antibodies, and methods of making and using the same. In a specific embodiment, the reporter or target protein is an ergosterol-pathway protein. In another specific embodiment, the reporter or target protein is a PKC-pathway protein. In another specific embodiment, the reporter or target protein is an Invasive Growth pathway protein.

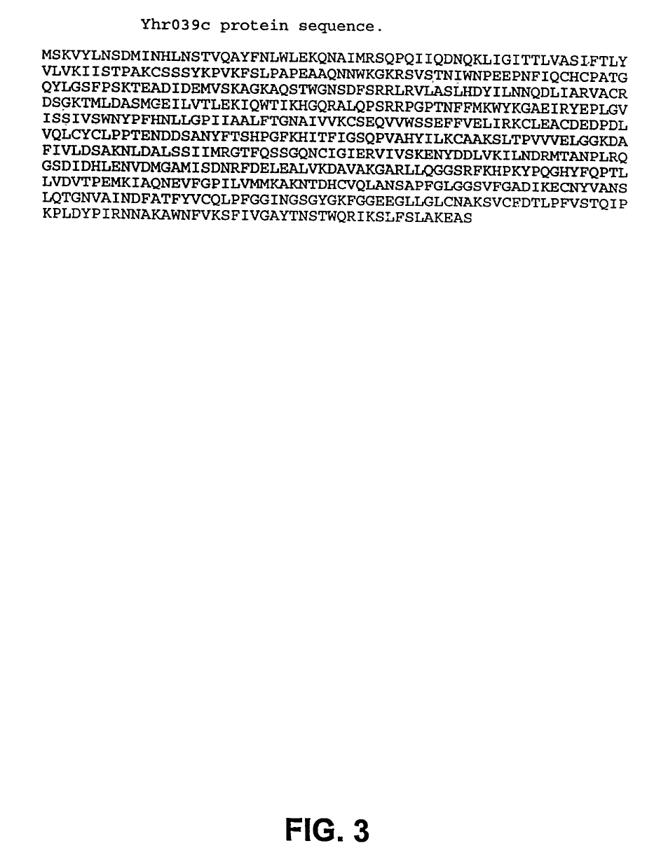

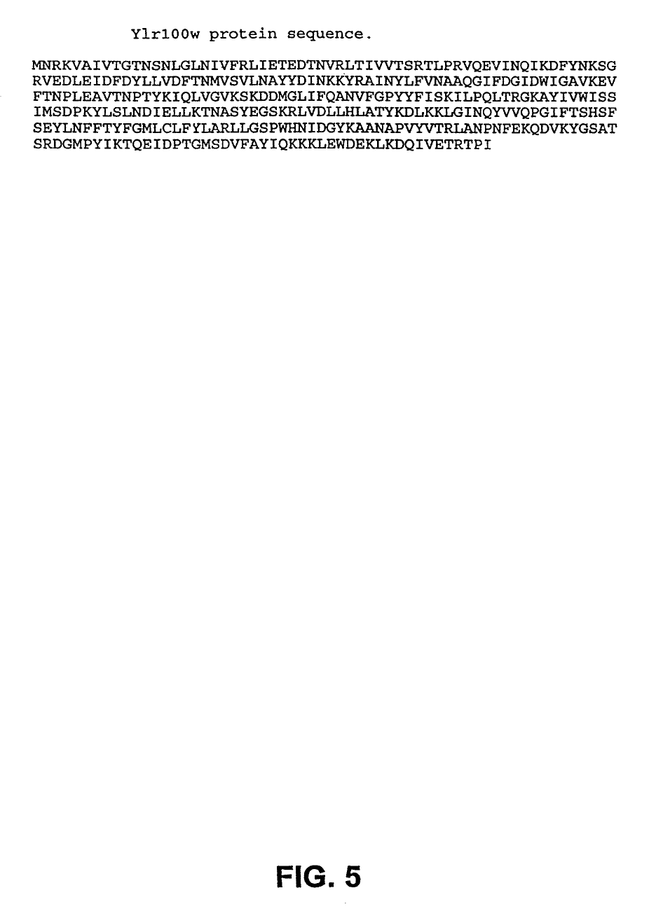

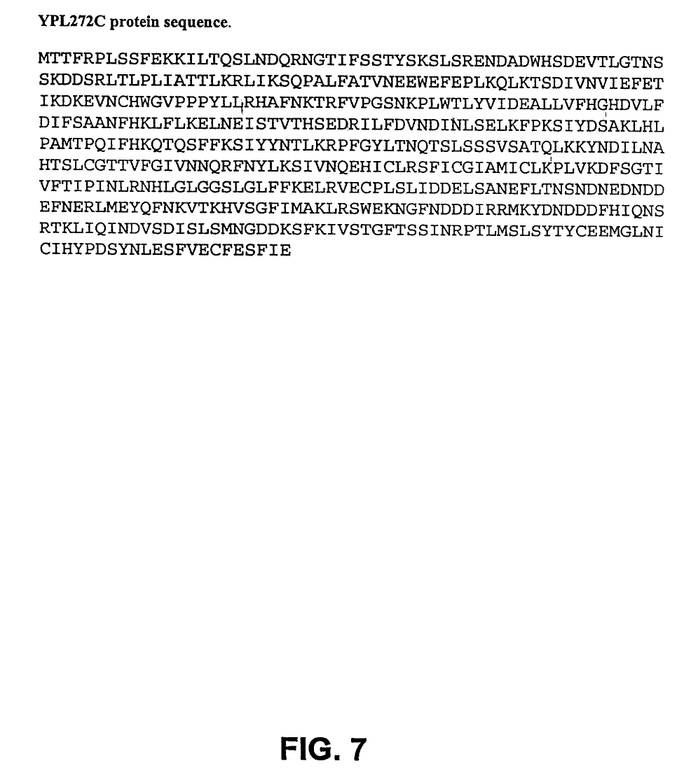

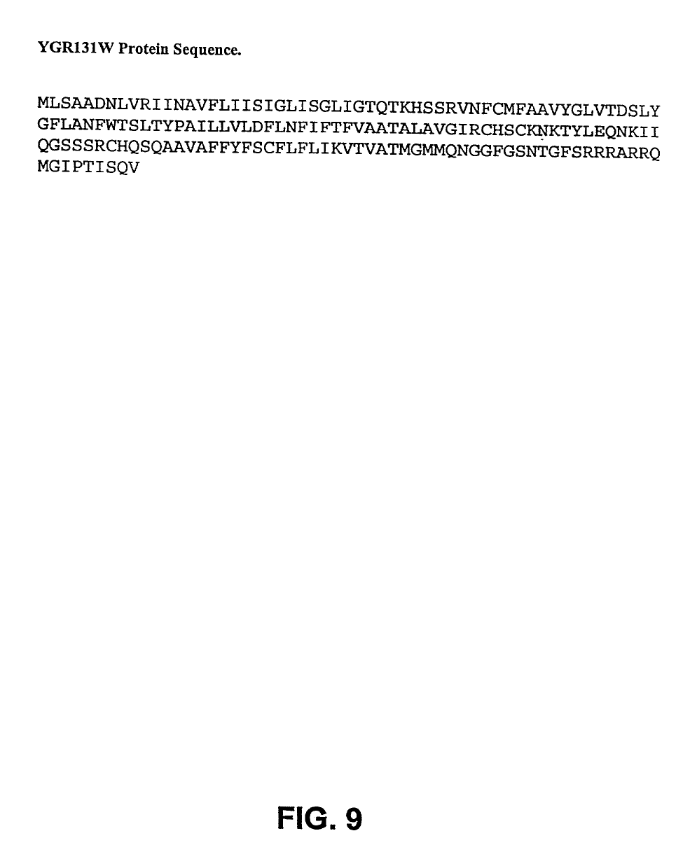

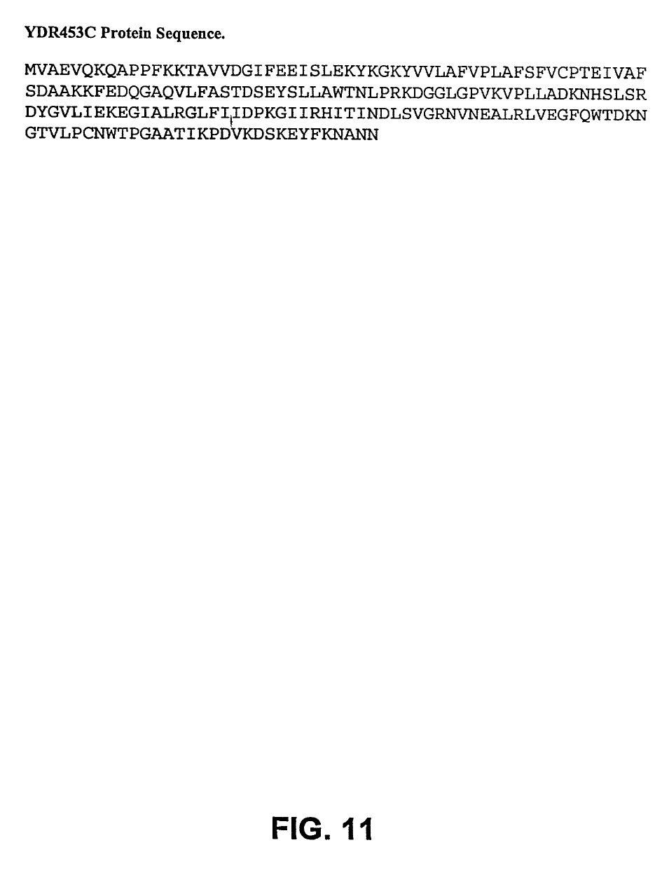

The invention provides a method of identifying a molecule that binds to a ligand selected from the group consisting of (i) an S. cerevisiae ergosterol-pathway protein selected from the group consisting of YHR039C (as depicted in FIG. 3, as set forth in SEQ ID NO:2), YLW100W (as depicted in FIG. 5, as set forth in SEQ ID NO:4), YPL272C (as depicted in FIG. 7, as set forth in SEQ ID NO:6), YGR131W (as depicted in FIG. 9, as set forth in SEQ ID NO:8), and YDR453C (as depicted in FIG. 11, as set forth in SEQ ID NO:10), (ii) a fragment of the S. cerevisiae ergosterol-pathway protein, and (iii) a nucleic acid encoding the S. cerevisiae ergosterol-pathway protein or fragment, the method comprising: (a) contacting the ligand with a plurality of molecules under conditions conducive to binding between the ligand and the molecules; and (b) identifying a molecule within the plurality that binds to the ligand.

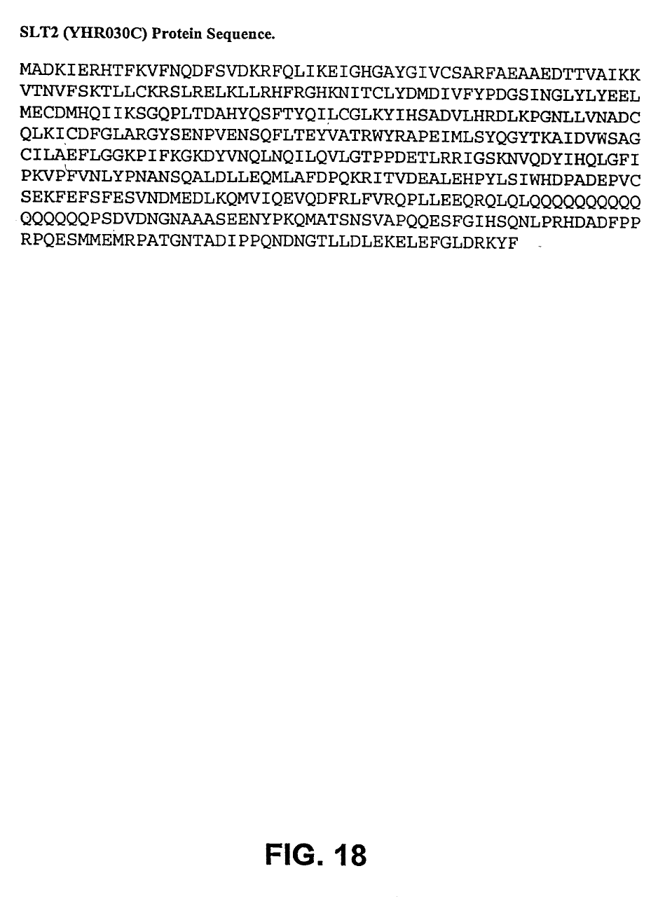

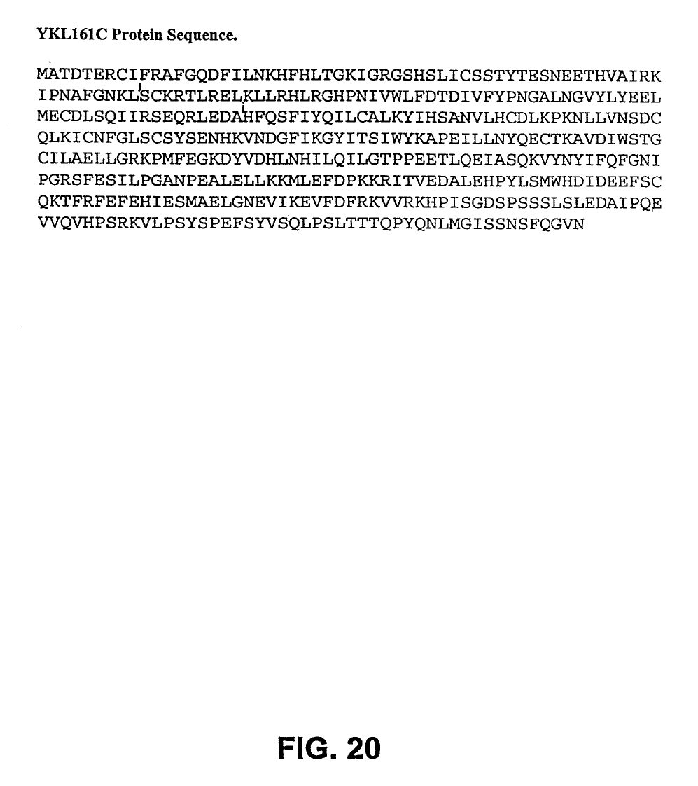

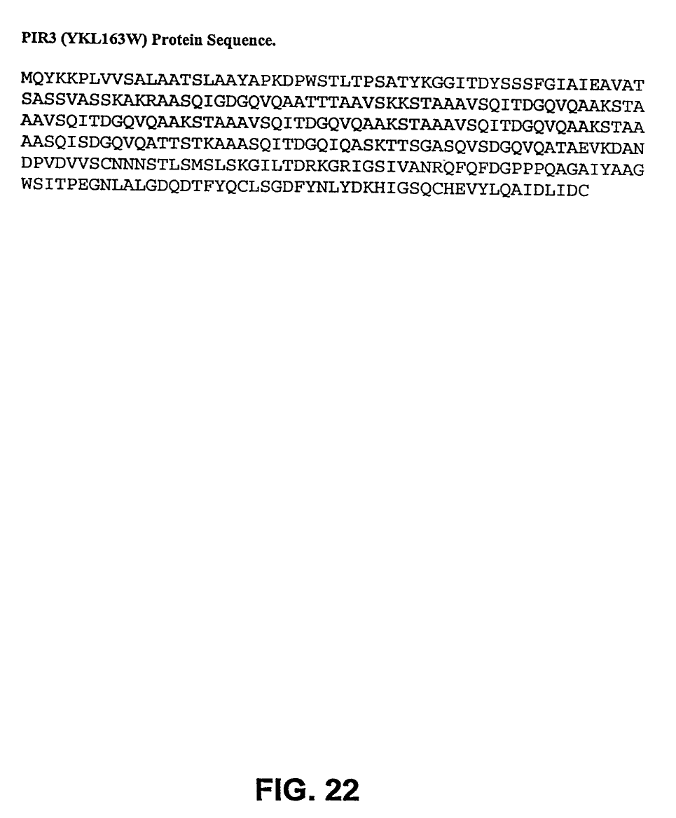

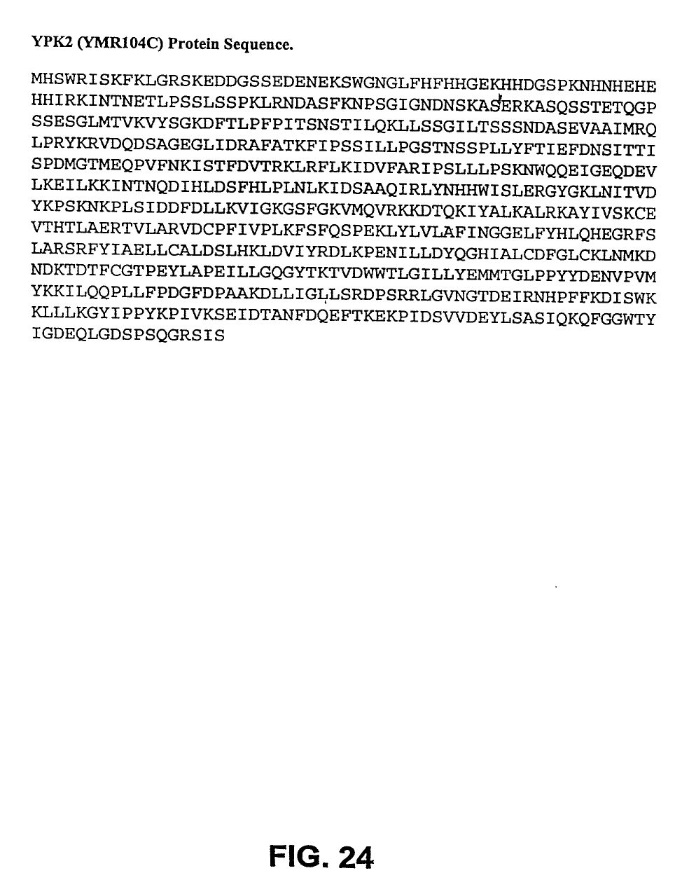

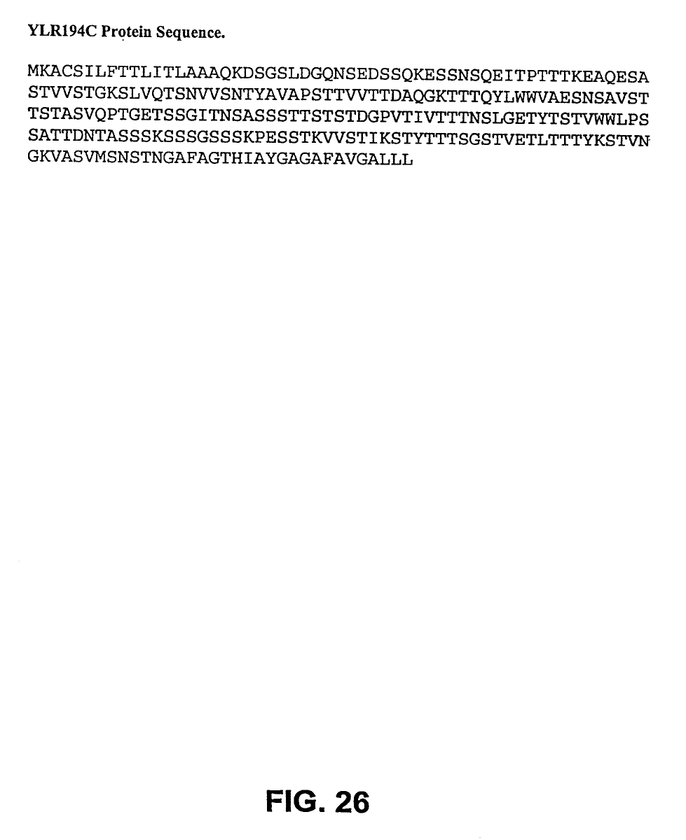

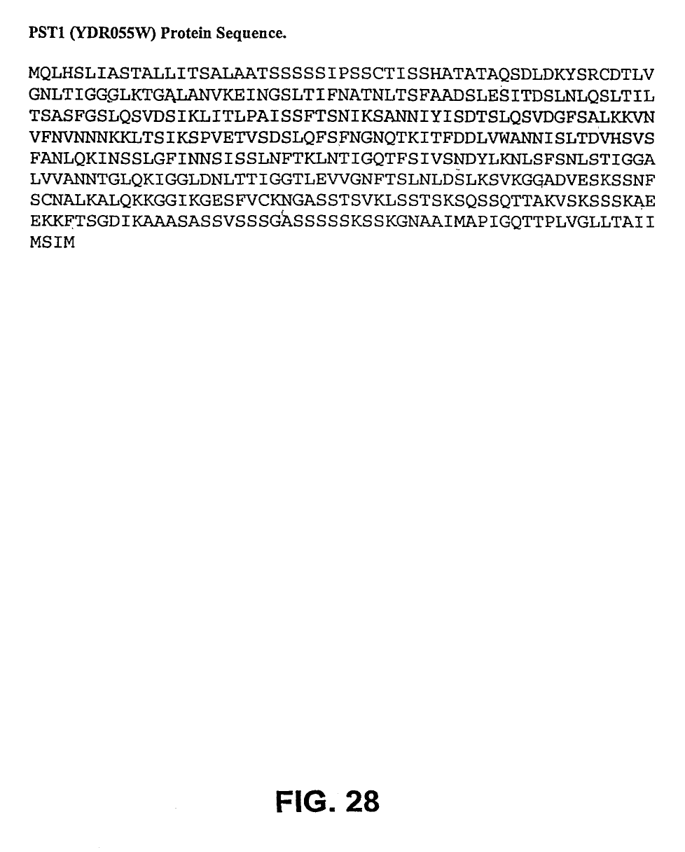

The invention provides a method of identifying a molecule that binds to a ligand selected from the group consisting of (i) an S. cerevisiae PKC-pathway protein selected from the group consisting of SLT2(YHR030C) (as depicted in FIG. 18, as set forth in SEQ ID NO:12), YKR161C (as depicted in FIG. 20, as set forth in SEQ ID NO:14), PIR3(YKL163W) (as depicted in FIG. 22, as set forth in SEQ ID NO:16), YPK2(YMR104C) (as depicted in FIG. 24, as set forth in SEQ ID NO:18), YLR194C (as depicted in FIG. 26, as set forth in SEQ ID NO:20), and ST1(YDR055W) (as depicted in FIG. 28, as set forth in SEQ ID NO:22), (ii) a fragment of the S. cerevisiae PKC-pathway protein, and (iii) a nucleic acid encoding the S. cerevisiae PKC-pathway protein or fragment, the method comprising: (a) contacting the ligand with a plurality of molecules under conditions conducive to binding between the ligand and the molecules; and (b) identifying a molecule within the plurality that binds to the ligand.

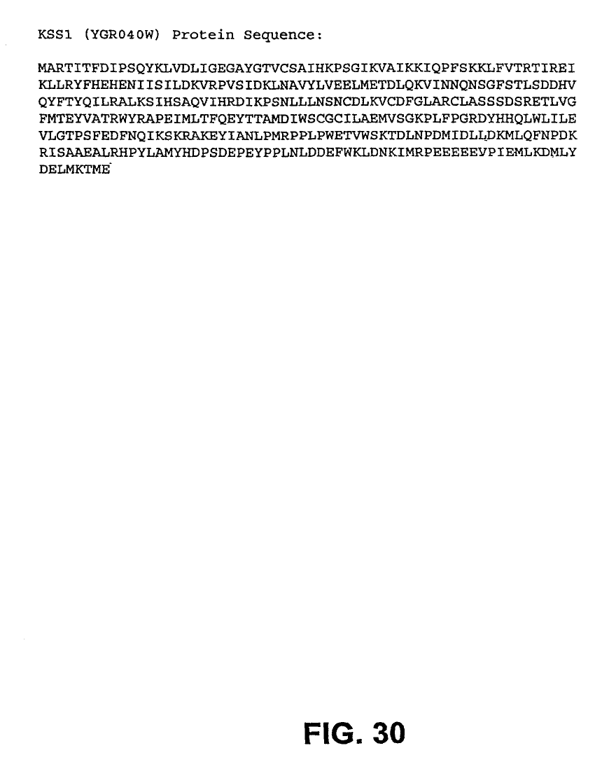

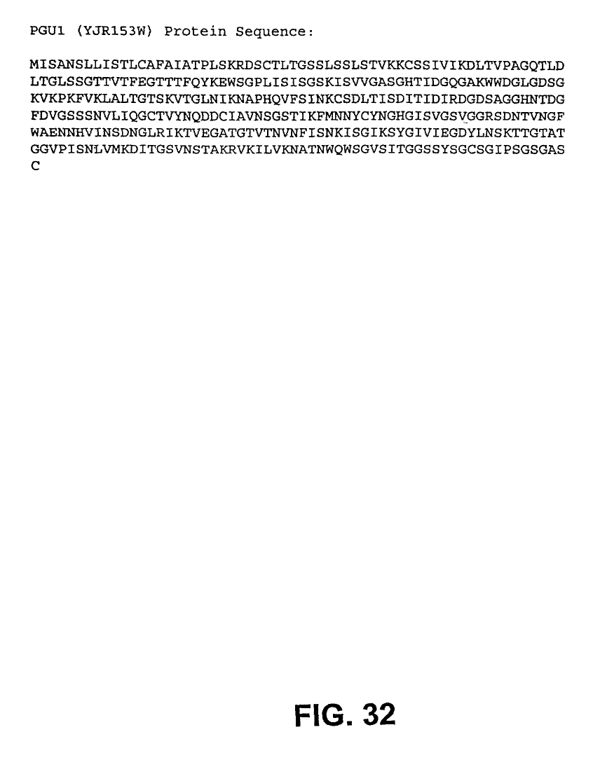

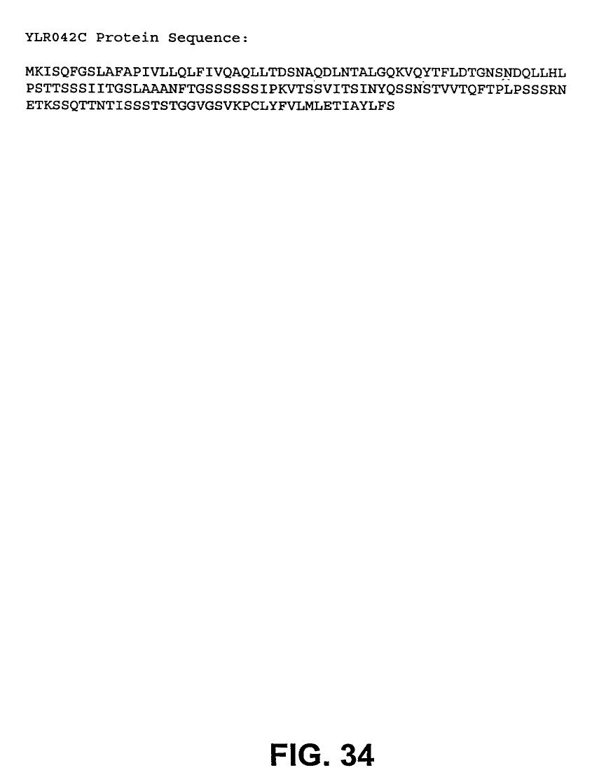

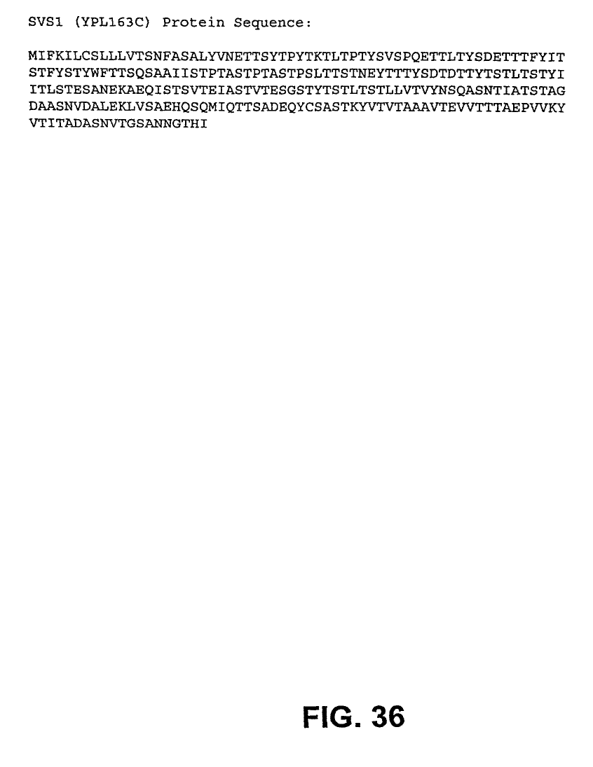

The invention provides a method of identifying a molecule that binds to a ligand selected from the group consisting of (i) an S. cerevisiae Invasive Growth pathway protein selected from the group consisting of KSS1(YGR040W) (as depicted in FIG. 30, as set forth in SEQ ID NO:24), PGU1(YJR153W) (as depicted in FIG. 32, as set forth in SEQ ID NO:26), YRL042C (as depicted in FIG. 34, as set forth in SEQ ID NO:28), and SVS1(YPL163C) (as depicted in FIG. 36, as set forth in SEQ ID NO:30), (ii) a fragment of the S. cerevisiae Invasive Growth pathway protein, and (iii) a nucleic acid encoding the S. cerevisiae Invasive Growth pathway protein or fragment, the method comprising (a) contacting the ligand with a plurality of molecules under conditions conducive to binding between the ligand and the molecules; and (b) identifying a molecule within the plurality that binds to the ligand.

5.8.1. Proteins which Interact with Pathway-Specific Proteins

The present invention further provides methods of identifying or screening for proteins which interact with reporter or target proteins of a biological pathway of interest, or derivatives, fragments, or analogs thereof. In specific embodiments, the method of identifying a molecule that binds to a ligand (e.g., an ergosterol-pathway protein) comprises contacting the ligand with a plurality of molecules under conditions conducive to binding between the ligand and the molecules; and identifying a molecule within the plurality that binds to the ligand. The ligand or protein in the method can either be a purified or non-purified form. Preferably, the method of identifying or screening is a yeast two-hybrid assay system or a variation thereof, as further described below. In this regard, the yeast two-hybrid method has been used to analyze protein-protein interactions (see e.g. Zhu and Kahn, 1997, Proc. Natl. Acad. Sci. U.S.A. 94:13063-13068). Derivatives (e.g., fragments) and analogs of a protein can also be assayed for binding to a binding partner by any method known in the art, for example, immunoprecipitation with an antibody that binds to the protein in a complex followed by analysis by size fractionation of the immunoprecipitated proteins (e.g., by denaturing or nondenaturing polyacrylamide gel electrophoresis), Western analysis, non-denaturing gel electrophoresis, etc.

One aspect of the present invention provides methods for assaying and screening fragments, derivatives and analogs of reporter or target proteins of the invention for interacting proteins (e.g., for binding to an S. cerevisiae ergosterol peptide). Derivatives, analogs and fragments of proteins that interact with a reporter or target protein can preferably identified by means of a yeast two hybrid assay system (Fields and Song, 1989, Nature 340:245-246; U.S. Pat. No. 5,283,173). Because the interactions are screened for in yeast, the intermolecular protein interactions detected in this system occur under physiological conditions that mimic the conditions in eukaryotic cells, including vertebrates or invertebrates (Chien et al., 1991, Proc. Natl. Acad. Sci. U.S.A. 88:9578-9581). By way of illustration, this feature facilitates identification of proteins capable of interaction with an S. cerevisiae ergosterol-pathway protein from species other than S. cerevisiae.

Identification of interacting proteins by the improved yeast two-hybrid system is based upon the detection of expression of a “marker” gene, the transcription of which is dependent upon the reconstitution of a transcriptional regulator by the interaction of two proteins, each fused to one half of the transcriptional regulator. In some embodiments of the invention, the “marker” genes as described below, act as a read-out for the interaction of two test proteins called the bait and the prey. The “bait” (i.e., a pathway-specific reporter or target protein of a or derivative or analog thereof) and “prey” (proteins to be tested for ability to interact with the bait) proteins are expressed as fusion proteins to a DNA binding domain, and to a transcriptional regulatory domain, respectively, or vice versa. In various specific embodiments, the prey has a complexity of at least about 50, about 100, about 500, about 1,000, about 5,000, about 10,000, or about 50,000; or has a complexity in the range of about 25 to about 100,000, about 100 to about 100,000, about 50,000 to about 100,000, or about 100,000 to about 500,000. For example, the prey population can be one or more nucleic acids encoding mutants of a protein (e.g., as generated by site-directed mutagenesis or another method of making mutations in a nucleotide sequence). Preferably, the prey populations are proteins encoded by DNA, e.g., cDNA or genomic DNA or synthetically-generated DNA. For example, the populations can be expressed from chimeric genes comprising cDNA sequences from an un-characterized sample of a population of cDNA from mRNA.

One characteristic of the yeast two-hybrid system is that proteins examined in this system are expressed as cytoplasmic proteins, and therefore do not pass through the secretory pathway. However, several methods are incorporated in the present invention to examine derivatives of reporter or target proteins of the invention that mimic processed forms of these proteins.

In a specific embodiment, recombinant biological libraries expressing random peptides can be used as the source of prey nucleic acids.

In another embodiment, the invention provides methods of screening for inhibitors or enhancers of the protein interactants identified herein. Briefly, the protein-protein interaction assay can be carried out as described herein, except that it is done in the presence of one or more candidate molecules. An increase or decrease in marker gene activity relative to that present when the one or more candidate molecules are absent indicates that the candidate molecule has an effect on the interacting pair. In a preferred method, inhibition of the interaction is selected for (i.e., inhibition of the interaction is necessary for the cells to survive), for example, where the interaction activates the URA3 gene, causing yeast to die in medium containing the chemical 5-fluoroorotic acid (Rothstein, 1983, Meth. Enzymol. 101:167-180). The identification of inhibitors of such interactions can also be accomplished, for example, but not by way of limitation, using competitive inhibitor assays, as described above.

In general, proteins of the bait and prey populations are provided as fusion (chimeric) proteins (preferably by recombinant expression of a chimeric coding sequence) comprising each protein contiguous to a pre-selected sequence. For one population, the pre-selected sequence is a DNA binding domain. The DNA binding domain can be any DNA binding domain, as long as it specifically recognizes a DNA sequence within a promoter. For example, the DNA binding domain is of a transcriptional activator or inhibitor. For the other population, the pre-selected sequence is an activator or inhibitor domain of a transcriptional activator or inhibitor, respectively. The regulatory domain alone (not as a fusion to a protein sequence) and the DNA-binding domain alone (not as a fusion to a protein sequence) preferably do not detectably interact (so as to avoid false positives in the assay). The assay system further includes a reporter gene operably linked to a promoter that contains a binding site for the DNA binding domain of the transcriptional activator (or inhibitor).

Accordingly, in the present method of the invention, binding of a bait fusion protein containing a reporter or target protein of the invention (such as an S. cerevisiae ergosterol-pathway protein) to a prey fusion protein leads to reconstitution of a transcriptional activator (or inhibitor) which activates (or inhibits) expression of the marker gene. The activation (or inhibition) of transcription of the marker gene occurs intracellularly, e.g., in prokaryotic or eukaryotic cells, preferably in cell culture.

The promoter that is operably linked to the marker gene nucleotide sequence can be a native or non-native promoter of the nucleotide sequence, and the DNA binding site(s) that are recognized by the DNA binding domain portion of the fusion protein can be native to the promoter (if the promoter normally contains such binding site(s)) or non-native to the promoter. Thus, for example, one or more tandem copies (e.g. four or five copies) of the appropriate DNA binding site can be introduced upstream of the TATA box in the desired promoter (e.g., in the area of about position −100 to about −400). In a preferred aspect, 4 or 5 tandem copies of the 17 bp UAS (GAL4 DNA binding site) are introduced upstream of the TATA box in the desired promoter, which is upstream of the desired coding sequence for a selectable or detectable marker. In a preferred embodiment, the GAL1-10 promoter is operably fused to the desired nucleotide sequence; the GAL1-10 promoter already contains 4 binding sites for GAL4.

Alternatively, the transcriptional activation binding site of the desired gene(s) can be deleted and replaced with GAL4 binding sites (Bartel et al., 1993, BioTechniques 14:920-924; Chasman et al., 1989, Mol. Cell. Biol. 9:4746-4749). The marker gene preferably contains the sequence encoding a detectable or selectable marker, the expression of which is regulated by the transcriptional activator, such that the marker is either turned on or off in the cell in response to the presence of a specific interaction. Preferably, the assay is carried out in the absence of background levels of the transcriptional activator (e.g., in a cell that is mutant or otherwise lacking in the transcriptional activator).

In one embodiment, more than one marker gene is used to detect transcriptional activation, e.g., one marker gene encoding a detectable marker and one or more marker genes encoding different selectable markers. The detectable marker can be any molecule that can give rise to a detectable signal, e.g., a fluorescent protein or a protein that can be readily visualized or that is recognizable by a specific antibody. The selectable marker can be any protein molecule that confers the ability to grow under conditions that do not support the growth of cells not expressing the selectable marker, e.g., the selectable marker is an enzyme that provides an essential nutrient and the cell in which the interaction assay occurs is deficient in the enzyme and the selection medium lacks such nutrient. The marker gene can either be under the control of the native promoter that naturally contains a binding site for the DNA binding protein, or under the control of a heterologous or synthetic promoter.

The activation domain and DNA binding domain used in the assay can be from a wide variety of transcriptional activator proteins, as long as these transcriptional activators have separable binding and transcriptional activation domains. For example, the GAL4 protein of S. cerevisiae (Ma et al., 1987, Cell 48:847-853), the GCN4 protein of S. cerevisiae (Hope and Struhl, 1986, Cell 46:885-894), the ARD1 protein of S. cerevisiae (Thukral et al., 1989, Mol. Cell. Biol. 9:2360-2369), and the human estrogen receptor (Kumar et al., 1987, Cell 51:941-951), have separable DNA binding and activation domains. The DNA binding domain and activation domain that are employed in the fusion proteins need not be from the same transcriptional activator. In a specific embodiment, a GAL4 or LEXA DNA binding domain is employed. In another specific embodiment, a GAL4 or herpes simplex virus VP16 (Triezenberg et al., 1988, Genes Dev. 2:730-742) activation domain is employed. In a specific embodiment, amino acids 1-147 of GAL4 (Ma et al., 1987, Cell 48:847-853; Ptashne et al., 1990, Nature 346:329-331) is the DNA binding domain, and amino acids 411-455 of VP16 (Triezenberg et al., 1988, Genes Dev. 2:730-742; Cress et al., 1991, Science 251:87-90) comprise the activation domain.

In a preferred embodiment, the yeast transcription factor GAL4 is reconstituted by protein-protein interaction and the host strain is mutant for GAL4. In another embodiment, the DNA-binding domain is Ace1N and/or the activation domain is Ace1, the DNA binding and activation domains of the Ace1 protein, respectively. Ace1 is a yeast protein that activates transcription from the CUP1 operon in the presence of divalent copper. CUP1 encodes metallothionein, which chelates copper, and the expression of CUP1 protein allows growth in the presence of copper, which is otherwise toxic to the host cells. The marker gene can also be a CUP1-lacZ fusion that expresses the enzyme beta-galactosidase (detectable by routine chromogenic assay) upon binding of a reconstituted Ace1N transcriptional activator (see Chaudhuri et al., 1995, FEBS Letters 357:221-226). In another specific embodiment, the DNA binding domain of the human estrogen receptor is used, with a marker gene driven by one or three estrogen receptor response elements (Le Douarin et al., 1995, Nucl. Acids. Res. 23:876-878).

The DNA binding domain and the transcriptional activator/inhibitor domain each preferably has a nuclear localization signal (see Ylikomi et al., 1992, EMBO J. 11:3681-3694; Dingwall and Laskey, 1991, TIBS 16:479-481) functional in the cell in which the fusion proteins are to be expressed.

To facilitate isolation of the encoded proteins, the fusion constructs can further contain sequences encoding affinity tags such as glutathione-S-transferase or maltose-binding protein or an epitope of an available antibody, for affinity purification (e.g., binding to glutathione, maltose, or a particular antibody specific for the epitope, respectively) (Allen et al., 1995, TIBS 20:511-516). In another embodiment, the fusion constructs further comprise bacterial promoter sequences for recombinant production of the fusion protein in bacterial cells.

The host cell in which the interaction assay occurs can be any cell, prokaryotic or eukaryotic, in which transcription of the marker gene can occur and be detected, including, but not limited to, mammalian (e.g., monkey, mouse, rat, human, bovine), chicken, bacterial, or insect cells, and is preferably a yeast cell. Expression constructs encoding and capable of expressing the binding domain fusion proteins, the transcriptional activation domain fusion proteins, and the marker gene product(s) are provided within the host cell, by mating of cells containing the expression constructs, or by cell fusion, transformation, electroporation, microinjection, etc. The host cell used should not express an endogenous transcription factor that binds to the same DNA site as that recognized by the DNA binding domain fusion population. Also, preferably, the host cell is mutant or otherwise lacking in an endogenous, functional form of the marker gene(s) used in the assay. Various vectors and host strains for expression of the two fusion protein populations in yeast are known and can be used (see e.g., U.S. Pat. No. 5,1468,614; Bartel et al., 1993, “Using the two-hybrid system to detect protein-protein interactions” In Cellular Interactions in Development, Hartley, ed., Practical Approach Series xviii, IRL Press at Oxford University Press, New York, N.Y., pp. 153-179; Fields and Sternglanz, 1994, Trends In Genetics 10:286-292). By way of example but not limitation, yeast strains or derivative strains made therefrom, which can be used are N105, N106, N1051, N1061, and YULH. Other exemplary strains that can be used in the assay of the invention also include, but are not limited to, the following:

Y190: MATa, ura3-52, his3-200, lys2-801, ade2-101, trpl-901, leu2-3,112, gal4α, gal80α, cyh^{r}2, LYS2::GALl_{UAS}-HIS3_{TATA}HIS3,URA3::GAL l_{UAS}-GALl_{TATA}-lacZ; Haper et al., 1993, Cell 75:805-816, available from Clontech, Palo Alto, Calif. Y190 contains HIS3 and lacZ marker genes driven by GAL4 binding sites.

CG-1945: MATa, ura3-52, his3-200, lys2-801, ade2-101, trpl-901, leu2-3,112, gal4-542, gal80-538, cyh^{r}2, LYS2::GALl_{UAS}-HIS3_{TATA}HIS3, URA3::GALl_{UAS17mers(x3)}-CYC1_{TATA}-lacZ, available from Clontech, Palo Alto, Calif. CG-1945 contains HIS3 and lacZ marker genes driven by GAL4 binding sites. Y187: MAT-α, ura3-52, his3-200, ade2-101, trp1-901, leu2-3,112, gal4α, gal80α, URA3::GAL1_{UAS}-GAL1_{TATA}-lacZ, available from Clontech, Palo Alto, Calif.

Y1 87 contains a lacZ marker gene driven by GAL4 binding sites.

SFY526: MATa, ura3-52, his3-200, lys2-801, ade2-101, trp1-901, leu2-3,112, gal4-542, gal80-538, can^{r}, URA3::GAL1-lacZ, available from Clontech, Palo Alto, Calif. SFY526 contains HIS3 and lacZ marker genes driven by GAL4 binding sites.

HF7c: MATa, ura3-52, his3-200, lys2-801, ade2-101, trp1-901, leu2-3,112, gal4-542, gal80-538, LYS2::GAL1-HIS3, URA3::GAL1_{UAS17MERS(x3)}-CYC1-lacZ, available from Clontech, Palo Alto, Calif. HF7c contains HIS3 and lacZ marker genes driven by GAL4 binding sites.

YRG-2: MATa, ura3-52, his3-200, lys2-801, ade2-101, trp1-901, leu2-3,112, gal4-542, gal80-538, LYS2::GAL1_{UAS}-GAL1_{TATA}-HIS3, URA3::GAL1_{UAS17mers(x3)}-CYC1-lacZ, available from Stratagene, La Jolla, Calif. YRG-2 contains HIS3 and lacZ marker genes driven by GAL4 binding sites. Many other strains commonly known and available in the art can be used.

If not already lacking in endogenous marker gene activity, cells mutant in the marker gene may be selected by known methods, or the cells can be made mutant in the marker gene by known gene-disruption methods prior to introducing the marker gene (Rothstein, 1983, Meth. Enzymol. 101:202-211).

In a specific embodiment, plasmids encoding the different fusion protein populations can be introduced simultaneously into a single host cell (e.g., a haploid yeast cell) containing one or more marker genes, by co-transformation, to conduct the assay for protein-protein interactions. Or, preferably, the two fusion protein populations are introduced into a single cell either by mating (e.g., for yeast cells) or cell fusions (e.g., of mammalian cells). In a mating type assay, conjugation of haploid yeast cells of opposite mating type that have been transformed with a binding domain fusion expression construct (preferably a plasmid) and an activation (or inhibitor) domain fusion expression construct (preferably a plasmid), respectively, will deliver both constructs into the same diploid cell. The mating type of a yeast strain may be manipulated by transformation with the HO gene (Herskowitz and Jensen, 1991, Meth. Enzymol. 194:132-146).

In a preferred embodiment, a yeast interaction mating assay is employed using two different types of host cells, strain-type a and alpha of the yeast Saccharomyces cerevisiae. The host cell preferably contains at least two marker genes, each with one or more binding sites for the DNA-binding domain (e.g., of a transcriptional activator). The activator domain and DNA binding domain are each parts of chimeric proteins formed from the two respective populations of proteins. One strain of host cells, for example the a strain, contains fusions of the library of nucleotide sequences with the DNA-binding domain of a transcriptional activator, such as GAL4. The hybrid proteins expressed in this set of host cells are capable of recognizing the DNA-binding site in the promoter or enhancer region in the marker gene construct. The second set of yeast host cells, for example, the alpha strain, contains nucleotide sequences encoding fusions of a library of DNA sequences fused to the activation domain of a transcriptional activator.

In a preferred embodiment, the fusion protein constructs are introduced into the host cell as a set of plasmids. These plasmids are preferably capable of autonomous replication in a host yeast cell and preferably can also be propagated in E. coli. The plasmid contains a promoter directing the transcription of the DNA binding or activation domain fusion genes, and a transcriptional termination signal. The plasmid also preferably contains a selectable marker gene, permitting selection of cells containing the plasmid. The plasmid can be single-copy or multi-copy. Single-copy yeast plasmids that have the yeast centromere may also be used to express the activation and DNA binding domain fusions (Elledge et al., 1988, Gene 70:303-312).

In another embodiment, the fusion constructs are introduced directly into the yeast chromosome via homologous recombination. The homologous recombination for these purposes is mediated through yeast sequences that are not essential for vegetative growth of yeast, e.g., the MER2, MER1, ZIPI, REC102, or ME14 gene.

Bacteriophage vectors can also be used to express the DNA binding domain and/or activation domain fusion proteins. Libraries can generally be prepared faster and more easily from bacteriophage vectors than from plasmid vectors.

In a specific embodiment, the present invention provides a method of detecting one or more protein-protein interactions combined with a negative selection step as described in PCT International Publication No. WO97/47763, published Dec. 18, 1997, which is incorporated by reference herein in its entirety.

In a preferred embodiment, the bait S. cerevisiae ergosterol sequence and the prey library of chimeric genes are combined by mating the two yeast strains on solid media, such that the resulting diploids contain both kinds of chimeric genes, i.e., the DNA-binding domain fusion and the activation domain fusion.

Preferred marker genes include the URA3, HIS3 and/or the lacZ genes (see e.g., Rose and Botstein, 1983, Meth. Enzymol. 101:167-180) operably linked to GAL4 DNA-binding domain recognition elements. Other marker genes include but are not limited to, Green Fluorescent Protein (GFP) (Cubitt et al., 1995, Trends Biochem. Sci. 20:448-455), luciferase, LEU2, LYS2, ADE2, TRP1, CAN1, CYH2, GUS, CUP1 or chloramphenicol acetyl transferase (CAT). Expression of the marker genes can be detected by techniques known in the art (see e.g. PCT International Publication No. WO97/47763, published Dec. 18, 1997, which is incorporated by reference herein in its entirety).

In a specific embodiment, transcription of the marker gene is detected by a linked replication assay. For example, as described by Vasavada et al., 1991, Proc. Natl. Acad. Sci. U.S.A. 88:10686-10690, expression of SV40 large T antigen is under the control of the E1B promoter responsive to GAL4 binding sites. The replication of a plasmid containing the SV40 origin of replication, indicates a protein-protein interaction. Alternatively, a polyoma virus replicon can be used (Vasavada et al., 1991, Proc. Natl. Acad. Sci. U.S.A. 88:10686-90).

In another embodiment, the expression of marker genes that encode proteins can be detected by immunoassay, i.e., by detecting the immunospecific binding of an antibody to such protein, which antibody can be labeled, or incubated with a labeled binding partner to the antibody, to yield a detectable signal. Alam and Cook disclose non-limiting examples of detectable marker genes that can be operably linked to a transcriptional regulatory region responsive to a reconstituted transcriptional activator, and thus used as marker genes (Alam and Cook, 1990, Anal. Biochem. 188:245-254).

The activation of marker genes like URA3 or HIS3 enables the cells to grow in the absence of uracil or histidine, respectively, and hence serves as a selectable marker. Thus, after mating, the cells exhibiting protein-protein interactions are selected by the ability to grow in media lacking a nutritional component, such as uracil or histidine (see Le Douarin et al., 1995, Nucl. Acids Res. 23:876-878; Durfee et al., 1993, Genes Dev. 7:555-569; Pierrat et al., 1992, Gene 119:237-245; Wolcott et al., 1966, Biochem. Biophys. Acta 122:532-534). In other embodiments of the present invention, the activities of the marker genes like GFP or lacZ are monitored by measuring a detectable signal (e.g., fluorescent or chromogenic, respectively) that results from the activation of these marker genes. LacZ transcription, for example, can be monitored by incubation in the presence of a substrate, such as X-gal (5-bromo-4-chloro-3-indolyl-β-D-galactoside), of its encoded enzyme, β-galactosidase. The pool of all interacting proteins isolated by this manner from mating the S. cerevisiae ergosterol-pathway sequence product and the library identifies the “ergosterol-pathway interactive population”.

In a preferred embodiment of the present invention, false positives arising from transcriptional activation by the DNA binding domain fusion proteins in the absence of a transcriptional activator domain fusion protein are prevented or reduced by negative selection prior to exposure to the activation domain fusion population (see e.g. PCT International Publication No. WO97/47763, published Dec. 18, 1997, which is incorporated by reference herein in its entirety). By way of example, if such cell contains URA3 as a marker gene, negative selection is carried out by incubating the cell in the presence of 5-fluoroorotic acid (5-FOA, which kills URA+ cells (Rothstein, 1983, Meth. Enzymol. 101:167-180). Hence, the metabolism of 5-FOA will lead to cell death of self-activating DNA-binding domain hybrids.

In a preferred aspect, negative selection involving a selectable marker as a marker gene can be combined with the use of a toxic or growth inhibitory agent to allow a higher rate of processing than other methods. Negative selection can also be carried out on the activation domain fusion population prior to interaction with the DNA binding domain fusion population, by similar methods, either alone or in addition to negative selection of the DNA binding fusion population. Negative selection can be carried out on the recovered protein-protein complex by known methods (see e.g., Bartel et al., 1993, BioTechniques 14:920-924; PCT International Publication No. WO97/47763, published Dec. 18, 1997).

In a preferred embodiment of the invention the DNA sequences encoding the pairs of interactive proteins are isolated by a method wherein either the DNA-binding domain hybrids or the activation domain hybrids are amplified, in separate respective reactions. Preferably, the amplification is carried out by polymerase chain reaction (PCR) (see U.S. Pat. Nos. 4,683,202; 4,683,195; and 4,889,818; Gyllenstein et al., 1988, Proc. Natl. Acad. Sci. U.S.A. 85:7652-7656; Ochman et al., 1988, Genetics 120:621-623; Loh et al., 1989, Science 243:217-220; Innis et al., 1990, PCR Protocols, Academic Press, Inc., San Diego, Calif.) using pairs of oligonucleotide primers specific for either the DNA-binding domain hybrids or the activation domain hybrids. Other amplification methods known in the art can be used, including but not limited to ligase chain reaction (see EP 320,308), use of Qβ replicase, or methods listed in Kricka et al., 1995, Molecular Probing, Blotting, and Sequencing, Academic Press, New York, Chapter 1 and Table IX.

The plasmids encoding the DNA-binding domain hybrid and the activation domain hybrid proteins can also be isolated and cloned by any of the methods well known in the art. For example, but not by way of limitation, if a shuttle (yeast to E. coli) vector is used to express the fusion proteins, the genes can be recovered by transforming the yeast DNA into E. coli and recovering the plasmids from E. coli (see e.g., Hoffman et al., 1987, Gene 57:267-272). Alternatively, the yeast vector can be isolated, and the insert encoding the fusion protein subcloned into a bacterial expression vector, for growth of the plasmid in E. coli.

### 5.9. Biochemical Assays Using Reporter or Target Proteins

The present invention provides for biochemical assays using the reporter or target proteins of the invention. In a specific embodiment, S. cerevisiae ergosterol-pathway proteins are useful for biochemical assays aimed at the identification and characterization of S. cerevisiae substrates or binding partners or the identification of ligands for ergosterol-pathway proteins that are yet to be assigned to the pathway. For any of the reporter or target genes of the invention, the cDNAs encoding reporter or target proteins can be individually subcloned into any of a large variety of eukaryotic expression vectors permitting expression in fungal, yeast, plant, insect, worm, mammalian, or other cell, as described above. The resulting genetically engineered cell lines expressing reporter or target proteins can be assayed for production, processing, and degradation of the reporter or target proteins, for example with antibodies to a specific reporter or target proteins, such as to an S. cerevisiae ergosterol-pathway protein, and Western blotting assays, or ELISA assays. For assays of specific binding and functional activation of binding-partner proteins, one can employ either crude culture medium or extracts containing secreted protein from genetically engineered cells (devoid of other ergosterol-pathway proteins), or partially purified culture medium or extracts, or preferably highly purified reporter or target protein fractionated, for example, by chromatographic methods. Alternatively, a reporter or target protein can be synthesized using chemical methods (Nagata, et al., 1992, peptides 13(4):653-62).

Specific protein binding of a reporter or target proteins to the reporter or target binding partners or substrates can be assayed as follows, for example, following the procedures of Yamaguchi et al. (Yamaguchi et al., 1995, Biochemistry 34:4962-4968). Chinese hamster ovary cells, COS cells, or any other suitable cell line, can be transiently transfected or stably transformed with expression constructs that direct the production of the reporter or target protein binding-partner or substrate. Direct binding of a reporter or target protein to such binding-partner or substrate-expressing cells can be measured using a “labeled” purified reporter or target protein derivative, where the label is typically a chemical or protein moiety covalently attached to the reporter or target polypeptide which permits the experimental monitoring and quantitation of the labeled reporter or target protein in a complex mixture.

Specifically, the label attached to the reporter or target protein can be a radioactive substituent such as an ¹²⁵I-moiety or ³²P-phosphate moiety, a fluorescent chemical moiety, or labels which allow for indirect methods of detection such as a biotin-moiety for binding by avidin or streptavidin, an epitope-tag such as a Myc- or FLAG-tag, or a protein fusion domain which allows for direct or indirect enzymatic detection such as an alkaline phosphatase-fusion or Fc-fusion domain. Such labeled reporter or target proteins can be used to test for direct and specific binding to binding-partner or substrate-expressing cells by incubating the labeled reporter or target protein with the binding-partner or substrate-expressing cells in serum-free medium, washing the cells with ice-cold phosphate buffered saline to remove unbound reporter or target protein, lysing the cells in buffer with an appropriate detergent, and measuring label in the lysates to determine the amount of bound reporter or target protein. Alternatively, in place of whole cells, membrane fractions or cell lysates obtained from binding-partner or substrate-expressing cells may also be used. Also, instead of a direct binding assay, a competition binding assay may be used. For example, crude extracts or purified reporter or target protein (such as an S. cerevisiae ergosterol-pathway protein) can be used as a competitor for binding of labeled purified reporter or target binding-partner or substrate-expressing cells, by adding increasing concentrations of reporter or target protein to the mixture. The specificity and affinity of binding of the reporter or target protein can be judged by comparison with other reporter or target proteins tested in the same assay.

5.9.1. Identification of Additional Binding-Partners

The invention described herein provides for methods in which reporter or target proteins are used for the identification of novel reporter or target protein binding-partners, using biochemical methods well known to those skilled in the art for detecting specific protein-protein interactions (Current Protocols in Protein Science, 1998, Coligan et al., eds., John Wiley & Sons, Inc., Somerset, N.J.). In particular, it is possible that some reporter or target proteins interact with binding-partners that have not yet been discovered, or binding-partners that are specific to a particular organism (e.g., fungi). The identification of either novel binding-partners or specific binding-partners is of great interest with respect to human therapeutic applications, such as, for example, antifungal applications. By way of example, the novel cognate binding-partners for ergosterol-pathway proteins can be investigated and identified as follows. Labeled S. cerevisiae ergosterol-pathway proteins can be used for binding assays in situ to identify cells possessing cognate binding-partners, for example as described elsewhere (Gorczyca et al., 1993, J. Neurosci. 13:3692-3704). Also, labeled S. cerevisiae ergosterol-pathway proteins can be used to identify specific binding proteins including binding-partner proteins by affinity chromatography of S. cerevisiae protein extracts using resins, beads, or chips with bound S. cerevisiae ergosterol-pathway protein (Formosa, et al., 1991, Methods Enzymol 208:24-45; Formosa, et al., 1983, Proc. Natl. Acad. Sci. USA 80(9):2442-6). Further, specific ergosterol-binding proteins can be identified by cross-linking of radioactively-labeled or epitope-tagged ergosterol-pathway protein to specific binding proteins in lysates, followed by electrophoresis to identify and isolate the cross-linked protein species (Ransone, 1995, Methods Enzymol 254:491-7). Still further, molecular cloning methods can be used to identify novel binding-partners and binding proteins for S. cerevisiae ergosterol-pathway proteins including expression cloning of specific binding-partners using S. cerevisiae cDNA expression libraries transfected into mammalian cells, expression cloning of specific binding proteins using S. cerevisiae cDNA libraries expressed in E. coli (Cheng and Flanagan, 1994, Cell 79(1):157-68), and yeast two-hybrid methods (as described above) using an S. cerevisiae ergosterol-pathway protein fusion as a “bait” for screening activation-domain fusion libraries derived from S. cerevisiae cDNA (Young and Davis, 1983, Science 222:778-82; Young and Davis, 1983, Proc. Natl. Acad. Sci. USA 80(5): 1194-8; Sikela and Hahn, 1987, Proc. Natl. Acad. Sci. USA 84(9):3038-42; Takemoto, et al., 1997, DNA Cell Biol. 16(6):797-9).

5.9.2. Assays of Pathway Proteins

The functional activity of reporter or target proteins, derivatives and analogs can be assayed by various methods known to one skilled in the art.

For example, in one embodiment, where one is assaying for the ability to bind to or compete with a wild-type reporter or target protein for binding to an antibody directed to the specific reporter or target protein, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in situ immunoassays (e.g., using colloidal gold, enzyme or radioisotope labels), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. In another embodiment, where a reporter or target protein is identified, the binding can be assayed, e.g., by means well-known in the art. In another embodiment, physiological correlates of reporter or target protein binding to its substrates and/or binding-partners (e.g., signal transduction) can be assayed.

In another embodiment, using insect (e.g., Sf9 cells), fly (e.g., D. melanogaster), or other model systems (such as other yeast or fungal systems, e.g., S. pombe), genetic studies can be done to study the phenotypic effect of a particular reporter or target gene mutant that is a derivative or analog of a wild-type reporter or target gene. Other such methods will be readily apparent to the skilled artisan and are within the scope of the invention.

The invention provides a method for identifying a molecule that activates the ergosterol pathway in yeast comprising contacting a yeast cell with one or more candidate molecules, and detecting a change in the RNA expression of a reporter gene for the ergosterol-pathway relative to the expression of the reporter gene in a yeast cell not contacted by the one or more candidate molecules, wherein the reporter gene is selected from the group consisting of: YHR039C (as depicted in FIG. 2, as set forth in SEQ ID NO:1), YLW100W (as depicted in FIG. 4, as set forth in SEQ ID NO:3), YPL272C (as depicted in FIG. 6, as set forth in SEQ ID NO:5), YGR131W (as depicted in FIG. 8, as set forth in SEQ ID NO:7), and YDR453C (as depicted in FIG. 10, as set forth in SEQ ID NO:9).

The invention provides a method for identifying a molecule that activates the ergosterol pathway in yeast comprising contacting a yeast cell with one or more candidate molecules, and detecting a change in the protein expression of a reporter gene for the ergosterol-pathway relative to the expression of the reporter gene in a yeast cell not contacted by the one or more candidate molecules, wherein the reporter gene is selected from the group consisting of: YHR039C (as depicted in FIG. 2, as set forth in SEQ ID NO:1), YLW100W (as depicted in FIG. 4, as set forth in SEQ ID NO:3), YPL272C (as depicted in FIG. 6, as set forth in SEQ ID NO:5), YGR131W (as depicted in FIG. 8, as set forth in SEQ ID NO:7), and YDR453C (as depicted in FIG. 10, as set forth in SEQ ID NO:9).

The invention provides a method for identifying a molecule that activates the PKC pathway in yeast comprising contacting a yeast cell with one or more candidate molecules, and detecting a change in the RNA expression of a reporter gene for the PKC-pathway relative to the expression of the reporter gene in a yeast cell not contacted by the one or more candidate molecules, wherein the reporter gene is selected from the group consisting of: SLT2(YHR030C) (as depicted in FIG. 17A-B, as set forth in SEQ ID NO:11), YKR161C (as depicted in FIGS. 19A-B, as set forth in SEQ ID NO:13), PIR3(YKL163W) (as depicted in FIGS. 21A-B, as set forth in SEQ ID NO:15), YPK2(YMR104C) (as depicted in FIGS. 23A-B, as set forth in SEQ ID NO:17), YLR194C (as depicted in FIGS. 25A-B, as set forth in SEQ ID NO:19), and ST1(YDR055W) (as depicted in FIGS. 27A-B, as set forth in SEQ ID NO:21).

The invention provides a method for identifying a molecule that activates the PKC pathway in yeast comprising contacting a yeast cell with one or more candidate molecules, and detecting a change in the protein expression of a reporter gene for the PKC-pathway relative to the expression of the reporter gene in a yeast cell not contacted by the one or more candidate molecules, wherein the reporter gene is selected from the group consisting of: SLT2(YHR030C) (as depicted in FIGS. 17A-B, as set forth in SEQ ID NO:11), YKR161C (as depicted in FIGS. 19A-B, as set forth in SEQ ID NO:13), PIR3(YKL163W) (as depicted in FIGS. 21A-B, as set forth in SEQ ID NO:15), YPK2(YMR104C) (as depicted in FIGS. 23A-B, as set forth in SEQ ID NO:17), YLR194C (as depicted in FIGS. 25A-B, as set forth in SEQ ID NO:19), and ST1(YDR055W) (as depicted in FIGS. 27A-B, as set forth in SEQ ID NO:21).

The invention provides a method for identifying a molecule that activates the Invasive Growth pathway in yeast comprising contacting a yeast cell with one or more candidate molecules, and detecting a change in the RNA expression of a reporter gene for the Invasive Growth pathway relative to the expression of the reporter gene in a yeast cell not contacted by the one or more candidate molecules, wherein the reporter gene is selected from the group consisting of KSS1(YGR040W) (as depicted in FIG. 29, as set forth in SEQ ID NO:23), PGU1(YJR153W) (as depicted in FIG. 31, as set forth in SEQ ID NO:25), YRL042C (as depicted in FIG. 33, as set forth in SEQ ID NO:27), and SVS1(YPL163C) (as depicted in FIG. 35, as set forth in SEQ ID NO:29).

The invention provides a method for identifying a molecule that activates the Invasive Growth pathway in yeast comprising contacting a yeast cell with one or more candidate molecules, and detecting a change in the protein expression of a reporter gene for the Invasive Growth pathway relative to the expression of the reporter gene in a yeast cell not contacted by the one or more candidate molecules, wherein the reporter gene is selected from the group consisting of: KSS1(YGR040W) (as depicted in FIG. 29, as set forth in SEQ ID NO:23), PGU1(YJR153W) (as depicted in FIG. 31, as set forth in SEQ ID NO:25), YRL042C (as depicted in FIG. 33, as set forth in SEQ ID NO:27), and SVS1(YPL163C) (as depicted in FIG. 35, as set forth in SEQ ID NO:29).

5.9.3. Proliferation & Cell Cycle Assays

A reporter or target gene, such as those of the invention may have potential implications in the ability of a cell to proliferate. The present invention provides for cell cycle and cell proliferation analysis by a variety of techniques known in the art, including but not limited to the following:

Bromodeoxyuridine (BRDU) incorporation may be used as an assay to identify proliferating cells. The BRDU assay identifies a cell population undergoing DNA synthesis by incorporation of BRDU into newly-synthesized DNA. Newly-synthesized DNA may then be detected using an anti-BRDU antibody (see Hoshino et al., 1986, Int. J. Cancer 38, 369; Campana et al., 1988, J. Immunol. Meth. 107, 79).

Cell Proliferation may also be examined using [³H]-thymidine incorporation (see e.g., Chen, J., 1996, Oncogene 13:1395-403; Jeoung, J., 1995, J. Biol. Chem. 270:18367-73). This assay allows for quantitative characterization of S-phase DNA snythesis. In this assay, cells synthesizing DNA will incorporate[³H]-thymidine into newly synthesized DNA. Incorporation can then me measured by standard techniques in the art such as by counting of radioisotope in a Scintillation counter (e.g. Beckman LS 3800 Liquid Scintillation Counter).

Cell proliferation may be measured by the counting samples of a cell population over time (e.g. daily cell counts). Cells may be counted using a hemacytometer and light microscopy (e.g. HyLite hemacytometer, Hausser Scientific). Cell number may be plotted against time in order to obtain a growth curve for the population of interest. In a preferred embodiment, cells counted by this method are first mixed with the dye Trypan-blue (Sigma), such that living cells exclude the dye, and are counted as viable members of the population. Alternatively, cells in a liquid solution may be counted by absorbency techniques known in the art.

DNA content and/or mitotic index of the cells may be measured, for example, based on the DNA ploidy value of the cell. For example, cells in the G1 phase of the cell cycle generally contain a 2N DNA polidy value. Cells in which DNA has been replicated but have not progressed thru mitosis (e.g. cells in S-phase) will exhibit polidy value higher than 2N and up to 4N DNA content. Ploidy value and cell cycle kinetics may further be measured using propidum iodide assay (see e.g. Turner, T., et al., 1998, Prostate 34:175-81). In an another embodiment, DNA content may be analyzed by preparation of a chromosomal spread (Zabalou, S., 1994, Hereditas. 120:127-40; Pardue, 1994, Meth. Cell Biol. 44:333-351).

Further assays include but are not limited to detection of changes in length of the cell cycle or speed of cell cycle. In one embodiment the length of the cell cycle is determined by the doubling time of a population of cells. In another embodiment, FACS analysis is used to analyze the phase of cell cycle progression, or purify G1, S, and G2/M fractions (see e.g., Delia, D., et al., 1997, Oncogene 14:2137-47). In a further embodiment, length or speed of the cell cycle of a test population is compared to wildtype populations.

Lapse of cell cycle checkpoint(s), and/or induction of cell cycle checkpoint(s), may be examined by the methods described herein, or by any method known in the art. Without limitation, a cell cycle checkpoint is a mechanism which ensures that a certain cellular events occur in a particular order. Checkpoint genes are defined by mutations that allow late events to occur without prior completion of an early event (Weinert, T., and Hartwell, L., 1993, Genetics, 134:63-80). Induction or inhibition of cell cycle checkpoint genes may be assayed, for example, by Western blot analysis, or by immunostaining, etc. Lapse of cell cycle checkpoints may be further assessed by the progression of a cell thru the checkpoint without prior occurrence of specific events (e.g. progression into mitosis without complete replication of the genomic DNA).

Other methods will be apparent to one skilled in the art and are within the scope of the invention.

5.9.4. Other Functional Assays

For functional assays of a reporter or target protein, beyond substrate binding, the following activities can be investigated using cells expressing a reporter or target protein of the invention after exposing said cells to crude or purified fractions of reporter or target protein and comparing these results with those obtained with other reporter or target proteins described above (Yamaguchi et al., 1995, Biochemistry 34:4962-4968). Assayable functional activities include but are not limited to stimulation of cell proliferation; inhibition of cell proliferation; cell death; cell membrane rupture; alterations in cell membrane integrity; stimulation of overall tyrosine kinase activity by immunoblotting of cell extracts with an anti-phosphotyrosine antibody; alteration of specific substrates in the biological-pathway in which the reporter or target are associated and immunoprecipitation with antibodies that specifically recognize the substrate protein; and stimulation of other enzymatic activities linked to the biological-pathway.

### 5.10. Assays for Changes in Gene Expression

This invention provides assays for detecting changes in the expression of the reporter or target genes and proteins. Assays for changes in gene expression are well known in the art (see e.g.,PCT Publication No. WO 96/34099, published Oct. 31, 1996, which is incorporated by reference herein in its entirety). Such assays may be performed in vitro using transformed cell lines, immortalized cell lines, or recombinant cell lines, or in vivo using animal models.

In particular, the assays may detect the presence of increased or decreased expression of a reporter or target gene or protein on the basis of increased or decreased mRNA expression (using, e.g., nucleic acid probes), increased or decreased levels of related protein products (using, e.g., the antibodies disclosed herein), or increased or decreased levels of expression of a marker gene (e.g., β-galactosidase or luciferase) operably linked to a 5′ regulatory region in a recombinant construct.

In yet another series of embodiments, various expression analysis techniques may be used to identify genes which are differentially expressed between two conditions, such as a cell line or animal expressing a normal reporter or target gene compared to another cell line or animal expressing a mutant reporter or target gene. Such techniques comprise any expression analysis technique known to one skilled in the art, including but not limited to differential display, serial analysis of gene expression (SAGE), nucleic acid array technology, subtractive hybridization, proteome analysis and mass-spectrometry of two-dimensional protein gels. In a specific embodiment, nucleic acid array technology (e.g., microarrays) may be used to determine a global (i.e., genome-wide) gene expression pattern in a normal S. cerevisiae animal for comparison with an animal having a mutation in one or more S. cerevisiae reporter or target genes.

To elaborate further, the various methods of gene expression profiling mentioned above can be used to identify other genes (or proteins) that may have a functional relation to (e.g., may participate in a signaling pathway with) a known gene. For example, gene identification of such other genes is made by detecting changes in their expression levels following mutation, i.e., insertion, deletion or substitution in, or overexpression, underexpression, mis-expression or knock-out, of an S. cerevisiae ergosterol-pathway gene, as described herein. Expression profiling methods thus provide a powerful approach for analyzing the effects of mutation in an S. cerevisiae ergosterol-pathway gene, or any reporter or target gene of the invention.

Methods of gene expression profiling are well-known in the art, as exemplified by the following references describing subtractive hybridization (Wang and Brown, 1991, Proc. Natl. Acad. Sci. U.S.A. 88:11505-11509), differential display (Liang and Pardee, 1992, Science 257:967-971), SAGE (Velculescu et al., 1995, Science 270:484-487), proteome analysis (Humphery-Smith et al., 1997, Electrophoresis 18:1217-1242; Dainese et al., 1997, Electrophoresis 18:432-442), and hybridization-based methods employing nucleic acid arrays (Heller et al., 1997, Proc. Natl. Acad. Sci. U.S.A. 94:2150-2155; Lashkari et al., 1997, Proc. Natl. Acad. Sci. U.S.A. 94:13057-13062; Wodicka et al., 1997, Nature Biotechnol. 15:1259-1267).

In a preferred specific embodiment of the invention expression analysis techniques are used to identify genes which are differentially expressed upon treatment of a cell with a drug, or by other perturbations. In a further specific embodiment, genes which are co-regulated (e.g., up-regulated upon treatment with a particular drug or antifungal agent) are mapped to gene sets using deletion mutants (See, e.g., Section 6.2) and microarray technology described herein. Still further, labeled cDNAs corresponding to a deletion mutant from drug treated or untreated cells are hybridized to a single microarray.

### 5.11. Reporter or Target Gene Regulatory Elements

This invention provides methods for using reporter or target gene regulatory DNA elements to identify cells, genes, and factors that specifically control reporter or target protein production. In one embodiment, regulatory DNA elements, such as enhancers/promoters, from S. cerevisiae ergosterol-pathway genes are useful for identifying and manipulating specific cells that synthesize an ergosterol-pathway protein. Such cells are of considerable interest since they are likely to have an important regulatory function within the fungus in controlling growth, development, reproduction, and/or metabolism. Analyzing components that are specific to a reporter or target secreting cells is likely to lead to an understanding of how to manipulate these regulatory processes, either for therapeutic applications, such as antifungal or fungicide applications, as well as an understanding of how to diagnose dysfunction in these processes. For example, it is of specific interest to investigate whether there are pathways genes in S. cerevisiae that might have a function related to that of the mammalian cholesterol pathway in sensing and controlling metabolic activity through the production of an ergosterol-pathway-like protein. Regulatory DNA elements derived from reporter or target genes provide a means to mark and manipulate such cells, and further, identify regulatory genes and proteins, as described below.

5.11.1. Protein-DNA Binding Assays

In a third embodiment, reporter or target gene regulatory DNA elements are also useful in protein-DNA binding assays to identify gene regulatory proteins that control the expression of such reporter or target genes. Such gene regulatory proteins can be detected using a variety of methods that probe specific protein-DNA interactions well known to those skilled in the art (Kingston, 1998, In Current Protocols in Molecular Biology, Ausubel et al, John Wiley & Sons, Inc., sections 12.0.3-12.10) including in vivo footprinting assays based on protection of DNA sequences from chemical and enzymatic modification within living or permeabilized cells, in vitro footprinting assays based on protection of DNA sequences from chemical or enzymatic modification using protein extracts nitrocellulose filter-binding assays and gel electrophoresis mobility shift assays using radioactively labeled regulatory DNA elements mixed with protein extracts. In particular, it is of interest to identify those DNA binding proteins whose presence or absence is specific to a reporter or target protein as judged by comparison of the DNA-binding assays described above using cells/extracts which express one or more reporter or target gene(s) versus other cells/extracts that do not express the same reporter or target genes. For example, a DNA-binding activity that is specifically present in cells that normally express an ergosterol-pathway protein might function as a transcriptional activator of an ergosterol-pathway reporter or target gene; conversely, a DNA-binding activity that is specifically absent in cells that normally express an ergosterol-pathway reporter or target protein might function as a transcriptional repressor of the ergosterol-pathway gene. Having identified candidate reporter or target gene regulatory proteins using the above DNA-binding assays, these regulatory proteins can themselves be purified using a combination of conventional and DNA-affinity purification techniques. In this case, the DNA-affinity resins/beads are generated by covalent attachment to the resin of a small synthetic double stranded oligonucleotide corresponding to the recognition site of the DNA binding activity, or a small DNA fragment corresponding to the recognition site of the DNA binding activity, or a DNA segment containing tandemly iterated versions of the recognition site of the DNA binding activity. Alternatively, molecular cloning strategies can be used to identify proteins that specifically bind a reporter or target gene regulatory DNA elements. For example, an S. cerevisiae cDNA library in an E. coli expression vector, such as the lambda-gt11 vector, can be screened for S. cerevisiae cDNAs that encode ergosterol-pathway gene regulatory element DNA-binding activity by probing the library with a labeled DNA fragment, or synthetic oligonucleotide, derived from the ergosterol-pathway gene regulatory DNA, preferably using a DNA region where specific protein binding has already been demonstrated with a protein-DNA binding assay described above (Singh et al., 1989, Biotechniques 7:252-61). Similarly, the yeast “one-hybrid” system can be used as another molecular cloning strategy (Li and Herskowitz, 1993, Science 262:1870-4; Luo, et al., 1996, Biotechniques 20(4):564-8; Vidal, et al., 1996, Proc. Natl. Acad. Sci. U.S.A. 93(19):10315-20). In this case, the ergosterol-pathway gene regulatory DNA element, for example, is operably fused as an upstream activating sequence (UAS) to one, or typically more, yeast marker genes such as the lacZ gene, the URA3 gene, the LEU2 gene, the HIS3 gene, or the LYS2 gene, and the marker gene fusion construct(s) inserted into an appropriate yeast host strain. It is expected that in the engineered yeast host strain the reporter genes will not be transcriptionally active, for lack of a transcriptional activator protein to bind the UAS derived from, for example, the S. cerevisiae ergosterol-pathway gene regulatory DNA. The engineered yeast host strain can be transformed with a library of S. cerevisiae cDNAs inserted in a yeast activation domain fusion protein expression vector, e.g. pGAD, where the coding regions of the S. cerevisiae cDNA inserts are fused to a functional yeast activation domain coding segment, such as those derived from the GAL4 or VP16 activators. Transformed yeast cells that acquire S. cerevisiae cDNAs that encode proteins that bind the gene regulatory element can be identified based on the concerted activation the marker genes, either by genetic selection for prototrophy (e.g., LEU2, HIS3, or LYS2 reporters) or by screening with chromogenic substrates (lacZ reporter) by methods known in the art.

### 6. EXAMPLES

The following examples are provided merely as illustrative of various aspects of the invention and shall not be construed to limit the invention in any way.

### 6.1. Characterization of S. Cerevisiae Ergosterol-Pathway Genes

A group of S. cerevisiae genes have been discovered as novel reporters of the ergosterol-pathway in the model organism S. cerevisiae. This invention provides the following examples of characterization of five S. cerevisiae ergosterol-pathway reporter genes described in detail below.

6.1.1. The Ergosterol Pathway

Ergosterol is the primary membrane sterol in fungi and in some trypanosomes. Ergosterol serves a structural role comparable to that of cholesterol in mammalian cells, and is essential for the integrity and structure of the fungal cell membrane. As depicted in FIG. 9, the ergosterol synthesis pathway contains at least 18 genes designated ERG1 though EGR26. Several different classes of antifungal agents exist which target the ergosterol-pathway.

6.1.2. Construction of Deletion Mutant

Deletion mutants were constructed by standard techniques, essentially as described by Rothstein, B., 1991, Meth. Enzymol. 194:281-301, which is incorporated herein by reference in its entirety. Specifically, a deletion mutant of the entire coding region of YER044C of S. cerevisiae was constructed in which the ORF YER044C was replaced by a dominant selectable marker (the kanamycin resistance gene) from Escherichia coli (Shoemaker, D. et al., 1996, Nature Gen. 14: 450-56; Rothstein, B., 1991, Meth. Enzymol. 194:281-301; Baudin, A, et al., 1993, Nuci. Acids Res. 21:3329-30). This deletion mutant (R711) has been deposited with with Research Genetics (Huntsville, Ala.) Deletion Consortium Strain #177. Briefly, the bacterial kanamycin resistance cassette (Wach, A et al., 1994, Yeast 10:1793-1808) was PCR amplified with primers that added homology to the YER044C locus, to direct homologous integration of the dominant selectable marker. Cell were then transformed with the PCR product. Cell were then selected for G418 resistance, and the gene replacement was confirmed by PCR with the appropriate primers flanking the YER044C locus.

The other genes deletions described in subsections below (e.g., BAR1, FUS3, DIG1, and DIG2) genes were constructed using the same techniques as for YER044C.

6.1.3. Growth of Yeast Strains and Drug Treatment

To assess the effects of pharmacologic inhibition of ergosterol biosynthesis, wild-type S. cerevisiae strain R174, (also known as strain BY4741, Brachmann, C., et al., 1998, Yeast, 14(2):115-32) was grown to early log-phase in YPD rich medium at 30° C. The culture was then split into 5 flasks and clotrimazole was added to a cultures at a final concentration of 0.03, 0.1, 1.0, and 3.0 ug/ml. The cultures were then incubated at 30° C. for 12 hours. Cells were then harvested, lysed and poly A+ RNA extracted, by methods known in the art. Specifically, cells were harvested and lysed by standard methods (In Current Protocols in Molecular Biology, Ausubel et al., John Wiley & Sons, Inc.) with the following modifications: Cell pellets were resuspended in breaking buffer (0.2M Tris HCl, pH 7.6/0.5M NaCl/10 mL EDTA/1% SDS), mixed for 2 minutes on a multi-tube vortex mixer at setting 8 in the presence of 60% (v/v) glass beads (425-600 urn mesh; Sigma, St. Louis, Mo.) and phenol:chloroform (50:50 v/v). Following separation of the phases, the aqueous phase, containing the total RNA, was reextracted and ethanol precipitated. Poly A+ RNA was isolated by two sequential chromatographic purifications over oligo dT cellulose (New England Biololabs Inc, Beverly, Mass.), as described In Current Protocols in Molecular Biology, Ausubel et al., John Wiley & Sons, Inc.

To assess the effects on the ergosterol pathway of deleting the YER044C gene, yeast strains R174 (wild type) and R711 (yer044c::kanR) were grown to early log phase in YPD medium, and harvested for preparation of polyA mRNAs.

6.1.4. Preparation and Hybridization of the Labeled cDNA

Fluorescentlylabeled cDNA was prepared by reverse transcription of polyA+ RNA in the presence of Cy3-(+drug) or Cy5-(−drug) deoxynucleotide triphosphates. Fluorescently labeled cDNAs were also purified, and hybridized essentially as described in DeRisi, J., 1997, Science 278:680-86, which is incorporated herein by reference in its entirety. Briefly, Cy3- or Cy5-dUTP (Amersham) was incorporated into cDNA during reverse transcription (Superscript II, Life Technologies, Inc., Gaithersburg, Md.). Labeled cDNAs were then concentrated to less than 10 ul using Microcon-30 microconcentrators (Amicon, Millipore, Corp,. Bedford, Mass.). Labeled cDNAs from drug treated or untreated cells were then resuspended in 20-26 ul hybridization solution (3×55G. 0.75 ug/ml poly A DNA, 0.2% SDS) and applied to the microarray (described below in section 6.2.3) under a 22×30 mm coverslip for 6 h. Both drug treated and untreated samples were simultaneously hybridized to the microarray as described in U.S. patent Ser. No. 179,569, filed Oct. 27, 1998 now pending, U.S. patent Ser. No. 09/220,275 filed Dec. 23, 1998, now pending, and U.S. patent Ser. No. 09/220,142, filed Dec. 23, 1998 now pending, which are incorporated herein by reference in their entirety. Under these conditions, drug treatment resulted in a signature pattern of altered gene expression in which mRNA levels of about 500 ORFs changed by at least twofold.

Alternatively, fluorescently-labeled cDNA was prepared, as above, by reverse transcription of polyA+ RNA from the YER044C deletion mutant and hybridized to the microarray. The signature of the deletion mutant was then compared to the signature of the drug-treated cells, as described below.

6.1.5. Fabrication of Microarrays

PCR products containing common 5′ and 3′ sequences were obtained from Research Genetics (Huntsville, Ala.), and used as templates with amino-modified forward primers and unmodified reverse primers to amplify 6065 ORFs from the yeast genome. Amplification reactions that gave products of unexpected sizes were excluded from subsequent analysis. ORFs that could not be amplified from purchased templates were amplified from genomic DNA. DNA samples from 100 ul reactions were precipitated with isopropanol, resuspended in water, brought up to a total volume of 15 ul in 3×SSC, and transferred to 384-well microtiter plates (Genetix Ltd, Dorset, United Kingdon). PCR products were robotically spotted onto 1×3 inch polylysine-coated glass slides. After printing, slides were processed as described in DeRisi et al. supra. 100% of the total ORFs of the yeast geneone were amplified and attached to the mircoarray, thus a DNA microarray consisting of more than 6000 oligonucleotides representing each of the known or predicted ORFs in the yeast genome was prepared.

6.1.6. Scanning and Imaging of Microarrays

Microarrays to which labeled cDNAs had been hybridized were then imaged on a prototype multi-frame charge-coupled device (CCD) camera (Applied Precision, Seattle, Wash.). Each CCD image frame was approximately 2 mm square. Exposure times of 2 sec in the Cy5 channel (white light through a Chroma 618-648 nm excitation filter, Chroma 657-727 mn emission filter) and 1 sec in the Cy3 channel (Chroma 53 5-560 nm excitation filter, Chroma 570-620 nm emission filter) were taken consecutively in each frame before moving to the next, spatially contiguous frame. Color isolation between the Cy3 and Cy5 channels was 100:1 or better. Frames were knitted together in software to make the complete images as in U.S. patent Ser. No. 179,569, filed Oct. 27, 1998 now pending, U.S. patent Ser. No. 09/220,275 filed Dec. 23, 1998, now pending, and U.S. patent Ser. No. 09/220,142, filed Dec. 23, 1998 now pending, which are incorporated herein by reference in their entirety. The intensity of each spot was quantified from the 10 um pixels by frame-by-frame background subtraction and intensity averaging in each channel. Normalization between the channels was accomplished by normalizing each channel to the mean intensities of all genes.

6.1.7. Assignment of Yeast ORFs to the Ergosterol Pathway Using DNA Microarray

The ORFs which are the subject of the present invention were discovered to be within the ergosterol pathway using DNA microarray technology (U.S. patent Ser. No. 179,569, filed Oct. 27, 1998 now pending, U.S. patent Ser. No. 09/220,275 filed Dec. 23, 1998, now pending, and U.S. patent Ser. No. 09/220,142, filed Dec. 23, 1998 now pending, which are incorporated herein by reference in their entirety).

Clotrimazole treatment of yeast resulted in the upregulation of aproximately 500 genes, many of which were induced by a wide variety of different types of perturbations of yeast. To determine which of these genea specifically assocoated with the ergosterol-pathway, the clotrimazole transcriptional signatures were compared with many other drug treatments and mutant signatures.

The similarity of signatures was quantified using the correlation coefficient. Correlation coefficients between the signature ORFs of various experiments were calculated according to Equation 4 in section 5.1 above, i.e., by the equation:

$${r}_{i , j} = \frac{{v}_{i} \cdot {v}_{j}}{\vert {v}_{i} ‖ {v}_{j} \vert} = \frac{{\sum }_{n} ( {v}_{i}^{( n )} \times {v}_{j}^{( n )} )}{{[ {\sum }_{n} {( {v}_{i}^{( n )} )}^{2} {\sum }_{n} {( {v}_{j}^{( n )} )}^{2} ]}^{1 / 2}} \qquad ( 10 )$$

where vᵢ⁽ⁿ⁾ and vⱼ⁽ⁿ⁾ are the log₁₀ of the expression ratio for the genes i and j, respectively, in response to perturbation n. The summation was over those genes that were either up- or down-regulated in either experiment at the 95% confidence level. These genes each had less than a 5% chance of being actually unregulated, that is, having expression ratios departing from unity due to measurement errors alone. This confidence level was assigned based on an error model which assigns a log normal probability distribution to each gene's expression ratio with characteristic width based on the observed scatter in its repeated measurements and on the individual array hybridization quality. This latter dependence was derived from control experiments in which both Cy3 and Cy5 samples were derived from the same RNA sample. As negative controls, deletion mutants known to affect pathways unrelated to ergosterol biosynthesis were analyzed. However, the mutant deleted in YER044C, which had not previously been assigned any function in the yeast genome, also gave a signature that correlated positively with the signature of drug-treated cells.

Using this analysis, two genes designated YHR039C and YLR100w were discovered to cluster on the same branch (as seen in FIG. 14) and were associated with the ergosterol pathway. These genes have been assigned as reporters of the ergosterol pathway. Three other genes have also been discovered to co-cluster on a second branch (as seen in FIG. 14) and have been discovered to be associated with the ergosterol pathway. These three genes YPL272c , YGR131c, and YDR453c were found to tightly cluster and have therefore been discovered to be associated with the ergosterol-pathway and act as novel reporters for the ergosterol pathway.

Taken together, these data indicated that five S. cerevisiae genes, designated YLR100W, YHR039C, YGL001C, YPL272c, YGR131c, and YDR453c were involved in the ergosterol biosynthesis pathway and were novel reporters for the pathway. One or a combination of these genes may also serve as targets for antifungal drug development.

### 6.2. Characterization of S. cerevisiae PKC-Pathway Genes

A group of S. cerevisiae genes have been discovered as novel reporters and/or targets of the PKC-pathway in the model organism S. cerevisiae. This invention provides the following examples of characterization of six S. cerevisiae PKC-pathway reporter genes described in detail below. Two of these S. cerevisiae PKC-pathway reporter genes have been further validated as target genes and are described in detail below.

6.2.1. The PKC Pathway

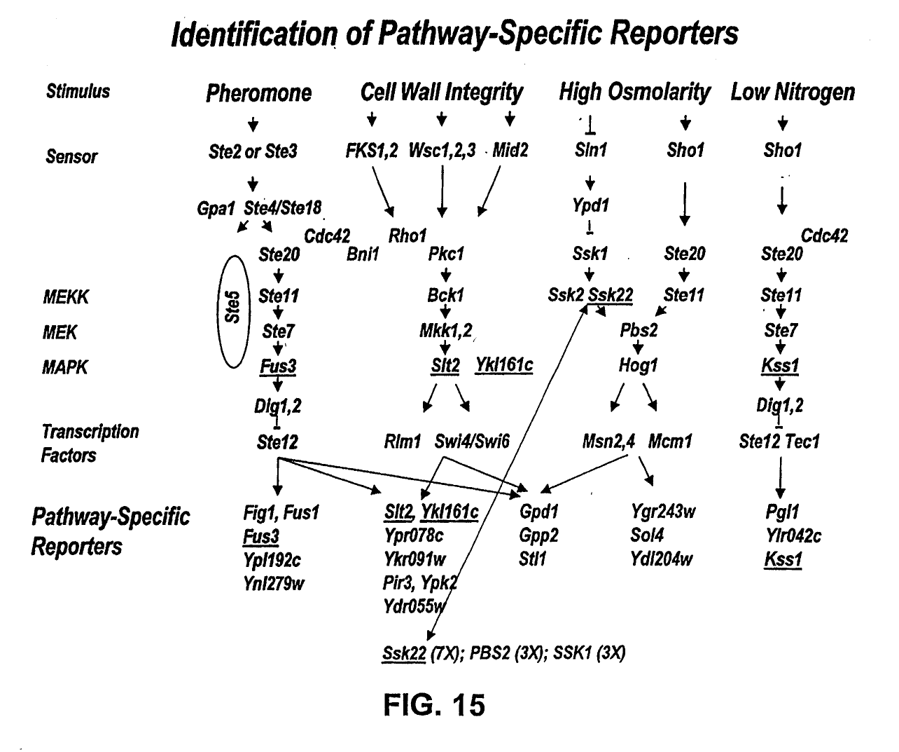

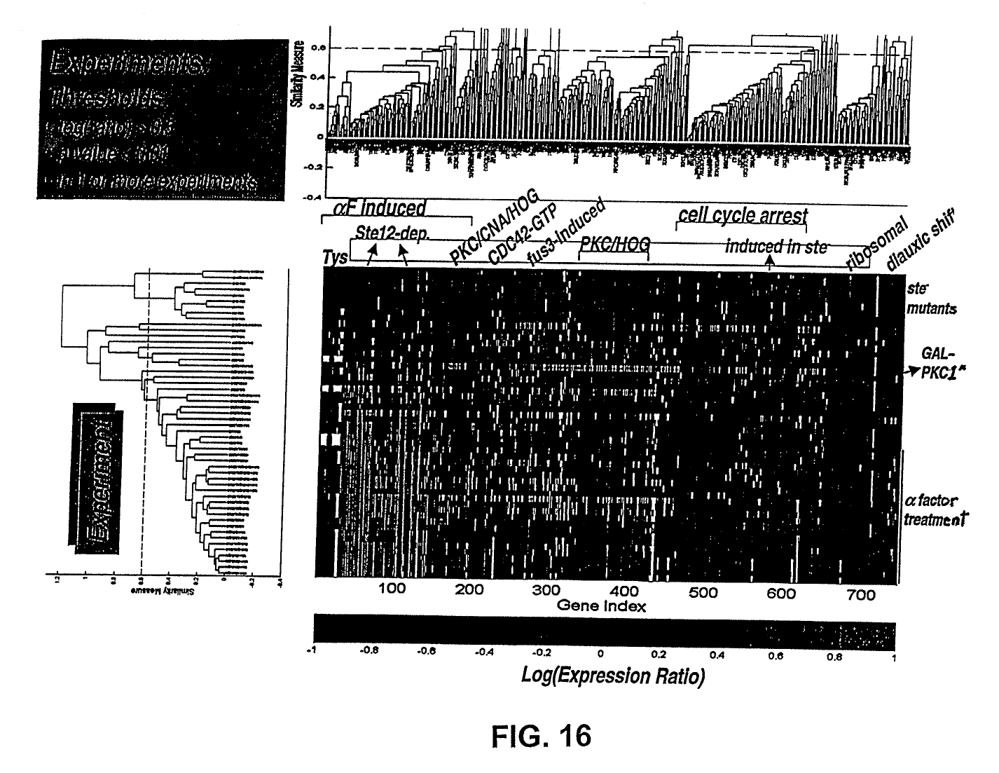

Protein kinase C (PKC) is a highly conserved protein throughout all eukaryotes. In the yeast S. cerevisiae PKC regulates the (MAP) kinase cascade, which is required for maintenance of cell integrity during periods of asymmetric or polarized growth. FIG. 15 shows a diagram of the PKC pathway in yeast, and demonstrates the reporters and target genes in the PKC pathway that have been discovered by the methods of the invention.

PKC plays a role in regulating the formation of a mating projection. The mating signal is transmitted to PKC through the activities of another Rho-GTPases, CDC42, and BNI1, and RHO1.

6.2.2. Novel PKC Reporter and Target Genes

In order to illustrate the methods of the invention, DNA microarray analysis was used to find reporters ans target genes of the PKC pathway. The transcriptional activity of yeast genes across a diverse number of experimental treatments of yeast, including a large number of drug treatments and mutations, as well as many experiments involving activation of the yeast mating process were used in the clustering analysis methods of the invention. Perturbation of the cells for PKC experiments was performed by constructing constitutively activated alleles of PKC (PKC1-R398A) or RHO1 (RHO-Q68H). Expression of these alleles were placed under the control of the inducible GAL1/10 promoter, and served as the perturbation. Cells containing constitutively activated alleles of PKC or RHO1 were compared to control cells lacking such activated alleles.

The yeast strains used to find reporter of the PKC pathway as are follows:

R4084=MATa bar1::kanR trp1-63 his3-200 leu2-0 met15-0 ura3-0 pRS316 (CEN URA3)

R4081=MATa bar1:kanR trp1-63 his 3-200 leu2-0 met15-0 ura3-0 pGAL-RHO1 (GAL1p-RHO1-Q68H, CEN, URA3)

R4075=MATa bar1::kanR leu2-0 his3-1 ura3-0 trp1-63 pGAL-PKC (GAL1p-PKC1-R398A, 2 micron, URA3)

R4081 contained the plasmid pGAL-RHO1, with the RHO1-Q68H gene controlled by the GAL1 promoter, on a low copy CEN, URA3-based plasmid. R4084 was a similar strain, only contained the plasmid pRS316, which is similar to pGAL-RHO1 except it lacks the RHO1-Q68H gene. R4075 was also similar to R4081,except it contained the plasmid pGAL-PKC, with the PKC1-R398A gene on a high copy 2 micron, URA3-based plasmid.

For PKC experiments, R4084 and R4075 or R4084 and R4081 were grown as pairs of cultures that were treated identically. The strains were grown as overnight cultures at 30C. in SC-ura (synthetic complete medium minus uracil; yeast nitrogen base, ammonium sulfate, and the complete set of amino acid supplements except uracil) with raffinose as the carbon sources. The cells were then subcultured at a low density in fresh medium for 2 hours, then galactose was directly added to the medium at a final concentration of 2%, and incubation continued for 3 hours. The cells were then harvested and total RNAs were prepared as labeled cDNAs for hybridization to microarrays. Pairs of hybridizations were done for each comparison, with the Cy3 and Cy5 fluors reserved for each pair to eliminate color biases due to differential fluor incorporation, as described above. The competitive hybridization pairs were as follows:

GAL-PKC1-R398A

1. Cy3=R4084(pRS316) vs Cy5=R4075 (pGAL-PKC1-R398A)

2. Cy3=R4075 (pGAL-PKC1-R398A) vs Cy5=R4084 (pRS316)

pGAL-RHO1-Q68H:

1. Cy3=R4084 (pRS316) vs Cy5=R4081 (pGAL-RHO1-Q68H)

2. Cy3=R4081 (pGAL-RHO1-Q68H) vs Cy5=R4084 (pRS316)

Results of cell perturbation by PKC activated alleles resulted in a large transcriptional response and co-clustered genesets. Comparison of the activated allele experiments to other experiments in the database (e.g., controls) using 2D clustering as described in U.S. patent Ser. No. 09/220,275 filed Dec. 23, 1998, now pending, and U.S. patent Ser. No. 09/220,142, filed Dec. 23, 1998 now pending, revealed novel reporter genes whose expression is activated only under conditions of PKC activation. These genes included PIR3, YPK2, YLR194C, YDR055W, SLT2 and YKL161C were discovered to be novel reporters of the PKC pathway. These four genes may serve as novel targets for inhibiting or modulating activation of the PKC pathway. Further, two of the genes, SLT2 and YKL161c were found to be located in the PKC pathway, and have therefore been discovered to serve as target genes of the PKC pathway.

Such novel PKC pathway-specific reporters have a wide variety of uses, including for example use in high throughput, cell based assays for general compounds activate PKC. Target genes have a wide variety of uses such as providing a target for which a drug designed to activate, inhibit or modify the PKC pathway may be designed and tested. Such target genes may also serve as the substrate or binding partner for a drug or compound which is tested for activity in activating, inhibiting or modifing the PKC pathway, or cellular responses and phenotypes associated with the PKC pathway, including for example, cell wall integrity.

### 6.3. Characterization of S. cerevisiae Invasive Growth Pathway Genes

A group of S. cerevisiae genes have been discovered as novel reporters and/or targets of the Invasive Growth pathway in the model organism S. cerevisiae. This invention provides the following examples of characterization of four S. cerevisiae Invasive Growth pathway reporter genes described in detail below. Two of these S. cerevisiae pathway reporter genes have been further validated as target genes.

6.3.1. The Invasive Growth Pathway

The yeast S. cerevisiae is dimorphic in that it can either proliferate either by budding or by forming multicellular filaments called pseudohyphae, which can invade the agar (Madhani and Fink, 1998, Trends Cell Biol 1998 September; 8(9):348-53). Diploid cells undergo the Invasive Growth pathway in response to nitrogen starvation, whereas haploid cells undergo the Invasive Growth pathway and form invasive filaments on rich medium. The mitogen-activated protein (MAP) kinase cascade is diagramed in FIG. 15.

6.3.2. Novel Invasive Growth Reporter and Target Genes

DNA microarray analysis of the genome of normal and mutant yeast strains was combined with two dimensional (2D) clustering analysis of the behaviors of 6000 genes across many perturbations. Using cluster analysis, a group of genes were identified to be indued transcriptionally in response to perturbations of the Invasive Growth pathway. Genes which were indued specifically to perturbations of the Invasive Growth pathway, were therefore discovered to be reporters for the Invasive Growth pathway. These genes included PGU1, YLR042C, SVS1, and KSS1 gene.

In order to search for Reporter genes of the Invasive Growth pathway, yeast strains with particular mutations (e.g., perturbations) were used as follows. The fus3 strain R500 (MATa bar1::kanR ura3-0 leu2-0 his3-1 met15-0 fus3::URA3) or the dig1 dig2 strain R4063 (MATa bar1::kanR ura3-0 leu2-0 his3-1 met15-0 dig1::LEU2 dig2::URA3), or the isogenic wild type parent, R276 (MATa bar1::kanR ura3-0 leu2-0 his3-1 met15-0), were grown as overnight cultures by standard methods in the art. Each culture was then diluted and grown to log phase. Alpha factor treatment was performed by adding 50 nM alpha factor directly to the cultures and incubating for 30 minutes. The cells were then harvested, total RNA was prepared by standard methods in the art, and polyA mRNAs were selected on oligo-dT cellulose. Next, fluorescently labeled cDNAs were prepared for DNA microarray experiments as described above. The following hybridizations were performed:

1. Strain R276 (wild type) vs. R500 (fus3), no alpha factor.

2. Strain R276 (wild type)+50 nM alpha factor, 30 minutes, vs strain R500 (fus3)+50 nM alpha factor, 30 min.

3. R276 vs. R4063 (dig1 dig2), neither with alpha factor.

The results of the hybridization experiments were examined by correlating the signatures to the signatures from a wide variety of other experiments, and by cluster analysis of gene behaviors across all these experiments. Four genes were found to be induced specifically in experiments in which the Invasive Growth pathway was activated, including KSS1, PGU1, YLR042C, and SVS1. Surprisingly, the MAPK KSS1 gene serves as a specific reporter and target for experiments in which KSS1 is active.

These target genes provide useful for screening for compounds that block invasive growth in S. cerevisiae. Because many aspects of the invasive growth pathway are conserved between S. cerevisiae and other pathogenic fungi, such as Candida albicans, and the switch to filamentous growth is essential for C. albicans virulence, such drugs will serves as novel antifungal agents.

The KSS1 gene will serve as a useful reporter for activation of the invasive growth pathway, since it has been discovered that induction of this gene is highly specific for this pathway. The use of combinations of two or more of the four invasive growth reporter genes will serve to greatly increase the sensitivity of such a reporter assay.

Each of the other genes have been discovered to be induced by other cellular perturbations. Specifically, PGU1 and YLR042C were found to be induced by treatment (e.g., perturbation) with the peptide pheromone, alpha factor. SVS1 was found to be repressed by alpha factor perturbation. Mutants deleted for the DIG1 and DIG2, in the absence of alpha factor, also showed increased transcription of the four genes PGU1, YLR042C, SVS1, and KSS1. Mutants deleted for the FUS3 MAPK, also showed several fold upregulation of the PGU1, YLR042C, SVS1, and KSS1 genes. Additionally, each of the PGU1, YLR042C, SVS1, and KSS1 genes were induced by activation of KSS1.

Such target genes may also serve as a substrate or binding partner for a drug or compound which is tested for activity in activating, inhibiting or modifying the Invasive Growth pathway, or cellular responses and phenotypes associated with the Invasive Growth pathway, including for example, invasion of fungus or pathogenicity of fungus.

### 6.4. Novel Reporter and Target Genes

A group of S. cerevisiae genes have been discovered by the methods of the invention as novel reporters and/or targets of the for pathways in the model organism S. cerevisiae. Table I, below lists such genes and there associated pathways, as well as the corresponding SEQ ID NOs.

TABLE 1

| Gene Name | Pathway | FIG. | SEQ ID NO. | |
|---|---|---|---|---|
| YHR039C | Ergosterol | 2 | 1 | DNA |
| | | 3 | 2 | Protein |
| YLR100W | Ergosterol | 4 | 3 | DNA |
| | | 5 | 4 | Protein |
| YPL272C | Ergosterol | 6 | 5 | DNA |
| | | 7 | 6 | Protein |
| YGR131W | Ergosterol | 8 | 7 | DNA |
| | | 9 | 8 | Protein |
| YDR453C | Ergosterol | 10 | 9 | DNA |
| | | 11 | 10 | Protein |
| SLT2(YHR030C) | PKC | 17A-B | 11 | DNA |
| | | 18 | 12 | Protein |
| YKL161C | PKC | 19A-B | 13 | DNA |
| | | 20 | 14 | Protein |
| PIR3(YKL163W) | PKC | 21A-B | 15 | DNA |
| | | 22 | 16 | Protein |
| YPK2(YMR104C) | PKC | 23A-B | 17 | DNA |
| | | 24 | 18 | Protein |
| YLR194C | PKC | 25A-B | 19 | DNA |
| | | 26 | 20 | Protein |
| PST1(YDR055W) | PKC | 27A-B | 21 | DNA |
| | | 28 | 22 | Protein |
| KSS1(YGR040W) | Invasive | 29 | 23 | DNA |
| | Growth | 30 | 24 | Protein |
| PGU1(YJR153W) | Invasive | 31 | 25 | DNA |
| | Growth | 32 | 26 | Protein |
| YLR042C | Invasive | 33 | 27 | DNA |
| | Growth | 34 | 28 | Protein |
| SVS1(YPL163C) | Invasive | 35 | 29 | DNA |
| | Growth | 36 | 30 | Protein |

The present invention is not to be limited in scope by the specific embodiments described herein. Indeed, various modifications of the invention in addition to those described herein will become apparent to those skilled in the art from the foregoing description and accompanying drawings. Such modifications are intended to fall within the scope of the appended claims.

Various references are cited herein above, including patent applications, patents, and publications, the disclosures of which are hereby incorporated by reference in their entireties.

What is claimed is:

1. A method of identifying a reporter gene for a particular biological pathway in a cell comprising identifying a gene which clusters to a geneset associated with the biological pathway, wherein said gene which clusters to the geneset associated with the particular biological pathway is a reporter gene.

2. The method of claim 1, wherein a geneset associated with the particular biological pathway is identified by a method comprising identifying one or more genes in a geneset which are associated with the particular biological pathway, wherein said geneset having one or more genes associated with the particular biological pathway is a geneset associated with the particular biological pathway.

3. The method of claim 1, wherein a geneset associated with the particular biological pathway is identified by identifying a geneset which is activated or inhibited by perturbations which target the biological pathway, wherein a geneset which is activated or inhibited by perturbations which target the biological pathway is a geneset associated with the particular biological pathway.

4. The method of claim 1, further comprising identifying a gene which clusters specifically to a geneset associated with the particular biological pathway, wherein said gene which clusters specifically to the geneset associated with the particular biological pathway is a reporter gene.

5. The method of claim 4, wherein the reporter gene is further identified as a gene whose expression is not altered by perturbations which effect other biological pathways, said other biological pathways being different from said particular biological pathway.

6. The method of claim 1, wherein geneset is provided by a method comprising:
(a) measuring changes in expression of a plurality of genes in the cell in response to a plurality of perturbations to the cell; and
(b) grouping or re-ordering said plurality of genes into one or more co-varying sets,
wherein said one or more co-varying sets comprise said geneset.

7. The method of claim 6, wherein said plurality of genes are grouped or re-ordered into one or more co-varying sets by means of a pattern recognition algorithm.

8. The method of claim 7, wherein the pattern recognition algorithm is a clustering algorithm.

9. The method of claim 8, wherein the clustering algorithm analyzes arrays or matrices, said arrays or matrices representing said measured changes in expression of the plurality of genes in the cell in response to the plurality of perturbations to the cell, wherein said analysis determines dissimilarities between individual genes.

10. The method of claim 6, wherein said plurality of perturbations to the cell are also grouped or re-ordered according to their similarity.

11. The method of claim 10, wherein said plurality of perturbations to the cell are grouped or re-oredered by means of a pattern recognition algorithm.

12. The method of claim 11, wherein the pattern recognition algorithm is a clustering algorithm.

13. The method of claim 12, wherein the clustering algorithm analyzes arrays or matrices, said arrays or matrices representing said measured changes in expression of the plurality of genes in the cell in response to the plurality of perturbations to the cell.

14. The method of claim 1, wherein the reporter gene is further identified as has a high level of induction.

15. The method of claim 14, wherein expression of the reporter gene is further identified to change by at least a factor of two in response to perturbations of the particular biological pathway.

16. The method of claim 15, wherein expression of the reporter gene is further identified to change by at least a factor of 10 in response to perturbations to the particular biological pathway.

17. The method of claim 16, wherein expression of the reporter gene is further identified to change by at least a factor of 100 in response to perturbations to the particular biological pathway.

18. The method of claim 1, wherein expression of the reporter gene is further identified to change in response to slight perturbations to the particular biological pathway.

19. The method of claim 18, wherein the perturbation to the particular biological pathway comprises exposure to a drug, and said reporter gene is further identified to change in response to low levels of exposure to the drug.

20. The method of claim 1, wherein the reporter gene is further identified to respond to perturbations targeted to the entire particular biological pathway.

21. The method of claim 1, wherein the reporter gene is further identified to respond to perturbations targeted to one or more portions of the particular biological pathway.

22. The method of claim 21, wherein the reporter gene is further identified to respond to perturbations targeted to early steps of the particular biological pathway.

23. The method of claim 21, wherein the reporter gene is further identified to respond to perturbations targeted to late steps of the particular biological pathway.

24. The method of claim 1, wherein the reporter gene is further identified by identifying a gene which kinetically induces quickly in response to perturbations to the particular biological pathway.

25. The method of claim 24, wherein the reporter gene is further identified by identifying a gene which reaches steady state within about eight hours after a perturbation to the particular biological pathway.

26. The method of claim 24, wherein the reporter gene is further identified by identifying a gene which reaches steady state within about six hours after a perturbation to the particular biological pathway.

27. The method of claim 24, wherein the reporter gene is further identified by identifying a gene which is induced within about two hours after a perturbation to the particular biological pathway.

28. The method of claim 27, wherein the reporter gene is further identified by identifying a gene which is induced within about 90 minutes after a perturbation to the particular biological pathway.

29. The method of claim 28, wherein the reporter gene is further identified by identifying a gene which is induced within about 60 minutes after a perturbation to the particular biological pathway.

30. The method of claim 29, wherein the reporter gene is further identified by identifying a gene which is induced within about 30 minutes after a perturbation to the particular biological pathway.

31. The method of claim 30, wherein the reporter gene is further identified by identifying a gene which is induced within about 10 minutes after a perturbation to the particular biological pathway.

32. The method of claim 31, wherein the reporter gene is further identified by identifying a gene which is induced within about 7 minutes after a perturbation to the particular biological pathway.

33. A method of identifying a target gene for a particular biological pathway in a cell comprising identifying a gene which clusters to a geneset associated with the particular biological pathway, wherein said gene which clusters to a geneset associated with the particular biological pathway and is identified as a gene which is necessary for normal function of said particular biological pathway.

34. The method of claim 33, wherein a geneset associated with the particular biological pathway is identified by a method comprising identifying one or more genes in a geneset which are associated with the particular biological pathway, wherein said geneset having one or more genes associated with the particular biological pathway is a geneset associated with the particular biological pathway.

35. The method of claim 33, wherein a geneset associated with the particular biological pathway is identified by identifying a geneset which is activated or inhibited by perturbations which target the biological pathway, wherein a geneset which is activated or inhibited by perturbations which target the biological pathway is a geneset associated with the particular biological pathway.

36. The method of claim 33, wherein genesets are provided by a method comprising:
(a) measuring changes in expression of a plurality of genes in the cell in response to a plurality of perturbations to the cell; and
(b) grouping or re-ordering said plurality of genes into one or more co-varying sets,
wherein said one or more co-varying sets comprise said genesets.

37. The method of claim 36, wherein said plurality of genes are grouped or re-ordered into one or more co-varying sets by means of a pattern recognition algorithm.

38. The method of claim 37, wherein the pattern recognition algorithm is a clustering algorithm.

39. The method of claim 38, wherein the clustering algorithm analyzes arrays of matrices, said arrays or matrices representing said measured changes in expression of the plurality of genes in the cell in response to the plurality of perturbations to the cell, wherein said analysis determines dissimilarities between individual genes.

40. The method of claim 36, wherein the plurality of perturbations to the cell are also grouped or re-ordered according to their similarity.

41. The method of claim 40, wherein the plurality of perturbations to the cell are grouped or re-ordered by means of a pattern recognition algorithm.

42. The method of claim 41, wherein the pattern recognition algorithm is a clustering algorithm.

43. The method of claim 42, wherein the clustering algorithm analyzes arrays of matrices, said arrays or matrices representing said measured changes in expression of the plurality of genes in the cell in response to the plurality of perturbations to the cell.

44. The method of claim 1, wherein the biological pathway is selected from the group consisting of: a signaling pathway, a control pathway, a mating pathway, a cell cycle pathway, a cell division pathway, a cell repair pathway, a small molecule synthesis pathway, a protein synthesis pathway, a DNA synthesis pathway, a RNA synthesis pathway, a DNA repair pathway, a stress-response pathway, a cytoskeletal pathway, a steroid pathway, a receptor-mediated signal transduction pathway, a transcriptional pathway, a translational pathway, an immune response pathway, a heat-shock pathway, a motility pathway, a secretion pathway, an endocytotic pathway, a protein sorting pathway, a phagocytic pathway, a photosynthetic pathway, an excretion pathway, an electrical response pathway, a pressure-response pathway, a protein modification pathway, a small-molecule response pathway, a toxic-molecule response pathway, and a transformation pathway.

45. The method of claim 1, wherein the reporter gene is a reporter for the ergosterol-pathway, and the reporter gene is selected from the group consisting of: YHR039C (as depicted in FIG. 2, as set forth in SEQ ID NO:1), YLW100W (as depicted in FIG. 4, as set forth in SEQ ID NO:3), YPL272C (as depicted in FIG. 6, as set forth in SEQ ID NO:5), YGR131W (as depicted in FIG. 8, as set forth in SEQ ID NO:7), and YDR453C (as depicted in FIG. 10, as set forth in SEQ ID NO:9).

46. The method of claim 1, wherein the reporter gene is a reporter for the PKC-pathway, and the reporter gene is selected from the group consisting of: SLT2(YHR030C) (as depicted in FIGS. 17A-B, as set forth in SEQ ID NO:11), YKR161C (as depicted in FIGS. 19A-B, as set forth in SEQ ID NO:13), PIR3(YKL163W) (as depicted in FIGS. 21A-B, as set forth in SEQ ID NO:15), YPK2(YMR104C) (as depicted in FIGS. 23A-B, as set forth in SEQ ID NO:17), YLR194C (as depicted in FIGS. 25A-B, as set forth in SEQ ID NO:19), and ST1(YDR055W) (as depicted in FIGS. 27A-B, as set forth in SEQ ID NO:21).

47. The method of claim 33, wherein the biological pathway is selected from the group consisting of: a signaling pathway, a control pathway, a mating pathway, a cell cycle pathway, a cell division pathway, a cell repair pathway, a small molecule synthesis pathway, a protein synthesis pathway, a DNA synthesis pathway, a RNA synthesis pathway, a DNA repair pathway, a stress-response pathway, a cytoskeletal pathway, a steroid pathway, a receptor-mediated signal transduction pathway, a transcriptional pathway, a translational pathway, an immune response pathway, a heat-shock pathway, a motility pathway, a secretion pathway, an endocytotic pathway, a protein sorting pathway, a phagocytic pathway, a photosynthetic pathway, an excretion pathway, an electrical response pathway, a pressure-response pathway, a protein modification pathway, a small-molecule response pathway, a toxic-molecule response pathway, and a transformation pathway.

48. The method of claim 33, wherein the target gene of the PKC-pathway is selected from the group consisting of: SLT2(YHR030C) (as depicted in FIGS. 17A-B, as set forth in SEQ ID NO:11), and YKR161C (as depicted in FIGS. 19A-B, as set forth in SEQ ID NO:13).

49. A method for determining whether a molecule affects the function or activity of an ergosterol pathway in a cell comprising:
(a) contacting the cell with, or recombinantly expressing within a cell the molecule; and
(b) determining whether the expression of one or more of the genes selected from the group consisting of: YHR039C (as depicted in FIG. 2, as set forth in SEQ ID NO:1), YLW100W (as depicted in FIG. 4, as set forth in SEQ ID NO:3), YPL272C (as depicted in FIG. 6, as set forth in SEQ ID NO:5), YGR131W (as depicted in FIG. 8, as set forth in SEQ ID NO:7), and YDR453C (as depicted in FIG. 10, as set forth in SEQ ID NO:9) is changed relative to said expression in the absence of the molecule.

50. The method according to claim 49 which is a method for determining whether the molecule inhibits ergosterol synthesis such that a cell contacted with the molecule exhibits a lower level of ergosterol than a cell which is not contacted with said molecule.

51. The method according to claim 49 wherein step (b) comprises determining whether YPL272c expression increases.

52. A kit comprising in one or more containers a) a substance selected from the group consisting of an antibody against an ergosterol-pathway protein, a gene probe capable of hybridizing to RNA of an ergosterol-pathway gene, and pairs of gene primers capable of priming amplification of at least a portion of an ergosterol-pathway gene, and b) a molecule known to be capable of perturbing the ergosterol pathway.

53. A method for identifying a molecule that activates the ergosterol pathway in yeast comprising contacting a yeast cell with one or more candidate molecules, and detecting a change in the RNA expression of a reporter gene for the ergosterol-pathway relative to the expression of the reporter gene in a yeast cell not contacted by the one or more candidate molecules, wherein the reporter gene is selected from the group consisting of: YHR039C (as depicted in FIG. 2, as set forth in SEQ ID NO:1), YLW100W (as depicted in FIG. 4, as set forth in SEQ ID NO:3), YPL272C (as depicted in FIG. 6, as set forth in SEQ ID NO:5), YGR131W (as depicted in FIG. 8, as set forth in SEQ ID NO:7), and YDR453C (as depicted in FIG. 10, as set forth in SEQ ID NO:9).

54. A method for identifying a molecule that activates the ergosterol pathway in yeast comprising contacting a yeast cell with one or more candidate molecules, and detecting a change in the protein expression of a reporter gene for the ergosterol-pathway relative to the expression of the reporter gene in a yeast cell not contacted by the one or more candidate molecules, wherein the reporter gene is selected from the group consisting of: YHR039C (as depicted in FIG. 2, as set forth in SEQ ID NO:1), YLW100W (as depicted in FIG. 4, as set forth in SEQ ID NO:3), YPL272C (as depicted in FIG. 6, as set forth in SEQ ID NO:5), YGR131W (as depicted in FIG. 8, as set forth in SEQ ID NO:7), and YDR453C (as depicted in FIG. 10, as set forth in SEQ ID NO:9).

55. The method according to claim 53, wherein the fungal cell is a transgenic cell.

56. The method according to claim 54, wherein the fungal cell is a transgenic cell.

57. A method for identifying a molecule that modulates the expression of an ergosterol-pathway gene selected from the group consisting of YHR039C (as depicted in FIG. 2, as set forth in SEQ ID NO:1), YLW100W (as depicted in FIG. 4, as set forth in SEQ ID NO:3), YPL272C (as depicted in FIG. 6, as set forth in SEQ ID NO:5), YGR131W (as depicted in FIG. 8, as set forth in SEQ ID NO:7), and YDR453C (as depicted in FIG. 10, as set forth in SEQ ID NO:9), comprising recombinantly expressing in a fungal cell one or more candidate molecules, and detecting the expression of said ergosterol-pathway gene; wherein an increase or decrease in the gene expression relative to the expression in the absence of candidate molecules indicates that the molecules modulates ergosterol-pathway gene expression.

58. The method according to claim 57, wherein the fungal cell is a transgenic cell.

59. A method for identifying a molecule that modulates the activity of an ergosterol-pathway protein selected from the group consisting of YHR039C (as depicted in FIG. 3, as set forth in SEQ ID NO:2), YLW100W (as depicted in FIG. 5, as set forth in SEQ ID NO:4), YPL272C (as depicted in FIG. 7, as set forth in SEQ ID NO:6), YGR131W (as depicted in FIG. 9, as set forth in SEQ ID NO:8), and YDR453C (as depicted in FIG. 11, as set forth in SEQ ID NO:10), comprising contacting a fungal cell with one or more candidate molecules, detecting said protein; wherein an increase or decrease in the protein level relative to the level in the absence of candidate molecules indicates that the molecule modulates ergosterol-pathway gene expression.

60. A method of identifying a molecule that binds to a ligand selected from the group consisting of (i) an S. cerevisiae ergosterol-pathway protein selected from the group consisting of YHR039C (as depicted in FIG. 3, as set forth in SEQ ID NO:2), YLW100W (as depicted in FIG. 5, as set forth in SEQ ID NO:4), YPL272C (as depicted in FIG. 7, as set forth in SEQ ID NO:6), YGR131W (as depicted in FIG. 9, as set forth in SEQ ID NO:8), and YDR453C (as depicted in FIG. 11, as set forth in SEQ ID NO:10), (ii) a fragment of the S. cerevisiae ergosterol-pathway protein, and (iii) a nucleic acid encoding the S. cerevisiae ergosterol-pathway protein or fragment, the method comprising:
(a) contacting the ligand with a plurality of molecules under conditions conducive to binding between the ligand and the molecules; and
(b) identifying a molecule within the plurality that binds to the ligand.

61. A method for determining whether a molecule affects the function or activity of an PKC pathway in a cell comprising:
(a) contacting the cell with, or recombinantly expressing within a cell the molecule; and
(b) determining whether the expression of one or more of the genes selected from the group consisting of: SLT2(YHR030C) (as depicted in FIG. 17A-B, as set forth in SEQ ID NO:11), YKR161C (as depicted in FIGS. 19A-B, as set forth in SEQ ID NO:13), PIR3(YKL163W) (as depicted in FIGS. 21A-B, as set forth in SEQ ID NO:15), YPK2(YMR104C) (as depicted in FIGS. 23A-B, as set forth in SEQ ID NO:17), YLR194C (as depicted in FIGS. 25A-B, as set forth in SEQ ID NO:19), and ST1(YDR055W) (as depicted in FIGS. 27A-B, as set forth in SEQ ID NO:21) is changed relative to said expression in the absence of the molecule.

62. The method according to claim 61 wherein step (b) comprises determining whether SLT2 expression increases.

63. A kit comprising in one or more containers a) a substance selected from the group consisting of an antibody against a PKC-pathway protein, a gene probe capable of hybridizing to RNA of a PKC-pathway gene, and pairs of gene primers capable of priming amplification of at least a portion of a PKC-pathway gene, and b) a molecule known to be capable of perturbing the PKC pathway.

64. A method for identifying a molecule that activates the PKC pathway in yeast comprising contacting a yeast cell with one or more candidate molecules, and detecting a change in the RNA expression of a reporter gene for the PKC-pathway relative to the expression of the reporter gene in a yeast cell not contacted by the one or more candidate molecules, wherein the reporter gene is selected from the group consisting of: SLT2(YHR030C) (as depicted in FIGS. 17A-B, as set forth in SEQ ID NO:11), YKR161C (as depicted in FIGS. 19A-B, as set forth in SEQ ID NO:13), PIR3(YKL163W) (as depicted in FIGS. 21A-B, as set forth in SEQ ID NO:15), YPK2(YMR104C) (as depicted in FIGS. 23A-B, as set forth in SEQ ID NO:17), YLR194C (as depicted in FIGS. 25A-B, as set forth in SEQ ID NO:19), and ST1(YDR055W) (as depicted in FIGS. 27A-B, as set forth in SEQ ID NO:21).

65. A method for identifying a molecule that activates the PKC pathway in yeast comprising contacting a yeast cell with one or more candidate molecules, and detecting a change in the protein expression of a reporter gene for the PKC-pathway relative to the expression of the reporter gene in a yeast cell not contacted by the one or more candidate molecules, wherein the reporter gene is selected from the group consisting of: SLT2(YHR030C) (as depicted in FIGS. 17A-B, as set forth in SEQ ID NO:11), YKR161C (as depicted in FIGS. 19A-B, as set forth in SEQ ID NO:13), PIR3(YKL163W) (as depicted in FIGS. 21A-B, as set forth in SEQ ID NO:15), YPK2(YMR104C) (as depicted in FIGS. 23A-B, as set forth in SEQ ID NO:17), YLR194C (as depicted in FIGS. 25A-B, as set forth in SEQ ID NO:19), and ST1(YDR055W) (as depicted in FIGS. 27A-B, as set forth in SEQ ID NO:21)

66. The method according to claim 64, wherein the fungal cell is a transgenic cell.

67. The method according to claim 65, wherein the fungal cell is a transgenic cell.

68. A method for identifying a molecule that modulates the expression of a PKC-pathway gene selected from the group consisting of SLT2(YHR030C) (as depicted in FIGS. 17A-B, as set forth in SEQ ID NO:11), YKR161C (as depicted in FIGS. 19A-B, as set forth in SEQ ID NO:13), PIR3(YKL163W) (as depicted in FIGS. 21A-B, as set forth in SEQ ID NO:15), YPK2(YMR104C) (as depicted in FIG. 23A-B, as set forth in SEQ ID NO:17), YLR194C (as depicted in FIG. 25A-B, as set forth in SEQ ID NO:19), and ST1(YDR055W) (as depicted in FIG. 27A-B, as set forth in SEQ ID NO:21), comprising recombinantly expressing in a fungal cell one or more candidate molecules, and detecting the expression of said PKC-pathway gene; wherein an increase or decrease in the gene expression relative to the expression in the absence of candidate molecules indicates that the molecules modulates PKC-pathway gene expression.

69. The method according to claim 68, wherein the fungal cell is a transgenic cell.

70. A method for identifying a molecule that modulates the activity of a PKC-pathway protein selected from the group consisting of SLT2(YHR030C) (as depicted in FIG. 18, as set forth in SEQ ID NO:12), YKR161C (as depicted in FIG. 20, as set forth in SEQ ID NO:14), PIR3(YKL163W) (as depicted in FIG. 22, as set forth in SEQ ID NO:16), YPK2(YMR104C) (as depicted in FIG. 24, as set forth in SEQ ID NO:18), YLR194C (as depicted in FIG. 26, as set forth in SEQ ID NO:20), and ST1(YDR055W) (as depicted in FIG. 28, as set forth in SEQ ID NO:22), comprising contacting a fungal cell with one or more candidate molecules, detecting said protein; wherein an increase or decrease in the protein level relative to the level in the absence of candidate molecules indicates that the molecule modulates PKC-pathway gene expression.

71. A method of identifying a molecule that binds to a ligand selected from the group consisting of (i) an S. cerevisiae PKC-pathway protein selected from the group consisting of SLT2(YHR030C) (as depicted in FIG. 18, as set forth in SEQ ID NO:12), YKR161C (as depicted in FIG. 20, as set forth in SEQ ID NO:14), PIR3(YKL163W) (as depicted in FIG. 22, as set forth in SEQ ID NO:16), YPK2(YMR104C) (as depicted in FIG. 24, as set forth in SEQ ID NO:18), YLR194C (as depicted in FIG. 26, as set forth in SEQ ID NO:20), and ST1(YDR055W) (as depicted in FIG. 28, as set forth in SEQ ID NO:22), (ii) a fragment of the S. cerevisiae PKC-pathway protein, and (iii) a nucleic acid encoding the S. cerevisiae PKC-pathway protein or fragment, the method comprising:
(a) contacting the ligand with a plurality of molecules under conditions conducive to binding between the ligand and the molecules; and
(b) identifying a molecule within the plurality that binds to the ligand.

72. A method for determining whether a molecule affects the function or activity of an Invasive Growth pathway in a cell comprising:
(a) contacting the cell with, or recombinantly expressing within a cell the molecule; and
(b) determining whether the expression of one or more of the genes selected from the group consisting of: KSS1(YGR040W) (as depicted in FIG. 29, as set forth in SEQ ID NO:23), PGU1(YJR153W) (as depicted in FIG. 31, as set forth in SEQ ID NO:25), YRL042C (as depicted in FIG. 33, as set forth in SEQ ID NO:27), and SVS1(YPL163C) (as depicted in FIG. 35, as set forth in SEQ ID NO:29), is changed relative to said expression in the absence of the molecule.

73. The method according to claim 72 wherein step (b) comprises determining whether KSS1(YGR040W) (as depicted in FIG. 29, as set forth in SEQ ID NO:23), expression increases.

74. A kit comprising in one or more containers a) a substance selected from the group consisting of an antibody against an Invasive Growth pathway protein, a gene probe capable of hybridizing to RNA of an Invasive Growth pathway gene, and pairs of gene primers capable of priming amplification of at least a portion of an Invasive Growth pathway gene, and b) a molecule known to be capable of perturbing the Invasive Growth pathway.

75. A method for identifying a molecule that activates the Invasive Growth pathway in yeast comprising contacting a yeast cell with one or more candidate molecules, and detecting a change in the RNA expression of a reporter gene for the Invasive Growth pathway relative to the expression of the reporter gene in a yeast cell not contacted by the one or more candidate molecules, wherein the reporter gene is selected from the group consisting of KSS1(YGR040W) (as depicted in FIG. 29, as set forth in SEQ ID NO:23), PGU1(YJR153W) (as depicted in FIG. 31, as set forth in SEQ ID NO:25), YRL042C (as depicted in FIG. 33, as set forth in SEQ ID NO:27), and SVS1(YPL163C) (as depicted in FIG. 35, as set forth in SEQ ID NO:29).

76. A method for identifying a molecule that activates the Invasive Growth pathway in yeast comprising contacting a yeast cell with one or more candidate molecules, and detecting a change in the protein expression of a reporter gene for the Invasive Growth pathway relative to the expression of the reporter gene in a yeast cell not contacted by the one or more candidate molecules, wherein the reporter gene is selected from the group consisting of: KSS1(YGR040W) (as depicted in FIG. 29, as set forth in SEQ ID NO:23), PGU1(YJR153W) (as depicted in FIG. 31, as set forth in SEQ ID NO:25), YRL042C (as depicted in FIG. 33, as set forth in SEQ ID NO:27), and SVS1(YPL163C) (as depicted in FIG. 35, as set forth in SEQ ID NO:29).

77. The method according to claim 75, wherein the fungal cell is a transgenic cell.

78. The method according to claim 76, wherein the fungal cell is a transgenic cell.

79. A method for identifying a molecule that modulates the expression of an Invasive Growth pathway gene selected from the group consisting of KSS1(YGR040W) (as depicted in FIG. 29, as set forth in SEQ ID NO:23), PGU1(YJR153W) (as depicted in FIG. 31, as set forth in SEQ ID NO:25), YRL042C (as depicted in FIG. 33, as set forth in SEQ ID NO:27), and SVS1(YPL163C) (as depicted in FIG. 35, as set forth in SEQ ID NO:29), comprising recombinantly expressing in a fungal cell one or more candidate molecules, and detecting the expression of said Invasive Growth pathway gene; wherein an increase or decrease in the gene expression relative to the expression in the absence of candidate molecules indicates that the molecules modulates Invasive Growth pathway gene expression.

80. The method according to claim 79, wherein the fungal cell is a transgenic cell.

81. A method for identifying a molecule that modulates the activity of an Invasive Growth pathway protein selected from the group consisting of KSS1(YGR040W) (as depicted in FIG. 30, as set forth in SEQ ID NO:24), PGU1(YJR153W) (as depicted in FIG. 32, as set forth in SEQ ID NO:26), YRL042C (as depicted in FIG. 34, as set forth in SEQ ID NO:28), and SVS1(YPL163C) (as depicted in FIG. 36, as set forth in SEQ ID NO:30), comprising contacting a fungal cell with one or more candidate molecules, detecting said protein; wherein an increase or decrease in the protein level relative to the level in the absence of candidate molecules indicates that the molecule modulates Invasive Growth pathway gene expression.

82. A method of identifying a molecule that binds to a ligand selected from the group consisting of (i) an S. cerevisiae Invasive Growth pathway protein selected from the group consisting of KSS1(YGR040W) (as depicted in FIG. 30, as set forth in SEQ ID NO:24), PGU1(YJR153W) (as depicted in FIG. 32, as set forth in SEQ ID NO:26), YRL042C (as depicted in FIG. 34, as set forth in SEQ ID NO:28), and SVS1(YPL163C) (as depicted in FIG. 36, as set forth in SEQ ID NO:30), (ii) a fragment of the S. cerevisiae Invasive Growth pathway protein, and (iii) a nucleic acid encoding the S. cerevisiae Invasive Growth pathway protein or fragment, the method comprising:
(a) contacting the ligand with a plurality of molecules under conditions conducive to binding between the ligand and the molecules; and
(b) identifying a molecule within the plurality that binds to the ligand.

83. The method of claim 1, wherein the reporter gene is a reporter for the Invasive Growth pathway, and the reporter gene selected from the group consisting of KSS1(YGR040W) (as depicted in FIG. 29, as set forth in SEQ ID NO:23), PGU1(YJR153W) (as depicted in FIG. 31, as set forth in SEQ ID NO:25), YRL042C (as depicted in FIG. 33, as set forth in SEQ ID NO:27), and SVS1(YPL163C) (as depicted in FIG. 35, as set forth in SEQ ID NO:29).


SEQUENCE LISTING (30 sequences)

SEQ ID NO: 1 - DNA, 2385 bp, Saccharomyces cerevisiae
  CDS (351)..(2282)
tctagttttctaatcatatatctttttataatataataccaatagaataaaaatgtataaactgacattgcattcggtctttacgactctcgctttatccattcagccttttttttttttttttttttttctctatctgctaaacgagtagtagtataatcaaaaatgtgttatttagtatatcggttgtaaaggagaaagtatggtctctctatttttattttattaacgaaaaatactaaacgccgatggggattactatataattataatagtatttgcagaatagtagaattcttttcacagttcacgttcagtttctcctctgttttatcgaacgtttattcatcatgtccaaggtctatctgaattcagacatgattaaccatttgaactccacagttcaagcttactttaacttatggttggagaagcaaaacgcaataatgcgttctcaaccccaaattattcaagataaccaaaaactgataggcattacaacgctagttgcctcaattttcactctgtatgttttggtcaagataatctccaccccagcaaagtgttcctcgtcctataagccagtcaaattctcccttcctgcaccagaggccgctcaaaataattggaagggcaagaggtctgtttccactaacatatggaatcctgaagaaccaaactttattcaatgtcattgtcccgccacaggtcaatatctaggttcttttccatcgaaaacggaagctgacatagatgaaatggtttctaaggcaggcaaagctcaatctacttggggcaattctgatttctcaagaagattgagagttttggcttctttgcatgattatattctaaataatcaagatcttattgcgagagtagcgtgcagggattcaggaaagacaatgttagacgcatcgatgggtgaaatcttggttactttagaaaaaattcaatggactataaagcacggccaaagagcgttgcaaccttcgagacgtccgggccccactaattttttcatgaagtggtataaaggtgcagaaatccgttatgaaccactgggtgtgatcagttctatcgtttcctggaactatccattccataacttattgggtccaattattgcagcattgttcacagggaatgccattgtagtaaaatgttcagaacaagttgtctggtcttcggaatttttcgtcgagctgatccgcaaatgtttggaagcttgtgatgaagatccagatttggttcagttgtgctattgtttacctccaactgaaaatgatgattccgcaaattatttcacctctcatcctggtttcaaacatatcacttttattggcagtcagcccgtagcgcactatattctaaaatgcgctgccaaatcattgacacccgtagttgtggagcttggtggtaaggatgcgtttattgtcctagactcagctaagaatttagatgctttatcttctatcatcatgaggggtactttccaatcatccggtcaaaattgtattggtattgagagggttattgtcagtaaggaaaattatgatgatttagtcaagattttgaatgaccgtatgactgcaaatccactacgccaagggtctgatattgatcatttagaaaatgttgatatgggggcaatgatatctgacaacagattcgatgaactagaagctttggttaaagatgctgttgcaaagggagctcgtttacttcaaggtggttcccgcttcaaacatccaaagtatccacaaggtcattatttccaaccaactcttttggtggatgtcactccagaaatgaaaatagcacaaaacgaagtgtttggcccaattttagtcatgatgaaagctaagaatactgaccattgtgtacaactagccaactctgcgccatttggtctaggtggttctgtgtttggtgcggatatcaaggaatgcaattacgtcgcaaatagcctacaaactggtaatgtagccattaatgattttgctacattctatgtttgtcaattaccatttggtggtatcaatggttcaggttacggtaaatttggtggtgaagaaggtcttttgggtttgtgcaatgccaaaagtgtctgttttgatactttgccttttgtctccactcaaattccaaaaccattagactaccctattcgtaacaatgctaaggcttggaattttgtaaagagtttcatcgtaggagcttatacaaattccacatggcaaagaataaagtcactgttctctttagctaaagaagccagctagtttactttagaggaagcaacaaacttatcaataatttggtatttattattatataaaatgaactttttatgtacaagatttatgattttttgattctata

SEQ ID NO: 2 - protein, 644 aa, Saccharomyces cerevisiae
MSKVYLNSDMINHLNSTVQAYFNLWLEKQNAIMRSQPQIIQDNQKLIGITTLVASIFTLYVLVKIISTPAKCSSSYKPVKFSLPAPEAAQNNWKGKRSVSTNIWNPEEPNFIQCHCPATGQYLGSFPSKTEADIDEMVSKAGKAQSTWGNSDFSRRLRVLASLHDYILNNQDLIARVACRDSGKTMLDASMGEILVTLEKIQWTIKHGQRALQPSRRPGPTNFFMKWYKGAEIRYEPLGVISSIVSWNYPFHNLLGPIIAALFTGNAIVVKCSEQVVWSSEFFVELIRKCLEACDEDPDLVQLCYCLPPTENDDSANYFTSHPGFKHITFIGSQPVAHYILKCAAKSLTPVVVELGGKDAFIVLDSAKNLDALSSIIMRGTFQSSGQNCIGIERVIVSKENYDDLVKILNDRMTANPLRQGSDIDHLENVDMGAMISDNRFDELEALVKDAVAKGARLLQGGSRFKHPKYPQGHYFQPTLLVDVTPEMKIAQNEVFGPILVMMKAKNTDHCVQLANSAPFGLGGSVFGADIKECNYVANSLQTGNVAINDFATFYVCQLPFGGINGSGYGKFGGEEGLLGLCNAKSVCFDTLPFVSTQIPKPLDYPIRNNAKAWNFVKSFIVGAYTNSTWQRIKSLFSLAKEAS

SEQ ID NO: 3 - DNA, 1944 bp, Saccharomyces cerevisiae
  CDS (801)..(1841)
acgtacaaaaaagagcacgctgctttatttatacttttgtgccacaagaatgatcaacatcaacataaatatcaactagtatctgcaacacatctgctccacggaactaaacccgttgagcagtgccccgtggaaacgtaaactatcgcaaattgggattaacaagccaaaaacagccaagcaagattcacgaaaccgcgcctcgtttggaccccgaaggcccatttaacggccggccgttacaagcaagatcggcagagcaaaccactccccagcaccacagcacatcactgcacgagcaacaataactagaacatggcagatagcgaggatacctctgtgatcctgcagggcatcgacacaatcaacagcgtggagggcctggaagaagatggttacctcagcgacgaggacacgtcactcagcaacgagctcgcagatgcacagcgtcaatgggaagagtcgctgcaacagttgaacaagctgctcaactgggtcctgctgcccctgctgggcaagtatataggtaggagaatggccaagactctatggagtaggttcattgaacactttgtataagtgtttgttgtttatgtatccgcatatagcagttataacagataaatggcacttttcgcacacccgttgttttatctccgatagtacgtgggcctttatttatggtcgtttaacgaaagaacggcatcttgaattgagcaggtatttaaaagataggacgagaaacaagcacatgatctgtgtcgaaaaaaagtagcaaagagaaaaagtaggaggataggatgaacaggaaagtagctatcgtaacgggtactaatagtaatcttggtctgaacattgtgttccgtctgattgaaactgaggacaccaatgtcagattgaccattgtggtgacttctagaacgcttcctcgagtgcaggaggtgattaaccagattaaagatttttacaacaaatcaggccgtgtagaggatttggaaatagactttgattatctgttggtggacttcaccaacatggtgagtgtcttgaacgcatattacgacatcaacaaaaagtacagggcgataaactaccttttcgtgaatgctgcgcaaggtatctttgacggtatagattggatcggagcggtcaaggaggttttcaccaatccattggaggcagtgacaaatccgacatacaagatacaactggtgggcgtcaagtctaaagatgacatggggcttattttccaggccaatgtgtttggtccgtactactttatcagtaaaattctgcctcaattgaccaggggaaaggcttatattgtttggatttcgagtattatgtccgatcctaagtatctttcgttgaacgatattgaactactaaagacaaatgcctcttatgagggctccaagcgtttagttgatttactgcatttggccacctacaaagacttgaaaaagctgggcataaatcagtatgtagttcaaccgggcatatttacaagccattccttctccgaatatttgaattttttcacctatttcggcatgctatgcttgttctatttggccaggctgttggggtctccatggcacaatattgatggttataaagctgccaatgccccagtatacgtaactagattggccaatccaaactttgagaaacaagacgtaaaatacggttctgctacctctagggatggtatgccatatatcaagacgcaggaaatagaccctactggaatgtctgatgtcttcgcttatatacagaagaagaaactggaatgggacgagaaactgaaagatcaaattgttgaaactagaacccccatttaatatatctctgcgtacatatgtatatatatatatgtgtgtatatacatgtatgtctgtatagaaaacgcatatcaactgatatatatacacgtgaagcaaa

SEQ ID NO: 4 - protein, 347 aa, Saccharomyces cerevisiae
MNRKVAIVTGTNSNLGLNIVFRLIETEDTNVRLTIVVTSRTLPRVQEVINQIKDFYNKSGRVEDLEIDFDYLLVDFTNMVSVLNAYYDINKKYRAINYLFVNAAQGIFDGIDWIGAVKEVFTNPLEAVTNPTYKIQLVGVKSKDDMGLIFQANVFGPYYFISKILPQLTRGKAYIVWISSIMSDPKYLSLNDIELLKTNASYEGSKRLVDLLHLATYKDLKKLGINQYVVQPGIFTSHSFSEYLNFFTYFGMLCLFYLARLLGSPWHNIDGYKAANAPVYVTRLANPNFEKQDVKYGSATSRDGMPYIKTQEIDPTGMSDVFAYIQKKKLEWDEKLKDQIVETRTPI

SEQ ID NO: 5 - DNA, 2754 bp, Saccharomyces cerevisiae
  CDS (1001)..(2551)
gatggcaaacctccgcaatgattggcgttctagcggctatccgaattcacaatcgacaagaagtacttctaacttacacaaggcaacgaaataatatcactctatgaaactgccatttgggtaataggagtatattgaacgacaccgggtcaacaagcaactttcctaagccttttacacttcttcacatcattcaagatcgccttttaacgagctacaaaccttcacgttcgttcttctatggaaacgtttaagataacgttaaaacgttctcaatcacagaatttaagatgattagaaatgttttccaagggatagggcgaagcacaacctcgaaaaatggcaaaattttagaatcttagccaccttaacgtctacttagagccttagaaaagccatcaagattggtggaatagttgttgagggaacttagccgccacattctcgtagccaaataaagcgaatctgaccattgtatgtttctttttcactggtatgatagcccaatgtgtttaaggaaagttaggacaacacacccgaagaaggacgtcacccctgcattcccaaacgagctatgaaatagctctttcctctacaagtaataacaacaacttttttgtctgttttccgaccgtttaacttcagagattaattttttcaacgcgctttcgttgaacgtcgcaaattcgtttagaataaacgaaaggtgacagaaatagaagattatagccatgcatacgcacataaattgaaaactgtttcgaggctgagtattccctgcgtctgcagccatcaggggtatgactctgctacacgtttactatattcttggctaaacgattcattaacgaagcgatgagtagatcacactcggcatacgagcacaaatttgtatggggggacggtcatatataaaagggtgtatacgttatccttgttatacctgtccaaagaagtgcatttgtaactcacaacacagacacatcctcactttatcataatgactacgtttaggccactatcaagttttgaaaaaaaaattctcactcaatctttgaatgaccaaagaaatggaactattttttcgagtacatattcaaaatctttaagtagagaaaatgacgctgactggcattctgatgaagtcacgctcggaacaaattcttccaaagatgattctcgtctgactctgcccctaatagcaacaactttgaagagattgattaaatcgcaaccggcattgtttgcaactgtaaacgaagaatgggaattcgagccattgaagcagctgaaaacttccgatattgttaatgtgattgagtttgaaaccataaaagataaggaggtcaattgccattggggtgttccacctccttatctcttgcgtcatgccttcaacaagactagatttgttcccggatcaaataaacctttatggacactatatgtaattgacgaagcgctattggtttttcatggtcacgacgtattgtttgatatattttcagcagctaactttcacaaattatttttaaaagagttaaacgaaatcagcacagtaacacactctgaagataggattttgtttgatgtcaatgacatcaatctctcagaattaaaatttcccaaatcgatatatgatagcgcaaaattacacctgcccgctatgacaccacaaatcttccacaagcaaactcagtcatttttcaaatcaatatactataacactttaaaaagacctttcggctatttaaccaatcaaacttccctcagctcgtcagtatctgcaacacagctgaaaaagtataatgatattctaaatgcgcacacctcattatgcgggacaacagtatttgggatagtaaacaaccaaaggtttaactatttaaagtcaatcgttaatcaagagcatatatgtctaagaagtttcatctgtggtattgcaatgatatgtttaaaacctctcgttaaggatttcagcggtacaatagtatttactattcccataaatttaagaaaccacttaggcttaggtgggtcattgggtctcttcttcaaagaactaagggtcgaatgtccactttctctaattgatgacgaactttccgccaacgaatttttgaccaacagtaacgataacgaggataatgatgatgagtttaatgaaagattgatggaatatcaatttaataaagttacaaagcacgttagcggttttattatggcaaaactgaggagttgggaaaagaatgggtttaatgatgacgatataaggaggatgaagtatgacaatgacgacgatttccatatccaaaattcaaggacaaaattgattcaaatcaatgatgtttccgacatatcgttatcgatgaacggcgatgacaaatctttcaaaattgtaagtacgggatttacaagttcgataaatcgccccacattaatgtctctttcctatacatactgtgaagagatgggcctgaatatctgtattcactaccctgattcgtataatttagaatcttttgtagaatgcttcgaatcctttattgaataggcaggtgacgcattaaatatatgtctgtatagtacgtattttttccattttatttattcttatcaaaatttaatcaacatatatgctaaagaaactattgataggagatatgacaggaaattgcactgtttctggaactttggcatgccgaggccgtcatttccagtataactgagcaaaaagaagtgacggtaaataca

SEQ ID NO: 6 - protein, 517 aa, Saccharomyces cerevisiae
MTTFRPLSSFEKKILTQSLNDQRNGTIFSSTYSKSLSRENDADWHSDEVTLGTNSSKDDSRLTLPLIATTLKRLIKSQPALFATVNEEWEFEPLKQLKTSDIVNVIEFETIKDKEVNCHWGVPPPYLLRHAFNKTRFVPGSNKPLWTLYVIDEALLVFHGHDVLFDIFSAANFHKLFLKELNEISTVTHSEDRILFDVNDINLSELKFPKSIYDSAKLHLPAMTPQIFHKQTQSFFKSIYYNTLKRPFGYLTNQTSLSSSVSATQLKKYNDILNAHTSLCGTTVFGIVNNQRFNYLKSIVNQEHICLRSFICGIAMICLKPLVKDFSGTIVFTIPINLRNHLGLGGSLGLFFKELRVECPLSLIDDELSANEFLTNSNDNEDNDDEFNERLMEYQFNKVTKHVSGFIMAKLRSWEKNGFNDDDIRRMKYDNDDDFHIQNSRTKLIQINDVSDISLSMNGDDKSFKIVSTGFTSSINRPTLMSLSYTYCEEMGLNICIHYPDSYNLESFVECFESFIE

SEQ ID NO: 7 - DNA, 1725 bp, Saccharomyces cerevisiae
  CDS (1001)..(1522)
tgcaaaaactgataagggctttcctgctgatgcgcttgctgattttgcgtatttgccgaagattgattgatcaattgcgtaaaggggtcgtcttcttgacggttgatattgaatagcatgttttgaatacgtagttgattgacctctttcttttaattgcgtgcagctgctctcaggtttaagatgtacgagggtccacggggtagcaagcacaagaacgatgatatatatgacagaacgatggataagaatggtatgttgtctgcactgttcagcattcgactacccctctcccggttcttttctcctcgtttcaatttaaaaaagcaactcgctacccggccgcacaccccttattcctgttcagccgtttaaggtgagaaccctttacttcatagcctttgtagatctttctattgctaccattgaagggtcggtgacgtggaaattttgacatttatcagtggcgtattgggaggcaagcaattgaaagaactgtgatttatttccgcttgttcgaaattattgatgtttagcactttgcagtagcgacaatacaatatatgtgcttttagtgctgggatagttcgtagctccatttcggggcgcttgttacatttattgtatatgcgcggatgtggcacatgctgttgagatctcactcctttggtatctctttcctgcgccgcattgtgccggcagaatgtcgcgcttgtattctcatgaacttttcctctttacgaaccctttggcggcatgccgtttaaaatctgttgaagatttcctttacgaacaatgagcaatgttttgcacaggcaggtgggaagtagggcctatcgcgccttggatgcagatataagtataaatataaattataataattggctgtatcagtaaatccttcttgcgatgggaggaagcacgatagagtatgttaagcttttgagaggcttcatattcattggaattttaaataacaataaagcaacaacaataataaatgctatcagctgcagataatttagtgcgcatcataaatgctgtttttcttattatatccataggtctaatcagcggcctgataggtacacagacaaagcatagttctcgagtgaacttttgtatgtttgccgccgtttatggtctggttacggattcattatatgggtttttggctaatttctggacatcattaacatacccagcaattttgcttgttttggattttttaaatttcatatttacgtttgtagcagccaccgctttggctgtaggtataagatgccattcgtgtaaaaacaaaacatatctggaacagaataagatcatacaaggctcaagctccagatgtcatcaatctcaggctgctgttgcgtttttttacttttcctgttttctattcctcatcaaagtgactgtggccacgatgggtatgatgcaaaatggtggatttggctctaataccggattcagcagaaggagggcaagaagacaaatgggcatacctacaatttcccaggtttaagcctactggactgaaaaaaaggcaattcgcgtacaattttcgttgatcgttctttatataacctttgcattaaataaatttaacaaaaaaagttctttctaaaataatattatggtgatacatgaatgtgctttagttttttcgtaggctcatccatgtatatatataaatgataaaaaactaagttacgatattgatag

SEQ ID NO: 8 - protein, 174 aa, Saccharomyces cerevisiae
MLSAADNLVRIINAVFLIISIGLISGLIGTQTKHSSRVNFCMFAAVYGLVTDSLYGFLANFWTSLTYPAILLVLDFLNFIFTFVAATALAVGIRCHSCKNKTYLEQNKIIQGSSSRCHQSQAAVAFFYFSCFLFLIKVTVATMGMMQNGGFGSNTGFSRRRARRQMGIPTISQV

SEQ ID NO: 9 - DNA, 1791 bp, Saccharomyces cerevisiae
  CDS (1001)..(1588)
gtagatgaattcaaatctatgattaagaacaatgaattcattgaatgggcgcaattctccggtaactactatggtagtactgtcgcttccgtcaaacaagtcagtaaatctggtaagacttgtattttagatattgatatgcagggtgtcaaatctgtcaaggctatcccagagttaaatgccaggtttttgtttattgctccaccatcggtcgaggatttgaaaaaaagattagaaggtagaggtacggagaccgaagaatccatcaacaagaggttaagcgccgctcaagctgaattggcatatgctgagacaggtgcccatgacaaagttattgtcaatgatgatttggacaaggcctacaaggaattgaaggattttatctttgcagaaaaatgatgtagccctatatagacattactaagtatgtacctggtaggagagtgctgtcgcaaagcgacaaaacgtccaattattcaattaatatagtgtaaaagttctcaacgggcttatgctagttttttttgttagtaagcgctacgacgactagaaccatctcttgaatttccaagtgccaaaatcaatgaccacggatactgtggccaggaatctgttggttggtcatcctcaagatctagacaatatcatattgggccagtatctgattatcttaactatatgcgcccctctagtttacaagttttagtcattgggggttggaagggctgatccccccttacaattggcgtcgtttaggagcgggcgaggctctcctttctcttacacatctgctaaggtgtttgttacccgagtaatcaaggatcaactatggatgagatttagattaacgtatttagagcagacgattgtaagaatatattttgtaatttcgattgttttttgctacttacattgtttatcttgaaatatccaaagtgaacactattactgttttttgctcaagaatatattagccttacaagaacgtaaaaaaccaatcatggtagcagaagttcaaaaacaagccccaccatttaagaaaaccgccgtagtcgacggtatcttcgaggaaatttcactggaaaagtataaaggtaagtacgttgttctagcttttgtcccattggctttttcatttgtctgtccaactgagattgttgcgttttccgatgccgccaagaaattcgaagatcagggcgcccaagttttatttgcctccaccgactctgaatattccttactggcatggaccaaccttcccagaaaagacggtggattaggtccagttaaagttcctttgcttgctgataagaatcattccttatccagagactatggcgttttgattgaaaaagaaggtatagctttaagaggtttgttcataatcgacccgaagggaatcattagacatatcactatcaatgatttatctgttggcagaaacgtcaatgaagctttgagattagtcgaaggtttccagtggactgacaaaaatggtacagttttgccatgcaactggaccccaggagccgccaccatcaaacctgacgttaaagattccaaggagtatttcaaaaatgccaataattaatcttcgcacgataacgctaggccctattaaataattaaaaatacatcaccctatatatgataagaaagatggttttgtattattatgaaattgacttgaaagaatagtgtaacaaaagaaaaagaaactgtaattgaagaatgatatgcatttctatgtgtatattaacttaatcatctttatatccagaagacgcaaat

SEQ ID NO: 10 - protein, 196 aa, Saccharomyces cerevisiae
MVAEVQKQAPPFKKTAVVDGIFEEISLEKYKGKYVVLAFVPLAFSFVCPTEIVAFSDAAKKFEDQGAQVLFASTDSEYSLLAWTNLPRKDGGLGPVKVPLLADKNHSLSRDYGVLIEKEGIALRGLFIIDPKGIIRHITINDLSVGRNVNEALRLVEGFQWTDKNGTVLPCNWTPGAATIKPDVKDSKEYFKNANN

SEQ ID NO: 11 - DNA, 3455 bp, Saccharomyces cerevisiae
  CDS (1001)..(2452)
gtggtgaaaatgaaggaaatttacaagattgtggatgacgaagttgtcatggacatgagattagtgagtcgggtcattggtaatcccttgttaaaggaatcaaaggagtttcgtcaagatttgaatgccaggccattagctagattggaacgtttgaaaatcttgataaactatgcagttaagatctctccgcataaggaaaaattcccctatgtgaggtggacagtgggtaaaaacaagtacatacatgagctcatggtcccagagcgctttcccattgatattcccagagaaaatgtcgggttagaaagaactcagattccattaatgctatgctgggcactgtccattcataaggcacagggtcaaactattcaaagactaaaggtcgacttgaggagaattttcgaagccggccaagtttatgttgcactgtcaagagcggtaactatggacaccttacaggtcctaaactttgatccaggaaagattcgcaccaatgaaagagtaaaagatttctataaacgtttagaaactttgaaatgacttgcaacgaataaatgcatatactctagttgaagttttcttttcttgttctatacaggttcgaatacttgtgagcctatctgtataatttaacagaatcccgaaatattcatctagaagccatctatttagctaagcctacgtatgcggcgatttttatattatctttttttttttttatagaagactgcgaaatgttggcagaatggaaagtttcagtgttaaaaatagaaactgaaaaaggagatctagccaggaatatatcgaaaaaaaaagtgagggaaatcagatcctacacaaatatttagatttaattgaagaccctggtctgccagatatatatatatattagacgaactgtgcattcagtcagcaaatctaggccacagattttcttattgaagctatcaaaatagtagaaataattgaagggcgtgtataacaattctgggagatggctgataagatagagaggcatactttcaaggtcttcaatcaagatttcagtgtagataagaggtttcaacttatcaaagaaatagggcatggagcatacggcatagtgtgttcagcgcggtttgcagaagctgccgaagataccacagttgccatcaagaaagtgacaaacgttttttcgaagaccttactatgtaaaagatccctacgtgagctaaagcttttgagacatttcagaggccacaaaaatattacatgtctttatgatatggatattgttttttatccagacgggtctatcaatggactatatctttatgaggaacttatggaatgtgatatgcaccaaatcatcaaatccggtcaacctttgacggatgctcactatcaaagtttcacataccaaatattatgtggtttaaagtatattcattctgcagatgtcttgcatcgtgatttgaagcccggcaatttgcttgtcaatgcagattgtcaattgaaaatctgtgattttgggttagctagaggttattcggagaatcctgtcgaaaacagtcaatttttgacggagtacgtggccactagatggtatagagctccggaaataatgttgagttaccaaggatataccaaggcgattgacgtatggtcagctggctgtattttagcggagtttcttggtggaaagccaatcttcaaaggaaaggattacgttaatcaattgaatcaaatattacaagttttagggacacccccagacgaaactttaagaaggattggttctaaaaatgttcaggactacatacatcaattaggtttcattccaaaagtaccttttgtcaatttatacccaaatgccaattcacaagcattagacttattggagcaaatgctcgcgtttgaccctcaaaagagaattaccgtggatgaggccctggagcatccttacttgtctatatggcatgatccagctgacgaacctgtgtgtagtgaaaaattcgaatttagttttgaatcggttaatgatatggaggacttaaaacaaatggttatacaagaagtgcaagatttcaggctgtttgtgagacaaccgctattagaagagcaaaggcaattacaattacagcagcagcaacagcagcagcaacagcaacagcaacagcaacagcagccttcagatgtggataatggcaacgccgcagcgagtgaagaaaattatccaaaacagatggccacgtctaattctgttgcgccacaacaagaatcatttggtattcactcccaaaatttgccaaggcatgatgcagatttcccacctcgacctcaagagagtatgatggagatgagacctgccactggaaataccgcagatattccgcctcagaatgataacggcacgcttctagaccttgaaaaagagctggagtttggattagatagaaaatatttttaggacaaaaaactataagtaaccggggaagtatagaatcaccatagatgtaagcttacagacaatgtgtatatatgatgtatatgaacgtatacaaatatatatatatatacgtgctcttgttgtagctcgtatatcaaattcctcctccgacgcttatcttaatcgtactccgcggaagtttgttatcgcctcttgaattctttcttttcgttcatttatgattagtcatctatagacaatattcattatttaagcacctagaatactaaactaaatgtctaaatatgacacaaggaagataagataaaaaaaaccaagcgcttagaatatgactttaatggtacctttcaaacaagttgatgtattcactgagaagccctttatgggaaatccagtagcagtaataaacttcttggaaattgatgaaaatgaagtcagtcaagaagaattgcaggcaattgccaactggacaaacttatcagaaacaacgtttttatttaaaccatctgataaaaagtatgattacaagttgaggatctttactccaagaagtgaattgccatttgctggtcacccaaccattggttcatgtaaggctttccttgagttcaccaaaaacaccactgcgacttctctcgtccaggaatgtaaaataggcgctgttccaataacaattaatgagggactaattagcttcaaagctccgatggctgattacgaaagtatatcgagtgagatgattgctgattatgaaaaagcgattggtttgaaattcataaagcctcctgctcttttacatactgggccagagtggatcgtggcgctagtagaagatgcagaaacttgcttcaatgcaaacccaaattttgctatgcttgcacaccagacaaaacagaatgaccatgtgggaattatcctagcgggccctaaaaaggaagccgccatcaaaaactcctacgaaatgagggcgtttgctccggtgataaacgtttatgaagat

SEQ ID NO: 12 - protein, 484 aa, Saccharomyces cerevisiae
MADKIERHTFKVFNQDFSVDKRFQLIKEIGHGAYGIVCSARFAEAAEDTTVAIKKVTNVFSKTLLCKRSLRELKLLRHFRGHKNITCLYDMDIVFYPDGSINGLYLYEELMECDMHQIIKSGQPLTDAHYQSFTYQILCGLKYIHSADVLHRDLKPGNLLVNADCQLKICDFGLARGYSENPVENSQFLTEYVATRWYRAPEIMLSYQGYTKAIDVWSAGCILAEFLGGKPIFKGKDYVNQLNQILQVLGTPPDETLRRIGSKNVQDYIHQLGFIPKVPFVNLYPNANSQALDLLEQMLAFDPQKRITVDEALEHPYLSIWHDPADEPVCSEKFEFSFESVNDMEDLKQMVIQEVQDFRLFVRQPLLEEQRQLQLQQQQQQQQQQQQQQQQPSDVDNGNAAASEENYPKQMATSNSVAPQQESFGIHSQNLPRHDADFPPRPQESMMEMRPATGNTADIPPQNDNGTLLDLEKELEFGLDRKYF

SEQ ID NO: 13 - DNA, 3302 bp, Saccharomyces cerevisiae
  CDS (1001)..(2299)
agtaatacttgcaaatattgcaaaacttggaagaatgttaatgaatcatttcttgcaccattctttcaatcatctcaatctcctgctgtgatgtttaagtataacattgaagactatgccctaatttccaatgttatttagttttaagcatatctttgtttctaacaggaaactcaggcccacatccgcaaaaaaatatgtgccaaaaaactttcaacacttcaaagatacttaccactgcaggaaaataatctacgtgtaacggtttgaaaataaatttgacttcataattggacataagtactccatcgccatccctttttaaagaagtttccacaagaatgaatggctaatcgcaactaaatcttttccttgcaaacgtaacacagtatcgacattttcttactcaatccaacgaaggaataacctatctaaaaaataaacgccgtagttttcagcccacaagacgtcattaaaagatttgttaattataaaaatagaaatatttctaccagcatgattattcgttacttgaaagtccccaataaatttcactgtttccgttaactgttgtagttattaaacgcagcaaacagattattttgaacaacaccggagaaacacgcgcagacccattcgagttaaaaatagtaactcgcgatcaatcaatgcaggaagcaccgtaggaattagtaagaactcgtattttgattgaaaatgccatgaaagcaattgacttgctgcagtaaaaagcgctgccacaaactttgtaattttcgacaatgacgttcttttcagatggttactgtctttttttggaagaaacaaaagaaggtacttttatgatgttatactaggcaaaaagcctatttaatgtaagtcctaattgtcgtttgagactggatgaaaagggacaaaatggaaggataactaaaggtgacttaccgccagattaattcggcctggaatagtttgatatcgaagaaagattcacaattaaatggcgactgacaccgagaggtgtattttccgtgcattcggccaagattttatcctaaataaacattttcatttgacaggtaagattggtcggggctcacacagccttatttgttcttcaacttacacagaatcgaacgaggaaactcacgtggctatcagaaaaataccaaacgcgtttggcaataaactatcttgcaagagaactcttcgtgaattgaaactactaagacatttaagagggcacccaaatatagtgtggctcttcgatactgatatagtattttacccaaatggggcactaaatggcgtttatttatatgaagaactaatggaatgtgacctttctcaaattataaggtccgaacaacgcctggaagacgcacactttcaaagcttcatatatcagatactgtgtgctctgaaatacatacattctgctaatgttttacattgtgacctgaaaccaaaaaacttacttgttaatagtgattgccaactaaaaatttgtaattttgggctatcgtgtagttattcagaaaaccacaaggttaacgacggcttcattaagggttatataacctcgatatggtataaagcaccagaaattttgctgaattatcaagaatgcacaaaagctgtcgatatttggtcaacaggctgtatcttggccgaactacttggtaggaaaccaatgtttgaagggaaggattatgtagatcatttgaatcatattctacaaatacttggaacaccacctgaggaaacattgcaggaaattgcctctcaaaaggtgtataattatatctttcagttcggtaatatcccgggaagatcgtttgaaagcatactacctggtgctaatccagaagcgcttgaattgctaaagaaaatgctagaatttgatcctaaaaaaaggattactgtagaggatgcactagagcatccatatttgtcaatgtggcatgatatagatgaggaattctcatgtcaaaagacctttagattcgaattcgagcatatcgaaagtatggcggaattaggaaacgaagttataaaggaagtatttgatttcaggaaagttgttagaaaacatcctattagcggtgattccccatcatcatcactatctttagaggatgccattcctcaagaagttgtacaggtccatccttctaggaaagttttacccagttatagtcctgaattttcctatgtaagccaacttccatcactaactacaacccagccatatcaaaaccttatgggaataagctctaattcatttcagggtgttaactaaaaggaaaacaccttcaaacaagatactaagcatgaaaatagtgaactactgaacggacctactgagccaaatataacaaaaatgagcccagtttcatcgtctcccccaggtcacgatataaatgtcaatgatggtacaaaccaaaatacaaatgaggatgacagcgattttttcttcgacctagaaaaagaacttgaattatttagacgataaatttttgtagcagaaaaccacaactaatagatgcgcacatacactatctataatgaatatgtaaaatgcctgttcaccttcttaattattggtatatacttcaaatattgcaaaaagagaaagtcctctcggcggttttgcagttccttccgaaagcgggaaaaaccaaaatgtgagaaagtaggatacaccattgcgtagattcgcgatgatccgaatataaacatgattccctcgtcagtcctctctcaagttttctttcccgttttaaatagcttactaatattttcacaaaaaagttgatatcatttaaaggtgcttttggcgggattgaatgatgaaaagattacaccccttgagaattcaagttcatctgaaatctgattacccactgtttactttcgagcaattactctctacaaatgggataagaagaggccaaactgcgagaatttctttgaaagattacatagagtggcaaaatttcccaaacataatgaaaagagaaaatttttttacgcaaaggaagcctgtaactacaaccgcaaaagaagaacccttttcatttgataacattcttgactgtgagccacaatttagcaaatgccttgccaaatggctactggttaattacaaattaaatgactatccttattacgatcttaacattgtgaatatttacacggatttaccccaagcaattcagatttgcaaaaatttaatgtcatatctcaagtctactttatctgataacatgttccagaaaataaaatatttcatggtacct

SEQ ID NO: 14 - protein, 433 aa, Saccharomyces cerevisiae
MATDTERCIFRAFGQDFILNKHFHLTGKIGRGSHSLICSSTYTESNEETHVAIRKIPNAFGNKLSCKRTLRELKLLRHLRGHPNIVWLFDTDIVFYPNGALNGVYLYEELMECDLSQIIRSEQRLEDAHFQSFIYQILCALKYIHSANVLHCDLKPKNLLVNSDCQLKICNFGLSCSYSENHKVNDGFIKGYITSIWYKAPEILLNYQECTKAVDIWSTGCILAELLGRKPMFEGKDYVDHLNHILQILGTPPEETLQEIASQKVYNYIFQFGNIPGRSFESILPGANPEALELLKKMLEFDPKKRITVEDALEHPYLSMWHDIDEEFSCQKTFRFEFEHIESMAELGNEVIKEVFDFRKVVRKHPISGDSPSSSLSLEDAIPQEVVQVHPSRKVLPSYSPEFSYVSQLPSLTTTQPYQNLMGISSNSFQGVN

SEQ ID NO: 15 - DNA, 2978 bp, Saccharomyces cerevisiae
  CDS (1001)..(1975)
tctggcttcgaggaattattacctaaataggaaaggcagaatatattagaaaaaaaagaaaaaccaaatgagaaaagtgctggtgctaaataaaacattattgaggggccaagaggggacaaaagaagatataactagatcattaagttttcgctctagtaacaggaacaaagattgtgagatacactgttatgctaagagacggtgcgatattctgtacgaaaattatttaactattaactaaatgtataccacttcacgtgccaccgagtaggtttctaaaatgtgcaaccattttaggtatgtgcgcagctctttattctaaacgggagtcactacattactattatcgtgtttttgcccatgtactttcttataatcttaagacaacaacgggatgataggcgcattcggactttcattgatgcaaatgtgtgaaaaatgcatccaaaagacaacttttgtacagaatacaattgcaaaaatactttacgggcatagatcggtaaggtcaccgggaagctagcgtaagagaccttattcggaaccgagcaaccatttccgaatgtagtagtagttgaaggagtaaatcgaccttattgtacactacttcctttaaatttgatttctggccccgcgcaatttcttggcggttaagctgtatttttacctcatcgggaaaagttattgcaagttaaaggggatcaaacgattagcaaactaattatagatcaaaggccgagggctttctaaatttggcatatttcgccgtcgactgaaatagaagggataaatcatgcatctccaggattatccctactccattcattacaacatgcgccaaatcaagcctatataagattctcgtcatttagcatgctctattgatttgtgtcttgttttgtctaacactgaaactgtaacctaagatttctttagataattattacatttacatcaataagaaatctcataaaacaagtactgtttataagtaaaaatgcaatataaaaagccattagtcgtctccgctttagctgctacatctttagctgcctatgctccaaaggacccgtggtccactttaactccatcagctacttacaagggtggtataacagattactcttcgagtttcggtattgctattgaagccgtggctaccagtgcttcctccgtcgcctcatctaaagcaaagagagccgcctctcagataggtgatggtcaagtacaggctgccactactactgctgctgtttctaagaaatccaccgctgctgctgtttctcaaataactgacggtcaagttcaagctgctaagtctactgccgctgctgtttcccaaataactgacggtcaagttcaagctgctaagtctactgccgctgccgtttctcaaataactgacggtcaagttcaagctgctaagtctactgccgctgccgtttctcaaataactgatggtcaagttcaagctgccaagtctactgctgccgctgcctctcagatttctgacggccaagttcaggccactacctctactaaggctgctgcatcccaaattacagatgggcagatacaagcatctaaaactaccagtggcgctagtcaagtaagtgatggccaagtccaggctactgctgaagtgaaagacgctaacgatccagtcgatgttgtttcctgtaataacaatagtaccttgtcaatgagtttaagcaagggtatcttaaccgataggaagggtagaattggctctatcgttgccaacagacagttccaattcgatggtcctccaccacaagctggtgctatctatgctgctggttggtccatcaccccagaaggtaacttagctcttggtgaccaggatactttttaccaatgtttgtctggtgacttctataacttgtatgataagcacattggttctcagtgccatgaagtttatttgcaagctatagatttaattgactgttgaacgatgcatcgatcaatcggagtcgtcctcctttaacttcacgaattagttgccactctcattccccacacataaacttgttttatggcatccttttcatttagcatgtctttatttccaaacctttcctcgttctttgcattcatttagcgtttgctcgagaaagcatcacgttttcacacattatcgttcgtcgctataataaaaatagttatagaatttactcagatttacatgtcgtacctttttaattgtaaaaaaaaaaattttatgatacataattacctaaatataattcagaatcaaacatacttatagctatttgtatgctattaggtggtcctgctataaaaatatcgtttataatactttatattttatctttcaacttagtcgcaattgcagaagctttccctgagaaaaaatttgtgaagctagctgcgatagcaaaggagcgcttaaggtatagaaaagcactcagctggaatgccaaaagatagtttagcaactgaccaaggaaaaagcttgtaggtagacttaacttcattgttctctaatcctttcgtcgtgtatattgtaaaaactgctgaacgagtattgataaaagatatcttggccactaaggggcagatccccttctggtgtgatagacaaccccaggagcatagataacaccaacttgtggtggagggtcatcgaattggaattgtctgttggcaacagtagaacagatgctgcccttgctatctgtcaaaatgccgctcttcaaagttactttcaaagcgctgtcactgctacgacagctttttttttagaaacagcagcaatgccttgacatgtaacgtaagaaaagaaaaaagagatggcagaagaaatactaagcgataacggcaatgtagaggtgctttttttatcggaataaatagagaagtcagtaacagtgattgctgtggctccctctttaatcgtatctatgtaggttccgattaaagtggtcgtg

SEQ ID NO: 16 - protein, 325 aa, Saccharomyces cerevisiae
MQYKKPLVVSALAATSLAAYAPKDPWSTLTPSATYKGGITDYSSSFGIAIEAVATSASSVASSKAKRAASQIGDGQVQAATTTAAVSKKSTAAAVSQITDGQVQAAKSTAAAVSQITDGQVQAAKSTAAAVSQITDGQVQAAKSTAAAVSQITDGQVQAAKSTAAAASQISDGQVQATTSTKAAASQITDGQIQASKTTSGASQVSDGQVQATAEVKDANDPVDVVSCNNNSTLSMSLSKGILTDRKGRIGSIVANRQFQFDGPPPQAGAIYAAGWSITPEGNLALGDQDTFYQCLSGDFYNLYDKHIGSQCHEVYLQAIDLIDC

SEQ ID NO: 17 - DNA, 4034 bp, Saccharomyces cerevisiae
  CDS (1001)..(3031)
ccaaccacgtaagggaaaaggacggtgtttgggccattatggcgtggttgaacatcttggccatttacaacaagcatcatccggagaacgaagcttctattaagacgatacagaatgaattctgggcaaagtacggccgtactttcttcactcgttatgattttgaaaaagttgaaacagaaaaagctaacaagattgtcgatcaattgagagcatatgttaccaaatcgggtgttgttaattccgccttcccagccgatgagtctcttaaggtcaccgattgtggtgatttttcatacacagatttggacggttctgtttctgaccatcaaggtttatatgtcaagctttccaatggtgcaagattcgttctaagattgtcaggtacaggttcttcaggtgctaccattagattgtacattgaaaaatactgcgatgataaatcacaataccaaaagacagctgaagaatacttgaagccaattattaactcggtcatcaagttcttgaactttaaacaagttttaggaactgaagaaccaacggttcgtacttaaaacgaatgatttactaatggcttaatgattttcacctttttcaatgaatattaacggtaaagaagaaaatttcaattttttgaacacatactttatatacttaatagatccatatttcgacatattagcaaacgattgcataggtttctgagtcttttttttttttttttcataaggaggagaatattttggttaatcgcagtatcttcttcataagtgctgtttctaattatatctaattcacgaatttttcccaaattagcgtatccccgaattcagattacctaccccgagttttttattatatttccctcgagaaatctgtaaaatggccgtcatccttagatttataaataaaatgataaaattcagccaaagtgctcctaaaccagaattgttcaactgggtcaaattatcgcgtatacaaatatacatatagtaacatgcattcctggcgaatatccaagtttaagttaggaaggtccaaagaagatgatgggagtagtgaagatgaaaatgaaaaatcgtggggtaatggcctgtttcatttccaccatggagaaaaacatcacgatggtagcccgaagaatcataatcatgaacacgaacaccatataagaaagatcaatacaaatgagactctcccaagttccttaagttctccaaaattacgtaatgatgcatccttcaagaatccatcggggataggaaatgacaattctaaggcttccgaaaggaaagctagtcagtcgtctactgagacgcagggaccgagttcggaatccggactaatgacagtgaaggtgtattctggtaaagattttactcttcccttccctatcacctctaactctactattttacaaaaactactaagttccggcatccttacttcatcatccaatgacgcttccgaagttgcagccataatgcggcagctaccacgatacaagagagtggatcaagattcagcaggggaaggcttgatagatagagcttttgccactaaattcattccttcctctatattgttacctgggtcaacaaattcaagcccattactttattttacaattgaatttgataattctattactactattagtccagatatgggaacgatggagcaaccagtgtttaacaaaatatcgacatttgatgtaacaagaaaattacgatttttaaaaatcgatgtctttgcaaggattccatccctacttttaccctctaaaaactggcaacaggagattggcgagcaggacgaagtactgaaggagattttaaaaaaaatcaatacaaatcaggatatccatttggactccttccatttacctttgaatttaaaaatcgattctgcagcccaaataagactatacaatcaccattggatttctttagaaaggggatatggtaaattaaatatcacggtggactacaaaccttctaagaacaagcctctctccattgatgactttgatctattgaaggttatcgggaagggttcgttcggcaaagtgatgcaagtaaggaaaaaagatacccaaaagatttacgctttgaaggctctgagaaaagcatatattgtatcgaaatgtgaagtgacacatactttagcggagaggactgtcctagcaagagttgactgcccctttattgttccgttgaagttctcattccaatctccggagaagttgtacctagtattagctttcattaatggcggtgaactgttctaccatttacaacacgagggacgattcagtctagcacgctcccgtttttatattgcagaactattatgtgctctcgattcattacacaaacttgacgtcatttatcgtgacctaaagcctgaaaacattctattggattaccaaggacatattgcactgtgtgattttgggctttgcaagctgaacatgaaggataatgacaaaacagacactttctgtggtactcccgaatatttggcaccagaaatcttgttggggcagggctatactaaaacagttgactggtggacattaggtatcttactgtatgagatgatgacagggctgccaccatactatgatgagaacgttcctgttatgtacaagaaaattctgcagcaaccgctactatttcctgatggatttgaccctgcggcaaaagacctattaattggcctcttaagcagagacccaagcagaagactcggcgttaacggtacagatgaaattcgtaaccatcctttctttaaagacatctcatggaaaaagctacttttgaagggctatattccgccttacaagccaattgtaaagagtgaaatagatactgcaaattttgatcaagagttcactaaggaaaaaccgatcgatagtgtagtggacgagtacttaagtgcaagtattcaaaagcagtttggtgggtggacgtacattggtgacgaacagttgggtgattctccttcgcaggggagaagcattagttagaagcaagccgaagcaagccgagccgagccggacggaatttatagctatagccgcaagaggttgcaattttcaaaaatggatagttcaagtagattgcgatacgcactccgttactattgtggttaacggggacaagaagaactacagaaaatagaatggtccgcagaggctgcgctcttcttttagcaactctcacacgacttatgttgcttattcatttcttttacagcattatcagaattcttccatctacggaattgagatcaaagaccgacctgttgtcggccgaaggacggacgcttatacccgcggatgtcaaagcgaagcccgcggggcgcaagtcgaggttaccggaattcgccaaacggcaaaggacccttgcattgcctgaaaggaaagattcgcttttctgtttgttgccacttttcttacatagtctgggccgggagcagcttatttcttccgcggatgatcctggatttccttgcgcgggctcagccatggggagccttacctagtcccgtaaagggaaaaagctaacctcattcgcctcacagggtgaaagcgtgaacaaaaaaaaaagaaaagcttaatgattaaaatttacagtatatatatatttgtatttacgtattaaactatatataaatagatatgtatgccgaaaaagtaaagtctgggtgatgcctagtccaatctttcttactactgtccagtttctatcgtagcagttaattatacatagaactgtgtaaattcaacgcattaatttttttttttttcactttcgcagttaggggggacacattttttttgccctttcttaagcttcgtaagcgagttacatcattatttcttcctgggatacaatacgcgttcgtacaagtcacagctggaccgtatagggaacaagactgcaactctctccaacttgttaaacagaggaggaaaagaaagagggaaaagaggaacaaagacaatcaaagaaaaagaatagaaa

SEQ ID NO: 18 - protein, 677 aa, Saccharomyces cerevisiae
MHSWRISKFKLGRSKEDDGSSEDENEKSWGNGLFHFHHGEKHHDGSPKNHNHEHEHHIRKINTNETLPSSLSSPKLRNDASFKNPSGIGNDNSKASERKASQSSTETQGPSSESGLMTVKVYSGKDFTLPFPITSNSTILQKLLSSGILTSSSNDASEVAAIMRQLPRYKRVDQDSAGEGLIDRAFATKFIPSSILLPGSTNSSPLLYFTIEFDNSITTISPDMGTMEQPVFNKISTFDVTRKLRFLKIDVFARIPSLLLPSKNWQQEIGEQDEVLKEILKKINTNQDIHLDSFHLPLNLKIDSAAQIRLYNHHWISLERGYGKLNITVDYKPSKNKPLSIDDFDLLKVIGKGSFGKVMQVRKKDTQKIYALKALRKAYIVSKCEVTHTLAERTVLARVDCPFIVPLKFSFQSPEKLYLVLAFINGGELFYHLQHEGRFSLARSRFYIAELLCALDSLHKLDVIYRDLKPENILLDYQGHIALCDFGLCKLNMKDNDKTDTFCGTPEYLAPEILLGQGYTKTVDWWTLGILLYEMMTGLPPYYDENVPVMYKKILQQPLLFPDGFDPAAKDLLIGLLSRDPSRRLGVNGTDEIRNHPFFKDISWKKLLLKGYIPPYKPIVKSEIDTANFDQEFTKEKPIDSVVDEYLSASIQKQFGGWTYIGDEQLGDSPSQGRSIS

SEQ ID NO: 19 - DNA, 2765 bp, Saccharomyces cerevisiae
  CDS (1001)..(1762)
ggatatgattgctgagaatgcgttaccggccaaaacaaagacagcgggattgagaaaattaaagaaggaagatattgaccaagtttttgagttgttcaaaagatatcaatccaggttcgaactaattcaaattttcacaaaagaagaattcgaacataatttcattggtgaagaatcgttaccattggataaacaagtaattttctcatatgtagtcgaacagcccgatggaaaaattacagacttcttctcattttactcattgccattcacaatcctaaataacacaaaatataaggacctaggcatcgggtacttgtattattatgccaccgatgcagatttccaattcaaagacaggtttgatccaaaagctactaaggctttgaaaacaagattgtgtgaattgatttatgacgcttgtattttggccaaaaacgctaatatggatgtttttaacgcgttgacttcgcaagataatacattgttcttggatgatttgaagttcgggcccggtgacgggttcttgaacttctatttatttaattatagagcaaagccgattaccggtggcttgaatcccgacaatagtaacgacattaaaaggcgtagcaatgtcggtgttgttatgttgtagtggctgaaaggacgaggcgtatatagttttcgtgtacatagccgacagaatttgaccacatttagtttttccgcatagtcaattgacgaagtgaaaaaataattaatccaatggctggctttagagtgtcagcctccaaaataaatccaaaaatagacaaagagaatcactataattaccgccttggagtccaagttggcttgagaactcgcatttatttttagcgactgaggtagctgaaaaacgcctactttctcagaaggcggtagtgagcatatataagtatgtaagaaagatcaactcttctggactagatactcaccgatctagtgaaaatataaacaaacccaacatatatataaaatgaaggcctgttccatattatttaccaccttaattactctagccgctgctcaaaaagactctggttccttagatggccagaactctgaagatagctcacaaaaggaaagctcaaactctcaagagatcacacctaccacgacaaaggaagcccaagaaagcgcatcaactgtagtttctaccggaaaaagcttagtacaaactagcaacgtcgtcagcaacacctatgctgtggctccaagtaccaccgtagtgacgacggatgcacaaggcaaaaccacgacacagtacctatggtgggtggccgaaagcaactctgccgtaagcacaacttcaactgcctctgtgcagcccaccggagagacgtcaagcggaatcaccaactccgcatcctcctcaacgacatcaacatcaacggacgggccagttactatagtaactaccacgaattcgttaggtgagacttacacatctactgtttggtggctaccgtcctcagccacaactgacaacacggcttcatcaagtaaatcatcttcgggatcctcatcaaaaccggaatcaagcaccaaggtagtaagcactatcaaatcaacttataccactacgtcaggttctacagtagagacactgaccactacatacaagtctacagtcaacggtaaggtagcgtccgtaatgtccaattctaccaatggcgcctttgccggcactcacatagcttatggtgcgggtgcattcgccgttggtgcccttttgttatagaatgtataatcagttctgtataccaccacatagttctgcattttaataaaactctttctttttatacactgtaggtaaccaataatataactattgttatcatcgtgcttgcgtattttttttctttcgggtgaaaaactccgcagtatttctcgctctccctggataataagctagaaaaaaaaaatatatatgacagatggatgagtaatcatattcaataagtattgtctggcttctgagacggcggtaagatatccttaagagttgcaatggtccttttacacaaaagcacacatatatttcctaccgattttgcctctgtttcacgcgccttttttaatagataccccaatccatactccccccatgtactatccatagacacaatatcaaggaacgttgatcaagaaggaaatttgcgcacaacgaggctgttgaaaaagtccggaaagctgcccacatgggtcaaaccctttttaagaggtataacagaaacatggataatcgaagtttccgtagtgaaccccgctaactccacaatgaaaacttacactaggaatctggatcacactggaatcatgaaggttgaagaatatactacctatcaatttgacagtgctacaagtagtacgatagcagacagccgggtgaagttttcaagtggcttcaatatgggtatcaaatctaaggtagaggattggtcgcgcactaaatttgacgaaaacgttaagaaaagcagaatgggcatggcatttgttatccaaaaactcgaagaggcgagaaatcctcagttttgatgttcccatttaaagatctttaaagatatcaccatgggcgagcgaaattgagaaaactagtgcagctcgcatttggtcacgtcctaaaaattgtaaataagcgctggttcaacaaaatttaatatacacacatatataattatttatttataacagtcattctgctaaactatacatcaaatgtcactaatcttgatatt

SEQ ID NO: 20 - protein, 254 aa, Saccharomyces cerevisiae
MKACSILFTTLITLAAAQKDSGSLDGQNSEDSSQKESSNSQEITPTTTKEAQESASTVVSTGKSLVQTSNVVSNTYAVAPSTTVVTTDAQGKTTTQYLWWVAESNSAVSTTSTASVQPTGETSSGITNSASSSTTSTSTDGPVTIVTTTNSLGETYTSTVWWLPSSATTDNTASSSKSSSGSSSKPESSTKVVSTIKSTYTTTSGSTVETLTTTYKSTVNGKVASVMSNSTNGAFAGTHIAYGAGAFAVGALLL

SEQ ID NO: 21 - DNA, 3335 bp, Saccharomyces cerevisiae
  CDS (1001)..(2332)
tcttgttctttacagattcaagaggaaaccaaaaaaaaatcaaagaaaaagaatcgaatttttcccaaaatgaaagtgtaaggaaaaaaaaagaggagatagaaaatccgaagaaccccaagggacggacaaacacaagacgatgctgcacgtggttagttttgtaagcgcaggttacgataaagagcataaacaaatcattactaagagcggtatacaagaataaagtgacaaacagttctccctatttaacgcacttaacgtaggttccatcattatgatgctattgccacatcaaatctcctttggactgaacccgcattagtaattgcccgcttttcttttcttccgcgggtgggccccataaatagaaaaaaaaagaaagaaagcgtttaaataaatagagtgagcggatttctattatctgaaaaccgggttataatgcacgtgatatgcacgtgggagctgggcggctatttttttctttttcaaatgtatttgagtcgtttaaaatagcactccccgttgacccgccaactcatttttgttttctctttacggaaaaggctttaaattaaggcccgcattttcggtatccttgagggaaaaaaaccaaagaaacccaaaaaagaccacaaagctgggatatcttaattagtagagagggcttttagttttaatagtgttacgagtctctaaaaatagcgtaggcacactgccctgattcggactttgatcagagtttattactacaaagagtaatgttgaatgattgggctgggttttcatagcattaactctaagtaatatcattcaaccgctcaaggttccttacgagcaaacccatatatgctctacagataaacatataaatagcgtgcatattcttctctattcaactcttgctctgtatagttcaatagaatcttacagtacatcacgctgcaatagatctaatccaagagagaagcaaaaaaaaaaagctcgctataaaaatatcatgcaattacattcacttatcgcttcaactgcgctcttaataacgtcagctttggctgctacttcctcttcttccagcataccctcttcctgtaccataagctcacatgccacggccacagctcagagtgacttagataaatatagccgctgtgatacgttagtcgggaacttaactattggtggtggtttgaagactggtgctttggctaatgttaaagaaatcaacgggtctctaactatatttaacgctacaaatctaacctcattcgctgctgattccttggagtccatcacagattctttgaacctacagagtttgacaatcttgacttctgcttcatttgggtctttacagagcgttgatagtataaaactgattactctacccgccatctccagttttacttcaaatatcaaatctgctaacaacatttatatttccgacacttcgttacaatctgtcgatggattctcagccttgaaaaaagttaacgtgttcaacgtcaataacaataagaaattaacctcgatcaaatctccagttgaaacagtcagcgattctttacaattttcgttcaacggtaaccagactaaaatcaccttcgatgacttggtttgggcaaacaatatcagtttgaccgatgtccactctgtttccttcgctaacttgcaaaagattaactcttcattgggtttcatcaacaactccatctcaagtttgaatttcactaagctaaacaccattggccaaaccttcagtatcgtttccaatgactacttgaagaacttgtcgttctctaatttgtcaaccataggtggtgctcttgtcgttgctaacaacactggtttacaaaaaattggtggtctcgacaacctaacaaccattggcggtactttggaagttgttggtaacttcacctccttgaacctagactctttgaagtctgtcaagggtggcgcagatgtcgaatcaaagtcaagcaatttctcctgtaatgctttgaaagctttgcaaaagaaagggggtatcaagggtgaatcttttgtctgcaaaaatggtgcatcatccacatctgttaaactatcgtccacttccaaatctcaatcaagccaaactactgccaaggtttccaagtcatcttctaaggccgaggaaaagaagttcacttctggcgatatcaaggctgctgcttctgcctctagtgtttctagttctggcgcttccagctctagctctaagagttccaaaggcaatgccgctatcatggcaccaattggccaaacaacccctttggtcggtcttttgacggcaatcatcatgtctataatgtaatggaatgaagaaatattcttcatttttgataactagtacctgtcattcacgacatgtgaacaaataaaaacatttatttaaaaattttatgtattcaaatattttcgggaaagagataaaagtaacgacacttaaaaatttaaaaaatcacaatactttatttactcagtcttttgatcagctccggcacctccttgttgttgcttctttgctgagcccgcaacaaaattgtaaatcaataggcctaaaagtaacattttccagttcttttgaaaccaagacacctccttaacctcttcatcttcttcgaattgtgcagtgcttccatccttattcttactagcttttttatcagcataagttttggttttcttctttaatttggtgactggtgcagtaggtcccgcttctggatatctgactgtagcagtaatagcatcgttagtctcgtcatatgataacgacacttgtttgacctcattatcttcatctacatccacaattaaatcgtattttagtggtgtcctcagcttcatgtagctaaaacatggcatatccagcttaccttcaatctgggcattcaaacagtattctccagaaacttcaacatcctgtatattaacggttgttactgtaacattcccatcggatgtactatcaatctcaaatgttcctaggggtatagcgtctttcgcatcatctgaatagcttaattgtaaaatatcagcacagaaaaccatgctggccaataaaatcacacgcaacagccgcacaagcatctttccttcaatgagtattgtacagttcttgttagatagtgttgaatagtaccaccttgtttttttactcaaagtgtcttttatatacttctaattattcctatatttggttgggtttttaagttaccaatgcaaatacagtggttagagacccagcgcacgtatgcaaaaaaatacagcggaaatttcaagtaaaaatgtagcttcataaaaaagaagca

SEQ ID NO: 22 - protein, 444 aa, Saccharomyces cerevisiae
MQLHSLIASTALLITSALAATSSSSSIPSSCTISSHATATAQSDLDKYSRCDTLVGNLTIGGGLKTGALANVKEINGSLTIFNATNLTSFAADSLESITDSLNLQSLTILTSASFGSLQSVDSIKLITLPAISSFTSNIKSANNIYISDTSLQSVDGFSALKKVNVFNVNNNKKLTSIKSPVETVSDSLQFSFNGNQTKITFDDLVWANNISLTDVHSVSFANLQKINSSLGFINNSISSLNFTKLNTIGQTFSIVSNDYLKNLSFSNLSTIGGALVVANNTGLQKIGGLDNLTTIGGTLEVVGNFTSLNLDSLKSVKGGADVESKSSNFSCNALKALQKKGGIKGESFVCKNGASSTSVKLSSTSKSQSSQTTAKVSKSSSKAEEKKFTSGDIKAAASASSVSSSGASSSSSKSSKGNAAIMAPIGQTTPLVGLLTAIIMSIM

SEQ ID NO: 23 - DNA, 3107 bp, Saccharomyces cerevisiae
  CDS (1001)..(2104)
ttgggattccattttttataaggcgataatattaggtatgtagatatactagaagttctcctcgaggatttaggaatccataaaagggaatctgcaattctacacaattctataaatattattatcatcattttatatgttaatattcattgatcctattacattatcaatccttgcgtttcagcttccactaatttagatgactatttttcatcatttgcgtcatcttctaacaccgtatatgataatatactagtaacgtaaatactagttagtagatgatagttgatttttattccaacagtatttatgttttgtcattcttttctacataatcttgaaactaggtagatctacaattgaaaagtaaatactaacattatttactaaatttaagttagaaatcggcacgaaaaaaatttgacagattacgagagtccagccaaaatatgagtatattactatttccccttggtgaaagaaatgaaagatgttattttttaccggcttagtaatactgagctacttacttgggggaaagaaagattggctacttattatgtatgaagcctcagattaccttgaattcctcaaccgtttgagcagtatgctcttcaaattcgaactttttgaacatctttcctccacattcctgattttttcacattcaaaacgcgctgtgaagctgttagaaatttacagatcgaggcatatttctatatataatgtatttttattaagacacccaaagtacttccaatctgtagatattgcactttatctgaccagaagccagacttgaacagttacatattgtgctttgcagtcgttaaatttcccgaactgttttcgtatttttttttcttttcctcttttccactggatcagatcaaaagccgactaaaaatttggcaatttaaagaaagcatcttttaaagatagaaaaggttatttcaacaaaaaagtatcttttcttcacttttctttcaacaattcaaagatggctagaaccataacttttgatatcccttcccaatataaactcgtagatttaataggtgagggagcgtacggaacagtatgttcagcaattcataagccttccggcataaaggtagctatcaagaaaatacaaccgtttagcaaaaaattgtttgttacaagaactatacgtgagatcaagcttttacggtatttccatgaacacgaaaacataataagtatattggataaagtaaggccagtatccatagacaaactaaacgctgtttatttagtcgaagagttgatggaaaccgatttacaaaaagtaattaataatcagaatagcgggttttccactttaagtgatgaccatgttcaatactttacataccaaatcctcagagccttaaagtctattcacagtgcacaagttatccatagagacataaagccatcaaacctgttactaaattccaattgtgatctcaaagtctgcgattttggactagcgaggtgtttagctagcagtagcgattcaagagaaacattggtaggattcatgacggagtacgtcgcaacgcgatggtacagggcacccgagataatgctaacttttcaagagtacacaactgcgatggatatatggtcatgcggatgcattttggctgaaatggtctccgggaagcctttgttcccaggcagagactatcatcatcaattatggctaattctagaagtcttgggaactccatctttcgaagactttaatcagatcaaatccaagagggctaaagagtatatagcaaacttacctatgaggccacccttgccatgggagaccgtctggtcaaagaccgatctgaatccagatatgatagatttactagacaaaatgcttcaattcaatcctgacaaaagaataagcgcagcagaagctttaagacacccttacctggcaatgtaccatgacccaagtgatgagccggaatatcctccacttaatttggatgatgaattttggaaactggataacaagataatgcgtccggaagaggaggaagaagtgcccatagaaatgctcaaagacatgctttacgatgaactaatgaagaccatggaatagtattcacaagaacatttctgccatacttctaaaatttccctatattcagcttagcagtgacacgttgtggtctgtaggtcaatatgtaagtaagaaacttcaactcacatatgcacgatgcatgccaatggaaaaatgcaaggaacgaaatggcgccacggcaacaagtttttttttttcgccagcagaagtacacgaaatgcggcttcatgagcctcttcactgctttgcctaaacgggaaatgcagagaaaaaccagccatcgcgtgtgcttggagagctgacgcgactgtaatcaaagaggcgatatcaacaccttttatccagcactattcaacagtgaatgggctcccaagtaagtcttggcattgtgctttctattcttaagtattaagtagaagttttgtttactgggtttgtttattcctggctagatgttcgcattcgttttctagttgaccatatttaccaaatattcacaactaatacccagccaaggtagtctaaaagctaatttctctaaaagggagaaagttggtgattttttatctcgcattattatatatgcaagaatagttaaggtatagttataaagttttatcttaattgccacatacgtacattgacacgtagaaggactccattatttttttcattctagcatactattattccttgtaacgtcccagagtattccatttaattgtcctccatttcttaacggtgacgaaggatcaccatacaacaactactaaagattatagtacactctcaccttgcaactatttatctgacatttgccttacttttatctccagcttcccctcgattttatttttcaatttgatttctaaagctttttgcttaggcataccaaaccatccactcatttaacaccttattttttttttcgaagacagcatccaactttatacgttcactacctttttttttacaacaatttcattcttcatcctatgaaatgacgaaaataaccaga

SEQ ID NO: 24 - protein, 368 aa, Saccharomyces cerevisiae
MARTITFDIPSQYKLVDLIGEGAYGTVCSAIHKPSGIKVAIKKIQPFSKKLFVTRTIREIKLLRYFHEHENIISILDKVRPVSIDKLNAVYLVEELMETDLQKVINNQNSGFSTLSDDHVQYFTYQILRALKSIHSAQVIHRDIKPSNLLLNSNCDLKVCDFGLARCLASSSDSRETLVGFMTEYVATRWYRAPEIMLTFQEYTTAMDIWSCGCILAEMVSGKPLFPGRDYHHQLWLILEVLGTPSFEDFNQIKSKRAKEYIANLPMRPPLPWETVWSKTDLNPDMIDLLDKMLQFNPDKRISAAEALRHPYLAMYHDPSDEPEYPPLNLDDEFWKLDNKIMRPEEEEEVPIEMLKDMLYDELMKTME

SEQ ID NO: 25 - DNA, 3086 bp, Saccharomyces cerevisiae
  CDS (1001)..(2083)
aatactgaatagaatcacgctactacgacaagactcggttactgtgcctaaaataatcctgtgataaacgagttatgttaaacgcagtacaggggttaaagggcattgagtttttgtgagtggaaatgcccccgttatagcttccagtttaattacaaattatcaatttaagcaaatataactggaggattggggaggcgactaaaaatggctaccacgctattagacatacaacattgagtattttatgtaattttgttactgctagcacggccatgcaattggcaactgaaagctatctgacaacttaaatgattcttaaaacaatgacgactataatcttctctaagaagtttcatatccatcttcctcattattcagtttctttttcctcttgaaagtatcgtaaagaacaacgtcttcacattagctattagaagaccattgaactaccggatatgagtaagagtgatcttgccggagagataatagctgcacaaaggccaaggattagattaatgggtgcattgtacgaaaaaaaatagtttacagtcatttattcgcaataaatcaatttttttttcaaaaaatatgtaagtctgataaaaaattcttcactgaagagagatgcttacattctaattcttgaataaaagactctctaacgctgtgaattctctttagctgtaacggaaacagagagttattccgtagtcactgaattttttttttttgacgctattatttaaaacctaggatatccgtcccatacaaaacggccacgagtttcaatcccagaatgtacgagttataattctcctagatgcatgatactcgtgcattcgtttaacaatcataccaatttcccattttcgggatattaaacatgaacatacttttttactgtgagaatgtggtttcacaattattccatacaggtataaaaacgcacagaacttcaaacgggaagactatctacccacattgatggacaaacgcaatgatttctgctaattcattacttatttccactttgtgcgcttttgcgatcgcaacacctttgtcaaaaagagattcctgtaccctaacaggatcttctttgtcttcactctcaaccgtgaaaaaatgtagcagcatcgttattaaagacttaactgtcccagctggacagactttagatttaactgggttaagcagtggtactactgttacgtttgaaggcacaaccacatttcagtacaaggaatggagcggccctttaatttcaatctcagggtctaaaatcagcgttgttggtgcttcgggacataccattgatggtcaaggagcaaaatggtgggatggcttaggtgatagcggtaaagtcaaaccgaagtttgtaaagttggcgttgacgggaacatctaaggtcaccggattgaatattaaaaatgctccacaccaagtcttcagcatcaataaatgttcagatttaaccatcagcgacataacaattgatatcagagacggtgattcggctggtggtcataatacggatgggtttgatgttggtagttctagtaacgtcttaattcaaggatgtactgtttataatcaggatgactgtattgctgtgaattccggttcaactattaaatttatgaacaactactgctacaatggccatggtatttctgtaggttctgttggtggccgttctgataatacagtcaatggtttctgggctgaaaataaccatgttatcaactctgacaacgggttgagaataaaaaccgtagaaggtgcgacaggcacagtcactaatgtcaactttatcagtaataaaattagcggcataaaaagttatggtattgttatcgaaggcgattatttgaatagtaagactactggaactgctacaggtggcgttcccatttcgaatttagtaatgaaggatatcaccgggagcgtgaactccacagcgaagagggttaaaattttggtgaaaaacgctactaactggcaatggtctggggtgtcaattaccggtggttcttcctattctggatgttctggaatcccatctggatctggtgcaagctgttaatcctcttttaaagtactcatatgactatacataccttcttttcttttctttactattcaatacataacagaacaaagatgcaggaaaatattggtatttgttcggcaatttatgctgggtttttttgtaaattcaggtctaattattactgttgatttgtatcaagttggtatcttttttgccatttaataatagagatacgctatgctcatccggatagcaacaatgagagcctaaaagtcctaattgagaagaaaatctctgttcaagactatagtttatgtttcattctggacccttgggatcgtctgaaacaggaaggtcaataattggtaaaaaaaatggtaaatgcgactaagtactacaattgaaacgaatgagcgcacttcatcttcctacaaaacgctgcggctgaaaaagttacataaaaaaccgtcctcaatagcgttaatccagcgtacatgagaaagtaatgacaaagtcttcggtaatatcagtgcatctaccaatatgacacaattgtgaaacttcgctgactcaaataatagccctgtttttttgaccattgttacccatcgagccagtgagaaaaaagccaaaatatctttaaggccttctccattttatgtttatcgatattgtgttgtctgcaatattgaaattttaaaggctatttactttgcctcttgttataaactaagtctgccgaattatgcaatatatagcaaaagctgaaaatagatgtaattacataattcgcagttgtatatgagtatccttaactcgtacattccagttcatctgtgacaaggcactgttttccctaataattattagggaaacgtccttcaaaaatcaaaataattttagagagtctcatcaaccttcgccatagttcgtgatgaaaactttacggtacgtcagactttagatattgatttttttattatttctcccatcgtgagtacaattaccctagttcgaactatatctttcatta

SEQ ID NO: 26 - protein, 361 aa, Saccharomyces cerevisiae
MISANSLLISTLCAFAIATPLSKRDSCTLTGSSLSSLSTVKKCSSIVIKDLTVPAGQTLDLTGLSSGTTVTFEGTTTFQYKEWSGPLISISGSKISVVGASGHTIDGQGAKWWDGLGDSGKVKPKFVKLALTGTSKVTGLNIKNAPHQVFSINKCSDLTISDITIDIRDGDSAGGHNTDGFDVGSSSNVLIQGCTVYNQDDCIAVNSGSTIKFMNNYCYNGHGISVGSVGGRSDNTVNGFWAENNHVINSDNGLRIKTVEGATGTVTNVNFISNKISGIKSYGIVIEGDYLNSKTTGTATGGVPISNLVMKDITGSVNSTAKRVKILVKNATNWQWSGVSITGGSSYSGCSGIPSGSGASC

SEQ ID NO: 27 - DNA, 2486 bp, Saccharomyces cerevisiae
  CDS (1001)..(1483)
ttctcgagcattagatgattaaatcaaaatgacatagtatttcgcaacctttcagttgggctttgtttaagaagtggaatacttttgcttgagttgtttagttttattttatccactgttgtcttaacaaatattttcaagaccggtaagccgaagatgaaaaatcattattaactcattttttgaacaaaaatataaacaaaagaaaggcaacgcacaattttagagatacataaaacgcagtggatgttaaaaataacagcggtacagaaacgcctgtctcgctccaataataattatacaaatttgaaaccgaacgcaatgtgccaagaaatgtaaacacactatagaaaaaaatagaacggtgcacattgtgctagcatatctgcttggttctgaacaagaagcacctggccactttctcctagcccaattcttgccaagttttcaacctcaatcttttgtgtttgaacaagcatgtatgaggggtcaaaatttagtggaggccgcttacaatccttctatttcctctggacctcattagccgtctggccagacctaagcgtcataatctggagaatttcattgcatgcgagaatatgataagtaagaacttgtttatttatacaagttccacccactcatacacggctacaattatgacgtataataacgtttcgtctagcccaccttttttacttttgacgttttatttctttcgaggatttggccaagaatgccccgaacagcggaaaaaatggcgtcgcagtttcagatgtatagactcatcttgtagaaaaaagaatgcaagaatgaagtcttttcgtggtgttttgaaaacactataaacaaaccgtcaacaaacattttgtataaatatttagctatatattgaatatcttgaccagtaaagcaccttgagaaattgtaagcttgaagaacgtactttgatatccctccgtttcatcatcctatagctcgtcaacaaatcaaaaaaaatatgaagatcagtcaatttggctctttagctttcgccccaattgtgctactacaactgttcattgttcaagcgcaacttctcacagattcaaatgctcaggatttgaatactgcccttggacagaaagtgcaatacacctttcttgacactggaaattctaacgatcaactacttcatcttccaagcaccacctcttccagcattattactggttcattagctgctgctaatttcaccggttcttcatcatcgtcgtctataccaaaagtcacttccagcgtcataacatctataaattaccaatcctcaaattctacggtagtcacccagttcacgccattgccttcttcgtcgagaaatgaaacaaaaagctctcaaacaactaatactataagttcaagtacaagcacaggaggtgtaggttcagtcaagccatgtctttacttcgttttaatgttagaaacaatcgcttatttgttttcttaaacaaatatattaggttcaaggtcttcgcaggtgtaagaaaacccgtggtctccatattcttaagtatgataaataaaaaaaaacttaataaattattaattgcttcaaacctttttctttttttagtttttaatatttcaaacgttatcttcattgaacgcccaaatagggaaaaatcctggcaaattttttattgctgtcatccaaggctatgctagaaaattcaagagcttggatgatttaaaaagacactctcaatcgagaaagtttattctttgttattctgctttacctgatcatattccggcgtattgtttctaatcaagtgatttcgatatccagttacgaaccatttacaacattcctgaaaatattgcgtatcaatgatatttgctccttctttctccctcattaaaaatattctcctggtaagctttctaatcagccacagttttgctgccaaaactttaacgtctagttccaatgacgatacacttgccaggtccgcagctgcagatgcagacatggcattcttcatggagtttttaaacgatttcgacaccgcttttccacagtatacctcatacatgatgcaaaaccatttaaccctacctcaacctgttgctgactactactatcacatggttgatttggcctcaacagcagatttacaatctgatattgctcagagttttccgttcactcaattccaaacattcattacggcctttccatggtatacctctttgctaaacaaagcctccgccaccaccatataccttccccaacacttcataacaggtgagacagaagctaccatgactaactcatcttatgccagccaaaaaaactccgtttccaattctgttcctttctcgacagcgaacgcaggccagtccatgatttccatggctaatgaagaaaacagtacaacagcacttatatccgcatcaaactcttcttcaacatccagaactagtcaatcacagaatggtgccca

SEQ ID NO: 28 - protein, 161 aa, Saccharomyces cerevisiae
MKISQFGSLAFAPIVLLQLFIVQAQLLTDSNAQDLNTALGQKVQYTFLDTGNSNDQLLHLPSTTSSSIITGSLAAANFTGSSSSSSIPKVTSSVITSINYQSSNSTVVTQFTPLPSSSRNETKSSQTTNTISSSTSTGGVGSVKPCLYFVLMLETIAYLFS

SEQ ID NO: 29 - DNA, 2783 bp, Saccharomyces cerevisiae
  CDS (1001)..(1780)
attctttggttgtgcttatacataattgaaaaagtgctaaaatccttacacttccaaaacattgaaagtggtaattattttccatctaaaaccgttgggagccaccccagaaaacccttattctctgccttcgtgaaacagctgcttatattcattgttgggctgggcgtgatgaagttctgcgtgtttctaatactaaactacttagaagacttggcatactggttcgccgatcttatccttggctggtcagattcatggccaaactttcaagtttttctggtcatgtttgtctttcctatcttactgaattgcttccagtacttttgtgtcgacaatgtcatcaggttacattctgagagcctaacgataaccaatgcagagaattttgaaacgaacacattcctaaatgacgaaattcctgatttatcggaagtctcaaatgaagtgcctaacaaggataacaacatttccagctatggtagcataatatagtattccaaggataaggaaagcatgcactgtttatttcctttccttgcttaattgattttttttaaagggaacaaacattttgatttcaatttccacaagcctagactcttcaacacataatctgtgggttattgtttgggaaagcattctccgctagaagaatgaaactggcgctcaggtttgattctataactacggcagtttttcctattctattttcgttttttgattttcccgccgcattggatattcaattcgcgacgctaataattggcatttcgtgttcttaagtaatttcgtgtttcaaataaccgtaaacagagaaagacccaagaatttcagatggcttagaagaggtagacattaaatcaatctgtatgtgatggagagggagtgtatttaaaagacgtaagaaaatgaattatcaagatccgttatggccatctagtctctttcttgtacactagttgtctaacacaaccaacaaattagaatatatatcgcaatgattttcaaaatattgtgtagtttactactggtaacctccaacttcgcttctgccttatatgtcaatgaaactacgagctatacaccatacacgaagacattaactccaacatactctgtttcacctcaagagacaacattaacgtacagcgatgaaacaaccaccttctacataacatctactttttactctacctactggttcactacctcccaatcagctgctattattagtacacctactgcaagtacacctactgcaagcacgcctagcctaactacgtccacaaatgaatacaccaccacctattctgacacagacaccacctacacgtctactctgacctctacttacataataactctatctacggaatccgccaacgagaaggctgaacagatttccacgagcgtcacagaaattgcttctacagtaaccgaatcgggcagtacatacacctctactttgacctcaaccttattggttactgtatataattcccaagctagtaatacaatagcgacatccacagctggggacgccgcctccaatgttgatgccttagaaaagttagtctctgctgaacatcaatctcagatgattcaaaccacatccgccgatgaacagtactgtagtgcgtctaccaagtatgttacagttacagctgctgcagttaccgaagtggttactactacggcggagcctgttgttaaatacgttactataactgccgatgctagtaatgttacaggttctgctaacaacggtacccacatttaatgcgtgacgttgaatcgagaaaaaaagctacttttaacgaaacctttactagttatcctatatgggatcactagtattttttgatttacgattcaataaatagactagagacaactttcatatcattccttaaaaaatacataaagcgcaaattcaaccccattgatacatatataagtagttctattatgactttcaagaacaatagtagcttttctaaataatcaataagtagcacaaaatctgtctgtttgtacgcttatatttagtttgcgtttatttgcgagcgccacgagaaggggcaggaaaaaaagatcaatagtttgcaataaacatcgaatgatgatttcaaccaccgatacataaaccagcgaggctttcaaggaagaatgaacgtgaactcgtcaactcaaaaagaaaatgagccagcatattaggaaattagattctgatgtttctgaaagacttaaatctcaggcatgcacggtatcgctagcatcagcggttagagaaatagttcaaaattctgtagatgcacacgctaccactatcgacgtcatgatcgacctccctaatttgagctttgcagtttacgatgatggtattggtttgactcgaagtgacctaaatatattggccacacaaaattatacttccaaaatacgaaagatgaatgatttagtaacgatgaaaacctacggttacagaggagacgccctatatagcatttctaatgtctctaatctgtttgtttgttccaagaaaaaggattacaactctgcatggatgagaaaatttccatccaaaagcgtcatgttgagtgagaataccatactcccaatagatcctttttggaaaatttgtccttggagccgaacaaagtctggtactgttgttattgttgaagatatgctgtataatttacctgtccggcgcagaatactaaaggaagaaccccctttcaagacttttaacacaataaaggcagatatgctacaga

SEQ ID NO: 30 - protein, 260 aa, Saccharomyces cerevisiae
MIFKILCSLLLVTSNFASALYVNETTSYTPYTKTLTPTYSVSPQETTLTYSDETTTFYITSTFYSTYWFTTSQSAAIISTPTASTPTASTPSLTTSTNEYTTTYSDTDTTYTSTLTSTYIITLSTESANEKAEQISTSVTEIASTVTESGSTYTSTLTSTLLVTVYNSQASNTIATSTAGDAASNVDALEKLVSAEHQSQMIQTTSADEQYCSASTKYVTVTAAAVTEVVTTTAEPVVKYVTITADASNVTGSANNGTHI